SEQUENCE LISTING (175 sequences)

SEQ ID NO: 1 - DNA, 1662 bp, Methylococcus capsulatus
  source 1..1662: mol_type = other DNA
atgcgtgaaacgatacctccccgcaccggcgccgacctgctggtcgactccctccaggcgctgggcgtcgaatacgtcttcggcgtgcccggcggcgcgatactcccgatcctgaacgtgctggccgaccgcggcccgcgcttcatcgtttgccgggacgaaaccggcgccgccttcatggcccagtcctggggccggatcaccggccggcccggcgtggtgctcaccacctccggccccggcctcatcaacgccgtctgtggcgtcgctaccgccacagaggaccgcgacccgctggtcgtcatcaccggccaggtgccgcgggccgtgcaattcaagcagagccacatgaacctggattcggtcggcctgttcgcgccgatcaccaaatggagcgtcgaggtcgaggaaccgaatactgtatcggaaatcctggtcaacgccttccgcaccgcgcagacgccgtgcgccggagccgtccacgtctcggtaccgaacgacatgctcaccgcgccggtcaccgcgcaggccctggcgccggccgaacccgccgtctggggaacggccccggccgccgtcgtcgaacgcgcggcgtccctgctgaacgatgccaaagccccggccatcctgctcggattgcgggccagcacacctggagcggcggcggcggtccggcgtttcctggagcggcatccgctgccggtggcgatgaccttcgaagccgccggcaccctgtcccgcgatctggtcgatcagttcgtcggccgggtcggctacgtgctcaaccagccgggcgacgaggtgctgcgccaagccgatctggtactcacgatcggctacgacccgatcgaatacgaaccttccgcctggatctcaccgcagtcgcaggcgatccacctggatgccctgcccgccgccgtcgaccgggcctaccaccctgccgccgaactggtcggcgacatcgccgccaacctggccgcgctcggcagcctgctccgaatcgaggatcgagccggacgccccgccgtcgccgcggcgcggcggcgtctgctggaggagcaagcccgcggcgcagcactgaccggtatgccgatccaccccttgcgcttcattcacgaccttcgggccacgctggacgacgaggcgacggtgacctgcgacgtcggcgcccacgagatctggatggcccgctacttcttctgctacgccccgcgtcacctgctgttcagcatgggccaccagaccatgggcgtcgccctgccctgggccatcggcgcggccctggcccggcccggcaagaaagtggtttcggtatccggcgacggctccttcctcatgacctgcatggaactggaaaccgcggtgcgcctcaaactgccgatcgtgcacatcgtctggaaagacggcggctacaacctgatccacagcctgcagatgcgcgactatgggcgcagcttcggcgccgagttcggccccaccgacttcgtcaaactggcggaggccttcggcgcgatcgggtaccggatcgagtccgcggacgggatcgtccctgtgctgaaccgggcgctcgcggccgacgcgccggtgctgatcgaagtgcccatcgactacagcgacaacgtccacctggtcgaggcgatcgacgcctcggcgcagcactga

SEQ ID NO: 2 - protein, 553 aa, Methylococcus capsulatus
MRETIPPRTGADLLVDSLQALGVEYVFGVPGGAILPILNVLADRGPRFIVCRDETGAAFMAQSWGRITGRPGVVLTTSGPGLINAVCGVATATEDRDPLVVITGQVPRAVQFKQSHMNLDSVGLFAPITKWSVEVEEPNTVSEILVNAFRTAQTPCAGAVHVSVPNDMLTAPVTAQALAPAEPAVWGTAPAAVVERAASLLNDAKAPAILLGLRASTPGAAAAVRRFLERHPLPVAMTFEAAGTLSRDLVDQFVGRVGYVLNQPGDEVLRQADLVLTIGYDPIEYEPSAWISPQSQAIHLDALPAAVDRAYHPAAELVGDIAANLAALGSLLRIEDRAGRPAVAAARRRLLEEQARGAALTGMPIHPLRFIHDLRATLDDEATVTCDVGAHEIWMARYFFCYAPRHLLFSMGHQTMGVALPWAIGAALARPGKKVVSVSGDGSFLMTCMELETAVRLKLPIVHIVWKDGGYNLIHSLQMRDYGRSFGAEFGPTDFVKLAEAFGAIGYRIESADGIVPVLNRALAADAPVLIEVPIDYSDNVHLVEAIDASAQH

SEQ ID NO: 3 - DNA, 1017 bp, Methylococcus capsulatus
  source 1..1017: mol_type = other DNA
atgcagatttactacgacaaagacgccgacctttccatcatccagggaaagaaggttgccatcatcggctacggctcgcagggccacgcccacgccaacaacctcaaggattccggagtgcaggtcgtggtggggctgcgtccgggttcggcttccgccaagaaggccgagaacgccggcctcgcggtcgcctcggtcgaggatgcggtcaaacaggcggacgtcatcatgatcctggcgccggacgagcatcaggcccgcctctacaatgaacagatcgcgccgaacatcaagcagggcgccgccctcgccttcgcccacggcttcaacatccacttcgagcagatcaccccgcgcgccgacctcgacgtgatcatgatcgcgcccaagggtcccggccatctggtacgttccacctacacccagggcggcggcgtgccctcgctgatcgccgtgtaccagaatgccagcgggcgcgccaaggaactcgcgctgtcctatgcttcggccaatggcggcggtcgggctggtatcatcgagaccaccttccgcgaagagaccgaaaccgatctgttcggcgaacaggccgtcctgtgtggcggcgccaccgcactggtgcaggcgggtttcgagacgctggtcgaagccggttatgcgcccgagatggcctatttcgagtgtctgcacgaactcaagctgatcgtcgacctgatgtacgaaggcggcatcgccaacatgcgttattcgatctccaatacggcagagtacggcgacctgacccgtggtccgcgcatcgtcaccgagcagaccaagcaggaaatgaagaaaatcctgcgcgagatccagaccggcgaattcgcccgtgagttcattttggaaaaccaggccggagccgccaccctgaaagcgaaacgccgtctcggccgagagcatctcatcgagagcgtgggcgccaggctgcgcgacatgatgccgtggatcaaggccaaccgcattgtggacacgagcaagaactga

SEQ ID NO: 4 - protein, 338 aa, Methylococcus capsulatus
MQIYYDKDADLSIIQGKKVAIIGYGSQGHAHANNLKDSGVQVVVGLRPGSASAKKAENAGLAVASVEDAVKQADVIMILAPDEHQARLYNEQIAPNIKQGAALAFAHGFNIHFEQITPRADLDVIMIAPKGPGHLVRSTYTQGGGVPSLIAVYQNASGRAKELALSYASANGGGRAGIIETTFREETETDLFGEQAVLCGGATALVQAGFETLVEAGYAPEMAYFECLHELKLIVDLMYEGGIANMRYSISNTAEYGDLTRGPRIVTEQTKQEMKKILREIQTGEFAREFILENQAGAATLKAKRRLGREHLIESVGARLRDMMPWIKANRIVDTSKN

SEQ ID NO: 5 - DNA, 1689 bp, Methylococcus capsulatus
  source 1..1689: mol_type = other DNA
atgaccgacaagcacccccgtccccattcgtcccaggtcgtcgacggcatggagcgcgccccgagccgcgcgatgctgcacgccgtcggcttcgccgatgccgacttcgccaaaccgcagatcggcatcgcttccacctgggcgatggtgacgccgtgcaacatgcacatcaacaagctcgccgaggacgcagcacgcggcgtcgacggcggcggcggcaaggcagtgatcttcaacaccatcaccatttccgacggcatctcgatgggcaccgaaggaatgaaatactccctcgtgtcgcgggaagtcatcgccgactcgatcgaaaccgtggtggcctgtcagggttatgacggcgtggtcgccatcggcggctgcgacaagaacatgcccggctgcctgatcgccctcgcccgcctcaaccgtccggcggtgttcgtctatggcggcaccatcctgccgggctgccacgacggcaagaagctggacgtggtgtcggtgttcgaagcggtcggcgcccgcgccaaccaccgcatcgacgatgccgaactgcacgccatcgaatccaatgccatccccggtccgggctcctgcggtggcatgtataccgccaacaccatggcctccgccatcgaggcattagggatgagcctgccgggcagttcggcccaggtggccatttcccgcgccaaggaactggattgcgagcgggccggcgcgcaggtcctcaagctcctggacctggggctcaaaccccgcgacatcatgaccaagaaggcgttcgagaacgccatcacggtggtgatcgccctgggcggctccaccaacgccgtgctgcacctcctggccatggccaacgcctgcggcgtcgacctgaagctcgacgatttcacccgcatcgggcgcaaagtgccgatgctggcggatctgaaacccagcggcagatactccatggccgaactggtggaaatcggcggcatccagccgctgatgaagaccttgctggacgcgggactcctgcacggcgactgcatgaccgtaaccggcaagaccctggaagaaaacctggccgacgcgcccgactacccggccggacaagacatgatccggtcgctggacaaccccatcaaaaaggacagccatctggtgatcctcaagggcaacctggcgccggaaggcgcggtcgccaagatcaccggcaaggaaggactgagcttcaccggcaccgcccgcgtattcgactgcgaggaagcggcgctcacggccatcctcgacggcacgatcgtgaaaggcgacgtcatcgtcatccgctatgaaggccccaagggcggccccggcatgcgcgagatgctctcgccgacctcggcggtcatgggcaagggattgggcaaggaggtcgccctcatcaccgacggccgcttttccggcggcacccacggcttcgtggtcggccacatcacgccggaagcctacaccggcggccccctggcgatcgtccgggacggcgataccatcaccatcgacgccgagacccgcgaattgagcctgcacgtcaccgacgatgaaatcggccggcgcctggcgcagtggactcaaccggcgccgcgctacaccaagggcgtgctggccaaatacgccaggttggtgagcccggcctcggaaggcgccgtcaccgacgacggcctctga

SEQ ID NO: 6 - protein, 562 aa, Methylococcus capsulatus
MTDKHPRPHSSQVVDGMERAPSRAMLHAVGFADADFAKPQIGIASTWAMVTPCNMHINKLAEDAARGVDGGGGKAVIFNTITISDGISMGTEGMKYSLVSREVIADSIETVVACQGYDGVVAIGGCDKNMPGCLIALARLNRPAVFVYGGTILPGCHDGKKLDVVSVFEAVGARANHRIDDAELHAIESNAIPGPGSCGGMYTANTMASAIEALGMSLPGSSAQVAISRAKELDCERAGAQVLKLLDLGLKPRDIMTKKAFENAITVVIALGGSTNAVLHLLAMANACGVDLKLDDFTRIGRKVPMLADLKPSGRYSMAELVEIGGIQPLMKTLLDAGLLHGDCMTVTGKTLEENLADAPDYPAGQDMIRSLDNPIKKDSHLVILKGNLAPEGAVAKITGKEGLSFTGTARVFDCEEAALTAILDGTIVKGDVIVIRYEGPKGGPGMREMLSPTSAVMGKGLGKEVALITDGRFSGGTHGFVVGHITPEAYTGGPLAIVRDGDTITIDAETRELSLHVTDDEIGRRLAQWTQPAPRYTKGVLAKYARLVSPASEGAVTDDGL

SEQ ID NO: 7 - DNA, 1650 bp, Methylococcus capsulatus
  source 1..1650: mol_type = other DNA
atgggcacggttgagcctggcgctatcggacaacatctgctcgcctgcctttaccaggcgggcgtcgggcacatcttcggcgttcccggcgattacgtgctgggcttctatgatctgatggccaaaggtcccgtccggcatatcgggaccacgcgggaggacaccgccgccttcgccgccgacggctatgcccgctgccggggcatgggcgcgctggcggtgacttacggggtcggtgcgctcaacaccgtcaacgccgtcgccggcgcctatgcggaatcctcgccggtggtggtcatcagcggtgcgccgggggtgcgcgagcaaagggaagacccgttgatccaccaccgcttcgggccgttccggttccagcgcgagatattcgaacggatcacctgcgccgccgtggtgctggacgatccggtgatcgccttccggcaggtggagcgtgcgctcgcggccgcccgtcagcactgcaagccggtgtacatcgagattcccgccgaccgggtgatggcgccgggatatccgattccacaggaaaccccggaaacgccttccagcgacgattcggccctggcggaggcggtcgccgaggccgcggagctcctgggccgtgcggtgtcgccggtgatccttgcaggcgtcgagttgcaccggcgagggctccaggacgccctcgtcggcctcgtcgagcaggcgcgcctgccggtggcggcgaccttgaccggcaagtcggtgttcgccgagcgccatcccgcctatctgggggtgtacgagggtgcgatgagcacggaaaacgcgcgctacatggtcgagcagtccgacctcctgctgatgctcggggtcacgctgaacgatgtcgacacgggcatctacacggcgcgtctcgatccgcagcgcatcgtccgcgcagcccagaacgaggtcgtgattcgccatcaccgctatccccgcgtcctgctcgcggacttcgtcacggccctggcgcggtccgtcaaggcccggggcgaggcgtttccgatgccggcggggccggaaccgtgggactttcccgcgccggaccggccgatgacgatcgcccggctggtggagcggctcgaccgcgcgctgacctccgacatgatcgtagtgtgcgacgtcggcgactgcctgttcgcagccaccgacctgcgcgtgcacgagcgcagcgaattcctggcgtccgccttctatacctcgatggggttcgcggtgcccgccgccctcggggcccagatcgcccgtccggaccaccgggcgctgatcctggtcggcgacggtgccttccagatgaccggaacggagctgtcgacccatgcccgtctcggcctggcgcccatcgtggtggtgctcgacaatcgcggttacagcaccgagcgcttcatcctcgacggagccttcaacgacatcgccgactggcgcttccaccggctgggcgaggtgttcggccccctacagggctacgacgcgcccgacgaagcggcgttcgaaaacgcgctcagcgaagcgctggtcaaccgaaacatgccgagcctcatcaacgtccgtctttcccccggcgatgcctcgatagccatgaagcgtctcgccgggcatctgcagtgccgggtcaagggcgagggctga

SEQ ID NO: 8 - protein, 549 aa, Methylococcus capsulatus
MGTVEPGAIGQHLLACLYQAGVGHIFGVPGDYVLGFYDLMAKGPVRHIGTTREDTAAFAADGYARCRGMGALAVTYGVGALNTVNAVAGAYAESSPVVVISGAPGVREQREDPLIHHRFGPFRFQREIFERITCAAVVLDDPVIAFRQVERALAAARQHCKPVYIEIPADRVMAPGYPIPQETPETPSSDDSALAEAVAEAAELLGRAVSPVILAGVELHRRGLQDALVGLVEQARLPVAATLTGKSVFAERHPAYLGVYEGAMSTENARYMVEQSDLLLMLGVTLNDVDTGIYTARLDPQRIVRAAQNEVVIRHHRYPRVLLADFVTALARSVKARGEAFPMPAGPEPWDFPAPDRPMTIARLVERLDRALTSDMIVVCDVGDCLFAATDLRVHERSEFLASAFYTSMGFAVPAALGAQIARPDHRALILVGDGAFQMTGTELSTHARLGLAPIVVVLDNRGYSTERFILDGAFNDIADWRFHRLGEVFGPLQGYDAPDEAAFENALSEALVNRNMPSLINVRLSPGDASIAMKRLAGHLQCRVKGEG

SEQ ID NO: 9 - DNA, 1083 bp, Saccharomyces cerevisiae
  source 1..1083: mol_type = other DNA
atgtcttatcctgagaaatttgaaggtatcgctattcaatcacacgaagattggaaaaacccaaagaagacaaagtatgacccaaaaccattttacgatcatgacattgacattaagatcgaagcatgtggtgtctgcggtagtgatattcattgtgcagctggtcattggggcaatatgaagatgccgctagtcgttggtcatgaaatcgttggtaaagttgtcaagctagggcccaagtcaaacagtgggttgaaagtcggtcaacgtgttggtgtaggtgctcaagtcttttcatgcttggaatgtgaccgttgtaagaatgataatgaaccatactgcaccaagtttgttaccacatacagtcagccttatgaagacggctatgtgtcgcagggtggctatgcaaactacgtcagagttcatgaacattttgtggtgcctatcccagagaatattccatcacatttggctgctccactattatgtggtggtttgactgtgtactctccattggttcgtaacggttgcggtccaggtaaaaaagttggtatagttggtcttggtggtatcggcagtatgggtacattgatttccaaagccatgggggcagagacgtatgttatttctcgttcttcgagaaaaagagaagatgcaatgaagatgggcgccgatcactacattgctacattagaagaaggtgattggggtgaaaagtactttgacaccttcgacctgattgtagtctgtgcttcctcccttaccgacattgacttcaacattatgccaaaggctatgaaggttggtggtagaattgtctcaatctctataccagaacaacacgaaatgttatcgctaaagccatatggcttaaaggctgtctccatttcttacagtgctttaggttccatcaaagaattgaaccaactcttgaaattagtctctgaaaaagatatcaaaatttgggtggaaacattacctgttggtgaagccggcgtccatgaagccttcgaaaggatggaaaagggtgacgttagatatagatttaccttagtcggctacgacaaagaattttcagactag

SEQ ID NO: 10 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 11 - DNA, 783 bp, Methylococcus capsulatus
  source 1..783: mol_type = other DNA
atggcagcaacaaccattggtggtgcagctgcggcggaagcgccgctgctggacaagaagtggctcacgttcgcactggcgatttacaccgtgttctacctgtgggtgcggtggtacgaaggtgtctatggctggtccgccggactggactcgttcgcgccggagttcgagacctactggatgaatttcctgtacaccgagatcgtcctggagatcgtgacggcttcgatcctgtggggctatctctggaagacccgcgaccgcaacctggccgcgctgaccccgcgtgaagagctgcgccgcaacttcacccacctggtgtggctggtggcctacgcctgggccatctactggggcgcatcctacttcaccgagcaggacggcacctggcatcagacgatcgtgcgcgacaccgacttcacgccgtcgcacatcatcgagttctatctgagctacccgatctacatcatcaccggttttgcggcgttcatctacgccaagacgcgtctgccgttcttcgcgaagggcatctcgctgccgtacctggtgctggtggtgggtccgttcatgattctgccgaacgtgggtctgaacgaatggggccacaccttctggttcatggaagagctgttcgtggcgccgctgcactacggcttcgtgatcttcggctggctggcactggccgtcatgggcaccctgacccagaccttctacagcttcgctcagggcgggctggggcagtcgctctgtgaagccgtggacgaaggcttgatcgcgaaataa

SEQ ID NO: 12 - protein, 260 aa, Methylococcus capsulatus
MAATTIGGAAAAEAPLLDKKWLTFALAIYTVFYLWVRWYEGVYGWSAGLDSFAPEFETYWMNFLYTEIVLEIVTASILWGYLWKTRDRNLAALTPREELRRNFTHLVWLVAYAWAIYWGASYFTEQDGTWHQTIVRDTDFTPSHIIEFYLSYPIYIITGFAAFIYAKTRLPFFAKGISLPYLVLVVGPFMILPNVGLNEWGHTFWFMEELFVAPLHYGFVIFGWLALAVMGTLTQTFYSFAQGGLGQSLCEAVDEGLIAK

SEQ ID NO: 13 - DNA, 744 bp, Methylococcus capsulatus
  source 1..744: mol_type = other DNA
atgagtgctgcgcaatctgcggttcgttcgcacgccgaagcggtccaggtatcccggaccatcgactggatggcgttgttcgtggtgtttttcgtgatcgtgggctcgtaccacattcatgccatgctcaccatgggtgactgggacttctggtcggactggaaagaccgtcgactgtgggtcacggtgaccccgatcgtactggtcaccttcccggcggccgtacaatcctacctgtgggagcggtatcgtctgccctggggagccaccgtgtgcgtcctgggtctgctgctgggcgagtggatcaaccgttatttcaacttctggggctggacctacttcccgatcaacttcgtgttccctgcctcgctggtgccgggcgccatcatcctggacaccgtgctgatgctgtcgggcagctacctgttcaccgcgatcgtcggtgcgatgggctggggtctgatcttctacccgggcaactggccgatcatcgcgccgctgcacgtgccggtggaatacaacggcatgctgatgtcgatcgccgacatccagggttacaactatgtgcgtacgggtacgcctgagtacatccgcatggtagagaagggcaccctgcgtaccttcggtaaggacgtggcgccggtatcggcattcttctccgcgttcatgtcgatcctgatctacttcatgtggcacttcatcggtcgctggttctccaacgaacggttcctgcagagcacctga

SEQ ID NO: 14 - protein, 247 aa, Methylococcus capsulatus
MSAAQSAVRSHAEAVQVSRTIDWMALFVVFFVIVGSYHIHAMLTMGDWDFWSDWKDRRLWVTVTPIVLVTFPAAVQSYLWERYRLPWGATVCVLGLLLGEWINRYFNFWGWTYFPINFVFPASLVPGAIILDTVLMLSGSYLFTAIVGAMGWGLIFYPGNWPIIAPLHVPVEYNGMLMSIADIQGYNYVRTGTPEYIRMVEKGTLRTFGKDVAPVSAFFSAFMSILIYFMWHFIGRWFSNERFLQST

SEQ ID NO: 15 - DNA, 1245 bp, Methylococcus capsulatus
  source 1..1245: mol_type = other DNA
atgaaaacaataaaggaccggattgcaaaatggtctgcaatcggactgctgtccgccgtggcagcgaccgccttctatgcgccgagcgccagcgcccacggtgagaaatcgcaggccgcgttcatgcgtatgcgtaccatccactggtacgacctgagctggtcgaaagagaaagtcaagatcaacgagaccgtggaaatcaaaggcaagttccacgtgttcgaaggctggccggaaacggtcgacgaaccggatgtggcgttcctgaacgtcggcatgccgggtccggtgttcatccgcaaggaatcgtacatcggcggtcagctggtgccgcgttccgtacgtctggaaatcggcaagacctatgacttccgggttgtcctcaaagcccgtcgtccgggtgactggcacgttcacaccatgatgaacgtccagggcggtggaccgatcatcggtcccggcaaatggatcaccgtggaaggctccatgagtgaattccgcaaccccgtcaccaccctgaccggtcagacggtggacctggagaactacaacgaaggcaacacctatttctggcacgccttctggttcgccatcggagttgcctggatcggctactggtcgcgtcgaccgatcttcatcccccgtctgctgatggtggatgccggtcgtgcggatgaactggtgtccgccaccgaccgcaaggtggcgatgggcttcctggccgccaccatcctgatcgtggtcatggccatgtccagcgccaacagcaagtacccgatcaccatcccgctgcaggccggcaccatgcgtggcatgaagccgctggaactgccggcgccgacggtatcggtgaaagtggaagacgccacctaccgggtaccgggccgcgccatgcggatgaagctgaccatcaccaaccacggcaacagcccgatccggctgggtgagttctacaccgcctcggtgcgtttcctggattccgacgtgtacaaggacaccaccggctatccggaagacctgctggccgaagacggcctgagcgtcagcgacaacagcccgctggctccgggtgagacgcgcacggtcgacgtgacggcgtccgacgcggcgtgggaagtgtaccgtctgtccgacatcatctacgatccggacagccgtttcgccggtctgctgttcttcttcgacgccactggcaaccgccaggtcgtccagatcgacgcaccgctgatcccgtcgttcatgtaa

SEQ ID NO: 16 - protein, 414 aa, Methylococcus capsulatus
MKTIKDRIAKWSAIGLLSAVAATAFYAPSASAHGEKSQAAFMRMRTIHWYDLSWSKEKVKINETVEIKGKFHVFEGWPETVDEPDVAFLNVGMPGPVFIRKESYIGGQLVPRSVRLEIGKTYDFRVVLKARRPGDWHVHTMMNVQGGGPIIGPGKWITVEGSMSEFRNPVTTLTGQTVDLENYNEGNTYFWHAFWFAIGVAWIGYWSRRPIFIPRLLMVDAGRADELVSATDRKVAMGFLAATILIVVMAMSSANSKYPITIPLQAGTMRGMKPLELPAPTVSVKVEDATYRVPGRAMRMKLTITNHGNSPIRLGEFYTASVRFLDSDVYKDTTGYPEDLLAEDGLSVSDNSPLAPGETRTVDVTASDAAWEVYRLSDIIYDPDSRFAGLLFFFDATGNRQVVQIDAPLIPSFM

SEQ ID NO: 17 - DNA, 783 bp, Methylococcus capsulatus; identical to SEQ ID NO: 11
  source 1..783: mol_type = other DNA

SEQ ID NO: 18 - protein, 260 aa, Methylococcus capsulatus; identical to SEQ ID NO: 12

SEQ ID NO: 19 - DNA, 744 bp, Methylococcus capsulatus
  source 1..744: mol_type = other DNA
atgagtgctgcgcaatctgcggttcgttcgcacgccgaagcggtccaggtatcccggaccatcgactggatggcgttgttcgtggtgtttttcgtgatcgtgggctcgtaccacattcacgccatgctcaccatgggtgactgggacttctggtcggactggaaagaccgtcgactgtgggtcacggtgaccccgatcgtactggtcaccttcccggcggccgtacaatcctacctgtgggagcggtatcgtctgccctggggagccaccgtgtgcgtcctgggtctgctgctgggcgagtggatcaaccgttatttcaacttctggggctggacctacttcccgatcaacttcgtgttccctgcctcgctggtgccgggcgccatcatcctggacaccgtgctgatgctgtcgggcagctacctgttcaccgcgatcgtcggtgcgatgggctggggtctgatcttctacccgggcaactggccgatcatcgcgccgctgcacgtgccggtggaatacaacggcatgctgatgtcgatcgccgacatccagggttacaactatgtgcgtacgggtacgcctgagtacatccgcatggtagagaagggcaccctgcgtaccttcggtaaggacgtggcgccggtatcggcattcttctccgcgttcatgtcgatcctgatctacttcatgtggcacttcatcggtcgctggttctccaacgaacggttcctgcagagcacctga

SEQ ID NO: 20 - protein, 247 aa, Methylococcus capsulatus; identical to SEQ ID NO: 14

SEQ ID NO: 21 - DNA, 1245 bp, Methylococcus capsulatus
  source 1..1245: mol_type = other DNA
atgaaaacaataaaggaccggattgcaaaatggtctgcaatcggactgctgtccgccgtggcagcgaccgccttctatgcgccgagcgccagcgcccacggtgagaaatcgcaggccgcgttcatgcgtatgcgtaccatccactggtacgacctgagctggtcgaaagagaaagtcaagatcaacgagaccgtggaaatcaaaggcaagttccacgtgttcgaaggctggccggaaacggtcgacgaaccggatgtggcgttcctgaacgtcggcatgccgggtccggtgttcatccgcaaggaatcgtacatcggcggtcagctggtgccgcgttccgtacgtctggaaatcggcaagacctatgacttccgggttgtcctcaaagcccgtcgtccgggtgactggcacgttcacaccatgatgaacgtccagggcggtggaccgatcatcggtcccggcaaatggatcaccgtggaaggctccatgagtgaattccgcaaccccgtcaccaccctgaccggtcagacggtggacctggagaactacaacgaaggcaacacctatttctggcacgccttctggttcgccatcggagttgcctggatcggctactggtcgcgtcgaccgatcttcatcccccgtctgctgatggtggatgccggtcgtgcggacgaactggtgtccgccaccgaccgcaaggtggcgatgggcttcctggccgccaccatcctgatcgtggtcatggccatgtccagcgccaacagcaagtacccgatcaccatcccgctgcaggccggcaccatgcgtggcatgaagccgctggaactgccggcgccgacggtatcggtgaaagtggaagacgccacctaccgggtaccgggccgcgccatgcggatgaagctgaccatcaccaaccacggcaacagcccgatccggctgggtgagttctacaccgcctcggtgcgtttcctggattccgacgtgtacaaggacaccaccggctatccggaagacctgctggccgaagacggcctgagcgtcagcgacaacagcccgctggctccgggtgagacccgcacggtcgacgtgacggcgtccgacgcggcgtgggaagtgtaccgtctgtccgacatcatctacgatccggacagccgtttcgccggtctgctgttcttcttcgacgccactggcaaccgccaggtcgtccagatcgacgcaccgctgatcccgtcgttcatgtaa

SEQ ID NO: 22 - protein, 414 aa, Methylococcus capsulatus; identical to SEQ ID NO: 16

SEQ ID NO: 23 - DNA, 1584 bp, Methylococcus capsulatus
  source 1..1584: mol_type = other DNA
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 24 - protein, 527 aa, Methylococcus capsulatus
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 25 - DNA, 1170 bp, Methylococcus capsulatus
  source 1..1170: mol_type = other DNA
atgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataa

SEQ ID NO: 26 - protein, 389 aa, Methylococcus capsulatus
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 27 - DNA, 426 bp, Methylococcus capsulatus
  source 1..426: mol_type = other DNA
atgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaa

SEQ ID NO: 28 - protein, 138 aa, Methylococcus capsulatus
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLED

SEQ ID NO: 29 - DNA, 513 bp, Methylococcus capsulatus
  source 1..513: mol_type = other DNA
atggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactga

SEQ ID NO: 30 - protein, 170 aa, Methylococcus capsulatus
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 31 - DNA, 312 bp, Methylococcus capsulatus
  source 1..312: mol_type = other DNA
atggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattga

SEQ ID NO: 32 - protein, 103 aa, Methylococcus capsulatus
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 33 - DNA, 1047 bp, Methylococcus capsulatus
  source 1..1047: mol_type = other DNA
atgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctga

SEQ ID NO: 34 - protein, 348 aa, Methylococcus capsulatus
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 35 - DNA, 1806 bp, Methylococcus capsulatus
  source 1..1806: mol_type = other DNA
atgcaaatttgcaaactggcaagtggctgcggcgggtcgatgctggcgatggccgccgtgctagccgcgcaatccacgcacgccaattcggagctggaccggctgtcgaaggacgaccggaactgggtcatgcagaccaaggactacagcgccacccacttcagccggctgacggaaatcaatagccacaacgtcaagaacctgaaggtggcctggaccctgtccaccggcacgttgcatggccacgaaggtgcgccgttggtggtggacggcatcatgtacatccacacgccgttccccaacaacgtctatgcagtcgacctgaacgacacccgcaagatgctgtggcagtacaagcccaagcagaatccggcggcccgcgcggtggcttgctgcgacgtggtcaaccgcggtctggcctacgtgccggccggcgagcacggtccggcgaagatcttcctcaaccagcttgacggccacatcgtcgcactcaacgccaagaccggcgaagagatatggaagatggaaaattccgacatcgccatgggctccaccctcaccggcgcgcctttcgtggtgaaggacaaggtactggtaggttcggccggggccgagctgggcgtgcgtggctacgtcacggcctataacatcaaggacggcaagcaggagtggcgggcctatgccaccggtcccgacgaagacttgttgctggacaaggacttcaacaaggacaacccgcattacggtcagttcggcctggggctctcaacctgggagggtgatgcctggaagatcggcggcggcaccaattggggctggtatgcctatgatcccaagttggacatgatctactacggttccggcaatccggcaccctggaacgagaccatgcggcccggcgacaacaaatggaccatgaccatctggggccgcgacgccgacaccggccgcgccaagttcggctaccagaagacgccgcacgacgagtgggattacgccggtgtcaactacatgggtctgtccgaacaggaagtggacggcaagctgacgccgctgctgacccatcccgaccgcaacggtctggtgtatacgctgaaccgggaaaccggcgccctggtcaatgccttcaagatcgatgacaccgtcaactgggtgaaaaaggtcgatctgaagaccggcctgccgatccgcgatccggagtacagcacccgcatggaccacaatgccaaaggcatctgtccctcggccatgggctatcacaaccagggcatcgagtcctacgatccggacaagaagctgttcttcatgggcgtgaaccacatctgcatggactgggagccgttcatgctgccctaccgcgccggccagttctttgtgggggcgaccctcaacatgtatccgggacccaaggggatgctgggtcaggtcaaggcgatgaacgcggtcaccggcaagatggaatgggaagtgccggagaagtttgcggtctggggtggcaccttggcgaccgccggcgacctcgtgttctacggtaccctcgacggcttcatcaaggcccgcgacacccgtaccggcgagctgaagtggcagttccagttgccctccggcgtgatcggccatcccatcacctatcagcacaacggcaagcaatacattgccatctactccggcgtcggcggctggccaggagtagggctggtattcgacctgaaggacccgaccgcaggtctgggagctgtgggtgcgttcagggaactggcgcattacacccagatgggtggatcggtgttcgtgttctcgctttga

SEQ ID NO: 36 - protein, 601 aa, Methylococcus capsulatus
MQICKLASGCGGSMLAMAAVLAAQSTHANSELDRLSKDDRNWVMQTKDYSATHFSRLTEINSHNVKNLKVAWTLSTGTLHGHEGAPLVVDGIMYIHTPFPNNVYAVDLNDTRKMLWQYKPKQNPAARAVACCDVVNRGLAYVPAGEHGPAKIFLNQLDGHIVALNAKTGEEIWKMENSDIAMGSTLTGAPFVVKDKVLVGSAGAELGVRGYVTAYNIKDGKQEWRAYATGPDEDLLLDKDFNKDNPHYGQFGLGLSTWEGDAWKIGGGTNWGWYAYDPKLDMIYYGSGNPAPWNETMRPGDNKWTMTIWGRDADTGRAKFGYQKTPHDEWDYAGVNYMGLSEQEVDGKLTPLLTHPDRNGLVYTLNRETGALVNAFKIDDTVNWVKKVDLKTGLPIRDPEYSTRMDHNAKGICPSAMGYHNQGIESYDPDKKLFFMGVNHICMDWEPFMLPYRAGQFFVGATLNMYPGPKGMLGQVKAMNAVTGKMEWEVPEKFAVWGGTLATAGDLVFYGTLDGFIKARDTRTGELKWQFQLPSGVIGHPITYQHNGKQYIAIYSGVGGWPGVGLVFDLKDPTAGLGAVGAFRELAHYTQMGGSVFVFSL

SEQ ID NO: 37 - DNA, 573 bp, Methylococcus capsulatus
  source 1..573: mol_type = other DNA
atgctcaccagcagtccttattaccggtccggctacgtattcgtctaccgcaaggacacgggactgagcatccaagattggaacagcgcggcactgaagaccgtgaagcggatcgcattcatgccggataccccggctgagacgatgatccgcaccatcggccgctacaacgacatgttcaactacatgcactctctggtcggtttcaagtcgcggcgtaaccagtacgtgcgctacgacccggccaagctggtggcggaagtcgccgacggcaacgcggaagtcgcggtgttgtgggggccggcggcggcgcgctatgtcagaggggcggggctggccatgaccgtcatccccgacgacaaccggcggtccgacggcgagaaagtgccccaccactattcgacttccgtcggcgtgcgcaagggcgaggaggccctgctcaagcagatcgaccaggttctggcccgcttcggcaaggaagtgaatgcggtgctggaggcggaaggcattccgctgttgcccatggatgaaaaaccggccaggacggcttcccatgatcgaaggaaaggctag

SEQ ID NO: 38 - protein, 190 aa, Methylococcus capsulatus
MLTSSPYYRSGYVFVYRKDTGLSIQDWNSAALKTVKRIAFMPDTPAETMIRTIGRYNDMFNYMHSLVGFKSRRNQYVRYDPAKLVAEVADGNAEVAVLWGPAAARYVRGAGLAMTVIPDDNRRSDGEKVPHHYSTSVGVRKGEEALLKQIDQVLARFGKEVNAVLEAEGIPLLPMDEKPARTASHDRRKG

SEQ ID NO: 39 - DNA, 480 bp, Methylococcus capsulatus
  source 1..480: mol_type = other DNA
atgaagctgaagaatgcgaggttcgacgtggctggcatgtgtgtcgccgggttgttggcgctgcccgcgcaggccgacattaccctgcggcatgccgtcaccggcgagacgctggagttgtcctacgccaaggcgggcggcgacacgcaagccgtcaagcagttcctgcagaccggcaagaacccttacaacggcaacaaggaggtagtggaacagggacatagtctgtatctgtcagcctgttccggctgccacggccatgaggccgaaggcaagctcggtccgggattggcggacgactattggacctatccccgcgcggccaccgacgtcggtttgttcgaaatcctgttcggcggcgcgcagggcatgatggggccgcagtacgtcaacctcaacaatgacgaaatgctcaagatcatggcctggatccgcagcctttaccggggcgatccagccaaggccgaatggctgaaatga

SEQ ID NO: 40 - protein, 159 aa, Methylococcus capsulatus
MKLKNARFDVAGMCVAGLLALPAQADITLRHAVTGETLELSYAKAGGDTQAVKQFLQTGKNPYNGNKEVVEQGHSLYLSACSGCHGHEAEGKLGPGLADDYWTYPRAATDVGLFEILFGGAQGMMGPQYVNLNNDEMLKIMAWIRSLYRGDPAKAEWLK

SEQ ID NO: 41 - DNA, 285 bp, Methylococcus capsulatus
  source 1..285: mol_type = other DNA
atgatgcagaaaacgagtttcgtcgcggccgccatggccgtttcgttcgcggcgggtgtccaggcctatgacggtacccactgcaaggcgcccggaaactgctgggagcccaagcccggttatccggacaaggtcgccggcagcaagtacgaccccaagcatgacccgaacgagctcaacaagcaggcggagtcgatcaaggcgatggaagcccgcaaccagaagcgcgtggagaactacgccaagaccggcaagttcgtctacaaggtcgaagacatcaaatga

SEQ ID NO: 42 - protein, 94 aa, Methylococcus capsulatus
MMQKTSFVAAAMAVSFAAGVQAYDGTHCKAPGNCWEPKPGYPDKVAGSKYDPKHDPNELNKQAESIKAMEARNQKRVENYAKTGKFVYKVEDIK

SEQ ID NO: 43 - DNA, 1020 bp, Methylococcus capsulatus
  source 1..1020: mol_type = other DNA
atgaatctagacaccccccttgccgacggcctggaacgcgccaaacgcttcgaacaggggctgcagcaagtcgtgctcgggcaggagcgcccgatccgcctgctgaccctggccgtgttcgcccgcggtcatgcgctgctcgaaggcggcgtcggcgtcgggaagaccaccttgctccgtgcggtggcgcgcggcatcggcggcgattacgagcggatcgagggcaccatcgacctgatgccgaacgatctggtctattacacctacctggacgagcaaggtaggccgggcgtcgcgccggggcctttgctcaagcacggggagcagctttccatttttttcttcaacgagatcaaccgcgcccggccccaggtgcattccctcctgctacgggtcatggccgagcgcagcgtgtcggctttcaaccgcgagtaccggtttccgtacctgcaggtgttcgccgaccgcaaccgggtggaaaaggaggagactttcgaattgcccgcggcggcgcgcgaccgcttcatgctcgaaatcgccatcgagccgccggccgatcctgcgcatatcgaccaaatcctgttcgacccgcgtttctacgatcccgaccggctggtcgcgtccgcgccggccgatacgctctcgttccgtgaactcaacggcattgccgaagccctgcaaggcggcatccacgtcagcgcccgtctcagatcctatgtccaggatctgtggcgcgcgacccggcggccggaggatttcggcatcgctctccacgaggcggattccggcgacatgatcgaggccggttccagtccccgcggcatgagctacttggtccggctggcgcgggtgcaggcgtggctcagtggccgggaccgggtcgagccggaggacgttcaatacgtgttcgctccggcggtcggccaccgcatcttcctcaagccggtctacgaataccgccgcgccgagctgatcccggagctggtcggcaagctgatccgccggatcgcggcgccatga

SEQ ID NO: 44 - protein, 339 aa, Methylococcus capsulatus
MNLDTPLADGLERAKRFEQGLQQVVLGQERPIRLLTLAVFARGHALLEGGVGVGKTTLLRAVARGIGGDYERIEGTIDLMPNDLVYYTYLDEQGRPGVAPGPLLKHGEQLSIFFFNEINRARPQVHSLLLRVMAERSVSAFNREYRFPYLQVFADRNRVEKEETFELPAAARDRFMLEIAIEPPADPAHIDQILFDPRFYDPDRLVASAPADTLSFRELNGIAEALQGGIHVSARLRSYVQDLWRATRRPEDFGIALHEADSGDMIEAGSSPRGMSYLVRLARVQAWLSGRDRVEPEDVQYVFAPAVGHRIFLKPVYEYRRAELIPELVGKLIRRIAAP

SEQ ID NO: 45 - DNA, 873 bp, Methylococcus capsulatus
  source 1..873: mol_type = other DNA
gtggtttggtctctcctgccggtcgcggccttggtatcggttccacttcatggcgccacttcgctctcgttcgacacgccccgcgccttcggctacgtcatcggtgatctcatccgccacgaggttcgggtcgaaaccgatgcggggcagggaatagaggctgcgtccctgcccaaggaaggctggatcaaccgctggctgctgctgcggcgggtcgaagtccgccgcgagggcaggcaccggatactgacgctggaataccagactttctacgccccgttggaagtgaagaacctcacgattcccggcttcgagctgcaactggccggttcgggcgaacggttggcggtcccggactggactttcaccaccgcgccgatccgggagctgtcggtgctgcgcgccgaaggcccgtcgatgcgtccggacgccgcaccggcgccgctgccgactctcggccccgccgccgcgagcgtcggttccggcctcgcagccacgggcgcgctggcctggtgggcctatctgagcgcctggctgccgttcgtgtcgcgcggccgtcatttcgccgaggcccgccgggtgctgcgggatctgcgcggcctgggagacagccgggaggcattgcgcagaggtttttcctgtctgcaccaggctttcaatcggacttcgggtgagccgctgttcatcgaagggctggacgagttcttccggagccatccggcctacgatctcttgcgggacgagatccaggacttcttcctggcctcgtatgaagtctttttcggagagggcgcaccggcgccgtcgttcgacctggcgcgcatggaggcgttggcccgttcgtgccagcttgccgaaaggaggcggccatga

SEQ ID NO: 46 - protein, 290 aa, Methylococcus capsulatus
VVWSLLPVAALVSVPLHGATSLSFDTPRAFGYVIGDLIRHEVRVETDAGQGIEAASLPKEGWINRWLLLRRVEVRREGRHRILTLEYQTFYAPLEVKNLTIPGFELQLAGSGERLAVPDWTFTTAPIRELSVLRAEGPSMRPDAAPAPLPTLGPAAASVGSGLAATGALAWWAYLSAWLPFVSRGRHFAEARRVLRDLRGLGDSREALRRGFSCLHQAFNRTSGEPLFIEGLDEFFRSHPAYDLLRDEIQDFFLASYEVFFGEGAPAPSFDLARMEALARSCQLAERRRP

SEQ ID NO: 47 - DNA, 984 bp, Methylococcus capsulatus
  source 1..984: mol_type = other DNA
atgaccgattgggcgctggacacgccgtacttgctgtggggcctgccgctggcgctgcttccgttgtggcggttgccgctgcgccctgccccgtgttcctggcatgcattgttgcccgccgatactgcgtcgcgggccgtcgacctgagtctgcgcctcgccggtgccggcgccatcctggcgctgctgctgggcagtgccggtctgcatcggcgcgagtacaccgtcgaacgcaccggctacggcgcccacatggtgctgctgctggaccgcagccgcagcatggatgacagcttcgcagggcgtactcccacgggcggcgaggaatccaagtccgccgcggcggagcgcctcctgagcggtttcgtctcgagcggacgcaacgatctggtcggggtcgccgccttcagcacctccccgttgttcgtgctgccgctgaccgacaacaaggctgcggtgctggcggcggtccacgccatgaagctgccgggtctggcgcagacgcatgtgagcaaggggctggcgatggcgctttcgtatttcggcgacgattcgaccgcgggttcgcgtatcgtcctgctggtgtccgacggtgccgccgaggtggacccggacagcgagctgaagctgcgccgctggttcaaggagaagggcgtacggctgtactggatattcctgcgcaccgcgggcagccacggtatcttcgaaactccggacaacccggaggaagacaacgcccaggcgcggcccgagcgctatctgcatctgtttttcaacagtctgggcatcccctaccgcgcctacgaggcggaagacgccgacgccctcaagcgcgccatcgccgacgtcgaccgcgaggagcagcggccgctgcgctatgccgagcgggtgccgcggcgggatctgcaagccttttgttatctggcggcggcgctggctctggcctggctggtcgccgcgaagggcatggaggtggcgcgatga

SEQ ID NO: 48 - protein, 327 aa, Methylococcus capsulatus
MTDWALDTPYLLWGLPLALLPLWRLPLRPAPCSWHALLPADTASRAVDLSLRLAGAGAILALLLGSAGLHRREYTVERTGYGAHMVLLLDRSRSMDDSFAGRTPTGGEESKSAAAERLLSGFVSSGRNDLVGVAAFSTSPLFVLPLTDNKAAVLAAVHAMKLPGLAQTHVSKGLAMALSYFGDDSTAGSRIVLLVSDGAAEVDPDSELKLRRWFKEKGVRLYWIFLRTAGSHGIFETPDNPEEDNAQARPERYLHLFFNSLGIPYRAYEAEDADALKRAIADVDREEQRPLRYAERVPRRDLQAFCYLAAALALAWLVAAKGMEVAR

SEQ ID NO: 49 - DNA, 519 bp, Methylococcus capsulatus
  source 1..519: mol_type = other DNA
atgctggccttgtcggcgttgctggagctcaggcaatggcggaaggccgcggcggccaatgccgatatcgccgagctgctgggggggcacgacatcgccccggaacggctggcggcggcatcgccccaagtcctgttggcgcgggccgtgtatttcgtgcggcacgagcgctacggcgacgcgctggagctgctgaacctgctggagacccggggcgatggcgccttccgcgccgacgtgtattacaaccagggcaatctgcagcttgcccaggctctggaccgcgtcgaaaaatcggaaatggaccaggcccgggtcttcgccgaactggccaaggaagcctaccggcgtgccttgtcgctggcacccggccactgggacgccaaatacaacctggaagtggccatgcgcctcatgcccgaaatggaccgggtcagccctgccgatgacgaggcgcccgcggctgaatccaaacggctgtggacaggtttgcccggactcccgcgaggcctgccttga

SEQ ID NO: 50 - protein, 172 aa, Methylococcus capsulatus
MLALSALLELRQWRKAAAANADIAELLGGHDIAPERLAAASPQVLLARAVYFVRHERYGDALELLNLLETRGDGAFRADVYYNQGNLQLAQALDRVEKSEMDQARVFAELAKEAYRRALSLAPGHWDAKYNLEVAMRLMPEMDRVSPADDEAPAAESKRLWTGLPGLPRGLP

SEQ ID NO: 51 - DNA, 972 bp, Methylococcus capsulatus
  source 1..972: mol_type = other DNA
ttgagcatctggcggcagcgcgttgccgatccggtttttgccggcctgattgtagccctccttctggcggtagccgcctgtttcccgctccggctggtgctggagcggctggtgttcagccacatcgtcgtcgtcgacatcacccgcagcatgaacgtcgaggactaccggcgaggcgcgcgcgccgtgtcgcggctggaattcgtcaggcagagcctgatcggcgccgtggccgacctgccctgcggctccgctgtgggggtgggcgttttcaccgaacgcgagccggcgctactgttcgagccgatcgaaacctgcgccggcttttccgccatcagcgccgccatcgaacagctcgactggcgcatggcctgggctgccgacagtctgatcgccgcaggtctgcacaacaccctggatttgctggggcgcggcgatgcggacgtgattttcgtcaccgacggccatgaggcgccgccactcaatccccgctactgcccggacttcagcgacctcagaggcaaggtccgggggctgatcgtcggagtgggaggactgagcctctcgcccatccccaagtacgacgagtcggggcggcgttcgggcgtttatggcgaggacgaagtcccgcagcgctcgagcttcggcctgtcggagctgccgcccgagcagatcgagggctaccacgcccgcaacgctcccttcggcagcgagagagccgggggcacggaacatctgtcccagctcaaggaaggatatttgcgccagctcgccgaagccgccggcctgggctaccaccgcctggaatcgcccgaaggactgggccgcgctctcacggcaccggccttggcgcggcgccagcggatcgccacagacgtccgctggattcccgccgccctggcgctcgccgtactgatggcggtgtatctgcgggtgctgctgccgcgtcctggattttcaacctcaaactga

SEQ ID NO: 52 - protein, 323 aa, Methylococcus capsulatus
LSIWRQRVADPVFAGLIVALLLAVAACFPLRLVLERLVFSHIVVVDITRSMNVEDYRRGARAVSRLEFVRQSLIGAVADLPCGSAVGVGVFTEREPALLFEPIETCAGFSAISAAIEQLDWRMAWAADSLIAAGLHNTLDLLGRGDADVIFVTDGHEAPPLNPRYCPDFSDLRGKVRGLIVGVGGLSLSPIPKYDESGRRSGVYGEDEVPQRSSFGLSELPPEQIEGYHARNAPFGSERAGGTEHLSQLKEGYLRQLAEAAGLGYHRLESPEGLGRALTAPALARRQRIATDVRWIPAALALAVLMAVYLRVLLPRPGFSTSN

SEQ ID NO: 53 - DNA, 525 bp, Methylococcus capsulatus
  source 1..525: mol_type = other DNA
atgaaaccgatgctcatcctgaccgcgttgctgttcgcctccgtttcgttggcgcacggacccaccccccaaaaggtcgtcgagaccgtggagatcgcggctcccgtggaccgggtctggaacgccgtgaaggatttcggtgccatcgcgcagtggaatcccgctctggccaagagcgaaagcaccggcggcaacaccaccggcgagaagcgcatcctccattttcccaacggcgagcagctcaccgaggaactcgatgcctacgacccggcagcccacgaatacacctaccggctgggcaaggacaacgtcaaggcgctgccggccagttcctactccgccgtgctcaaggtcaaggccaccgagacgggcagccagatcgaatggaagagtcggctctatcgcggcgataccggaaacttcccgccggacgagctgaacgacgaggccgccgttgcggcgatgcagaggtttttccgcgccgggctggacaatctcaagaaaagtcttgggcccctcgaatga

SEQ ID NO: 54 - protein, 174 aa, Methylococcus capsulatus
MKPMLILTALLFASVSLAHGPTPQKVVETVEIAAPVDRVWNAVKDFGAIAQWNPALAKSESTGGNTTGEKRILHFPNGEQLTEELDAYDPAAHEYTYRLGKDNVKALPASSYSAVLKVKATETGSQIEWKSRLYRGDTGNFPPDELNDEAAVAAMQRFFRAGLDNLKKSLGPLE

SEQ ID NO: 55 - DNA, 1530 bp, Methylococcus capsulatus
  source 1..1530: mol_type = other DNA
atgctgcaaaaatacatagagaagattctgcgcgcccgtgtctacgacgttgcccaggagaccccgctggacccggcgcccggcctgtcgcggcggctggacaacacggtgctgatcaagcgcgaggacctgcagccggtgttctcgttcaagctgcgcggcgcctacaacaagatcgcctcgctcacacccgaggcgcgcgcggccggcgtgatcgcggcctccgccggcaaccacgcccagggcgtggcactggcggcgcagcggctgggcatccgcgccgtgatcgtgatgccttgcaccaccccgcatatcaaggtcgatgcggtgcgcaaccgaggcggtgaggtcgtactgcatggcgacgcctatgacgaagcctacgaacatgcgctggaactggcccgcgaccagtgcctgaccttcgtccacccctacgacgatccggaagtcatcgccgggcaaggcaccatcggcatggaaatcctgcgccagcaccaggacgccatccacgccatcttcgtgcctgtgggcggcggcggattgatcgccggcatcgccgcctacgtcaagttcgtgcgcccggacatccgcgtcatcggcgtggaaccagtggactccgactgcctgcaccgggcgctgaaagccaagcggcgggtgatcctgaagcaggtgggcctgttcgccgacggcgtcgcggtgaagcaggtcggcaaggaaccgttccatctcgcccaccagtgggtggacgaggtcgtgaccgtcgacaccgacgaaatctgcgccgccatcaaggacatcttcgacgacacccgctccatcgccgagccggcgggcgcgctgggcatcgccgggctcaagaaatacgtggccgaaacaggaatcaagaacgcgtgcctggtggcgatcgaaagcggcgccaacatcaacttcgaccggctgcgccacgtcgctgagcgcgccgagatcggcgaaaagcgcgaactgctgctggcagtgacgatccccgagcggcccggcagcttcctcgaattctgccgggtgctgggccgccgcaacatcaccgaattcaactaccgcttcttcgacgaaaaggccgcccaggtgttcgtcggcctcccggtggcgagcggcgcgatcgaccgcgaaagcctggtccgcgaattcgaacgccagggtttcggcgtgctcgacctgaccggcaacgaactcgccatcgaacacatccgctacatggtcggcggccacgcgccgaaactgctggacgaacaggtctacagcttcgaattccccgagcgacccggcgcgctgctgcgcttcctgtccatcatgggcgggcgctggaacatcagcctgttccattaccgcaaccacggcgccgccttcggccgggtactgatgggcatccaggtgccgaaaccggaacgcaaggccttccgggaattcctcgaagccatcggctacgccttcaaggaggaaacccaaaatcccgcctaccggctgttcgcggggggcagcgagcgggggtga

SEQ ID NO: 56 - protein, 509 aa, Methylococcus capsulatus
MLQKYIEKILRARVYDVAQETPLDPAPGLSRRLDNTVLIKREDLQPVFSFKLRGAYNKIASLTPEARAAGVIAASAGNHAQGVALAAQRLGIRAVIVMPCTTPHIKVDAVRNRGGEVVLHGDAYDEAYEHALELARDQCLTFVHPYDDPEVIAGQGTIGMEILRQHQDAIHAIFVPVGGGGLIAGIAAYVKFVRPDIRVIGVEPVDSDCLHRALKAKRRVILKQVGLFADGVAVKQVGKEPFHLAHQWVDEVVTVDTDEICAAIKDIFDDTRSIAEPAGALGIAGLKKYVAETGIKNACLVAIESGANINFDRLRHVAERAEIGEKRELLLAVTIPERPGSFLEFCRVLGRRNITEFNYRFFDEKAAQVFVGLPVASGAIDRESLVREFERQGFGVLDLTGNELAIEHIRYMVGGHAPKLLDEQVYSFEFPERPGALLRFLSIMGGRWNISLFHYRNHGAAFGRVLMGIQVPKPERKAFREFLEAIGYAFKEETQNPAYRLFAGGSERG

SEQ ID NO: 57 - DNA, 1545 bp, Methylococcus capsulatus
  source 1..1545: mol_type = other DNA
atgcacgacagactgatcattttcgacacgaccttgcgcgacggagagcagagccccggcgcgtccatgacccgcgatgaaaaggtccgcatcgcccgggcgctggagcgtctgaaggtcgacgtcatcgaggcgggctttcccgccgccagccccggcgatttcgaggccgtccaggccgtggcccggaccatcaaggacagcagggtctgcggcctggcccgcgccctcgaccgcgacatcgaccgcgccggcgaagccctcaaggacgcccagcgcgcccgcatccacaccttcatcgccacctcgcccatccacatgcggcacaagctgcagatgtcgcccgaccaggtggtggaatacgcggtcaaggccgtcaagcgggcccgccagtacaccgacgacgtggaattctcgcccgaggacgccggacgctccgaggaggatttcctctgccgcatcctggaagccgtgatcgatgcgggggcgaccacgctgaacatccccgacaccgtcggctacgccttcccggaacagttcgggcacatgatcggccggctgatcgagcggattccgaactccgacaaggccgtgttctcggttcactgccacaacgacctgggactggcggtcgccaattcgctggccgccgtgctgcacggcgcgcgccaggtggaatgcaccatcaacgggctgggcgagcgggccggcaacgccgcgctggaagagatcgtcatggcggtgcgcacccgtaaagacatcttcccctgccacaccgacatcgagacacgggaaatcgtggcctgctccaaactggtctccagcatcaccggtttcccgatccagcccaacaaggccatcgtcggcgccaacgccttcgcccacgagtcgggcatccaccaggacggtgtgctcaagagccgggaaacctacgagatcatgagcgccgaggacgtggggtggagcaccaaccgcatggtgctgggcaaacattccggccgcaacgcgttccgtacccggatgcaggaactcggcatcgagttcgcctcggaagaggaactgaactcggtgttccagcgcttcaaggtgctggccgacaagaagcacgagatcttcgacgaggacctccaggccctcatcaccgaagccggcgcagaagccgaagacgaacgggtcaagctggtcgcgctgcgggtctgctcggaaacgggcgagattccccacgcccaggtcaccatcaaggtggacaacgaggaacgcaccggcacatcgagcggcggcggcgccgtggacgccagcctcaaggccatcgaatcgctgctgcacacggacaccgcgctgacgctgtactcggtcaacaacatcaccagcggcaccgacgcccagggcgaggtcaccgtgcggctcgagaaaggcgggcgcatcgtcaacggccagggcgccgataccgacatcgtgatcgcctcggccaaggcctacgtcaacgccgtgaacaagctgctggcgcccatccagcgcacccacccgcaagtcggggatgtgtga

SEQ ID NO: 58 - protein, 514 aa, Methylococcus capsulatus
MHDRLIIFDTTLRDGEQSPGASMTRDEKVRIARALERLKVDVIEAGFPAASPGDFEAVQAVARTIKDSRVCGLARALDRDIDRAGEALKDAQRARIHTFIATSPIHMRHKLQMSPDQVVEYAVKAVKRARQYTDDVEFSPEDAGRSEEDFLCRILEAVIDAGATTLNIPDTVGYAFPEQFGHMIGRLIERIPNSDKAVFSVHCHNDLGLAVANSLAAVLHGARQVECTINGLGERAGNAALEEIVMAVRTRKDIFPCHTDIETREIVACSKLVSSITGFPIQPNKAIVGANAFAHESGIHQDGVLKSRETYEIMSAEDVGWSTNRMVLGKHSGRNAFRTRMQELGIEFASEEELNSVFQRFKVLADKKHEIFDEDLQALITEAGAEAEDERVKLVALRVCSETGEIPHAQVTIKVDNEERTGTSSGGGAVDASLKAIESLLHTDTALTLYSVNNITSGTDAQGEVTVRLEKGGRIVNGQGADTDIVIASAKAYVNAVNKLLAPIQRTHPQVGDV

SEQ ID NO: 59 - DNA, 1413 bp, Methylococcus capsulatus
  source 1..1413: mol_type = other DNA
atgagcggaaaaaccctttacgacaagctgtgggacgaccacgtcgtgcatgtcgatgcggacggatcgtgcctgatctacatcgatcgtcatctaatccacgaggtgacctcgcctcaggcattcgaagggctgcggatggcggggcgtgtaccctggcgggtggatgccaatcttgcggtggccgaccacaacgtccccaccgccgaccgcgacaggggtatcgccgatccggtgtcgcgcctgcaggtggaaaccctggacaagaactgcgccgatttcggcatcaccgaattcgcgatggacgacgtgcgccagggtatcgtgcatgtgatcgggcccgagcagggcgcgaccctgccgggcatgaccatcgtttgcggcgattcgcatacttcgactcacggtgctttcggggcgctcgccttcgggatcggcacttccgaggtcgagcacgtactggccacgcaatgcctggtgcagcgcaaggcgaagaacatgctggtccgcgtccagggcaagctggcgccgggcgtgacggcgaaagatctggtactggcggtcatcggccgtatcggaaccgccggcggcaccggctacaccatcgaattcgctggcgaagccattcgcggcctgtcgatggaaggccggatgacggtctgcaacatggcgatcgaggcgggcgcacgtgccggcctggtggcggtggacgaagtcacgctcgactatctcgagggccgcccgttcgctccggcgggcgcgttgtgggagcgggcggtcgaggcatggaaagacctgcacagcgatccggatgcggtattcgacaaggtcgtcgagatcgatgccgccagcatcaagccgcaggtgacctggggaacttcgccggaacaggtcgtgccggtggatgccgaggtgcccgacccggccacggaagccgatcccgtgcggcgggaaagcatggagcgggcgctgcagtacatggatctcctgccgggcacgccaatcggcgcgatccgggtcgatcgggtgttcatcggctcctgcaccaatgccaggatcgaggatctgcgcgccgcggcggaagtcgtccgggggcacaagcgcgctgccagcgtgaagcaggcactggtggtgcccggctcgggtttggtcaagcggcaggcggagcaggaggggctggacaaggtgttcctcgaggccggtttcgaatggcgcgacccgggttgttccatgtgtctggcgatgaacgccgaccgcctggaacccggcgagcgttgcgcctcgacctccaaccggaattttgaggggcgccagggctatggcgggcgtacccatctggtgagtccggccatggcggctgcggcggccattcacgggcatttcgtcgacatcaccgaaggagggcgcgcatga

SEQ ID NO: 60 - protein, 470 aa, Methylococcus capsulatus
MSGKTLYDKLWDDHVVHVDADGSCLIYIDRHLIHEVTSPQAFEGLRMAGRVPWRVDANLAVADHNVPTADRDRGIADPVSRLQVETLDKNCADFGITEFAMDDVRQGIVHVIGPEQGATLPGMTIVCGDSHTSTHGAFGALAFGIGTSEVEHVLATQCLVQRKAKNMLVRVQGKLAPGVTAKDLVLAVIGRIGTAGGTGYTIEFAGEAIRGLSMEGRMTVCNMAIEAGARAGLVAVDEVTLDYLEGRPFAPAGALWERAVEAWKDLHSDPDAVFDKVVEIDAASIKPQVTWGTSPEQVVPVDAEVPDPATEADPVRRESMERALQYMDLLPGTPIGAIRVDRVFIGSCTNARIEDLRAAAEVVRGHKRAASVKQALVVPGSGLVKRQAEQEGLDKVFLEAGFEWRDPGCSMCLAMNADRLEPGERCASTSNRNFEGRQGYGGRTHLVSPAMAAAAAIHGHFVDITEGGRA

SEQ ID NO: 61 - DNA, 639 bp, Methylococcus capsulatus
  source 1..639: mol_type = other DNA
atgaagcctttcaagaaattcacttcgcgagtcgtgccgttggaccgcgccaatgtcgacaccgacgccatcattcccaagcagttcctgaagtccatccgccgcagcgggttcggtccctatctgttcgacgagtggcgttacctggaccgtggcgagcccgacatggattgcagccaccgtccgctcaacccggagttcgtgctcaacctgccctgttacgccggcgccaggatattgctggcccgcaagaacttcggctgtggctcctcgcgcgagcatgcgccctgggcgctggaggattacggcttccgcgccatcatcgcgccgagtttcgccgatatcttctacaacaactgcttcaagaacggcatcctgcccatcgtgctcgacgaggccacggtcgaccggctgtttagcgaggccgggcccggcttcgagctcaccgtcgacctggagtcgcagaccgtggcgacgccgttcggcgagaccttccatttcgacgtggatgcctcccgcaagcatcgtctgctgaacggcctggacgacatcggtctgacccttcagcatgccgatgccatccgcgcctacgaagccgcccgcaggaagtccgcaccctggctgtttgccgtcccttga

SEQ ID NO: 62 - protein, 212 aa, Methylococcus capsulatus
MKPFKKFTSRVVPLDRANVDTDAIIPKQFLKSIRRSGFGPYLFDEWRYLDRGEPDMDCSHRPLNPEFVLNLPCYAGARILLARKNFGCGSSREHAPWALEDYGFRAIIAPSFADIFYNNCFKNGILPIVLDEATVDRLFSEAGPGFELTVDLESQTVATPFGETFHFDVDASRKHRLLNGLDDIGLTLQHADAIRAYEAARRKSAPWLFAVP

SEQ ID NO: 63 - DNA, 1083 bp, Methylococcus capsulatus
  source 1..1083: mol_type = other DNA
atgactatcaaaatcgctgtcttgcccggtgacggcatcggtcccgaaatcgtcgccgaggccctgaaggttctggactgcctgcggtccgacttcggcctcgcggtcgaaaccgaacacgccctgatcggcggcgcagcctatgatgcgcacggcacgccgttccccaaggaaaccctggagctgtgccgggctgccgattcgatcctgcttggagcggtcggcggtcccaaatgggagccgttggattattcgctgcggcccgagcgggggctcctgggcttgcgttcggagctggaactgttttccaacctgcgcccggcggtgctctaccctcagctggtgtcggcttcgaccctcaagcccgaggtggtcgccggcctcgacatcatgatcgtgcgggagctgaccggcggcatatatttcggcaagccgcgcggtcgtcgcatcaacgaggacggagagcgggagggctacaacaccctggtatacagcgaatcggaaatccgccgcatagcccatagcgcgttccagatcgcccggaagcgtaacaggcgcctgtgcagcatcgacaaggccaatgtgctggaatgcacggaactgtggcgcgaggtggtgatcgaggtcggcaaggactatcccgacgtggcgctgagccacatgtacgtggacaacgccgcgatgcagctggtccgtaacccgaagcagttcgacgtgatgctgaccgacaacatgttcggcgacatcctgtccgactgtgccgccatgctgaccggctcgatcggcatgctgccttcggcttccctcgccgagagcggcaaggggatgtacgagcccatccacggttcggccccggatatcgccggccgcggcatcgccaacccgatcgccaccatcctgtcgctggccatgatgttgcgctacagcttcgatgacgcggtctcggcagagcggatcgggaaggcggtgcagacggcgctggatcagggtttccgcacggcggacatcgcctcggaaggcaccgtcgaggtcggtaccgctgcgatgggcgatgccatcgtcgccgccttgcgcgccgtctga

SEQ ID NO: 64 - protein, 360 aa, Methylococcus capsulatus
MTIKIAVLPGDGIGPEIVAEALKVLDCLRSDFGLAVETEHALIGGAAYDAHGTPFPKETLELCRAADSILLGAVGGPKWEPLDYSLRPERGLLGLRSELELFSNLRPAVLYPQLVSASTLKPEVVAGLDIMIVRELTGGIYFGKPRGRRINEDGEREGYNTLVYSESEIRRIAHSAFQIARKRNRRLCSIDKANVLECTELWREVVIEVGKDYPDVALSHMYVDNAAMQLVRNPKQFDVMLTDNMFGDILSDCAAMLTGSIGMLPSASLAESGKGMYEPIHGSAPDIAGRGIANPIATILSLAMMLRYSFDDAVSAERIGKAVQTALDQGFRTADIASEGTVEVGTAAMGDAIVAALRAV

SEQ ID NO: 65 - DNA, 1477 bp, Marinobacter algicola
  source 1..1477: mol_type = other DNA
atggcgacccagcagcagcagaacggcgcctcggcgagcggcgtcctggaacagttgcgcgggaagcatgtcctgataaccggtaccaccggtttccttggcaaggtagtcctggaaaagctgatccgcacagtcccggacatcggcggcatccacctcctgatccggggcaacaagaggcatccggccgcccgtgaacggttcttgaacgagatcgccagcagttcggtcttcgagcgtctgcgccacgacgacaacgaggccttcgaaaccttcctggaagaaagggtgcactgtataaccggagaggtcaccgagagtcgtttcggccttaccccggagcgcttccgcgcgctggcgggtcaggtggacgccttcatcaattcggccgcctccgtcaacttccgcgaggaactggacaaggcgctgaagatcaatacgctgtgcctggagaatgtcgcggcccttgctgaactcaacagtgcgatggcggtcatccaggtttcgacctgctacgttaacggcaagaatagcgggcagatcaccgaatcggtcatcaagcccgcgggggagtccatcccgcgtagcaccgatgggtactatgaaatcgaagaattggtgcacctgctgcaggacaaaatcagcgatgtgaaggcccgatactccgggaaggttctggaaaaaaaattggtggacctaggcatccgggaagccaataactacgggtggagcgatacatataccttcaccaagtggctgggcgaacagctcctcatgaaggccctgagcggcagatcgctgaccatcgtgcggccgtcgatcatcgagtcggcattggaagagcccagcccggggtggattgaaggcgtcaaggtcgccgatgccatcatactggcctacgcgagggagaaggtatcgctctttcctggcaagcggagcggcatcatcgacgtcatcccagtggatctggtggccaattcgatcattctgtccctggcggaggcgctctccggttcgggccagcggcgtatctatcagtgctgcagcggcggctcgaaccccatctccctcgggaagttcatcgactatctgatggcggaggcgaagaccaactacgcggcctacgatcagctgttctaccgccgccccaccaagccgttcgtggccgtcaaccgcaaactcttcgacgtcgtcgtgggcggcatgcgggtcccgctctcgatcgcgggcaaagccatgcgcctggcgggacaaaaccgcgaactgaaggtcctgaagaatctggatacgacccggtccctggccaccattttcgggttctacaccgctccggactacatctttcgcaatgacagcctgatggccctggcctcgcgcatgggcgagctggaccgcgtgttgttccccgttgacgcccgtcagatcgactggcagctgtatctgtgcaaaatccacctcggcgggctgaatcggtacg

SEQ ID NO: 66 - protein, 511 aa, Marinobacter algicola
MATQQQQNGASASGVLEQLRGKHVLITGTTGFLGKVVLEKLIRTVPDIGGIHLLIRGNKRHPAARERFLNEIASSSVFERLRHDDNEAFETFLEERVHCITGEVTESRFGLTPERFRALAGQVDAFINSAASVNFREELDKALKINTLCLENVAALAELNSAMAVIQVSTCYVNGKNSGQITESVIKPAGESIPRSTDGYYEIEELVHLLQDKISDVKARYSGKVLEKKLVDLGIREANNYGWSDTYTFTKWLGEQLLMKALSGRSLTIVRPSIIESALEEPSPGWIEGVKVADAIILAYAREKVSLFPGKRSGIIDVIPVDLVANSIILSLAEALSGSGQRRIYQCCSGGSNPISLGKFIDYLMAEAKTNYAAYDQLFYRRPTKPFVAVNRKLFDVVVGGMRVPLSIAGKAMRLAGQNRELKVLKNLDTTRSLATIFGFYTAPDYIFRNDSLMALASRMGELDRVLFPVDARQIDWQLYLCKIHLGGLNRYALKERKLYSLRAADTRKKA

SEQ ID NO: 67 - DNA, 1377 bp, Acinetobacter baylyi
  source 1..1377: mol_type = other DNA
atgcgccccctgcaccccatcgacttcatcttcctgagcctggaaaagcggcagcagcccatgcacgtcggcggcctgttcctgttccagatcccggacaacgcccccgacaccttcatccaggacctggtcaacgacatccgcatctccaagagcatcccggtgccgcccttcaacaacaagctgaacggcctgttctgggacgaagacgaggagttcgacctggaccaccatttccggcacatcgccctgccgcatcccggccgcatccgggaactgctgatctacatctcccaggagcacagcaccctgctggaccgcgcgaagccgctgtggacctgcaacatcatcgaaggcatcgagggcaaccggttcgccatgtatttcaagatccaccatgcgatggtcgacggcgtggccggcatgcgcctgatcgaaaagtcgctgtcccatgacgtcaccgagaagagcatcgtcccgccctggtgcgtggaaggcaagcgggcgaagcgcctgcgggagccgaagaccggcaagatcaagaagatcatgtcgggcatcaagtcccagctgcaggccacccccaccgtcatccaggaactgtcgcagaccgtgttcaaggacatcggccgcaacccggaccacgtcagctcgttccaggccccctgctccatcctgaaccagcgggtgtccagctcgcgccggttcgccgcgcagtcgttcgacctggaccgcttccggaacatcgcgaagtccctgaacgtcaccatcaacgacgtcgtgctggccgtgtgcagcggcgccctgcgcgcgtacctgatgagccacaactcgctgccgtccaagcccctgatcgcgatggtcccggcgtcgatccgcaacgacgacagcgacgtgtcgaaccggatcaccatgatcctggccaacctggcgacccataaggacgacccgctgcagcgcctggagatcatccgccggagcgtccagaactcgaagcagcgcttcaagcggatgacctccgaccagatcctgaactacagcgcggtcgtgtatggcccggccggcctgaacatcatcagcggcatgatgcccaagcgccaggccttcaacctggtcatctcgaacgtgccgggcccgcgcgagccgctgtactggaacggcgccaagctggacgcgctgtatcccgcctccatcgtcctggacggccaggccctgaacatcaccatgaccagctacctggacaagctggaggtcggcctgatcgcgtgccgcaacgccctgccgcggatgcagaacctgctgacccatctggaggaagagatccagctgttcgaaggcgtgatcgcgaagcaggaggacatcaagaccgccaactga

SEQ ID NO: 68 - protein, 458 aa, Acinetobacter baylyi
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 69 - DNA, 1428 bp, Psychrobacter articus
  source 1..1428: mol_type = other DNA
atgcgcctgctgaccgccgtcgaccagctgttcctgctgctggagtcccgcaagcacccgatgcacgtgggcggcctgttcctgttcgaactgccggagaacgccgacatctcgttcgtccaccagctggtgaagcagatgcaggactccgacgtcccgcccaccttccccttcaaccaggtgctggaacacatgatgttctggaaggaggacaagaacttcgacgtcgaacaccatctgcaccatgtggccctgccgaagcccgcgcgcgtccgggagctgctgatgtacgtgtcccgcgaacacggccggctgctggaccgcgcgatgccgctgtgggaatgccatgtcatcgagggcatccagccggaaaccgagggcagccccgagcggttcgccctgtatttcaagatccaccattcgctggtcgacggcatcgccgcgatgcgcctggtgaagaagagcctgtcgcagtcgccgaacgaacccgtgaccctgccgatctggagcctgatggcccaccatcggaaccagatcgacgcgatcttccccaaggagcggagcgccctgcgcatcctgaaggaacaggtctcgaccatcaagccggtgttcaccgagctgctgaacaacttcaagaactacaacgacgactcgtatgtctccaccttcgacgcgccccgcagcatcctgaaccgccggatcagcgcctcgcgccggatcgccgcgcagtcgtacgacatcaagcggttcaacgacatcgccgaacgcatcaacatctccaagaacgacgtcgtgctggccgtgtgcagcggcgcgatccgccgctacctgatcagcatggacgcgctgccgagcaagcccctgatcgccttcgtcccgatgtcgctgcgcaccgacgactccatcgcgggcaaccagctgtcgttcgtgctggccaacctgggcacccacctggacgaccccctgtcccggatcaagctgatccatcgctccatgaacaacagcaagcgccggttccgccggatgaaccaggcccaggtcatcaactacagcatcgtgtcgtatgcctgggagggcatcaacctggcgaccgacctgttcccgaagaagcaggccttcaacctgatcatctcgaacgtgccgggcagcgagaagcccctgtactggaacggcgcgcgcctggaaagcctgtatccggcctcgatcgtgttcaacggccaggccatgaacatcaccctggcgtcctacctggacaagatggagttcggcatcaccgcctgcagcaaggcgctgccgcacgtccaggacatgctgatgctgatcgaggaagagctgcagctgctggagtccgtcagcaaggaactggagttcaacggcatcaccgtgaaggacaagtcggaaaagaagctgaagaagctggccccgtga

SEQ ID NO: 70 - protein, 475 aa, Psychrobacter articus
MRLLTAVDQLFLLLESRKHPMHVGGLFLFELPENADISFVHQLVKQMQDSDVPPTFPFNQVLEHMMFWKEDKNFDVEHHLHHVALPKPARVRELLMYVSREHGRLLDRAMPLWECHVIEGIQPETEGSPERFALYFKIHHSLVDGIAAMRLVKKSLSQSPNEPVTLPIWSLMAHHRNQIDAIFPKERSALRILKEQVSTIKPVFTELLNNFKNYNDDSYVSTFDAPRSILNRRISASRRIAAQSYDIKRFNDIAERINISKNDVVLAVCSGAIRRYLISMDALPSKPLIAFVPMSLRTDDSIAGNQLSFVLANLGTHLDDPLSRIKLIHRSMNNSKRRFRRMNQAQVINYSIVSYAWEGINLATDLFPKKQAFNLIISNVPGSEKPLYWNGARLESLYPASIVFNGQAMNITLASYLDKMEFGITACSKALPHVQDMLMLIEEELQLLESVSKELEFNGITVKDKSEKKLKKLAP

SEQ ID NO: 71 - DNA, 1386 bp, Rhodococcus opacus
  source 1..1386: mol_type = other DNA
atgtccgtgatgtccccgaccgaggcgatgttcgtcctgttcgagaccccgagccacccgatgcacatgggcgcgctggagctgttcgagccgccgcgcgagtcgggcccggaccacgcccgcctgatgttcgaggcgctgatctcccaggaaggcgccagcgacaccttccgccggcgcgccgtccggccgctgcgcggcgcgtcgtacccctggtggtccgtcgacgaccgggtggacctgggctatcacgtccgccataccgccgtgccgggccggggccgcatggaggacctgctgtcgctggtgtcccagatgcacggcatgcccctggacccgcagcaccccatgtgggagatccatgtcatcgaaggcctggccgacggccgcaccgcggtgttcagcaagatccatctgtcgctgatggacggcccggccggcctgcggctgctgcaccatgcgctgagcaccgacccggacgcccgcgactgccccgcgccgtggacccccggcgtcagcggcacctcgcggcgcgaatcggccctgccggtcgccgcggtgcgggcgggcgtgcgcgccgcgacctccatcgtcggcgtgctgcccgccctggcgaaggtcgcctacgacggcgtgcgggaccagcacctgaccctgccgctgcagagcccgcccaccatgctgaacgtccccgtgggccgggcccgcaagctggccgcgcggagctggccgatccggcgcctggtctcggtggccgcggccgcgcgcaccaccatcaacgccgtcgtgctggcgatgtgctcgggcgccctgcgccactacctggtcgagcagtatgccctgccggaagcgcccctgaccgccatgctgcccgtgccgctggacctgggcggcaccatgatcggcccgcgtggccgcgaccacggcgtcggcgcgatggtcgtgggcctggcgaccgacgaggccgaccccgccgcgcggctggcccgcatcagcgagtcggtcgaacacaccaaccgcgtgttcggcgcgctgtcccatacccagttccaggtcatgtccgccctggcgatcagcccgatcctgctggaacccgtccggcgcttcgtggacgacaccccgcccccgttcaacgtgatgatctcgtacatgccgggtccgtcccggccgcgctattggaacggcgcgcggctggacgccgtctaccccgcgccgaccgtgctgggcggccaggccctgagcatcaccctgacctcccgcagcggccagctggacgtcggcgtcgtgggcgaccggcaggccgtgccgcacctgcagcgcatcatcacccatctggagacctccctgaccgacctggaaaacgccgtggccgcgagcggcacctga

SEQ ID NO: 72 - protein, 461 aa, Rhodococcus opacus
MSVMSPTEAMFVLFETPSHPMHMGALELFEPPRESGPDHARLMFEALISQEGASDTFRRRAVRPLRGASYPWWSVDDRVDLGYHVRHTAVPGRGRMEDLLSLVSQMHGMPLDPQHPMWEIHVIEGLADGRTAVFSKIHLSLMDGPAGLRLLHHALSTDPDARDCPAPWTPGVSGTSRRESALPVAAVRAGVRAATSIVGVLPALAKVAYDGVRDQHLTLPLQSPPTMLNVPVGRARKLAARSWPIRRLVSVAAAARTTINAVVLAMCSGALRHYLVEQYALPEAPLTAMLPVPLDLGGTMIGPRGRDHGVGAMVVGLATDEADPAARLARISESVEHTNRVFGALSHTQFQVMSALAISPILLEPVRRFVDDTPPPFNVMISYMPGPSRPRYWNGARLDAVYPAPTVLGGQALSITLTSRSGQLDVGVVGDRQAVPHLQRIITHLETSLTDLENAVAASGT

SEQ ID NO: 73 - DNA, 1356 bp, Rhodococcus opacus
  source 1..1356: mol_type = other DNA
atgccggtcaccgactccatcttcctgctgggcgaaagccgcgagcacccgatgcacgtgggctccctggaactgttcacccccccggacgacgccggcccggactacgtcaagtcgatgcacgagaccctgctgaagcataccgacgtggaccccaccttccgcaagaagccggcgggccccgtcggctcgctgggcaacgtgtggtgggccgacgagtccgacgtcgacctggaataccacgtgcgccatagcgcgctgccggccccctatcgcgtccgggaactgctgaccctgacctcgcggctgcacggcaccctgctggaccgccatcggccgctgtgggagatgtacctgatcgaaggcctgagcgacggccgcttcgccatctataccaagctgcaccatagcctgatggacggcgtctcgggcctgcgcctgctgatgcggaccctgtcgaccgacccggacgtgcgcgacgccccgcccccgtggaacctgccgcggcccgccgcggccaacggcgcggccccggacctgtggtcggtcgtgaacggcgtccgccggaccgtcggcgacgtggccggcctggcgcccgcctccctgcgcatcgcgcggaccgcgatgggccagcacgacatgcgcttcccgtacgaggcgccccggaccatgctgaacgtgccgatcggcggcgcccgccggttcgcggcccagtcctggcccctggaacgcgtccatgccgtgcggaaggcggccggcgtcagcgtgaacgacgtcgtgatggccatgtgcgcgggcgccctgcgcggctatctggaggaacagaacgcgctgccggacgagcccctgatcgcgatggtcccggtgtccctgcgggacgaacagcaggcggacgccggcggcaacgccgtcggcgtgaccctgtgcaacctggcgaccgacgtcgacgaccccgccgagcgcctgaccgcgatcagcgcctcgatgtcccagggcaaggaactgttcggcagcctgacctcgatgcaggcgctggcctggtcggcggtgaacatgtccccgatcgccctgaccccggtccccggcttcgtgcggttcacccccccgcccttcaacgtcatcatcagcaacgtgccgggcccccgcaagaccatgtactggaacggctcccggctggacggcatctatccgaccagcgtcgtgctggacggccaggccctgaacatcaccctgaccaccaacggcggcaacctggacttcggcgtcatcggctgccgccggtccgtgccgagcctgcagcgcatcctgttctacctggaagcggccctgggcgagctggaagcggccctgctgtga

SEQ ID NO: 74 - protein, 451 aa, Rhodococcus opacus
MPVTDSIFLLGESREHPMHVGSLELFTPPDDAGPDYVKSMHETLLKHTDVDPTFRKKPAGPVGSLGNVWWADESDVDLEYHVRHSALPAPYRVRELLTLTSRLHGTLLDRHRPLWEMYLIEGLSDGRFAIYTKLHHSLMDGVSGLRLLMRTLSTDPDVRDAPPPWNLPRPAAANGAAPDLWSVVNGVRRTVGDVAGLAPASLRIARTAMGQHDMRFPYEAPRTMLNVPIGGARRFAAQSWPLERVHAVRKAAGVSVNDVVMAMCAGALRGYLEEQNALPDEPLIAMVPVSLRDEQQADAGGNAVGVTLCNLATDVDDPAERLTAISASMSQGKELFGSLTSMQALAWSAVNMSPIALTPVPGFVRFTPPPFNVIISNVPGPRKTMYWNGSRLDGIYPTSVVLDGQALNITLTTNGGNLDFGVIGCRRSVPSLQRILFYLEAALGELEAALL

SEQ ID NO: 75 - DNA, 1395 bp, Rhodococcus opacus
  source 1..1395: mol_type = other DNA
atgcccctgccgatgtcccccctggactccatgttcctgctgggcgaaagccgcgagcacccgatgcacgtgggcggcgtcgaaatcttccagctgcccgagggcgccgacacctacgacatgcgggcgatgctggaccgcgccctggcggacggcgacggcatcgtcaccccgcggctggccaagcgcgcgcgccggtcgttcagctcgctgggccagtggtcctgggaaaccgtggacgacatcgacctgggccaccatatccggcacgacgccctgccggcccctggcggcgaggccgaactgatggcgctgtgctcgcgcctgcacggctccctgctggaccgcagccggccgctgtgggagatgcatctgatcgaaggcctgagcgacggccgcttcgccgtctataccaagatccaccatgccgtcgcggacggcgtgaccgccatgaagatgctgcggaacgcgctgagcgagaactcggacgaccgcgacgtgccggccccctggcagccgcgtggcccgcggccccagcgcaccccctccagcaagggcttctccctgagcggcctggccggctcgaccctgcggaccgcgcgcgagaccgtcggcgaagtggccggcctggtcccggccctggcgggcaccgtgagccgggccttccgcgaccagggcggcccgctggccctgtcggcgccgaagacccccttcaacgtccccatcaccggcgcccgccagttcgccgcgcagtcgtggccgctggaacgcctgcggctggtggccaagctgtcggactccaccatcaacgacgtcgtgctggccatgtcgtccggcgcgctgcggtcctacctggaggaccagaacgccctgccggcggaccccctgatcgcgatggtcccggtgtccctgaagagccagcgcgaagccgcgaccggcaacaacatcggcgtcctgatgtgcaacctgggcacccacctgcgggagccggccgaccgcctggaaaccatccggaccagcatgcgcgagggcaaggaagcctatggctcgatgaccgcgacccagatcctggccatgtccgcgctgggcgccgcgccgatcggcgccagcatgctgttcggccataactcgcgcgtccggccgcccttcaacctgatcatctccaacgtgccgggccccagctcgccgctgtactggaacggcgcccgcctggacgcgatctatccgctgagcgtccccgtggacggccagggcctgaacatcacctgcacctcgaacgacgacatcatctccttcggcgtcaccggctgccggtccgccgtgccggacctgaagagcatccccgcgcgcctgggccatgagctgcgggccctggaacgcgcggtgggcatctga

SEQ ID NO: 76 - protein, 464 aa, Rhodococcus opacus
MPLPMSPLDSMFLLGESREHPMHVGGVEIFQLPEGADTYDMRAMLDRALADGDGIVTPRLAKRARRSFSSLGQWSWETVDDIDLGHHIRHDALPAPGGEAELMALCSRLHGSLLDRSRPLWEMHLIEGLSDGRFAVYTKIHHAVADGVTAMKMLRNALSENSDDRDVPAPWQPRGPRPQRTPSSKGFSLSGLAGSTLRTARETVGEVAGLVPALAGTVSRAFRDQGGPLALSAPKTPFNVPITGARQFAAQSWPLERLRLVAKLSDSTINDVVLAMSSGALRSYLEDQNALPADPLIAMVPVSLKSQREAATGNNIGVLMCNLGTHLREPADRLETIRTSMREGKEAYGSMTATQILAMSALGAAPIGASMLFGHNSRVRPPFNLIISNVPGPSSPLYWNGARLDAIYPLSVPVDGQGLNITCTSNDDIISFGVTGCRSAVPDLKSIPARLGHELRALERAVGI

SEQ ID NO: 77 - DNA, 1407 bp, Rhodococcus opacus
  source 1..1407: mol_type = other DNA
atggccccgaccgactccctgttcctgctgggcgaatcccgcgagcacccgatgcacgtgggcggcctggcggtcttcaccccggcggagggcagctcggccgcggacgtccgcgccatgttcgacgccgcgctggtcggcgaccgggtggccgcgccgttccgcaagcgggcccgccggagcgtgacctcgctgggccagtggggctgggacaccctgcgcgacgacgaggtcgacctggaacaccatgtgcgccgggacgccctgccgcagccgggtggcatggcggaactgatgaccctggtctcccgcctgcatggcaccctgctggaccgcagccggccgctgtgggagatgcacctgatcgaaggcctggccgacggccggtacgcggtgtataccaagatccaccatgccctggcggacggcgccagcgcgatgcgcctgctgcgggactcgatgtccgaggacccgcatcgccggaacatgccgaccccctggcagccgcgcaaccccctgtcggccgtcccggacgccggcgtcgcggtgacccccggccccggcagcgccctgcccgcgatggcctgggacgccgcgcgctccgccgcgggcgaagtcgccggcctgctgccggccgcgctgggcaccgtggaccgggccctgcacggcaagggcggcgccctgtccctgaccgcgccgcataccctgttcaacgtccccatcagcggcgcccgccacgtggccgcgcggtcgttcccgatcgagcgcatccggctgctggccaagcatgccgacgcgaccatcaacgacatcgtgctgaccatgtgcgccggcaccctgcgcgcgtacctgcacacccgcgacgccctgccggacaaccccctgatcgcgatggtcccggtgagcctgcgcgcccccgaaaccggcaccggcgaccgcgcccctggcggcaaccgggtcggcgtgctgatgtgcaacctggccacccacctgccggaccccgcgcatcgcctggagaccgtccggaactgcatgaacgaaggcaaggccgcgctgcaggccatgtcgccggcgcaggtcctggccatgtccgcgctgggcgccgcgccgctgggcgtggagatgttcctgggccgccggggccccctgcgcccgcccttcaacgtcgtgatctcgaacgtggcgggcccgcgcacccccctgtactggaacggcgcccggctggaatccctgtatccgctgagcatccccaccaccggccaggccctgaacatcacctgcacctccagcgacgaccagatcgtcttcggcctgaccggctgccgccggaccgtgccggacctgcaccccatgctggaccagctggacgcggagctggacctgctggaaaccgcggtcggcctgtga

SEQ ID NO: 78 - protein, 468 aa, Rhodococcus opacus
MAPTDSLFLLGESREHPMHVGGLAVFTPAEGSSAADVRAMFDAALVGDRVAAPFRKRARRSVTSLGQWGWDTLRDDEVDLEHHVRRDALPQPGGMAELMTLVSRLHGTLLDRSRPLWEMHLIEGLADGRYAVYTKIHHALADGASAMRLLRDSMSEDPHRRNMPTPWQPRNPLSAVPDAGVAVTPGPGSALPAMAWDAARSAAGEVAGLLPAALGTVDRALHGKGGALSLTAPHTLFNVPISGARHVAARSFPIERIRLLAKHADATINDIVLTMCAGTLRAYLHTRDALPDNPLIAMVPVSLRAPETGTGDRAPGGNRVGVLMCNLATHLPDPAHRLETVRNCMNEGKAALQAMSPAQVLAMSALGAAPLGVEMFLGRRGPLRPPFNVVISNVAGPRTPLYWNGARLESLYPLSIPTTGQALNITCTSSDDQIVFGLTGCRRTVPDLHPMLDQLDAELDLLETAVGL

SEQ ID NO: 79 - DNA, 11299 bp, synthetic construct
  misc_feature 1..11299: note = pCM132
  source 1..11299: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcaattcggcttccaccggaattagcttgaaatagtacataatggatttccttacgcgaaatacgggcagacatggcctgcccggttattattatttttgacaccagaccaactggtaatggtagcgaccggcgctcagctgtaattccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccggtcgacccgtaatcttacgtcagtaacttccacagtagttcaccaccttttccctatagatcttccgtgcagtttaagccgaattgatccccgggtaccgagctcgaatctagaattccctgctttcctgatgcaaaaacgaggctagtttaccgtatctgtggggggatggcttgtagatatgacgacaggaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatgacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtccgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 80 - DNA, 7341 bp, synthetic construct
  misc_feature 1..7341: note = pJSvec
  source 1..7341: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcaggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaacccagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatcttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtccgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc

SEQ ID NO: 81 - DNA, 10705 bp, synthetic construct
  misc_feature 1..10705: note = pMZT3
  source 1..10705: mol_type = other DNA
tcaccctgtcgggcaatgccgaggcattctggcagcagcgccccctggcctgtagtggattacgtgccggtctgttccatcctaccaccggctattcactgccgctggcggttgccgtggccgaccgcctgagcgcacttgatgtctttacgtcggcctcaattcaccaggctattaggcattttgcccgcgagcgctggcagcagcagcgctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaagagacagaacgaagtgtgaccagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatcttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtccgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcagccagcgcttcgttaatacagatgtaggtgttccacagggtagccagcagcatcctgcgatgcagatccggaacataatggtgcagggcgctgactttatagctagctcagcccttggtacaatgctagcgagcaaccaacacttaaagaggagaaaatgtatccgtttataaggacagcccgaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggccctagggcggttgaagaacgtctgagacaacatcttcatcttgccagtgagcatctctctgcggcctgccaacacgggcacgccactcaacattttattcaggcctggtttgacaaaaaactcgctgccgtcagttaaaggtctctagacaggatgtgtcacacaggaaaccatgaaaccaactacggtaattggtgcaggcttcggtggcctggcactggcaattcgtctacaggctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggtgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgtatgagtaactctctgtttgtgctctattttggtttgaatcaccatcatgatcagctcgcgcatcacacggtttgtttcggcccgcgttaccgcgaactgattgacgagatttttaatcatgatggcctcgcagaagacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgagtaccttgagcagcattacatgcctggcttacggagtcagctggtcacgcaccagatgtttacgccgtttgattttcgcgaccagcttaatgcctatcagggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttaagtgatcgttgagtggtgaacttaaagaggagaaaatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgaccagacgctgggcttccaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggacgatacgctgcgctattgctatcacgttgcaggcgttgtcggcttgatgatggcgcaaatcatgggcgtacgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagcgggcctggctgggttgcccctgcgttcggcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctaatcacgtagcaagctgacagtttaaagaggagaaaatgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagcttcgctggtggttcatcactggcccgactatcaggtacgctttcccacacgccgtcgtaagctgaacagcggctacttctgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaactcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatatcgataatgcgacattagatcctgaacgcgcgcggcaaaatatttgcgactatgccgcgcaacagggttggcagcttcagacattgctgcgtgaagaacagggcgccttaccca

SEQ ID NO: 82 - DNA, 1773 bp, Methylosinus trichosporum
  source 1..1773: mol_type = other DNA
atggccagaaaaatgaccggagcggaaatggtcgtcgaagccctgaaggatcagggcgtcgagattatcttcggctatcccggcggcgccgtgcttccgatctatgacgcgctcttccaccaggagaaggtgcagcacattctcgtgcgccacgagcagggcgccgcccatgcggccgagggctatgcgcgctcctccggcaaggtcggcgtgctgctggtcacctccggacccggcgccaccaacaccatcaccggcctcaccgatgcgctgatggactccattcccgtggtctgcatcaccggccaggtgccgacgcatctcatcggctcggacgcctttcaagagtgcgatacggtcggcatcacccgtcactgcaccaagcataattatctggtgaagagcgtcgacgatctgccgcgcattctgcacgaggccttctatgtcgcctcgagcgggcggccgggccctgtggtcatcgacatccccaaggatgtgcaattcgccagcggaacctataccggcccgcgcaacgtccatcacaagacctatcagcccaagctcgagggcgacacggagtctatccgccgcgccgtgaagatgatggccgccgccaagcggccgatcttctacaccggcggcggcgtcatcaattccggtcccgcggcctcgacgctgctgcgcgagctggtgtcgctgaccggctttccgatcacctcgaccttgatgggcctcggcgcctatccgggctccggccccaattggctcggcatgctcggcatgcacggcaccttcgaggccaataatgcgatgcatgattgcgatctgatgatcgccgtcggcgcgcgtttcgacgatcgcatcaccggacggctcgacgccttctcgcccggctcgaagaagatccacatcgatatcgatcgctcctcgatcaataagaatgtgaagatcgatctgccgatcgtcggcgactgcggccatgtgctggagagtctggtgcgcgtctggcgctccgaggcgatgcacgccgagaagcagccgctcgacggctggtggaagacgatcgaccattggcgcgagcgcaagtcgctcgccttccgcaattcggacaaggtgatcaagccgcaatacgccgtgcagcggctctatgcgctcaccaaggatcgcgatccctacatcacgacggaagtcggccagcatcagatgtgggccgcgcagcattatcatttcgacgagcccaatcgctggatgacttccggcgggctcggcaccatgggctatggtctgccggcggcgatcggcgcgcagctcgcgcatccgaaatcgctggtcgtcgacatcgccggcgaggcctcgatcctgatgaacattcaggagatgtcgacggcgatccaatatcggctgccggtgaaggtgttcatcctcaacaatgaatatatgggcatggtgcgccagtggcaggagctgctgcacggcgggcgctactcgcactcctattcggaggcgctgcccgatttcgtgaagctcgccgaagccttcgggggcaagggcatccgctgctcggacccggcggagctcgatagcgcgattctcgagatgatcgactatgacgggccggtgatcttcgattgtctcgtcgagaaaaacgagaattgcttcccgatgatcccgtcgggcaaggcgcataacgacatgctgctcgccgatctcggcgacgacgccggcgtcgagctcggctcgatcatcgacgagaagggcaagatgctggtgtga

SEQ ID NO: 83 - DNA, 1056 bp, Methylosinus trichosporium
  source 1..1056: mol_type = other DNA
atgtccaccaaagcctatgccgttgcgtccgccgaggcgctcttcggcccgctcgcgatcgagcgccgcgcgctcgggcccgaggatgtagagatcgacatcctctattgcggcgtctgccattccgatctgcacacggcgcgcagtgaatggccgggcacgcgctacccatgcgtcccgggccacgagattgtcggccgcgtcaccgctgtcggcgcgaaggtgacgaaattttcggtcggcgatctcgccgccgtcggctgcatggtcgacagctgccggcgatgcttgtcctgcgacgacgggctcgaacaatattgcgagcacggtttcaccgccacctataacggcccgatctacggctcgggcgagaacacctttggcggctattcggagaaaatcgtcgtcgacgcgcatttcgtgctggcgatccaccattctgagacgcagcttgccggagtcgcgccgctgctctgcgccggcatcaccacttggtcgccgctcaagcattggggtgtcggcccgggaaaatcggtcggcatcgtcggcatcggcgggctcggccatatgggggtcaagctcgcccatgcgctcggcgcccatgtcgtcgccttcaccacctcgccgtcaaagcgcgacgcggccctcgcgctcggcgccgacgaggtcgtcgtctccacagatcctgccgctatggcggcgcgggcgggaagcctcgacttcattctcgatacggtcgccgtcgcccatgacctcgacgcttatgtgaatctgttgaagcgcgatggcgctctggtgctcgtcggcgtgccggcgacgccgcatccctcgccatcggcgggcgggttgatcttcaagcggcgccaggtcgccggctcgctgatcggcggcgtaaaggagacgcaggagatgctcgacttctgcgccgagcgcggcattgtcgcggacatagagacgatcgccatgcagcagatcgagaccgcctatgcgcgcatgctgaagaatgatgtgaaataccgcttcgtcatcgacatggcgacgctgaaggcggcgtga

SEQ ID NO: 84 - DNA, 1029 bp, Methylococcus capsulatus
  source 1..1029: mol_type = other DNA
atgaaagcttgggtgatcgaccgaatcggcccgctggactcgtcgcgaactctgctacgcgccaccgacctcccggtgccggagcccggccctggcgaaatcctgctgcaggtggcggtttgcggcgtctgccacaccgaaatcgacgagatcgagggccgcaccgcgccgccgcgcctgccggtcgtgcccggacaccaagcggtcggtcggatcgcggctctcggctccggcgtggcggaattcgctttgggcgaccgcgtcggcgtggcctggatcttttctgcctgcggagaatgcgaattctgccggtcgggacgggagaacctctgtttcgcattctgtgccaccgggcgcgatgtcgacggcggctacgcccagtacatgaccgtcccggcggcctttgctttccgcattccggagggattcaccgatgccgaagcggcgccgcttctgtgcgccggcgccatcggttaccgttcgctcaatctcagcgggctgaaaaacggccagccgctggggctcaccgggttcggggcttccgcccatctggtgctgatgatggcccggtaccggtttcccgattcggaagtctatgtctttgcgcgtcatcccgaggagcgcgcgttcgcgctgcagctgggcgcggtctgggccggcgacaccgcggacattgctcccgccccgctggccgccatcatcgacacgacgccggcgtggaagccggtggtcgcagcgctcgccaacctcgctcccggtggccggctggtcgttaatgcgatccgcaaggcgccggacgatcgcgcctgtctcgccgaactcgactatgcccggcacttgtggatggaacgggaaatcaagtcggtcgccaacgtggcgcgcagtgacgtggccgggttcctggcgctggcggcggaaatgggcatccgtcccgagacggaggagtacccgttcgaggatgccgaccgggcgctgctcgacctcaagcaacgccggattcgcggggcgaaggtgttgcgggtgacttga

SEQ ID NO: 85 - DNA, 1068 bp, Methylococcus capsulatus
  source 1..1068: mol_type = other DNA
atgcctacagccaaagcctatgccgctttttccgcagactcggcgctggcgccgttcgtcctgcagcggcgcgacccactgccccaggacatccgcatcggaatcctgtactgcggtgtctgccattccgacctgcaccaggcacgcaatgagtggaatgcgaccacatatccttgtgtgccaggccatgagatcgtcggcaaggtccttgaagtcggccgcagcgtgacgaagttcaagcccggcgacacggtcgcggtgggctgcatggtggattcctgccggacctgcccgaactgcgtggacgccctggaacagcactgcgagcacggccccgtcttcacctacaacagccccgatccgcacggcggcggcatgaccttcggtggctatgccgagagcatcgtggtcgacgaggccttcgtgctgcggataccggacggactggacctcgcggccgccgccccgctgttgtgcgccgggattaccacctattcgcccctgcggcactggaaagtgggggcgggtcagcgggtcggggtcgtcggtctgggtggactgggacacatggcgctcaagttcgcgcataccttcggcgccgaaacggtgctgttcacgacgacgccggacaaggcggaggatgcccgtcggctgggagcggacgaggtcgtcgtgtcgagggatcccgaggccatggcgcggcaggccggccggttcgatttcatcctcgacaccgtctcggcgccccatgacatcgatgcctatctgaacctgctgaggcgggacggcacgctgaccctggtcggcgtacctccgcaaggggtacaggtcatgcccttcagcctgatcggcgggcgccggcgactggctggttcattgatcggcggcatccgggaaacccaggagatgctggatttctgcggcgaacacggcatcgtctgcgacatcgagctgattccgatccaaggaatcaacgacgccttcgagcgcatgctcaaaagcgacgtgaaataccgtttcgtgatcgacatggcgacgctgaacggggagtcgtccggagggcgatga

SEQ ID NO: 86 - DNA, 1647 bp, Lactococcus lactis
  source 1..1647: mol_type = other DNA
atgtacaccgtgggcgactatctgctggaccggctgcatgaactgggcatcgaggaaatcttcggcgtccccggcgactataacctgcagttcctggaccagatcatcagccgcaaggacatgaagtgggtgggcaacgccaacgagctgaacgcctcgtacatggcggacggctatgcccggaccaagaaggccgcggccttcctgaccaccttcggcgtcggcgaactgagcgccgtgaacggcctggcgggctcgtacgccgagaacctgccggtcgtggaaatcgtcggctcccccaccagcaaggtgcagaacgagggcaagttcgtccaccataccctggccgacggcgacttcaagcacttcatgaagatgcatgaaccggtgaccgcggcccgcaccctgctgaccgccgagaacgcgaccgtcgaaatcgaccgcgtgctgagcgcgctgctgaaggagcggaagccggtctatatcaacctgcccgtcgacgtggcggccgcgaaggccgagaagccgtccctgcccctgaagaaggaaaaccccacctcgaacacctccgaccaggagatcctgaacaagatccaggaaagcctgaagaacgccaagaagccgatcgtgatcaccggccacgagatcatctcgttcggcctggaaaacaccgtcacccagttcatctccaagaccaagctgccgatcaccaccctgaacttcggcaagagctcggtggacgagaccctgccctcgttcctgggcatctacaacggcaagctgtccgaaccgaacctgaaggagttcgtggaaagcgcggacttcatcctgatgctgggcgtcaagctgaccgactccagcaccggcgccttcacccaccatctgaacgagaacaagatgatctcgctgaacatcgacgagggcaagatcttcaacgaatccatccagaacttcgacttcgaaagcctgatctcgtccctgctggacctgtccggcatcgagtacaagggcaagtatatcgacaagaagcaggaagacttcgtcccgagcaacgcgctgctgtcgcaggaccgcctgtggcaggccgtggagaacctgacccagagcaacgagaccatcgtcgcggaacagggcacctcgttcttcggcgccagctcgatcttcctgaagccgaagtcgcacttcatcggccagcccctgtggggctccatcggctacaccttccccgccgcgctgggctcgcagatcgcggacaaggaatcccggcatctgctgttcatcggcgacggcagcctgcagctgaccgtgcaggagctgggcctggccatccgcgaaaagatcaacccgatctgcttcatcatcaacaacgacggctataccgtcgagcgggaaatccacggcccgaaccagtcgtacaacgacatccccatgtggaactattccaagctgccggagagcttcggcgccaccgaggaacgcgtcgtgtccaagatcgtccggaccgagaacgagttcgtcagcgtgatgaaggaagcccaggcggaccccaaccggatgtactggatcgagctggtgctggcgaaggaagacgccccgaaggtcctgaagaagatgggcaagctgttcgccgaacagaacaagagctga

SEQ ID NO: 87 - DNA, 1692 bp, Saccharomyces cerevisiae
  source 1..1692: mol_type = other DNA
atgtcggaaatcaccctgggcaagtacctgttcgagcggctgaagcaggtcaacgtcaacaccatcttcggcctgcccggcgacttcaacctgagcctgctggacaagatctacgaggtcgacggcctgcgctgggccggcaacgcgaacgaactgaacgccgcgtacgccgcggacggctatgcccggatcaagggcctgtcggtcctggtgaccaccttcggcgtgggcgagctgtcggccctgaacggcatcgccggctcctacgcggaacacgtcggcgtgctgcatgtcgtgggcgtcccgagcatctcggcccaggcgaagcagctgctgctgcaccataccctgggcaacggcgacttcaccgtgttccaccgcatgtccgccaacatcagcgagaccacctcgatgatcaccgacatcgccaccgcgccgagcgaaatcgaccgcctgatccggaccaccttcatcacccagcggccgtcgtacctgggcctgcccgccaacctggtcgacctgaaggtgccgggcagcctgctggagaagcccatcgacctgtcgctgaagccgaacgaccccgaggccgaaaaggaagtcatcgacaccgtgctggaactgatccagaacagcaagaacccggtcatcctgtccgacgcctgcgcgagccgccacaacgtgaagaaggagacccagaagctgatcgacctgacccagttcccggccttcgtcacccccctgggcaagggctccatcgacgagcagcatccgcggtacggcggcgtctatgtgggcaccctgagcaagcaggacgtcaagcaggccgtggaaagcgcggacctgatcctgtcggtgggcgccctgctgtccgacttcaacaccggctccttcagctactcgtataagaccaagaacgtcgtggagttccattcggactacgtcaaggtgaagaacgcgaccttcctgggcgtccagatgaagttcgccctgcagaacctgctgaaggtgatcccggacgtcgtgaagggctataagtccgtcccggtgcccaccaagacccccgccaacaagggcgtcccggcgtcgacccccctgaagcaggaatggctgtggaacgagctgtccaagttcctgcaggaaggcgacgtgatcatctcggagaccggcacctccgcgttcggcatcaaccagaccatcttcccgaaggacgcctacggcatcagccaggtcctgtggggctcgatcggcttcaccaccggcgccaccctgggcgccgcgttcgccgcggaggaaatcgacccgaacaagcgcgtcatcctgttcatcggcgacggctccctgcagctgaccgtgcaggaaatcagcaccatgatccggtggggcctgaagccctacctgttcgtgctgaacaacgacggctataccatcgagaagctgatccacggcccgcatgcggaatacaacgagatccagacctgggaccacctggccctgctgcccgccttcggcgcgaagaagtatgaaaaccataagatcgccaccaccggcgagtgggacgcgctgaccaccgactccgagttccagaagaacagcgtcatccgcctgatcgagctgaagctgccggtgttcgacgcccccgaaagcctgatcaagcaggcgcagctgaccgccgcgaccaacgccaagcagtga

SEQ ID NO: 88 - DNA, 1908 bp, Saccharomyces cerevisiae
  source 1..1908: mol_type = other DNA
atggcccccgtcaccatcgagaagttcgtcaaccaggaagagcggcatctggtgtccaaccggagcgcgaccatcccgttcggcgagtacatcttcaagcgcctgctgagcatcgacaccaagtcggtgttcggcgtgccgggcgacttcaacctgagcctgctggagtacctgtatagcccctcggtcgaatcggccggcctgcgctgggtgggcacctgcaacgaactgaacgccgcgtacgccgcggacggctactcccggtatagcaacaagatcggctgcctgatcaccacctatggcgtcggcgaactgtcggcgctgaacggcatcgcgggctccttcgccgagaacgtgaaggtcctgcacatcgtgggcgtcgccaagtcgatcgactcccgcagctcgaacttctcggaccggaacctgcaccatctggtcccgcagctgcatgactccaacttcaagggccccaaccacaaggtgtaccatgacatggtgaaggaccgcgtcgcgtgctccgtggcctatctggaggacatcgaaaccgcctgcgaccaggtggacaacgtcatccgggacatctacaagtatagcaagccgggttacatcttcgtccccgcggacttcgccgacatgtccgtgacctgcgacaacctggtgaacgtcccgcgcatcagccagcaggactgcatcgtgtacccctccgaaaaccagctgagcgacatcatcaacaagatcacctcgtggatctactccagcaagaccccggccatcctgggcgacgtcctgaccgaccggtatggcgtgagcaacttcctgaacaagctgatctgcaagaccggcatctggaacttctcgaccgtcatgggcaagtcggtgatcgacgaatccaacccgacctacatgggccagtataacggcaaggaaggcctgaagcaggtctacgagcacttcgaactgtgcgacctggtcctgcatttcggcgtggacatcaacgagatcaacaacggccactacaccttcacctataagccgaacgcgaagatcatccagttccatcccaactacatccgcctggtggacacccggcagggcaacgaacagatgttcaagggcatcaacttcgccccgatcctgaaggagctgtataagcgcatcgacgtcagcaagctgtcgctgcagtacgacagcaacgtgacccagtataccaacgagaccatgcggctggaagaccccaccaacggccagtcgtccatcatcacccaggtccacctgcagaagaccatgccgaagttcctgaaccccggcgacgtcgtggtctgcgagaccggctccttccagttcagcgtgcgcgacttcgcgttcccgagccagctgaagtacatctcgcagggcttcttcctgtccatcggcatggccctgcccgccgcgctgggcgtcggcatcgcgatgcaggaccactcgaacgcccatatcaacggcggcaacgtgaaggaagactacaagccgcggctgatcctgttcgaaggcgacggcgccgcgcagatgaccatccaggagctgtccaccatcctgaagtgcaacatcccgctggaagtcatcatctggaacaacaacggctacaccatcgagcgcgccatcatgggccccacccggagctataacgacgtgatgtcgtggaagtggaccaagctgttcgaagcgttcggcgacttcgacggcaagtacaccaactccaccctgatccagtgcccgagcaagctggccctgaagctggaggaactgaagaactcgaacaagcgctccggcatcgagctgctggaagtcaagctgggcgagctggacttccccgaacagctgaagtgcatggtggaggccgcggccctgaagcggaacaagaagtga

SEQ ID NO: 89 - DNA, 1047 bp, Saccharomyces cerevisiae
  source 1..1047: mol_type = other DNA
atgagcatccccgagacccagaaggccatcatcttctacgagagcaacggcaagctggaacataaggacatcccggtgcccaagcccaagccgaacgaactgctgatcaacgtgaagtacagcggcgtctgccacaccgacctgcacgcgtggcatggcgactggccgctgcccaccaagctgcccctggtgggcggccatgaaggcgccggcgtcgtggtcggcatgggcgagaacgtcaagggctggaagatcggcgactacgcgggcatcaagtggctgaacggcagctgcatggcctgcgagtattgcgaactgggcaacgaatcgaactgcccgcacgcggacctgtccggctacacccatgacggcagcttccaggagtatgccaccgcggacgccgtgcaggccgcgcacatcccgcagggcaccgacctggcggaggtggcccccatcctgtgcgccggcatcaccgtctacaaggcgctgaagagcgccaacctgcgcgcgggccattgggccgcgatctcgggcgccgccggtggcctgggctccctggccgtgcagtacgcgaaggcgatgggctaccgcgtcctgggcatcgacggcggtccgggcaaggaagagctgttcacctccctgggcggcgaagtgttcatcgacttcaccaaggagaaggacatcgtcagcgccgtggtcaaggcgaccaacggcggcgcccacggcatcatcaacgtgtcggtctccgaagccgcgatcgaggcgtcgacccgctactgccgggccaacggcaccgtggtcctggtgggcctgcccgcgggcgccaagtgcagctcggacgtcttcaaccatgtggtcaagagcatctcgatcgtgggctcgtatgtcggcaaccgcgccgacacccgcgaggccctggacttcttcgcccgtggcctggtcaagtccccgatcaaggtggtcggcctgtccagcctgcccgagatctacgaaaagatggagaagggccagatcgccggccgctatgtggtcgacacctccaagtga

SEQ ID NO: 90 - DNA, 1692 bp, Saccharomyces cerevisiae
  source 1..1692: mol_type = other DNA
atgagcgagatcaccctgggcaagtacctgttcgagcggctgaagcaggtcaacgtcaacaccgtcttcggcctgcccggcgacttcaacctgagcctgctggacaagatctacgaggtcgaaggcatgcgctgggcgggcaacgccaacgagctgaacgccgcgtacgccgcggacggctatgcccggatcaagggcatgtcgtgcatcatcaccaccttcggcgtgggcgagctgtccgccctgaacggcatcgcgggcagctacgccgaacacgtcggcgtgctgcatgtcgtgggcgtcccgagcatctcggcccaggcgaagcagctgctgctgcaccataccctgggcaacggcgacttcaccgtgttccaccgcatgtccgcgaacatcagcgagaccaccgccatgatcaccgacatcgccaccgcgccggccgaaatcgaccgctgcatccggaccacctacgtcacccagcggcccgtgtatctgggcctgccggccaacctggtcgacctgaacgtgcccgcgaagctgctgcagaccccgatcgacatgtcgctgaagcccaacgacgccgagtccgaaaaggaagtcatcgacaccatcctggcgctggtcaaggacgccaagaacccggtgatcctggcggacgcctgctgctcccgccacgacgtcaaggccgagaccaagaagctgatcgacctgacccagttccccgccttcgtgaccccgatgggcaagggctccatcgacgaacagcatccgcggtacggcggcgtctatgtgggcaccctgagcaagcccgaagtcaaggaagccgtggaaagcgccgacctgatcctgtcggtcggcgccctgctgtccgacttcaacaccggctccttcagctactcgtataagaccaagaacatcgtggagttccacagcgaccacatgaagatccgcaacgccaccttccccggcgtccagatgaagttcgtgctgcagaagctgctgaccaccatcgccgacgccgcgaagggctacaagccggtcgcggtgcccgcccggaccccggcgaacgccgcggtccccgcctcgaccccgctgaagcaggaatggatgtggaaccagctgggcaacttcctgcaggaaggcgacgtcgtgatcgcggaaaccggcacctccgccttcggcatcaaccagaccaccttcccgaacaacacctacggcatcagccaggtgctgtggggctcgatcggcttcaccaccggcgccaccctgggcgccgcgttcgccgcggaggaaatcgacccgaagaagcgcgtcatcctgttcatcggcgacggcagcctgcagctgaccgtgcaggaaatctcgaccatgatccggtggggcctgaagccctacctgttcgtcctgaacaacgacggctataccatcgagaagctgatccacggcccgaaggcccagtacaacgaaatccagggctgggaccatctgtcgctgctgcccaccttcggcgccaaggactatgagacccatcgcgtggcgaccaccggcgaatgggacaagctgacccaggacaagtcgttcaacgacaactccaagatccggatgatcgagatcatgctgcccgtcttcgacgcgccgcagaacctggtggaacaggccaagctgaccgccgcgaccaacgcgaagcagtga

SEQ ID NO: 91 - DNA, 3147 bp, Saccharomyces cerevisiae
  source 1..3147: mol_type = other DNA
atgaagtcggaatacaccatcggccgctatctgctggaccgcctgagcgagctgggcatccgccacatcttcggcgtccccggcgactacaacctgtcgttcctggactacatcatggagtataagggcatcgactgggtcggcaactgcaacgaactgaacgccggctacgccgcggacggctatgcccgcatcaacggcatcggcgcgatcctgaccaccttcggcgtcggcgagctgtccgccatcaacgccatcgcgggcgcctacgcggaacaggtgccggtcgtgaagatcaccggcatccccaccgccaaggtccgcgacaacggcctgtatgtgcaccataccctgggcgacggccgcttcgaccacttcttcgagatgttccgggaagtcaccgtggccgaggcgctgctgagcgaggaaaacgccgcgcaggaaatcgaccgcgtgctgatctcgtgctggcgccagaagcggccggtcctgatcaacctgcccatcgacgtgtacgacaagccgatcaacaagccgctgaagcccctgctggactataccatcagctcgaacaaggaagccgcgtgcgagttcgtcaccgagatcgtgccgatcatcaaccgcgccaagaagcccgtcatcctggcggactacggcgtgtaccggtatcaggtccagcacgtgctgaagaacctggcggagaagaccggcttcccggtcgccaccctgtcgatgggcaagggcgtgttcaacgaagcccatccgcagttcatcggcgtctacaacggcgacgtgtccagcccctatctgcgccagcgggtcgacgaggccgactgcatcatctcggtcggcgtgaagctgaccgactccaccaccggcggcttctcccacggcttcagcaagcgcaacgtgatccatatcgacccgttctccatcaaggccaagggcaagaagtacgcgcccatcaccatgaaggacgccctgaccgaactgacctcgaagatcgagcaccggaacttcgaagacctggacatcaagccgtacaagtccgacaaccagaagtatttcgcgaaggagaagcccatcacccagaagcgcttcttcgaacggatcgcccatttcatcaaggagaaggacgtcctgctggcggaacagggcacctgcttcttcggcgccagcaccatccagctgccgaaggacgcgaccttcatcggccagcccctgtggggctccatcggctacaccctgccggccctgctgggcagccagctggcggaccagaagcgtcgcaacatcctgctgatcggcgacggcgccttccagatgaccgcgcaggagatctcgaccatgctgcgcctgcagatcaagccgatcatcttcctgatcaacaacgacggctacaccatcgagcgcgccatccacggccgggaacaggtgtacaacaacatccagatgtggcggtatcataacgtcccgaaggtgctgggccccaaggaatgcagcctgaccttcaaggtccagtcggagaccgaactggagaaggccctgctggtcgccgacaaggactgcgagcacctgatcttcatcgaagtcgtgatggaccgctacgacaagccggagcccctggaacgcctgtccaagcggttcgccaaccagaacaacggctatgcgcggatcaacggcatcggcgccattttaaccaccttcggcgtgggcgagctgagcgcgatcaacgcgatcgccggcgcctacgcggagcaggtgccggtggtcaaaattaccggcatccccaccgcgaaggtgcgggacaacggcctgtacgtccatcacaccctgggcgacggccggttcgaccatttcttcgaaatgttccgggaggtgaccgtcgccgaggcgctgctgtcggaagagaacgcggcccaggagatcgaccgcgtcctgatcagctgctggcggcagaagcgccccgtgctgatcaacctgccgatcgacgtctatgacaagcccatcaacaagcccctgaagccgctgctggactacaccatctcgtccaacaaggaagccgcctgcgagttcgtcaccgaaatcgtccccatcatcaaccgcgcgaagaagccggtgatcctggccgactatggcgtctatcggtatcaggtgcagcatgtcctgaagaacctggccgaaaagaccggcttccccgtggccaccctgagcatgggcaagggcgtcttcaacgaggcgcacccccagttcatcggcgtgtataacggcgacgtgagctcgccgtacctgcggcagcgcgtggacgaagccgactgcatcatcagcgtcggcgtcaagctgaccgactcgaccaccggcggcttctcgcacggcttctcgaagcggaacgtcatccacatcgacccgttctcgatcaaggcgaagggcaagaagtatgccccgatcaccatgaaggacgcgctgaccgaactgaccagcaagatcgaacatcgcaacttcgaggacctggacatcaagccctacaagtcggacaaccagaagtacttcgccaaggaaaagccgattactcagaagcgcttcttcgagcgcatcgcgcacttcatcaaggaaaaggacgtcctgctggccgagcaaggcacctgcttcttcggtgcgtcgaccatccagctgcccaaggacgccaccttcatcggccagccgctgtggggctcgatcggctataccctgcccgcgctgctgggctcccagctggccgatcaaaaacgtcgcaatattttactgatcggcgacggcgcgttccagatgaccgcccaggagatcagcaccatgctgcggctgcagatcaagcccattatcttcctgattaacaacgacggctataccatcgaacgggcgatccacggccgcgagcaggtctataataatattcaaatgtggcggtatcataatgtgcccaaggtcctgggcccgaaggaatgctcgctgaccttcaaggtgcagagcgaaaccgagctggaaaaggccctgctggtcgccgataaggactgcgaacatctgatcttcatcgaggtggtcatggaccggtatgacaagcccgaacccctggaacggctgagcaagcgcttcgcgaaccagaacaactga

SEQ ID NO: 92 - DNA, 35 bp, synthetic construct
  misc_feature 1..35: note = JPS00082
  source 1..35: mol_type = other DNA
tgcaaggtacactgtcagaacgcagaagcggtctg

SEQ ID NO: 93 - DNA, 28 bp, synthetic construct
  misc_feature 1..28: note = JPS00031
  source 1..28: mol_type = other DNA
ggtttattcctccttatttaatcgatac

SEQ ID NO: 94 - DNA, 34 bp, synthetic construct
  misc_feature 1..34: note = JPS00032
  source 1..34: mol_type = other DNA
aaggaggaataaaccatgggcacggttgagcctg

SEQ ID NO: 95 - DNA, 33 bp, synthetic construct
  misc_feature 1..33: note = GMV257
  source 1..33: mol_type = other DNA
cacatcctgtctagatcagccctcgcccttgac

SEQ ID NO: 96 - DNA, 58 bp, synthetic construct
  misc_feature 1..58: note = JPS00118)
  source 1..58: mol_type = other DNA
tctagacaggatgtgtcacacaggaaaccatgtcttatcctgagaaatttgaaggtat

SEQ ID NO: 97 - DNA, 41 bp, synthetic construct
  misc_feature 1..41: note = JPS00119
  source 1..41: mol_type = other DNA
acagtgtaccttgcactagtctgaaaattctttgtcgtagc

SEQ ID NO: 98 - DNA, 70 bp, synthetic construct
  misc_feature 1..70: note = ESG00087
  source 1..70: mol_type = other DNA
gtgttggttgctcgctagcattgtaccaagggctgagctagctataaagtcagcgccctgcaccattatg

SEQ ID NO: 99 - DNA, 46 bp, synthetic construct
  misc_feature 1..46: note = GMV251
  source 1..46: mol_type = other DNA
gagcaaccaacacttaaagaggagaaaatgggcacggttgagcctg

SEQ ID NO: 100 - DNA, 149 bp, synthetic construct
  misc_feature 1..149: note = IDT gBlock synthesized rnpB
  source 1..149: mol_type = other DNA
gctagcactagtgatcacgtgcttaagccggcttatcggtcagtttcacctgatttacgtaaaaacccgcttcggcgggtttttgcttttggaggggcagaaagatgaatgactgtccacgacgctatacccaaaagaaaaccggtacc

SEQ ID NO: 101 - DNA, 35 bp, synthetic construct
  misc_feature 1..35: note = JPS00161
  source 1..35: mol_type = other DNA
gcctgatacagattattgtaggtggaccagttggt

SEQ ID NO: 102 - DNA, 34 bp, synthetic construct
  misc_feature 1..34: note = JPS00162
  source 1..34: mol_type = other DNA
ggtttattcctccttgatttgtcctactcaggag

SEQ ID NO: 103 - DNA, 34 bp, synthetic construct
  misc_feature 1..34: note = JPS00163
  source 1..34: mol_type = other DNA
aaggaggaataaaccgctagcactagtgatcacg

SEQ ID NO: 104 - DNA, 34 bp, synthetic construct
  misc_feature 1..34: note = JPS00164
  source 1..34: mol_type = other DNA
taatctgtatcaggcggtaccggttttcttttgg

SEQ ID NO: 105 - DNA, 34 bp, synthetic construct
  misc_feature 1..34: note = JPS00172
  source 1..34: mol_type = other DNA
atcagactaagccttgtgcttaagccggcttatc

SEQ ID NO: 106 - DNA, 51 bp, synthetic construct
  misc_feature 1..51: note = JPS00173
  source 1..51: mol_type = other DNA
aaggaggaataaaccgctagcactagtgatcacttgacggctagctcagtc

SEQ ID NO: 107 - DNA, 33 bp, synthetic construct
  misc_feature 1..33: note = JPS00174
  source 1..33: mol_type = other DNA
tgaacaggtctgacttcagtgctgcgccgaggc

SEQ ID NO: 108 - DNA, 51 bp, synthetic construct
  misc_feature 1..51: note = JPS00176
  source 1..51: mol_type = other DNA
agtcagacctgttcattaaagaggagaaaatgcagatttactacgacaaag

SEQ ID NO: 109 - DNA, 34 bp, synthetic construct
  misc_feature 1..34: note = JPS00177
  source 1..34: mol_type = other DNA
aagtgttggttgctctcagttcttgctcgtgtcc

SEQ ID NO: 110 - DNA, 45 bp, synthetic construct
  misc_feature 1..45: note = JPS00157
  source 1..45: mol_type = other DNA
gagcaaccaacacttaaagaggagaaaatgaccgacaagcacccc

SEQ ID NO: 111 - DNA, 33 bp, synthetic construct
  misc_feature 1..33: note = JPS00178
  source 1..33: mol_type = other DNA
aaggcttagtctgattcagaggccgtcgtcggt

SEQ ID NO: 112 - DNA, 430 bp, synthetic construct
  misc_feature 1..430: note = IDT gBlock synthesized Me-AM1 PmxaF
  source 1..430: mol_type = other DNA
atggtgcagggcgcttcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcgaggccgaaaggcgaggcggttcagcgaggagacgcaggatgagcaggtttgtgacatcagtctcggccttggcggctagcgagcaaccaacactt

SEQ ID NO: 113 - DNA, 34 bp, synthetic construct
  misc_feature 1..34: note = JPS00169
  source 1..34: mol_type = other DNA
agcgccctgcaccattatgttccggatctgcatc

SEQ ID NO: 114 - DNA, 46 bp, synthetic construct; identical to SEQ ID NO: 99
  misc_feature 1..46: note = GMV00251
  source 1..46: mol_type = other DNA

SEQ ID NO: 115 - DNA, 32 bp, synthetic construct
  misc_feature 1..32: note = (PS00170
  source 1..32: mol_type = other DNA
atggtgcagggcgcttcccgcttggtcgggcc

SEQ ID NO: 116 - DNA, 19 bp, synthetic construct
  misc_feature 1..19: note = JPS00171
  source 1..19: mol_type = other DNA
aagtgttggttgctcgcta

SEQ ID NO: 117 - DNA, 68 bp, synthetic construct
  misc_feature 1..68: note = JPS00153
  source 1..68: mol_type = other DNA
accactcaacgatcagctagcactgtacctaggactgagctagccgtcaagtcagcgccctgcaccat

SEQ ID NO: 118 - DNA, 48 bp, synthetic construct
  misc_feature 1..48: note = JPS00151
  source 1..48: mol_type = other DNA
tgatcgttgagtggtttaaagaggagaaaatgcgtgaaacgatacctc

SEQ ID NO: 119 - DNA, 33 bp, synthetic construct
  misc_feature 1..33: note = JPS00154
  source 1..33: mol_type = other DNA
aagtgttggttgctctcagtgctgcgccgaggc

SEQ ID NO: 120 - DNA, 33 bp, synthetic construct
  misc_feature 1..33: note = JPS00183
  source 1..33: mol_type = other DNA
tgagctagctataaagtgatcactagtgctagc

SEQ ID NO: 121 - DNA, 55 bp, synthetic construct
  misc_feature 1..55: note = JPS00185
  source 1..55: mol_type = other DNA
ttatagctagctcagcccttggtacaatgctagctgatcgttgagtggtttaaag

SEQ ID NO: 122 - DNA, 35 bp, synthetic construct
  misc_feature 1..35: note = J23100
  source 1..35: mol_type = other DNA
ttgacggctagctcagtcctaggtacagtgctagc

SEQ ID NO: 123 - DNA, 35 bp, synthetic construct
  misc_feature 1..35: note = J23100 hybrid
  source 1..35: mol_type = other DNA
ttgacggctagctcagcccttggtacaatgctagc

SEQ ID NO: 124 - DNA, 35 bp, synthetic construct
  misc_feature 1..35: note = J23115
  source 1..35: mol_type = other DNA
tttatagctagctcagcccttggtacaatgctagc

SEQ ID NO: 125 - DNA, 22 bp, synthetic construct
  misc_feature 1..22: note = GMV00233
  source 1..22: mol_type = other DNA
tgccagctgcattaatgaatcg

SEQ ID NO: 126 - DNA, 45 bp, synthetic construct
  misc_feature 1..45: note = GMV00235
  source 1..45: mol_type = other DNA
ttaatgcagctggcagccagcgcttcgttaatacagatgtaggtg

SEQ ID NO: 127 - DNA, 52 bp, synthetic construct
  misc_feature 1..52: note = GMV00433
  source 1..52: mol_type = other DNA
gatcgttgagtggtgaacttaaagaggagaaaatgggcacggttgagcctgg

SEQ ID NO: 128 - DNA, 19 bp, synthetic construct
  misc_feature 1..19: note = GMV00434
  source 1..19: mol_type = other DNA
tgcattcgattcctgtttg

SEQ ID NO: 129 - DNA, 19 bp, synthetic construct
  misc_feature 1..19: note = GMV00435
  source 1..19: mol_type = other DNA
caggaatcgaatgcaaccg

SEQ ID NO: 130 - DNA, 49 bp, synthetic construct
  misc_feature 1..49: note = GMV00436
  source 1..49: mol_type = other DNA
gagcaaccaacactcacacaggaaaccatgcatattacatacgatctgc

SEQ ID NO: 131 - DNA, 39 bp, synthetic construct
  misc_feature 1..39: note = GMV00437
  source 1..39: mol_type = other DNA
gttcaccactcaacgatcttaagcgtcaacgaaaccggt

SEQ ID NO: 132 - DNA, 69 bp, synthetic construct
  misc_feature 1..69: note = GMV00438
  source 1..69: mol_type = other DNA
tgaacaggtctgactgctagcattgtaccaagggctgagctagctataaagatttgtcctactcaggag

SEQ ID NO: 133 - DNA, 53 bp, synthetic construct
  misc_feature 1..53: note = GMV00439
  source 1..53: mol_type = other DNA
agtcagacctgttcattaaagaggagaaaatgagcggaaaaaccctttacgac

SEQ ID NO: 134 - DNA, 42 bp, synthetic construct
  misc_feature 1..42: note = GMV00440
  source 1..42: mol_type = other DNA
aaggcttagtctgattcagacggcgcgcaaggcggcgacgat

SEQ ID NO: 135 - DNA, 49 bp, synthetic construct
  misc_feature 1..49: note = GMV00441
  source 1..49: mol_type = other DNA
atcagactaagcctttcacacaggaaaccatgcacgacagactgatcat

SEQ ID NO: 136 - DNA, 35 bp, synthetic construct
  misc_feature 1..35: note = GMV00442
  source 1..35: mol_type = other DNA
ggtttattcctcctttcacacatccccgacttgcg

SEQ ID NO: 137 - DNA, 49 bp, synthetic construct
  misc_feature 1..49: note = ESG00084
  source 1..49: mol_type = other DNA
gagacagaacgaagtgtgaccagaacgcagaagcggtctgataaaacag

SEQ ID NO: 138 - DNA, 70 bp, synthetic construct
  misc_feature 1..70: note = ESG00088
  source 1..70: mol_type = other DNA
gtgttggttgctcgctagcactgtacctaggactgagctagccgtcaagtcagcgccctgcaccattatg

SEQ ID NO: 139 - DNA, 10705 bp, synthetic construct
  misc_feature 1..10705: note = pMZT37
  source 1..10705: mol_type = other DNA
tcaccctgtcgggcaatgccgaggcattctggcagcagcgccccctggcctgtagtggattacgtgccggtctgttccatcctaccaccggctattcactgccgctggcggttgccgtggccgaccgcctgagcgcacttgatgtctttacgtcggcctcaattcaccaggctattaggcattttgcccgcgagcgctggcagcagcagcgctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaagagacagaacgaagtgtgaccagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatcttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtccgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcagccagcgcttcgttaatacagatgtaggtgttccacagggtagccagcagcatcctgcgatgcagatccggaacataatggtgcagggcgctgacttgacggctagctcagtcctaggtacagtgctagcgagcaaccaacacttaaagaggagaaaatgtatccgtttataaggacagcccgaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggccctagggcggttgaagaacgtctgagacaacatcttcatcttgccagtgagcatctctctgcggcctgccaacacgggcacgccactcaacattttattcaggcctggtttgacaaaaaactcgctgccgtcagttaaaggtctctagacaggatgtgtcacacaggaaaccatgaaaccaactacggtaattggtgcaggcttcggtggcctggcactggcaattcgtctacaggctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctgaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggtgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgtatgagtaactctctgtttgtgctctattttggtttgaatcaccatcatgatcagctcgcgcatcacacggtttgtttcggcccgcgttaccgcgaactgattgacgagatttttaatcatgatggcctcgcagaagacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgagtaccttgagcagcattacatgcctggcttacggagtcagctggtcacgcaccagatgtttacgccgtttgattttcgcgaccagcttaatgcctatcagggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatttaagtgatcgttgagtggtgaacttaaagaggagaaaatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgaccagacgctgggcttccaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggacgatacgctgcgctattgctatcacgttgcaggcgttgtcggcttgatgatggcgcaaatcatgggcgtacgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagcgggcctggctgggttgcccctgcgttcggcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctaatcacgtagcaagctgacagtttaaagaggagaaaatgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagcttcgctggtggttcatcactggcccgactatcaggtacgctttcccacacgccgtcgtaagctgaacagcggctacttctgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaactcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatatcgataatgcgacattagatcctgaacgcgcgcggcaaaatatttgcgactatgccgcgcaacagggttggcagcttcagacattgctgcgtgaagaacagggcgccttaccca

SEQ ID NO: 140 - DNA, 411 bp, synthetic construct
  misc_feature 1..411: note = MaFAR-g1
  source 1..411: mol_type = other DNA
gagcaaccaacacttaaagaggagaaaatggcgacccagcagcagcagaacggcgcctcggcgagcggcgtcctggaacagttgcgcgggaagcatgtcctgataaccggtaccaccggtttccttggcaaggtagtcctggaaaagctgatccgcacagtcccggacatcggcggcatccacctcctgatccggggcaacaagaggcatccggccgcccgtgaacggttcttgaacgagatcgccagcagttcggtcttcgagcgtctgcgccacgacgacaacgaggccttcgaaaccttcctggaagaaagggtgcactgtataaccggagaggtcaccgagagtcgtttcggccttaccccggagcgcttccgcgcgctggcgggtcaggtggacgccttcatcaat

SEQ ID NO: 141 - DNA, 465 bp, synthetic construct
  misc_feature 1..465: note = MaFAR-g2
  source 1..465: mol_type = other DNA
gacgccttcatcaattcggccgcctccgtcaacttccgcgaggaactggacaaggcgctgaagatcaatacgctgtgcctggagaatgtcgcggcccttgctgaactcaacagtgcgatggcggtcatccaggtttcgacctgctacgttaacggcaagaatagcgggcagatcaccgaatcggtcatcaagcccgcgggggagtccatcccgcgtagcaccgatgggtactatgaaatcgaagaattggtgcacctgctgcaggacaaaatcagcgatgtgaaggcccgatactccgggaaggttctggaaaaaaaattggtggacctaggcatccgggaagccaataactacgggtggagcgatacatataccttcaccaagtggctgggcgaacagctcctcatgaaggccctgagcggcagatcgctgaccatcgtgcggccgtcgatcatcgagtcggca

SEQ ID NO: 142 - DNA, 374 bp, synthetic construct
  misc_feature 1..374: note = MaFAR-g3
  source 1..374: mol_type = other DNA
atcatcgagtcggcattggaagagcccagcccggggtggattgaaggcgtcaaggtcgccgatgccatcatactggcctacgcgagggagaaggtatcgctctttcctggcaagcggagcggcatcatcgacgtcatcccagtggatctggtggccaattcgatcattctgtccctggcggaggcgctctccggttcgggccagcggcgtatctatcagtgctgcagcggcggctcgaaccccatctccctcgggaagttcatcgactatctgatggcggaggcgaagaccaactacgcggcctacgatcagctgttctaccgccgccccaccaagccgttcgtggccgtcaaccgcaaactcttcgacgtcgt

SEQ ID NO: 143 - DNA, 376 bp, synthetic construct
  misc_feature 1..376: note = MaFAR-g4
  source 1..376: mol_type = other DNA
actcttcgacgtcgtcgtgggcggcatgcgggtcccgctctcgatcgcgggcaaagccatgcgcctggcgggacaaaaccgcgaactgaaggtcctgaagaatctggatacgacccggtccctggccaccattttcgggttctacaccgctccggactacatctttcgcaatgacagcctgatggccctggcctcgcgcatgggcgagctggaccgcgtgttgttccccgttgacgcccgtcagatcgactggcagctgtatctgtgcaaaatccacctcggcgggctgaatcggtacgcgctcaaggaacgtaagctgtactcgctccgggccgccgacactcgcaagaaggcagcctgagagacagaacgaagt

SEQ ID NO: 144 - DNA, 47 bp, synthetic construct
  misc_feature 1..47: note = GMV410
  source 1..47: mol_type = other DNA
gagcaaccaacacttaaagaggagaaaatgcgccccctgcaccccat

SEQ ID NO: 145 - DNA, 47 bp, synthetic construct
  misc_feature 1..47: note = GMV411
  source 1..47: mol_type = other DNA
gagcaaccaacacttaaagaggagaaaatgcgcctgctgaccgccgt

SEQ ID NO: 146 - DNA, 47 bp, synthetic construct
  misc_feature 1..47: note = GMV412
  source 1..47: mol_type = other DNA
gagcaaccaacacttaaagaggagaaaatgtccgtgatgtccccgac

SEQ ID NO: 147 - DNA, 47 bp, synthetic construct
  misc_feature 1..47: note = GMV413
  source 1..47: mol_type = other DNA
gagcaaccaacacttaaagaggagaaaatgccggtcaccgactccat

SEQ ID NO: 148 - DNA, 47 bp, synthetic construct
  misc_feature 1..47: note = GMV414
  source 1..47: mol_type = other DNA
gagcaaccaacacttaaagaggagaaaatggccccgaccgactccct

SEQ ID NO: 149 - DNA, 47 bp, synthetic construct
  misc_feature 1..47: note = GMV415
  source 1..47: mol_type = other DNA
gagcaaccaacacttaaagaggagaaaatgcccctgccgatgtcccc

SEQ ID NO: 150 - DNA, 35 bp, synthetic construct
  misc_feature 1..35: note = GMV416
  source 1..35: mol_type = other DNA
acttcgttctgtctctcagttggcggtcttgatgt

SEQ ID NO: 151 - DNA, 35 bp, synthetic construct
  misc_feature 1..35: note = ACTTCGTTCTGTCTCTCACGGGGCCAGCTTCTTCA
  source 1..35: mol_type = other DNA
acttcgttctgtctctcacggggccagcttcttca

SEQ ID NO: 152 - DNA, 35 bp, synthetic construct
  misc_feature 1..35: note = GMV418
  source 1..35: mol_type = other DNA
acttcgttctgtctctcaggtgccgctcgcggcca

SEQ ID NO: 153 - DNA, 35 bp, synthetic construct
  misc_feature 1..35: note = GMV419
  source 1..35: mol_type = other DNA
acttcgttctgtctctcacagcagggccgcttcca

SEQ ID NO: 154 - DNA, 35 bp, synthetic construct
  misc_feature 1..35: note = GMV420
  source 1..35: mol_type = other DNA
acttcgttctgtctctcacaggccgaccgcggttt

SEQ ID NO: 155 - DNA, 35 bp, synthetic construct
  misc_feature 1..35: note = GMV421
  source 1..35: mol_type = other DNA
acttcgttctgtctctcagatgcccaccgcgcgtt

SEQ ID NO: 156 - DNA, 1149 bp, Saccharomyces cerevisiae
  source 1..1149: mol_type = other DNA
atgcgggcgctggcgtatttcaagaagggcgacatccatttcaccaacgacatcccccggccggagatccagaccgacgacgaagtgatcatcgacgtctcctggtgcggcatctgcggcagcgacctgcacgagtacctggacggccccatcttcatgccgaaggacggcgaatgccacaagctgagcaacgccgcgctgcccctggcgatgggccatgagatgtcgggcatcgtctccaaagtgggcccgaaggtgaccaaggtcaaagtgggcgaccacgtcgtggtcgacgccgcgagctcgtgcgccgacctgcactgctggccccattccaagttctataacagcaagccgtgcgacgcctgccagcgcggctcggagaacctgtgcacccatgcgggcttcgtcggcctgggcgtgatcagcggcggcttcgccgaacaggtggtcgtgtcgcagcaccatatcatcccggtccccaaggagatccccctggacgtcgccgccctggtcgagccgctgtcggtcacctggcacgccgtgaagatctccggcttcaagaagggctccagcgccctggtcctgggcgcgggccccatcggcctgtgcaccatcctggtgctgaagggcatgggcgcgtcgaagatcgtcgtgtccgagatcgccgaacgtcgcatcgagatggcgaagaagctgggcgtcgaagtgttcaacccgagcaagcacggccataagtcgatcgagatcctgcggggcctgaccaagtcccacgacggcttcgactacagctatgactgctcgggcatccaggtcaccttcgaaaccagcctgaaggccctgaccttcaagggcaccgccaccaacatcgcggtctggggcccgaagcccgtgccgttccagccgatggacgtcaccctgcaggagaaggtgatgaccggctcgatcggctacgtcgtggaagacttcgaggaagtcgtgcgcgccatccataacggcgacatcgcgatggaggactgcaagcagctgatcaccggcaagcagcggatcgaggacggctgggaaaagggcttccaggagctgatggaccacaaggaatccaacgtgaagatcctgctgaccccgaacaaccacggcgaaatgaagtga

SEQ ID NO: 157 - protein, 382 aa, Saccharomyces cerevisiae
MRALAYFKKGDIHFTNDIPRPEIQTDDEVIIDVSWCGICGSDLHEYLDGPIFMPKDGECHKLSNAALPLAMGHEMSGIVSKVGPKVTKVKVGDHVVVDAASSCADLHCWPHSKFYNSKPCDACQRGSENLCTHAGFVGLGVISGGFAEQVVVSQHHIIPVPKEIPLDVAALVEPLSVTWHAVKISGFKKGSSALVLGAGPIGLCTILVLKGMGASKIVVSEIAERRIEMAKKLGVEVFNPSKHGHKSIEILRGLTKSHDGFDYSYDCSGIQVTFETSLKALTFKGTATNIAVWGPKPVPFQPMDVTLQEKVMTGSIGYVVEDFEEVVRAIHNGDIAMEDCKQLITGKQRIEDGWEKGFQELMDHKESNVKILLTPNNHGEMK

SEQ ID NO: 158 - DNA, 47 bp, synthetic construct
  misc_feature 1..47: note = GMV00268
  source 1..47: mol_type = other DNA
gagcaaccaacacttaaagaggagaaaatgcgggcgctggcgtattt

SEQ ID NO: 159 - DNA, 35 bp, synthetic construct
  misc_feature 1..35: note = GMV00271
  source 1..35: mol_type = other DNA
acttcgttctgtctctcacttcatttcgccgtggt

SEQ ID NO: 160 - DNA, 990 bp, Escherichia coli
  source 1..990: mol_type = other DNA
atgcatattacatacgatctgccggttgctattgatgacattattgaagcgaaacaacgactggctgggcgaatttataaaacaggcatgcctcgctccaactattttagtgaacgttgcaaaggtgaaatattcctgaagtttgaaaatatgcagcgtacgggttcatttaaaattcgtggcgcatttaataaattaagttcactgaccgatgcggaaaaacgcaaaggcgtggtggcctgttctgcgggcaaccatgcgcaaggggtttccctctcctgcgcgatgctgggtatcgacggtaaagtggtgatgccaaaaggtgcgccaaaatccaaagtagcggcaacgtgcgactactccgcagaagtcgttctgcatggtgataacttcaacgacactatcgctaaagtgagcgaaattgtcgaaatggaaggccgtatttttatcccaccttacgatgatccgaaagtgattgctggccagggaacgattggtctggaaattatggaagatctctatgatgtcgataacgtgattgtgccaattggtggtggcggtttaattgctggtattgcggtggcaattaaatctattaacccgaccattcgtgttattggcgtacagtctgaaaacgttcacggcatggcggcttctttccactccggagaaataaccacgcaccgaactaccggcaccctggcggatggttgtgatgtctcccgcccgggtaatttaacttacgaaatcgttcgtgaattagtcgatgacatcgtgctggtcagcgaagacgaaatcagaaacagtatgattgccttaattcagcgcaataaagtcgtcaccgaaggcgcaggcgctctggcatgtgctgcattattaagcggtaaattagaccaatatattcaaaacagaaaaaccgtcagtattatttccggcggcaatatcgatctttctcgcgtctctcaaatcaccggtttcgttgacgcttaa

SEQ ID NO: 161 - protein, 329 aa, Escherichia coli
MHITYDLPVAIDDIIEAKQRLAGRIYKTGMPRSNYFSERCKGEIFLKFENMQRTGSFKIRGAFNKLSSLTDAEKRKGVVACSAGNHAQGVSLSCAMLGIDGKVVMPKGAPKSKVAATCDYSAEVVLHGDNFNDTIAKVSEIVEMEGRIFIPPYDDPKVIAGQGTIGLEIMEDLYDVDNVIVPIGGGGLIAGIAVAIKSINPTIRVIGVQSENVHGMAASFHSGEITTHRTTGTLADGCDVSRPGNLTYEIVRELVDDIVLVSEDEIRNSMIALIQRNKVVTEGAGALACAALLSGKLDQYIQNRKTVSIISGGNIDLSRVSQITGFVDA

SEQ ID NO: 162 - protein, 590 aa, Methylosinus trichosporium
MARKMTGAEMVVEALKDQGVEIIFGYPGGAVLPIYDALFHQEKVQHILVRHEQGAAHAAEGYARSSGKVGVLLVTSGPGATNTITGLTDALMDSIPVVCITGQVPTHLIGSDAFQECDTVGITRHCTKHNYLVKSVDDLPRILHEAFYVASSGRPGPVVIDIPKDVQFASGTYTGPRNVHHKTYQPKLEGDTESIRRAVKMMAAAKRPIFYTGGGVINSGPAASTLLRELVSLTGFPITSTLMGLGAYPGSGPNWLGMLGMHGTFEANNAMHDCDLMIAVGARFDDRITGRLDAFSPGSKKIHIDIDRSSINKNVKIDLPIVGDCGHVLESLVRVWRSEAMHAEKQPLDGWWKTIDHWRERKSLAFRNSDKVIKPQYAVQRLYALTKDRDPYITTEVGQHQMWAAQHYHFDEPNRWMTSGGLGTMGYGLPAAIGAQLAHPKSLVVDIAGEASILMNIQEMSTAIQYRLPVKVFILNNEYMGMVRQWQELLHGGRYSHSYSEALPDFVKLAEAFGGKGIRCSDPAELDSAILEMIDYDGPVIFDCLVEKNENCFPMIPSGKAHNDMLLADLGDDAGVELGSIIDEKGKMLV

SEQ ID NO: 163 - protein, 351 aa, Methylosinus trichosporium
MSTKAYAVASAEALFGPLAIERRALGPEDVEIDILYCGVCHSDLHTARSEWPGTRYPCVPGHEIVGRVTAVGAKVTKFSVGDLAAVGCMVDSCRRCLSCDDGLEQYCEHGFTATYNGPIYGSGENTFGGYSEKIVVDAHFVLAIHHSETQLAGVAPLLCAGITTWSPLKHWGVGPGKSVGIVGIGGLGHMGVKLAHALGAHVVAFTTSPSKRDAALALGADEVVVSTDPAAMAARAGSLDFILDTVAVAHDLDAYVNLLKRDGALVLVGVPATPHPSPSAGGLIFKRRQVAGSLIGGVKETQEMLDFCAERGIVADIETIAMQQIETAYARMLKNDVKYRFVIDMATLKAA

SEQ ID NO: 164 - protein, 342 aa, Methylococcus capsulatus
MKAWVIDRIGPLDSSRTLLRATDLPVPEPGPGEILLQVAVCGVCHTEIDEIEGRTAPPRLPVVPGHQAVGRIAALGSGVAEFALGDRVGVAWIFSACGECEFCRSGRENLCFAFCATGRDVDGGYAQYMTVPAAFAFRIPEGFTDAEAAPLLCAGAIGYRSLNLSGLKNGQPLGLTGFGASAHLVLMMARYRFPDSEVYVFARHPEERAFALQLGAVWAGDTADIAPAPLAAIIDTTPAWKPVVAALANLAPGGRLVVNAIRKAPDDRACLAELDYARHLWMEREIKSVANVARSDVAGFLALAAEMGIRPETEEYPFEDADRALLDLKQRRIRGAKVLRVT

SEQ ID NO: 165 - protein, 355 aa, Methylococcus capsulatus
MPTAKAYAAFSADSALAPFVLQRRDPLPQDIRIGILYCGVCHSDLHQARNEWNATTYPCVPGHEIVGKVLEVGRSVTKFKPGDTVAVGCMVDSCRTCPNCVDALEQHCEHGPVFTYNSPDPHGGGMTFGGYAESIVVDEAFVLRIPDGLDLAAAAPLLCAGITTYSPLRHWKVGAGQRVGVVGLGGLGHMALKFAHTFGAETVLFTTTPDKAEDARRLGADEVVVSRDPEAMARQAGRFDFILDTVSAPHDIDAYLNLLRRDGTLTLVGVPPQGVQVMPFSLIGGRRRLAGSLIGGIRETQEMLDFCGEHGIVCDIELIPIQGINDAFERMLKSDVKYRFVIDMATLNGESSGGR

SEQ ID NO: 166 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 167 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 168 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 169 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 170 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 171 - protein, 1048 aa, Clostridium acetobutylicum
MKSEYTIGRYLLDRLSELGIRHIFGVPGDYNLSFLDYIMEYKGIDWVGNCNELNAGYAADGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNNGYARINGIGAILTTFGVGELSAINAIAGAYAEQVPVVKITGIPTAKVRDNGLYVHHTLGDGRFDHFFEMFREVTVAEALLSEENAAQEIDRVLISCWRQKRPVLINLPIDVYDKPINKPLKPLLDYTISSNKEAACEFVTEIVPIINRAKKPVILADYGVYRYQVQHVLKNLAEKTGFPVATLSMGKGVFNEAHPQFIGVYNGDVSSPYLRQRVDEADCIISVGVKLTDSTTGGFSHGFSKRNVIHIDPFSIKAKGKKYAPITMKDALTELTSKIEHRNFEDLDIKPYKSDNQKYFAKEKPITQKRFFERIAHFIKEKDVLLAEQGTCFFGASTIQLPKDATFIGQPLWGSIGYTLPALLGSQLADQKRRNILLIGDGAFQMTAQEISTMLRLQIKPIIFLINNDGYTIERAIHGREQVYNNIQMWRYHNVPKVLGPKECSLTFKVQSETELEKALLVADKDCEHLIFIEVVMDRYDKPEPLERLSKRFANQNN

SEQ ID NO: 172 - DNA, 645 bp, Methylococcus capsulatus
  source 1..645: mol_type = other DNA
atggcaagaccattgattcagctcgccctggacacgctggacatcccgcagaccctgaagctcgcaagcctcaccgcgccctatgtcgatatcttcgaaatcggcacccccagcatcaagcacaacggcatcgccctggtgaaggagttcaaaaaacgcttccccaacaagctgctcctggtcgacctcaaaaccatggacgccggtgaatacgaagccacccccttcttcgccgccggcgccgacatcaccaccgtcctcggcgtcgcaggactggccaccatcaagggcgtcatcaacgccgccaacaagcacaacgccgaggtccaggtcgacctgatcaacgtccccgacaaggccgcctgcgcccgtgagtccgccaaggccggcgcccagatcgtcggcatccacaccggcctcgacgcccaggccgccggccagacccccttcgccgacctccaggccatcgccaagctcggcctccccgtccgcatctccgtcgccggcggcatcaaggcctccaccgcccaacaggtcgtcaaaaccggtgccaacatcatcgtcgtcggagccgccatctacggcgccgcctcccccgccgatgccgcgcgcgaaatctacgaacaggtcgtcgccgcttccgcc

SEQ ID NO: 173 - protein, 215 aa, Methylococcus capsulatus
MARPLIQLALDTLDIPQTLKLASLTAPYVDIFEIGTPSIKHNGIALVKEFKKRFPNKLLLVDLKTMDAGEYEATPFFAAGADITTVLGVAGLATIKGVINAANKHNAEVQVDLINVPDKAACARESAKAGAQIVGIHTGLDAQAAGQTPFADLQAIAKLGLPVRISVAGGIKASTAQQVVKTGANIIVVGAAIYGAASPADAAREIYEQVVAASA

SEQ ID NO: 174 - DNA, 531 bp, Methylococcus capsulatus
  source 1..531: mol_type = other DNA
atgcatcagaaactgatcatagacaaaatctccggcatcctcgccgccaccgatgccggctatgatgcaaaactgactgccatgctcgaccaggcctcccgcatcttcgtcgcgggggccggccggtcggggctggtcgccaagttcttcgccatgcgcctcatgcacggcggctatgacgtcttcgtcgtcggcgaaatcgtcacccccagcatccgcaagggcgacttgctgatcgtgatctccggctccggtgaaaccgaaaccatgctcgccttcaccaaaaaagccaaggagcagggcgcctccatcgccctcatctccacccgcgacagctcctccctcggcgacctcgccgactccgtcttccgcatcggctccccagagctcttcggaaaagtcgtcggcatgcccatgggcaccgtcttcgagctctccaccctcctcttcctcgaggccaccatctctcacatcatccacgagaaaggcatccccgaagaagaaatgagaactcgtcacgccaacctggaa

SEQ ID NO: 175 - protein, 177 aa, Methylococcus capsulatus
MHQKLIIDKISGILAATDAGYDAKLTAMLDQASRIFVAGAGRSGLVAKFFAMRLMHGGYDVFVVGEIVTPSIRKGDLLIVISGSGETETMLAFTKKAKEQGASIALISTRDSSSLGDLADSVFRIGSPELFGKVVGMPMGTVFELSTLLFLEATISHIIHEKGIPEEEMRTRHANLE

(19) United States
(12) Patent Application Publication
(10) Pub. No.: US 2024/0368638 A1
(43) Pub. Date: Nov. 7, 2024

(54) Biological Production of Multi-Carbon Compounds from Methane

(71) Applicant: PRECIGEN, INC., BLACKSBURG, VA (US)

(72) Inventors: William J. COLEMAN, Redwood City, CA (US); Genevieve M. VIDANES, San Francisco, CA (US); Guillaume COTTAREL, Mountain View, CA (US); Sheela MULEY, Fremont, CA (US); Roy KAMIMURA, Daly City, CA (US); Akbar F. JAVAN, Chapel Hill, NC (US); Jianping SUN, Belmont, CA (US); Eli S. GROBAN, San Francisco, CA (US)

(21) Appl. No.: 18/379,079

(22) Filed: Oct. 11, 2023

Related U.S. Application Data
(63) Continuation of application No. 17/103,516, filed on Nov. 24, 2020, now Pat. No. 11,821,019.
Application No. 17/103,516 is a continuation of application No. 15/648,920, filed on Jul. 13, 2017, now Pat. No. 10,876,137.
Application No. 15/648,920 is a continuation of application No. 15/192,290, filed on Jun. 24, 2016, now Pat. No. 9,745,603.
Application No. 15/192,290 is a division of application No. 14/989,859, filed on Jan. 7, 2016, now Pat. No. 9,399,783.
Application No. 14/989,859 is a division of application No. 14/206,835, filed on Mar. 12, 2014, now Pat. No. 9,267,158.
(60) Provisional application No. 61/782,830, filed on Mar. 14, 2013.

(51) Int. Cl. C12P 7/16; C12N 9/04; C12N 9/10; C12N 9/88; C12N 15/52

(52) U.S. Cl. CPC C12P 7/16; C12N 9/0006; C12N 9/1022; C12N 9/1025; C12N 9/88; C12N 15/52; C12Y 101/01001; C12Y 101/01004; C12Y 101/01086; C12Y 202/01006; C12Y 203/03006; C12Y 401/01072; C12Y 402/01009; C12Y 402/01033; C12Y 403/01019; Y02E 50/10

(57) ABSTRACT

Multi-carbon compounds such as ethanol, n-butanol, sec-butanol, isobutanol, tert-butanol, fatty (or aliphatic long chain) alcohols, fatty acid methyl esters, 2,3-butanediol and the like, are important industrial commodity chemicals with a variety of applications. The present invention provides metabolically engineered host microorganisms which metabolize methane (CH₄) as their sole carbon source to produce multi-carbon compounds for use in fuels (e.g., bio-fuel, bio-diesel) and bio-based chemicals. Furthermore, use of the metabolically engineered host microorganisms of the invention (which utilize methane as the sole carbon source) mitigate current industry practices and methods of producing multi-carbon compounds from petroleum or petroleum-derived feedstocks, and ameliorate much of the ongoing depletion of arable food source “farmland” currently being diverted to grow bio-fuel feedstocks, and as such, improve the environmental footprint of future bio-fuel, bio-diesel and bio-based chemical compositions.

### CROSS-REFERENCE TO RELATED APPLICATION

This application claims priority benefit of U.S. provisional Application No. 61/782,830, filed Mar. 14, 2013, which is hereby incorporated by reference in its entirety.

### REFERENCE TO AN ELECTRONIC SEQUENCE LISTING

The contents of the electronic sequence listing (35016-001C4.xml; Size: 304,171 bytes; and Date of Creation: May 27, 2024) are herein incorporated by reference in its entirety.

### BACKGROUND OF THE INVENTION

### Field of the Invention

The present invention is generally related to the fields of molecular biology and methods of producing metabolically engineered microorganisms which utilize methane feedstocks for the biological production of bio-fuels and bio-chemicals such as 1-butanol, isobutanol, fatty alcohols, fatty acid esters, 2,3-butanediol and the like.

### Background Art

Traditional fossil fuels (e.g., gasoline, diesel, kerosene and the like) and numerous chemicals (e.g., for use in pharmaceuticals, solvents, fertilizers, pesticides, plastics and the like) are derived (and refined from) non-renewable petroleum (oil) resources. Current estimates suggest that the world's supply of non-renewable petroleum will likely be exhausted somewhere between the years 2045 and 2065 (U.S. Department of the Interior, U.S. Geological Survey World Petroleum Assessment, 2000), with concomitant extensions or reductions of these estimates dependent on variables such as increased (or reduced) global demand, more efficient petroleum refining processes, more efficient use of energy and products derived from petroleum and the discovery of new petroleum sources/reserves.

Independent of any current or future methods contemplated to mitigate petroleum consumption, there is no debate that the world's supply of petroleum is a finite and a constantly diminishing (non-renewable) energy source. Thus, to meet the ever increasing global demands for energy consumption, renewable, biologically produced fuels (i.e., “bio-fuels” and “bio-diesel”) have become an area of intense research, capital investment and government intervention.

For example, the U.S. “Energy Policy Act” of 2005 (42 USC, Title XV “Ethanol and Motor Fuels”, § 1501-§ 1533; enacted into law Aug. 8, 2005), sets forth parameters and definitions of “renewable fuels”, and established the “minimum ethanol” volume to gasoline volume blending requirements (presently E10: 10% ethanol: 90% gasoline), with E15 (15% ethanol: 85% gasoline) enacted as law and being “phased-in” across the U.S. The Energy Policy Act defines “renewable fuel” as a “motor vehicle fuel produced from grain, starch, oil-seeds, vegetable, animal, or fish materials including fats, greases, and oils, sugarcane, sugar beets, sugar components, tobacco, potatoes, or other biomass; or a natural gas produced from a biogas source, including a landfill, sewage waste treatment plant, feedlot, or other place where decaying organic material is found; and is used to replace or reduce the quantity of fossil fuel present in a fuel mixture used to operate a motor vehicle. The term “renewable fuel” includes (a) cellulosic biomass ethanol and waste derived ethanol; and (b) biodiesel, and any blending components derived from renewable fuel”.

In addition to the current E10 ethanol/gasoline blends and ongoing adoption of E15 ethanol/gasoline blends, ethanol volumes of up to E85 (i.e., 85% ethanol: 15% gasoline) are also presently being utilized in “flex-fuel” vehicles (i.e., vehicles with engines and fuel systems capable of combusting and delivering, respectively, 85% ethanol blended gasoline) and it is estimated that the production of E85 fuel will only continue to increase as the supply (i.e., production) of “flex-fuel” vehicles increase. However, an inherent limitation of “ethanol” blended fuels (due to the decreased or lower “energy content” of ethanol relative to gasoline) is that increasing the percentage of ethanol blended into gasoline reduces the overall fuel economy of the vehicle (e.g., fuel economy of vehicles operating on E85 is about 25-30% less than vehicles operating on E10 gasoline blends). This limitation of ethanol's total energy content has further facilitated research and development of alternative bio-fuel blending additives (e.g., terpenoid hydrocarbons, n-butanol, isobutanol and the like) to replace bio-ethanol. Also predicated on the assumption of a finite, diminishing supply of non-renewable petroleum resources, research in the areas of biologically derived (hereinafter, “bio-based”) chemicals (e.g., for use in pharmaceuticals, solvents, fertilizers, pesticides, plastics and the like) are being pursued, wherein these “bio-based”chemicals are contemplated as a means for reducing or eliminating their equivalents traditionally derived from petroleum feed stocks.

A considerable topic of ongoing debate is whether the ethanol fuel provisions of the Energy Policy Act of 2005 (and similar policies of other countries) have reduced (or will reduce) dependence on foreign oil/petroleum sources and/or have mitigated (or will mitigate) greenhouse gas emissions (two perceived benefits of the Act). For example, bio-fuels such as ethanol were initially seen as a solution to energy and environmental problems (i.e., considered carbon neutral) because the carbon dioxide emitted when ethanol is combusted is equivalent to the carbon dioxide absorbed from the atmosphere when the ethanol feed stock crop is grown (e.g., corn ethanol, sugarcane ethanol, cellulosic ethanol from switchgrass, etc.).

A recent study by economists at Oregon State University (Jaeger & Egelkraut, 2011) suggests however, that once additional factors/consequences are considered, such as (a) the use of fossil fuels to produce bio-fuel feedstocks and transport bio-fuels, (b) the use of nitrogen fertilizers to grow bio-fuel feedstocks and (c) that growing bio-fuel feedstock crops often pushes food production onto previously unfarmed land (which typically requires clearing tress and heavy tilling of the land), the perceived environmental benefits of ethanol derived bio-fuels may be lost. Likewise, another recent study on the environmental impact of bio-fuel production concludes that high corn and soybean prices, prompted largely by the demand for bio-fuel feedstocks (and partly by government incentives to use them as fuels instead of food), are driving one of the most important land cover/land use change events in recent US history; the accelerated conversion of grassland to cropland in the US Corn Belt (Wright and Wimberly, 2013).

The shift from petroleum based diesel fuel as a (transportation) energy source (e.g., used in automobiles, trucks and other heavy equipment) to renewable bio-diesel fuels is another source of scientific and policy disagreement similar to the arguments set forth above with regard to ethanol bio-fuels. Bio-diesel is generally made from plant oils or animal fats (triacylglycerides) by transesterification with methanol or ethanol, resulting in fatty acid methyl esters and fatty acid ethyl esters. However, the limited supply of bioresources to obtain triacylglycerides has become a major bottleneck for bio-diesel production, the primary reason being that vegetable oil feedstocks are also food sources and their planting is geographically limited.

There is therefore a pressing need in the art for novel methods of producing bio-fuel, bio-diesel and bio-based chemical compositions which reduce the world's dependence/utilization of petroleum products, ameliorate ongoing depletion of arable food source “farmland” currently being diverted to grow bio-fuel feedstocks and generally improve the environmental footprint of future bio-fuel, bio-diesel and bio-based chemical compositions.

As mentioned previously above, ethanol is currently the most abundant bio-fuel produced, but due to certain limitations (e.g., low energy content, high water solubility, incompatibility/corrosive with many fuel systems), ethanol based bio-fuels may not be the best option to meet future energy demands. Butanol, in comparison, has several advantages over ethanol as a bio-fuel, such as its high blending compatibility with gasoline, its low solubility in water allow it to be stored and distributed using the existing petrochemical infrastructure, it has a much higher energy content than ethanol (thereby improving fuel economy) and has a lower vapor pressure than ethanol blends, which is important in reducing evaporative hydrocarbon emissions. Isobutanol has the same advantages as butanol, with the additional advantage of having a higher octane number due to its branched carbon chain, and it is also useful as a commodity chemical.

Various methods for producing renewable bio-fuel, bio-diesel and other bio-based chemicals are known and described in the art. For example, traditional fermentation and distillation methods for producing and extracting bio-ethanol from starch or sugar rich biomass (e.g., corn) and the hydrolysis, fermentation and distillation methods of producing bio-ethanol from ligno-cellulosic biomass are well known in the art (Rudolph et al., 2009; Kim et al, 2013; Philips et al., 2013). The production of bio-diesel via extraction and esterification of vegetable oils, used cooking oils and animal fats using alcohols is also well known in the art (Saka & Kusdiana, 2001).

In more recent efforts, researchers have started to look at alternative methods for producing bio-fuels, bio-diesel and bio-based chemicals. For example, methods for producing bio-fuels such as butanol and isobutanol in various microorganisms such as Escherichia coli (Atsumi et al., 2010), Clostridium acetobutylicum (Jang et al., 2012) and Saccharomyces cerevisiae (Avalos et al., 2013) have been described in the art. Furthermore, the complete biosynthetic pathway for isobutanol production has been engineered in yeast (see, U.S. Pat. Nos. 8,232,089; 7,993,889) and bacteria (see, U.S. Patent Publication No. 2011/0301388). Similarly, de novo biosynthesis of bio-diesel using genetically engineered E. coli has been described in the art (Xingye et al., 2011; Yangkai et al., 2011).

However, each of the methods set forth above (i.e., traditional biomass fermentation methods and engineered biological/microorganism methods) for producing bio-fuel, bio-diesel, bio-based chemicals and the like, are limited by the choice of feedstock (or substrate) used to produce the end product (e.g., bio-ethanol, bio-butanol, bio-diesel, etc.). For example, the growth substrates utilized by each of the microorganisms set forth above (i.e., E. coli, C. acetobutylicum and S. cerevisiae) are dependent, in one way or another, on substrate feedstocks derived from crop-based food sources (e.g., glucose (growth) substrates fed to microorganisms are derived from plant sources).

Thus, as set forth previously, there is an ongoing need in the art for novel methods of producing bio-fuel, bio-diesel and bio-based chemical compositions, which not only reduce dependence/utilization of petroleum products, but also ameliorate the ongoing depletion of arable food source “farmland” currently being diverted to grow bio-fuel feedstocks and generally improve the environmental footprint of future bio-fuel, bio-diesel and bio-based chemical compositions.

Methane (CH₄) has great value as a chemical feedstock for the production of chemicals and food additives, due to its widespread availability, abundant supply and low price (Kidnay et al., 2011). Methane, in the form of natural gas, can be obtained from shale gas, oil drilling, municipal solid waste, biomass gasification/conversion, and methanogenic archaea. Wellhead natural gas varies in composition from about 40% to 95% methane, wherein the other components include ethane, propane, butane, pentane, and heavier hydrocarbons, along with hydrogen sulfide, carbon dioxide, helium and nitrogen. The proportion of methane in the gas feedstock can be increased by gas conditioning, which can produce natural gas consisting of 85-95% (v/v) methane (U.S. Pat. No. 4,982,023).

Current industrial methods for utilizing methane from natural gas include the Fischer-Tropsch process for converting methane into ethylene, steam-methane reforming from methane synthesis gas, as well as direct conversion from methane to methanol using inorganic catalysts (Veazey, 2012; Alayon et al., 2012; U.S. Pat. No. 4,982,023). Although the economics of syngas-to-liquids and methanol-to-gasoline from natural gas have become more favorable, these thermochemical methods for methane conversion still suffer from serious drawbacks (Arakawa et al., 2001). For example: (1) industrial plant construction requires high capital expenditure, (2) operating costs are high, (3) thermochemical conversion plants require elevated temperatures (150° C. to 300° C.) and high pressures (tens of atmospheres), which add to capital and operational costs, (4) the gas-to-liquids process is not always selective in producing liquid fuel and chemical products, further requiring expensive distillation costs and (5) the inorganic catalysts required for producing methanol and other products are susceptible to poisoning by contaminants in the process stream, and therefore the gas streams must be cleaned and the catalysts periodically replaced.

Certain embodiments of the present invention, as set forth below (see, “Detailed Description”), are directed to methods for biosynthetic production of multi-carbon compounds such as fuels (bio-fuels) and chemicals (bio-based) from methane. It is contemplated herein that the methods according to the present invention, using biological catalysts or biocatalysts (e.g., a genetically modified host microorganism) provide a number of economic advantages over current “industrial” methods for utilizing methane from natural gas. These advantages include (1) lower processing temperatures and pressures; (2) high selectivity for the reactions and (3) renewability, all of which lead to substantially lower capital and operational expenses.

A number of microorganisms, including bacteria and yeast, use single-carbon (C1) substrates as their sole source of carbon. These methylotrophs or C1-metabolizers can convert carbon compounds that do not contain carbon-carbon bonds, such as methane (CH₄) or methanol (CH₃OH) into biomass (Gellissen et al., 2005; Trotsenko & Murrell, 2008; Chistoserdova et al., 2009; Schrader et al., 2009; Chistoserdova, 2011). With regard to methane utilization, one particularly important group of bacteria known as the methanotrophs, the “obligate” members of which convert methane into methanol (CH₃OH), formaldehyde (H₂C═O), formic acid (HCOOH) and ultimately CO₂ by sequential enzymatic oxidation (Hanson & Hanson, 1996; Trotsenko & Murrell, 2008; Rosenzweig & Ragsdale, 2011 (a); Rosenzweig & Ragsdale 2011 (b)). Certain “facultative” methanotrophs (e.g., from the genus Methylocella) can also be cultivated using methane, methanol or methylamines as growth substrates (Dunfield et al., 2003; Rosenzweig & Ragsdale, 2011 (a); Rosenzweig & Ragsdale 2011 (b); Semrau et al., 2011).

The initial step of methane oxidation to methanol in methanotrophs is carried out by the enzyme methane monooxygenase (MMO) (Hakemian & Rosensweig, 2007; Rosenzweig & Ragsdale, 2011 (b)). Methane monooxygenase (MMO) activity is expressed in two different forms: a particulate form (pMMO), which contains copper and is membrane-bound (Culpepper & Rosenzweig, 2012), and a soluble form (sMMO), which contains iron and is expressed when copper becomes limiting (Murrel et al., 2000; Hakemian & Rosenzweig, 2007; Tinberg & Lippard, 2007). The second step of converting methanol to formaldehyde is catalyzed by the enzyme methanol dehydrogenase (MDH), another membrane-bound enzyme (Anthony & Williams, 2003). From this point, the formaldehyde can be dissimilated into formate (by formaldehyde dehydrogenase) and carbon dioxide (by formate dehydrogenase). The dissimilation reactions generate reducing equivalents for the cell, but do not directly contribute to the production of biomass or other multi-carbon products, since the carbon is released as CO₂. In some methanotrophs, however, carbon dioxide can be fixed through the serine pathway and/or the Calvin-Benson-Bassham cycle (see below), both of which depend on methane consumption to support growth (Stanley & Dalton, 1982; Chistoserdova et al., 2005). Among the oxidized C1 products that can be generated in the above described reactions, formaldehyde is the most important product (or imtermediate), as it serves as a metabolite that can be “fixed” into multi-carbon compounds via its introduction (or assimilation) into a central metabolism pathway of the host microorganism.

For example, the assimilation of the carbon in the formaldehyde formed can occur via various metabolic routes (Hanson & Hanson, 1996; Yurimoto et al., 2005; Yurimoto et al., 2009; Trotsenko & Murrell, 2008; Rosenzweig & Ragsdale, 2011 (a); Rosenzweig & Ragsdale, 2011 (b)). For example, the Type I methanotrophs, which are members of the Gammaproteobacteria, use the ribulose monophosphate (RuMP) pathway (see, Hanson & Hanson, 1996). The Type II methanotrophs, which are members of the Alphaproteobacteria, utilize the serine pathway (Hanson & Hanson, 1996). The bacterium Methylococcus capsulatus, strain Bath, however, uses elements of both these pathways, and is sometimes referred to as a “Type X” methanotroph (Hanson & Hanson, 1996; Chistoserdova et al., 2005). Methylococcus capsulatus (Bath), also expresses the enzymes needed to fix carbon dioxide via the Calvin-Benson-Bassham cycle (Chistoserdova et al., 2005).

Turnover of these pathways (i.e., Type I, Type II or Type X) ultimately supplies multi-carbon intermediates for other pathways of central metabolism. For example, the 3-phospho-glyceraldehyde generated by the RuMP cycle can be converted into pyruvate, and the 2-phospho-glycerate generated by the serine cycle can eventually be converted into phosphoenolpyruvate, oxaloacetate and acetyl-CoA, among other intermediates.

Substantial efforts have been expended over the past 40 years to exploit methanotrophs for chemical production and transformations on an industrial scale. However, to date there are still significant deficiencies and unmet needs in the art for improved host microorganisms which can utilize “non-traditional” carbon sources such as oxidized single-carbon compounds (e.g., methane, methanol or formaldehyde) and produce industrial, commercially relevant, multi-carbon compounds such as ethanol, n-butanol, sec-butanol, isobutanol, tert-butanol, fatty alcohols, fatty acid methyl esters, 2,3-butanediol and the like.

The present invention fulfills a need in the art for improved host microorganisms (which can utilize methane as a sole-carbon source in the production of multi-carbon compounds) for use in the biological production of bio-fuels and bio-based chemical compositions. The metabolically engineered host microorganisms and methods of producing the same, as set forth in the present invention, further address a long felt need in the art to reduce dependence/consumption of petroleum products and mitigate the depletion of farmland currently being diverted to grow bio-fuel and bio-based chemical feedstocks.

### SUMMARY OF THE INVENTION

The present invention provides metabolically engineered host microorganisms which metabolize methane (CH₄) as their sole carbon source to produce multi-carbon compounds for use in fuels (e.g., bio-fuel, bio-diesel) and bio-based chemicals. Furthermore, use of the metabolically engineered host microorganisms of the invention (which utilize methane as the sole carbon source) mitigate current industry practices and methods of producing multi-carbon compounds from petroleum or petroleum-derived feedstocks, and ameliorate much of the ongoing depletion of arable food source “farmland” currently being diverted to grow bio-fuel feedstocks, and as such, improve the environmental footprint of future bio-fuel, bio-diesel and bio-based chemical compositions.

Thus, in certain embodiments, the invention is directed to a method for producing isobutanol from a methane substrate comprising the steps of (a) providing a methanotrophic host microorganism that metabolizes methane (CH₄) to methanol (CH₃OH) and methanol to formaldehyde (H₂C═O); (b) introducing into the methanotroph host and expressing at least one polynucleotide open reading frame (ORF), under the control of suitable regulatory sequences, wherein the at least one polynucleotide ORF encodes a polypeptide that catalyzes a reaction in an isobutanol pathway; and (c) feeding the methanotroph host of step (b) a methane substrate under suitable growth conditions, wherein the host metabolizes methane to formaldehyde as set forth in step (a), wherein the formaldehyde is converted to pyruvate by means of an endogenous type I RuMP pathway or a type II serine pathway and the host metabolizes pyruvate to produce isobutanol. In certain embodiments, the one or more polynucleotide ORFs introduced in step (b) encode an isobutanol pathway polypeptide selected from an Enzyme Class (EC) comprising EC 2.2.1.6, EC 1.1.1.86, EC 4.2.1.9, EC 4.1.1.72 and EC 1.1.1.1. In other embodiments, the one or more polynucleotide ORFs introduced in step (b) encode an isobutanol pathway polypeptide selected from the group consisting of acetolactate synthase (ALS), ketol-acid reductoisomerase (KARI), dihydroxy-acid dehydratase (DHAD), ketoacid decarboxylase (KDC) and alcohol dehydrogenase (ADH). In yet other embodiments, the ALS polypeptide catalyzes the substrate to product conversion of pyruvate to acetolactate; the KARI polypeptide catalyzes the substrate to product conversion of acetolactate to 2,3-dihydroxyisovalerate; the DHAD polypeptide catalyzes the substrate to product conversion of 2,3-dihydroxyisovalerate to ketoisovalerate; the KDC polypeptide catalyzes the substrate to product conversion of ketoisovalerate to isobutryaldehyde and ADH polypeptide catalyzes the substrate to product conversion of isobutyraldehyde to isobutanol. In another embodiment, the ALS polypeptide comprises an amino acid sequence having at least 90% sequence homology to SEQ ID NO:2, the KARI polypeptide comprises an amino acid sequence having at least 90% sequence homology to SEQ ID NO:4, the DHAD polypeptide comprises an amino acid sequence having at least 90% sequence homology to SEQ ID NO:6, the KDC polypeptide comprises an amino acid sequence having at least 90% sequence homology to SEQ ID NO:8 and the ADH polypeptide comprises an amino acid sequence having at least 90% sequence homology to SEQ ID NO:10. In other embodiments, the one or more polynucleotide ORFs introduced in step (b) encode the complete isobutanol pathway comprising an ALS polypeptide, a KARI polypeptide, a DHAD polypeptide, a KDC polypeptide and an ADH polypeptide. In other embodiments a method for producing isobutanol from a methane substrate further comprises the step of recovering the isobutanol produced.

In another embodiment, the invention is directed to a method for producing isobutanol from a methane substrate comprising the steps of (a) providing a non-methanotroph host microorganism which has been genetically engineered to express a methane monooxygenase (MMO), (b) introducing into the host and expressing at least one polynucleotide open reading frame (ORF), under the control of suitable regulatory sequences, wherein the at least one polynucleotide ORF encodes a polypeptide that catalyzes a reaction in an isobutanol pathway, and (c) feeding the host of step (b) a methane substrate under suitable growth conditions, wherein the MMO polypeptide catalyzes the substrate to product conversion of methane to methanol, an endogenous methanol dehydrogenase (MDH) polypeptide catalyzes the substrate to product conversion of methanol to formaldehyde and the formaldehyde produced is converted to pyruvate through an endogenous RuMP or serine pathway, wherein the host metabolizes pyruvate to produce isobutanol. In certain embodiments, the one or more polynucleotide ORFs introduced in step (b) encode an isobutanol pathway polypeptide selected from an Enzyme Class (EC) comprising EC 2.2.1.6, EC 1.1.1.86, EC 4.2.1.9, EC 4.1.1.72 and EC 1.1.1.1. In other embodiments, the one or more polynucleotide ORFs introduced in step (b) encode an isobutanol pathway polypeptide selected from the group consisting of acetolactate synthase (ALS), ketol-acid reductoisomerase (KARI), dihydroxy-acid dehydratase (DHAD), ketoacid decarboxylase (KDC) and alcohol dehydrogenase (ADH). In yet other embodiments, the ALS polypeptide catalyzes the substrate to product conversion of pyruvate to acetolactate; the KARI polypeptide catalyzes the substrate to product conversion of acetolactate to 2,3-dihydroxyisovalerate; the DHAD polypeptide catalyzes the substrate to product conversion of 2,3-dihydroxyisovalerate to ketoisovalerate; the KDC polypeptide catalyzes the substrate to product conversion of ketoisovalerate to isobutryaldehyde and ADH polypeptide catalyzes the substrate to product conversion of isobutyraldehyde to isobutanol. In certain other embodiments, the ALS polypeptide comprises an amino acid sequence having at least 90% sequence homology to SEQ ID NO: 2, the KARI polypeptide comprises an amino acid sequence having at least 90% sequence homology to SEQ ID NO:4, the DHAD polypeptide comprises an amino acid sequence having at least 90% sequence homology to SEQ ID NO:6, the KDC polypeptide comprises an amino acid sequence having at least 90% sequence homology to SEQ ID NO: 8 and the ADH polypeptide comprises an amino acid sequence having at least 90% sequence homology to SEQ ID NO:10. In another embodiment, the one or more polynucleotide ORFs introduced in step (b) encode the complete isobutanol pathway comprising an ALS polypeptide, a KARI polypeptide, a DHAD polypeptide, a KDC polypeptide and an ADH polypeptide. In other embodiments, the methane monooxygenase (MMO) is a soluble MMO of Enzyme Class EC 1.14.13.25 or a particulate MMO of Enzyme Class 1.14.18.3. In certain embodiments, the MMO comprises an amino acid sequence comprising at least 90% sequence homology to a particulate methane monooxygenase (pMMO) selected from the group consisting of SEQ ID NO:12, SEQ ID NO: 14, SEQ ID NO: 16, SEQ ID NO: 18, SEQ ID NO:20 and SEQ ID NO:22 or at least 90% sequence homology to a soluble methane monooxygenase (sMMO) selected from the group consisting of SEQ ID NO:24, SEQ ID NO:26, SEQ ID NO:28, SEQ ID NO:30, SEQ ID NO: 32 and SEQ ID NO:34. In other embodiments a method for producing isobutanol from a methane substrate further comprises the step of recovering the isobutanol produced.

In another embodiment, the invention is directed to a method for producing 1-butanol from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing at least one polynucleotide open reading frame (ORF), under the control of suitable regulatory sequences, wherein the at least one polynucleotide ORF encodes a polypeptide that catalyzes a reaction in a 1-butanol pathway, and (c) feeding the methanotroph host of step (b) a methane substrate under suitable growth conditions, wherein the host metabolizes methane to formaldehyde as set forth in step (a), wherein the formaldehyde is converted to pyruvate by means of an endogenous type I RuMP pathway or a type II serine pathway and the host metabolizes pyruvate to produce 1-butanol. In certain embodiments, the one or more polynucleotide ORFs introduced in step (b) encode a 1-butanol pathway polypeptide selected from an Enzyme Class (EC) comprising EC 4.3.1.19, EC 2.3.3.6, EC 4.2.1.33, EC 4.1.1.72, and EC 1.1.1.1. In yet other embodiments, the one or more polynucleotide ORFs introduced in step (b) encode a 1-butanol pathway polypeptide selected from the group consisting of L-threonine ammonia lyase, 2-ethylmalate synthase, isopropylmalate isomerase, 3-isopropylmalate dehydratase, 3-isopropylmalate dehydrogenase, 2-ketoacid decarboxylase (KDC) and alcohol dehydrogenase (ADH). In another embodiment, the L-threonine ammonia lyase catalyzes the substrate to product conversion of L-threonine to 2-oxybutanoate and ammonia; the 2-ethylmalate synthase catalyzes the substrate to product conversion of 2-oxybutanoate and acetyl-CoA to 2-ethylmalate; the isopropylmalate isomerase catalyzes the substrate to product conversion of 2-ethylmalate to 3-ethylmalate; the 3-isopropylmalate dehydrogenase catalyzes the substrate to product conversion of 3-ethylmalate to 2-ketovalerate, CO₂ and NADH; the KDC catalyzes the substrate to product conversion of 2-ketovalerate to butryaldehyde and the ADH catalyzes the substrate to product conversion of butyraldehyde to 1-butanol. In another embodiment, the L-threonine ammonia lyase comprises an amino acid sequence having at least 90% sequence homology to SEQ ID NO:56, the 2-ethylmalate synthase comprises an amino acid sequence having at least 90% sequence homology to SEQ ID NO:58, the isopropylmalate isomerase comprises an amino acid sequence having at least 90% sequence homology to SEQ ID NO: 60 and SEQ ID NO:62, a 3-isopropylmalate dehydrogenase comprises an amino acid sequence having at least 90% sequence homology to SEQ ID NO:64, the KDC comprises an amino acid sequence having at least 90% sequence homology to SEQ ID NO:8 and the ADH comprises an amino acid sequence having at least 90% sequence homology to SEQ ID NO: 10. In certain other embodiments, the one or more polynucleotide ORFs introduced in step (b) encode the complete 1-butanol pathway comprising L-threonine ammonia lyase, 2-ethylmalate synthase, isopropylmalate isomerase, 3-isopropylmalate dehydrogenase, 2-ketoacid decarboxylase (KDC) and alcohol dehydrogenase (ADH). In other embodiments a method for producing 1-butanol from a methane substrate further comprises the step of recovering the 1-butanol produced.

In another embodiment, the invention is directed to a method for producing 1-butanol from a methane substrate comprising the steps of (a) providing a non-methanotroph host microorganism which has been genetically engineered to express a methane monooxygenase (MMO), (b) introducing into the host and expressing at least one polynucleotide open reading frame (ORF), under the control of suitable regulatory sequences, wherein the at least one polynucleotide ORF encodes a polypeptide that catalyzes a reaction in a 1-butanol pathway, and (c) feeding the host of step (b) a methane substrate under suitable growth conditions, wherein the MMO polypeptide catalyzes the substrate to product conversion of methane to methanol, an endogenous methanol dehydrogenase (MDH) polypeptide catalyzes the substrate to product conversion of methanol to formaldehyde and the formaldehyde produced is converted to pyruvate through an endogenous RuMP or serine pathway, wherein the host metabolizes pyruvate to produce 1-butanol. In certain embodiments, the one or more polynucleotide ORFs introduced in step (b) encode an 1-butanol pathway polypeptide selected from an Enzyme Class (EC) comprising EC 4.3.1.19, EC 2.3.3.6, EC 4.2.1.33, EC 1.1.1.85, EC 4.1.1.72, and EC 1.1.1.1. In another embodiment, the one or more polynucleotide ORFs introduced in step (b) encode a 1-butanol pathway polypeptide selected from the group consisting of L-threonine ammonia lyase, 2-ethylmalate synthase, isopropylmalate isomerase, 3-isopropylmalate dehydrogenase, 2-ketoacid decarboxylase (KDC) and alcohol dehydrogenase (ADH). In yet other embodiments, the L-threonine ammonia lyase catalyzes the substrate to product conversion of L-threonine to 2-oxybutanoate and ammonia; the 2-ethylmalate synthase catalyzes the substrate to product conversion of 2-oxybutanoate and acetyl-CoA to 2-ethylmalate; the isopropylmalate isomerase catalyzes the substrate to product conversion of 2-ethylmalate to 3-ethylmalate; the 3-isopropylmalate dehydrogenase catalyzes the substrate to product conversion of 3-ethylmalate to 2-ketovalerate, CO₂ and NADH; the KDC catalyzes the substrate to product conversion of 2-ketovalerate to butryaldehyde and the ADH catalyzes the substrate to product conversion of butyraldehyde to 1-butanol. In other embodiments, the L-threonine ammonia lyase comprises an amino acid sequence having at least 90% sequence homology to SEQ ID NO:56, the 2-ethylmalate synthase comprises an amino acid sequence having at least 90% sequence homology to SEQ ID NO: 58, the isopropylmalate isomerase comprises an amino acid sequence having at least 90% sequence homology to SEQ ID NO:60 and SEQ ID NO:62, a 3-isopropylmalate dehydrogenase comprises an amino acid sequence having at least 90% sequence homology to SEQ ID NO:64, the KDC comprises an amino acid sequence having at least 90% sequence homology to SEQ ID NO:8 and the ADH comprises an amino acid sequence having at least 90% sequence homology to SEQ ID NO:10. In another embodiment, the methane monooxygenase (MMO) is a soluble MMO of Enzyme Class EC 1.14.13.25 or a particulate MMO of Enzyme Class 1.14.18.3. In certain other embodiments, the MMO comprises an amino acid sequence comprising at least 90% sequence homology to a particulate methane monooxygenase (pMMO) selected from the group consisting of SEQ ID NO: 12, SEQ ID NO: 14, SEQ ID NO: 16, SEQ ID NO:18, SEQ ID NO:20 and SEQ ID NO: 22 or at least 90% sequence homology to a soluble methane monooxygenase (sMMO) selected from the group consisting of SEQ ID NO:24, SEQ ID NO:26, SEQ ID NO:28, SEQ ID NO:30, SEQ ID NO:32 and SEQ ID NO:34. In other embodiments a method for producing 1-butanol from a methane substrate further comprises the step of recovering the 1-butanol produced.

In certain other embodiments, the invention is directed to a method for producing fatty alcohols from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a fatty-acyl-CoA reductase (FAR), and (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes acetyl-CoA to produce a fatty alcohol. In one embodiment, the FAR polypeptide is further defined as a polypeptide from Enzyme Class EC 1.2.1.50. In another embodiment, the FAR polypeptide catalyzes the substrate to product conversion of fatty acetyl-CoA to a fatty alcohol. In yet other embodiments, the FAR polypeptide comprises an amino acid sequence having at least 90% sequence homology to SEQ ID NO:66. In other embodiments a method for producing fatty alcohols from a methane substrate further comprises the step of recovering the fatty alcohol produced.

In certain other embodiments, the invention is directed to a method for producing a fatty alcohol from a methane substrate comprising the steps of (a) providing a non-methanotroph host microorganism which has been genetically engineered to express a methane monooxygenase (MMO), (b) introducing into the host microorganism and expressing a polynucleotide open reading frame (ORF), under the control of suitable regulatory sequences, wherein the polynucleotide ORF encodes a fatty-acyl-CoA reductase (FAR), and (c) feeding the host microorganism of step (b) a methane substrate under suitable growth conditions, wherein the host metabolizes methane to formaldehyde as set forth in step (a), wherein the formaldehyde is converted to acetyl-CoA by means of an endogenous RuMP or serine pathway and the host metabolizes acetyl-CoA to produce a fatty alcohol. In certain embodiments, the FAR polypeptide catalyzes the substrate to product conversion of fatty acetyl-CoA to a fatty alcohol. In certain other embodiments, the FAR polypeptide comprises an amino acid sequence comprising at least 90% sequence homology to SEQ ID NO: 66. In another embodiment, the methane monooxygenase (MMO) is a soluble MMO of Enzyme Class EC 1.14.13.25 or a particulate MMO of Enzyme Class 1.14.18.3. In other embodiments, the MMO comprises an amino acid sequence having at least 90% sequence homology to a particulate methane monooxygenase (pMMO) selected from the group consisting of SEQ ID NO: 12, SEQ ID NO:14, SEQ ID NO: 16, SEQ ID NO: 18, SEQ ID NO: 20 and SEQ ID NO:22 or at least 90% sequence homology to a soluble methane monooxygenase (sMMO) selected from the group consisting of SEQ ID NO:24, SEQ ID NO: 26, SEQ ID NO:28, SEQ ID NO:30, SEQ ID NO:32 and SEQ ID NO:34. In other embodiments a method for producing fatty alcohols from a methane substrate further comprises the step of recovering the fatty alcohol produced.

In another embodiment, the invention is directed to a method for producing a fatty acid ester from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a wax ester synthase (WES) and (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes fatty acyl-CoA and alcohols to produce a fatty acid ester. In certain embodiments, the WES polypeptide is further defined as a polypeptide from Enzyme Class EC 2.3.1.75. In another embodiment, the WES polypeptide catalyzes the substrate to product conversion of fatty acyl-CoA and alcohols to fatty acid esters. In other embodiments, the WES polypeptide comprises an amino acid sequence having at least 90% sequence homology to a WES polypeptide selected from SEQ ID NO:68, SEQ ID NO: 70, SEQ ID NO: 72, SEQ ID NO:74, SEQ ID NO: 76 and SEQ ID NO: 78. In other embodiments a method for producing fatty acid esters from a methane substrate further comprises the step of recovering the fatty acid esters produced.

In another embodiment, the invention is directed to a method for producing a fatty acid ester from a methane substrate comprising the steps of (a) providing a non-methanotroph host microorganism which has been genetically engineered to express a methane monooxygenase (MMO), (b) introducing into the host microorganism and expressing at least one polynucleotide open reading frame (ORF), under the control of suitable regulatory sequences, wherein the at least one polynucleotide ORF encodes a wax ester synthase (WES) and (c) feeding the host of step (b) a methane substrate under suitable growth conditions, wherein the MMO polypeptide catalyzes the substrate to product conversion of methane to methanol, an endogenous MDH polypeptide catalyzes the substrate to product conversion of methanol to formaldehyde, the formaldehyde produced is converted to acetyl-CoA through an endogenous RuMP or serine pathway and the host metabolizes fatty acyl-CoA and alcohols to produce a fatty acid ester. In certain embodiments, the WES polypeptide catalyzes the substrate to product conversion of fatty acyl-CoA and alcohols to fatty acid esters. In certain other embodiments, the WES polypeptide comprises an amino acid sequence having at least 90% sequence homology to a WES polypeptide selected from SEQ ID NO:68, SEQ ID NO: 70, SEQ ID NO: 72, SEQ ID NO:74, SEQ ID NO: 76 and SEQ ID NO: 78. In another embodiment, the methane monooxygenase (MMO) is a soluble MMO of Enzyme Class EC 1.14.13.25 or a particulate MMO of Enzyme Class 1.14.18.3. In other embodiments, the MMO comprises an amino acid sequence having at least 90% sequence homology to a particulate methane monooxygenase (pMMO) selected from the group consisting of SEQ ID NO: 12, SEQ ID NO:14, SEQ ID NO: 16, SEQ ID NO: 18, SEQ ID NO:20 and SEQ ID NO:22 or at least 90% sequence homology to a soluble methane monooxygenase (sMMO) selected from the group consisting of SEQ ID NO:24, SEQ ID NO:26, SEQ ID NO:28, SEQ ID NO:30, SEQ ID NO:32 and SEQ ID NO:34. In other embodiments a method for producing fatty acid esters from a methane substrate further comprises the step of recovering the fatty acid esters produced.

In certain other embodiments, the invention is directed to a method for producing 2,3-butanediol from a methane substrate comprising the steps of (a) providing a methanotrophic host microorganism that metabolizes methane (CH₄) to methanol (CH₃OH) and methanol to formaldehyde (H₂C═O), (b) introducing into the host and expressing a polynucleotide ORF, under the control of suitable regulatory sequences, wherein the ORF encodes a (2R,3R)-2,3-butanediol dehydrogenase (BDH1), and (c) feeding the host microorganism of step (b) a methane substrate under suitable growth conditions, wherein host metabolizes methane to formaldehyde as set forth in step (a), wherein the formaldehyde is converted to pyruvate by means of an endogenous type I RuMP pathway or a type II serine pathway and the host metabolizes pyruvate to produce (R)-acetoin and the BDH1 catalyzes the substrate to product conversion of (R)-acetoin to 2,3-butanediol. In certain embodiments, the (2R,3R)-2,3-butanediol dehydrogenase (BDH1) has at least 90% sequence homology to a BDH1 polypeptide of SEQ ID NO:157. In other embodiments, the polynucleotide ORF comprises a nucleotide sequence of SEQ ID NO:156. In other embodiments a method for producing 2,3-butanediol from a methane substrate further comprises the step of recovering the 2,3-butanediol produced.

In certain embodiments, a methanotroph host microorganism of the invention is selected from genus consisting of Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, and Methyloacidophilum. In other embodiments, the methanotroph host microorganism is selected from the phylum Verrucomicrobia. In another embodiment, the methanotroph host is Methylococcus capsulatus, strain Bath.

In certain other embodiments, a non-methanotroph host microorganism of the invention is a yeast microorganism or bacterial microorganism. In certain embodiments, the non-methanotroph yeast microorganism is selected from Saccharomyces cerevisiae, Hansenuela polymorpha, Pichia pastoris and Kluyveromyces lactis. In one particular embodiment, the yeast microorganism is Pichia pastoris.

In certain other embodiments, a non-methanotrophic bacterial microorganism of the invention is Pseudomonas putida, Cupriavidus metallidurans or Rhodobacter sphaeroides.

In other embodiments, recovering the isobutanol produced according to the methods of the invention is a process selected from distillation, liquid extraction, flash evaporation, membrane separation and phase separation.

In other embodiments, recovering the 1-butanol produced according to the methods of the invention is a process selected from distillation, liquid extraction, flash evaporation, membrane separation and phase separation.

In another embodiment, recovering the fatty alcohol produced according to the methods of the invention is a process selected from flash evaporation, membrane separation, centrifugation and phase separation.

In certain other embodiments, recovering the fatty acid ester produced according to the methods of the invention is a process selected from flash evaporation, membrane separation, centrifugation and phase separation.

In another embodiment, recovering the 2,3-butanediol produced according to the methods of the invention is a process selected from steam stripping, solvent extraction, aqueous two-phase extraction, reactive extraction and pervaporation.

In certain other embodiments, a methane substrate is provided as a dry natural gas, as a wet natural gas or as a biogas.

In other embodiments, the host microorganism is grown by a batch process, a fed-batch process or a continuous perfusion process.

In another embodiment, the fatty alcohol composition produced according to the methods of the invention comprises a carbon chain of about 5 to about 40 carbon atoms. In certain embodiments, the fatty alcohol comprises a carbon chain of 8 to 22 carbon atoms.

In another embodiment, the fatty acid ester composition produced according to the methods of the invention has a fatty acid moiety comprising a carbon chain of about 5 to about 40 carbon atoms. In one particular embodiment, the fatty acid moiety comprises a carbon chain of 8 to 22 carbon atoms.

In yet other embodiments, the fatty acid ester composition produced according to the methods of the invention has an alcohol moiety comprising a carbon chain of about 5 to about 40 carbon atoms. In one particular embodiment, the alcohol moiety comprises a chain of 8 to 22 carbon atoms.

In yet other embodiments, a non-methanotroph host microorganism of the invention is further engineered to express an exogenous methanol dehydrogenase (MDH). In certain embodiments, the MDH is a polypeptide from Enzyme Class 1.14.18.3. In other embodiments, the MDH comprises an amino acid sequence having at least 90% sequence homology to a MDH polypeptide selected from the group consisting of SEQ ID NO:36, SEQ ID NO:38, SEQ ID NO:40, SEQ ID NO:42, SEQ ID NO:44, SEQ ID NO:46, SEQ ID NO: 48, SEQ ID NO:50, SEQ ID NO:52 and SEQ ID NO:54.

In other embodiments, the invention is directed to a substantially purified isobutanol composition produced according to the methods of the invention.

In another embodiment, the invention is directed to a substantially purified 1-butanol composition produced according to the methods of the invention.

In other embodiments, the invention is directed to a substantially purified fatty alcohol composition produced according to the methods of the invention.

In another embodiment, the invention is directed to a substantially purified fatty acid ester composition produced according to the methods of the invention.

In other embodiments, the invention is directed to a substantially purified 2,3-butanediol composition produced according to the methods of the invention.

In yet other embodiments, the invention is directed to an isobutanol producing methanotroph host microorganism manufactured according to the steps of (a) providing a methanotrophic host microorganism that metabolizes methane (CH₄) to methanol (CH₃OH) and methanol to formaldehyde (H₂C═O), (b) introducing into the methanotroph host and expressing at least one polynucleotide open reading frame (ORF), under the control of suitable regulatory sequences, wherein the at least one polynucleotide ORF encodes a polypeptide that catalyzes a reaction in an isobutanol pathway, and (c) feeding the methanotroph host of step (b) a methane substrate under suitable growth conditions, wherein the host metabolizes methane to formaldehyde as set forth in step (a), wherein the formaldehyde is converted to pyruvate by means of an endogenous type I RuMP pathway or a type II serine pathway and the host metabolizes pyruvate to produce isobutanol.

In another embodiment, the invention is directed to an isobutanol producing non-methanotroph host microorganism manufactured according to the steps of (a) providing a non-methanotroph host microorganism which has been genetically engineered to express a methane monooxygenase (MMO), (b) introducing into the host and expressing at least one polynucleotide open reading frame (ORF), under the control of suitable regulatory sequences, wherein the at least one polynucleotide ORF encodes a polypeptide that catalyzes a reaction in an isobutanol pathway, and (c) feeding the host of step (b) a methane substrate under suitable growth conditions, wherein the MMO polypeptide catalyzes the substrate to product conversion of methane to methanol, an endogenous methanol dehydrogenase (MDH) polypeptide catalyzes the substrate to product conversion of methanol to formaldehyde and the formaldehyde produced is converted to pyruvate through an endogenous RuMP or serine pathway, wherein the host metabolizes pyruvate to produce isobutanol.

In yet other embodiments, the invention is directed to a 1-butanol producing methanotroph host microorganism manufactured according to the steps of (a) providing a methanotrophic host microorganism that metabolizes methane (CH₄) to methanol (CH₃OH) and methanol to formaldehyde (H₂C═O), (b) introducing into the methanotroph host and expressing at least one polynucleotide open reading frame (ORF), under the control of suitable regulatory sequences, wherein the at least one polynucleotide ORF encodes a polypeptide that catalyzes a reaction in a 1-butanol pathway, and (c) feeding the methanotroph host of step (b) a methane substrate under suitable growth conditions, wherein the host metabolizes methane to formaldehyde as set forth in step (a), wherein the formaldehyde is converted to pyruvate by means of an endogenous type I RuMP pathway or a type II serine pathway and the host metabolizes pyruvate to produce 1-butanol.

In other embodiments, the invention is directed to a 1-butanol producing non-methanotroph host microorganism manufactured according to the steps of (a) providing a non-methanotroph host microorganism which has been genetically engineered to express a methane monooxygenase (MMO), (b) introducing into the host and expressing at least one polynucleotide open reading frame (ORF), under the control of suitable regulatory sequences, wherein the at least one polynucleotide ORF encodes a polypeptide that catalyzes a reaction in a 1-butanol pathway, and (c) feeding the host of step (b) a methane substrate under suitable growth conditions, wherein the MMO polypeptide catalyzes the substrate to product conversion of methane to methanol, an endogenous methanol dehydrogenase (MDH) polypeptide catalyzes the substrate to product conversion of methanol to formaldehyde and the formaldehyde produced is converted to pyruvate through an endogenous RuMP or serine pathway, wherein the host metabolizes pyruvate to produce 1-butanol.

In another embodiment, the invention is directed to a fatty alcohol producing methanotroph host microorganism manufactured according to the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a fatty-acyl-CoA reductase (FAR), and (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes acetyl-CoA to produce a fatty alcohol.

In other embodiments, the invention is directed to a fatty alcohol producing non-methanotroph host microorganism manufactured according to the steps of (a) providing a non-methanotroph host microorganism which has been genetically engineered to express a methane monooxygenase (MMO), (b) introducing into the host microorganism and expressing a polynucleotide open reading frame (ORF), under the control of suitable regulatory sequences, wherein the polynucleotide ORF encodes a fatty-acyl-CoA reductase (FAR), and (c) feeding the host microorganism of step (b) a methane substrate under suitable growth conditions, wherein the host metabolizes methane to formaldehyde as set forth in step (a), wherein the formaldehyde is converted to acetyl-CoA by means of an endogenous RuMP or serine pathway and the host metabolizes acetyl-CoA to produce a fatty alcohol.

In another embodiment, the invention is directed to a fatty acid ester producing methanotroph host microorganism manufactured according to the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a wax ester synthase (WES) and (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes fatty acyl-CoA and alcohols to produce a fatty acid ester.

In certain other embodiments, the invention is directed to a fatty acid ester producing non-methanotroph host microorganism manufactured according to the steps of (a) providing a non-methanotroph host microorganism which has been genetically engineered to express a methane monooxygenase (MMO), (b) introducing into the host microorganism and expressing at least one polynucleotide open reading frame (ORF), under the control of suitable regulatory sequences, wherein the at least one polynucleotide ORF encodes a wax ester synthase (WES) and (c) feeding the host of step (b) a methane substrate under suitable growth conditions, wherein the MMO polypeptide catalyzes the substrate to product conversion of methane to methanol, an endogenous MDH polypeptide catalyzes the substrate to product conversion of methanol to formaldehyde, the formaldehyde produced is converted to acetyl-CoA through an endogenous RuMP or serine pathway and the host metabolizes fatty acyl-CoA and alcohols to produce a fatty acid ester.

In certain other embodiments, the invention is directed to a 2,3-butanediol producing methanotroph host microorganism manufactured according to the steps of (a) providing a methanotrophic host microorganism that metabolizes methane (CH₄) to methanol (CH₃OH) and methanol to formaldehyde (H₂C═O), (b) introducing into the host and expressing a polynucleotide ORF, under the control of suitable regulatory sequences, wherein the ORF encodes a (2R,3R)-2,3-butanediol dehydrogenase (BDH1), and (c) feeding the host microorganism of step (b) a methane substrate under suitable growth conditions, wherein host metabolizes methane to formaldehyde as set forth in step (a), wherein the formaldehyde is converted to pyruvate by means of an endogenous type I RuMP pathway or a type II serine pathway and the host metabolizes pyruvate to produce (R)-acetoin and the BDH1 catalyzes the substrate to product conversion of (R)-acetoin to 2,3-butanediol.

### BRIEF DESCRIPTION OF THE DRAWINGS

Certain embodiments of the invention are illustrated in the drawings, in which:

FIG. 1 depicts five pathways for the biosynthetic production of n-butanol (1-butanol) and one pathway for the biosynthetic production of isobutanol.

FIG. 2 depicts a vector map of the broad host range expression plasmid pCM 132. The vector map shows the following components: (1) an origin of replication that is functional in E. coli (colE1); (2) an oriV/IncP origin of replication for the non-E. coli microbial host; (3) an oriT/IncP origin of transfer, which is needed for transferring a bacterial plasmid from a bacterial host such as E. coli to the recipient during bacterial conjugation; (4) a traJ gene, which codes for a transcriptional activator that initiates production of the proteins needed for conjugative transfer; (5) a trfA gene, the replication initiation protein gene of plasmid RK2 which binds to and activates oriV; (6) a lacZ (beta-galactosidase) gene for identifying plasmids with DNA inserts based on colony color using indolyl-galactoside-based substrates; and (7) a kanamycin resistance gene (kan). Genes of interest are inserted into the polylinker region that lies between the rrnB transcription terminator and the 5′-end of the lacZ gene.

FIG. 3 shows a schematic diagram of component DNA arrangements for cloning into an expression vector.

FIG. 4 shows the optimization of the 2-ketoisovalerate (2-KIV) concentration fed to various engineered host strains expressing the two-gene (isobutanol) pathway.

FIG. 5 shows the measured production of isobutanol in an M. capsulatus strain expressing plasmid pGMV145 (containing: promoter J23115, the gene for M. capsulatus (Bath) 2-ketoisovalerate decarboxylase (CapKDC) and the gene for S. cerevisiae alcohol dehydrogenase (ScADH6)), harvested at different time intervals after 2-KIV addition.

FIG. 6 is a comparison of isobutanol production after 2-KIV feeding in various engineered host strains expressing different combinations of two genes (i.e., isobutanol pathway genes) and with different promoters.

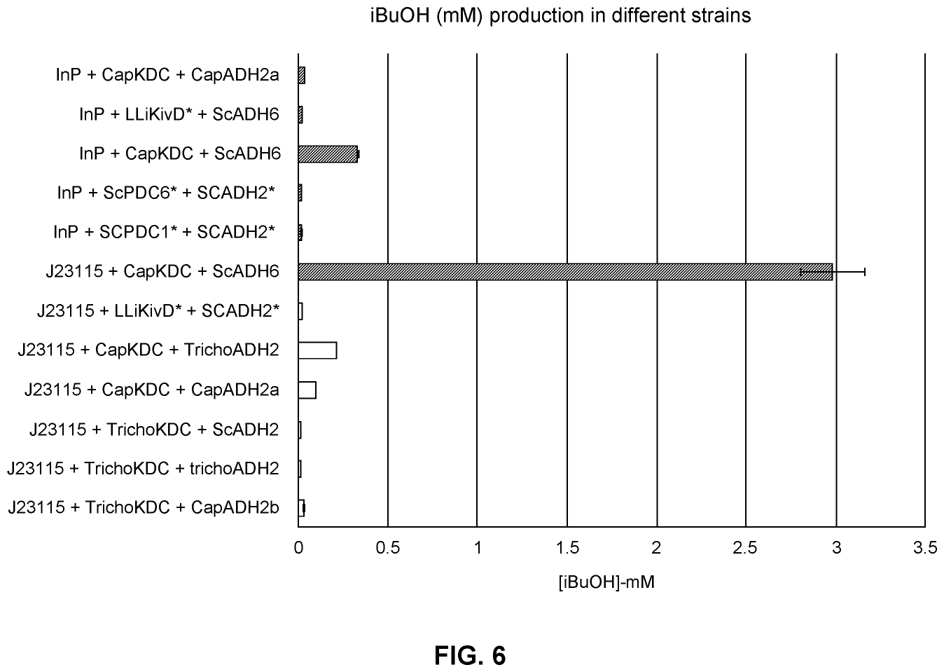

FIG. 7 compares production of isobutanol in the wild-type M. capsulatus str. Bath (no plasmid) and an engineered strain (pJS041) expressing the five-gene (isobutanol) pathway.

FIG. 8 is a gas chromatography (GC) analysis of fatty acid alcohol production by various engineered strains of M. capsulatus (Bath). The GC peak at about 5.2 minutes corresponds to a C16:0 fatty alcohol standard.

FIG. 9 shows an engineered pathway for 2,3-butanediol production from methane using a heterologously expressed BDH1 enzyme from Saccharomyces cerevisiae.

### DETAILED DESCRIPTION OF THE INVENTION

Unless defined otherwise, all technical and scientific terms used herein have the same meaning as commonly understood to one of ordinary skill in the art to which this disclosure pertains. Although methods and materials similar or equivalent to those described herein can be used in the practice of the disclosed methods and compositions, the exemplary methods, devices and materials are described herein.

In certain embodiments, the present invention is directed to host microorganisms metabolically engineered to produce multi-carbon compounds. Multi-carbon compounds such as ethanol, n-butanol, sec-butanol, isobutanol, tert-butanol, fatty (or aliphatic long chain) alcohols, fatty acid methyl esters, 2,3-butanediol and the like, are important industrial commodity chemicals with a variety of applications, including, but not limited to their use in fuels (e.g., bio-fuel, bio-diesel) and bio-based chemicals. The present invention addresses a number of commercial, industrial and environmental needs in the art related to the production of multi-carbon compounds.

As set forth herein, the metabolically engineered host microorganisms of the present invention utilize methane (CH₄) as their sole carbon source (i.e., the host microorganism does not require plant based feedstocks for growth and energy) and ameliorate much of the ongoing depletion of arable food source “farmland” currently being diverted to grow bio-fuel feedstocks, and as such, improve the environmental footprint of future bio-fuel, bio-diesel and bio-based chemical compositions. Furthermore, use of the metabolically engineered host microorganisms set forth in the present invention (which utilize methane as the sole carbon source) mitigate current industry practices and methods of producing multi-carbon compounds from petroleum or petroleum-derived feedstocks.

Thus, in certain embodiments of the invention, a host microorganism is genetically engineered to produce multi-carbon compounds. As is known in the art, methanotrophic organisms are able to metabolize methane as their primary source of carbon and energy, can grow aerobically or anaerobically, and require single-carbon compounds (e.g., methane, CH₄; methanol, CH₃OH and/or formaldehyde, H₂C═O) to survive. In particular embodiments, a host microorganism of the invention is a methanotroph. As defined herein, a “methanotroph”, a “methanotrophic” or a “methanophile” host microorganism of the invention is a “prokaryotic microorganism which can metabolize methane as its primary source of carbon and energy”.

In other embodiments, the host microorganism of the invention is a non-methanotrophic microorganism genetically engineered to metabolize methane as its only source of carbon and energy. As defined herein, a “non-methanotroph” host microorganism of the invention is a host microorganism which “cannot metabolize (or utilize) methane as its sole carbon source”, until the “non-methanotroph” host microorganism has been genetically modified or engineered according to the methods of the present invention. As further defined herein, a “non-methanotroph” host microorganism of the invention includes any prokaryotic and eukaryotic microbial species which comprise a complete or partial “endogenous ribulose monophosphate (RuMP) pathway, a serine pathway or a mixed RuMP/serine pathway” (e.g., see RuMP, serine and mixed (Type X) pathways described below). In certain embodiments, a “non-methanotroph” host microorganism of the invention includes prokaryotic and eukaryotic microbial species from the Domains Archaea, Bacteria and Eucarya, wherein the Domain Eucarya includes yeast, filamentous fungi, protozoa, algae or higher Protista. The terms “microbial” and “microbes” are used interchangeably with the term “microorganism”.

As defined herein, the phrase “providing a methanotrophic host microorganism that metabolizes methane to methanol and metabolizes methanol to formaldehyde” refers to an “endogenous enzymatic activity encoded by one or more endogenous genes of the methanotroph host microorganism”. For example, an endogenous enzyme (or polypeptide) encoded by one or more endogenous genes of a methanotroph host microorganism include a methane monooxygenase (MMO) enzyme (which metabolizes (or converts) methane to methanol) and a methanol dehydrogenase (MDH) enzyme (which metabolizes (or converts) methanol to formaldehyde). Stated another way, the phrase “providing a methanotrophic host microorganism that metabolizes methane to methanol and metabolizes methanol to formaldehyde” does not require the introduction of exogenous (or heterologous) genes encoding single-carbon (C1) oxidizing enzymes (or polypeptides), as such enzymes and the activity thereof are inherent (endogenous) attributes of a methanotrophic host microorganism of the invention.

Furthermore, as is known in the art, a “methanotrophic host microorganism” of the invention comprises endogenous genes encoding at least a Type I methanotroph RuMP pathway and/or a Type II methanotroph serine pathway. In general, Type I methanotrophs (e.g., Methylomonas, Methylomicrobium, Methylobacter, Methylocaldum, Methylosphaera) assimilate formaldehyde produced (i.e., from the oxidation of methane to methanol and methanol to formaldehyde), using the ribulose monophosphate pathway (RuMP), whereas Type II methanotrophs (e.g., Methylocystis and Methylosinus) assimilate formaldehyde produced (i.e., from the oxidation of methane to methanol and methanol to formaldehyde), using the serine pathway. Lastly, the genus Methylococcus are known to comprise a combination of characteristics of both Type I methanotroph (RuMP) pathway and Type II methanotroph (serine) pathway.

The ribulose monophosphate pathway (RuMP) was originally identified in methanotrophic bacteria, as described above. However, more recent genome sequence analysis of various microorganisms have revealed that the key enzymes of the RuMP pathway (e.g., 3-hexulose-6-phosphate (HPS), 6-phsopho-3-hexuloisomerase (PHI)) are widely distributed (i.e., endogenous) among “non-methanotrophic” bacteria and archaeal genomes (Orita et al., 2006).

As defined herein, the phrases “recombinant host microorganism”, “genetically engineered host microorganism”, “engineered host microorganism” and “genetically modified host microorganism” may be used interchangeably and refer to host microorganisms that have been genetically modified to (a) express one or more exogenous polynucleotides, (b) over-express one or more endogenous and/or one or more exogenous polynucleotides, such as those included in a vector, or which have an alteration in expression of an endogenous gene or (c) knock-out or down-regulate an endogenous gene. In addition, certain genes may be physically removed from the genome (e.g., knock-outs) or they may be engineered to have reduced, altered or enhanced activity.

The terms “engineer”, “genetically engineer” or “genetically modify” refer to any manipulation of a microorganism that results in a detectable change in the microorganism, wherein the manipulation includes, but is not limited to, introducing non-native metabolic functionality via heterologous (exogenous) polynucleotides or removing native-functionality via polynucleotide deletions, mutations or knock-outs. The term “metabolically engineered” generally involves rational pathway design and assembly of biosynthetic genes (or ORFs), genes associated with operons, and control elements of such polynucleotides, for the production of a desired metabolite. “Metabolically engineered” may further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway.

As defined herein, the term “introducing”, as used in phrases such as “introducing into the methanotroph host” or “introducing into the non-methanotroph host” at least one polynucleotide open reading frame (ORF) or a gene thereof or a vector thereof includes methods known in the art for introducing polynucleotides into a cell, including, but not limited to transformation (e.g., calcium chloride, electroporation), transduction, transfection, conjugation and the like.

The phrases “metabolically engineered microorganism” and “modified microorganism” are used interchangeably herein, and refer not only to the particular subject host cell, but to the progeny or potential progeny of such a cell. Because certain modifications may occur in succeeding generations due to either mutation or environmental influences, such progeny may not, in fact, be identical to the parent cell, but are still included within the scope of the term as used herein.

The term “mutation” as used herein indicates any modification of a nucleic acid and/or polypeptide which results in an altered nucleic acid or polypeptide (i.e., relative to the wild-type nucleic acid or polypeptide sequence). Mutations include, for example, point mutations, substitutions, deletions, or insertions of single or multiple residues in a polynucleotide (or the encoded polypeptide), which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In certain embodiments, a portion of a genetically modified microorganism's genome may be replaced with one or more heterologous (exogenous) polynucleotides. In some embodiments, the mutations are naturally-occurring. In other embodiments, the mutations are the results of artificial selection pressure. In still other embodiments, the mutations in the microorganism genome are the result of genetic engineering.

The term “expression” or “expressed” with respect to a gene sequence, an ORF sequence or polynucleotide sequence, refers to transcription of the gene, ORF or polynucleotide and, as appropriate, translation of the resulting mRNA transcript to a protein. Thus, as will be clear from the context, expression of a protein results from transcription and translation of the open reading frame sequence. The level of expression of a desired product in a host microorganism may be determined on the basis of either the amount of corresponding mRNA that is present in the host, or the amount of the desired product encoded by the selected sequence. For example, mRNA transcribed from a selected sequence can be quantitated by PCR or by northern hybridization (see Sambrook et al., Molecular Cloning: A Laboratory Manual, Cold Spring Harbor Laboratory Press, 1989). Protein encoded by a selected sequence can be quantitated by various methods (e.g., by ELISA, by assaying for the biological activity of the protein, or by employing assays that are independent of such activity, such as western blotting or radioimmunoassay, using antibodies that are recognize and bind reacting the protein).

The term “endogenous”, as used herein with reference to polynucleotides (and the polypeptides encoded therein), indicates polynucleotides and polypeptides that are expressed in the organism in which they originated (i.e., they are innate to the organism). In contrast, the terms “heterologous” and “exogenous” are used interchangeably, and as defined herein with reference to polynucleotides (and the polypeptides encoded therein), indicates polynucleotides and polypeptides that are expressed in an organism other than the organism from which they (i.e., the polynucleotide or polypeptide sequences) originated or where derived.

The term “feedstock” is defined as a raw material or mixture of raw materials supplied to a microorganism, or fermentation process, from which other products can be made. For example, as set forth in the present invention, a methane carbon source or a methanol carbon source or a formaldehyde carbon source, either alone or in combination, are feedstocks for a microorganism that produces a bio-fuel or bio-based chemical in a fermentation process. However, in addition to a feedstock (e.g., a methane substrate) of the invention, the fermentation media contains suitable minerals, salts, cofactors, buffers and other components, known to those skilled in the art, suitable for the growth of the cultures and promotion of the enzymatic pathways necessary for multi-carbon compound production.

The term “substrate” refers to any substance or compound that is converted, or meant to be converted, into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material (e.g., methane), but also intermediate and end product metabolites used in a pathway associated with a metabolically engineered microorganism as described herein.

The term “fermentation” or “fermentation process” is defined as a process in which a host microorganism is cultivated in a culture medium containing raw materials, such as feedstock and nutrients, wherein the microorganism converts raw materials, such as a feedstock, into products.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA. The term “nucleotide” refers to any of several compounds that consist of a ribose or deoxyribose sugar joined to a purine or a pyrimidine base and to a phosphate group, and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers, respectively, to a nucleotide or nucleoside in which one or more individual atoms have been replaced with a different atom or with a different functional group. Accordingly, the term polynucleotide includes nucleic acids of any length, including DNA, RNA, ORFs, analogs and fragments thereof.

As defined herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) of more codons representing amino acids, and (iii) a termination codon, the ORF being read (or translated) in the 5′ to 3′ direction.

It is understood that the polynucleotides described herein include “genes” and that the nucleic acid molecules described herein include “vectors” or “plasmids”. Accordingly, the term “gene”, refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence.

The term “promoter” refers to a nucleic acid sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

The term “codon-optimized” as it refers to genes or coding regions of nucleic acid molecules (or ORFs) for transformation of various hosts, refers to the alteration of codons in the gene or coding regions of the nucleic acid molecules to reflect the typical codon usage of the host organism without altering the polypeptide encoded by the DNA.

The term “operon” refers to two or more genes which are transcribed as a single transcriptional unit from a common promoter. In certain embodiments, the genes, polynucleotides or ORFs comprising the operon are contiguous genes. It is understood that transcription of an entire operon can be modified (i.e., increased, decreased, or eliminated) by modifying the common promoter. Alternatively, any gene, polynucleotide or ORF, or any combination thereof in an operon can be modified to alter the function or activity of the encoded polypeptide. The modification can result in an increase or a decrease in the activity or function of the encoded polypeptide. Further, the modification can impart new activities on the encoded polypeptide.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes”, that is, that replicate autonomously or can integrate into a chromosome of a host microorganism. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The term “homolog”, as used with respect to an original enzyme, polypeptide, gene or polynucleotide (or ORF encoding the same) of a first family or species, refers to distinct enzymes, genes or polynucleotides of a second family or species, which are determined by functional, structural or genomic analyses to be an enzyme, gene or polynucleotide of the second family or species, which corresponds to the original enzyme or gene of the first family or species. Most often, “homologs” will have functional, structural or genomic similarities. Techniques are known by which homologs of an enzyme, gene or polynucleotide can readily be cloned using genetic probes and PCR. Identity of cloned sequences as “homologs” can be confirmed using functional assays and/or by genomic mapping of the genes.

A polypeptide (or protein or enzyme) has “homology” or is “homologous” to a second polypeptide if the nucleic acid sequence that encodes the polypeptide has a similar sequence to the nucleic acid sequence that encodes the second polypeptide. Alternatively, a polypeptide has homology to a second polypeptide if the two proteins have “similar” amino acid sequences. Thus, the terms “homologous proteins” or “homologous polypeptides” is defined to mean that the two polypeptides have similar amino acid sequences. In certain embodiments of the invention, polynucleotides and polypeptides homologous to one or more polynucleotides and/or polypeptides set forth in Table 1 may be readily identified using methods known in the art for sequence analysis and comparison.

A homologous polynucleotide or polypeptide sequence of the invention may also be determined or identified by BLAST analysis (Basic Local Alignment Search Tool) or similar bioinformatic tools, which compare a query nucleotide or polypeptide sequence to a database of known sequences. For example, a search analysis may be done using BLAST to determine sequence identity or similarity to previously published sequences, and if the sequence has not yet been published, can give relevant insight into the function of the DNA or protein sequence.

### Bioconversion of Methane to Multi-Carbon Compounds

In general, the conversion of methane (CH₄) to multi-carbon compounds such as isobutanol ((CH₃)₂CHCH₂OH), 1-butanol or n-butanol (CH₃CH₂CH₂CH₂OH), ethanol (CH₃CH₂OH), fatty alcohols, fatty acid esters, 2,3-butanediol and the like, using a “methanotrophic host microorganism”, requires at least the following three steps, all of which are innate (or endogenous) with respect to methanotrophic organisms: (1) a methane (CH₄) substrate is oxidized to methanol (CH₃OH) via a methane monooxygenase (MMO) (e.g., particulate methane monooxygenase (pMMO) or soluble methane monooxygenase (sMMO)), (2) the methanol (CH₃OH) is oxidized to formaldehyde (H₂C═O) via methanol dehydrogenase (MDH) and (3) the formaldehyde (H₂C═O) produced in step (2) above is assimilated into a central metabolism pathway (e.g., see type I (RuMP) and type II (serine) pathways described below).

In certain embodiments of the invention, a host microorganism is a methanotroph, which endogenously expresses a methane monooxygenase (MMO) enzyme and a methanol dehydrogenase (MDH) enzyme. In other embodiments of the invention, a host microorganism of the invention is a “non-methanotrophic” prokaryotic microorganism (e.g., a non-methanotrophic bacteria or archaea) or a eukaryotic microorganism (e.g., fungi and algae) engineered to utilize a methane substrate (as sole carbon source) for growth and energy. Thus, in certain embodiments of the invention, a “non-methanotrophic” microorganism is engineered to express (or over-express) an exogenous methane monooxygenase (MMO), an enzyme requisite to metabolize methane to methanol. The non-methanotroph host microorganisms of the invention comprise an endogenous dehydrogenase (MDH) enzyme, which converts methanol to formaldehyde. However, in certain embodiments, the “non-methanotroph” microorganism is further engineered to express an exogenous methanol dehydrogenase (MDH) enzyme, which converts methanol to formaldehyde. The expression of the exogenous MDH enzyme in a non-methanotroph host is not a strict requirement for the utilization of the methane substrate, but it is contemplated in certain embodiments, that the introduction and expression of an exogenous MDH in a non-methanotroph host thereof may facilitate, under certain growth conditions, the production of one or more multi-carbon compounds of the invention.

As mentioned briefly above with regard to methanotrophic host organisms, there are at least two known pathways (i.e., the ribulose monophosphate (RuMP) pathway and the serine pathway; Hanson & Hanson, 1996) for the assimilation of formaldehyde into central metabolism. In the Type I methanotroph RuMP pathway, formaldehyde combines with ribulose-5-phosphate to form hexulose-6-phosphate (catalyzed via hexulose-6-phosphate synthase), the hexulose-6-phosphate is then isomerized to fructose-6-phosphate (catalyzed via hexulose phosphate isomerase), which is an intermediate of a central metabolic pathway (i.e., glycolysis pathway). In the type II methanotroph serine pathway, formaldehyde reacts with tetrahydrofolate (THF) to form methylene-THF, the methylene-THF is then transferred to L-glycine to form L-serine, and finally the L-serine is transferred to glyoxylate to form hydroxypyruvate. The hydroxypyruvate formed is subsequently converted to 2-phosphoglycerate (catalyzed via hydroxypruvate reductase), which is an central metabolism intermediate of the glycolytic pathway.

Likewise, as mentioned briefly above, an endogenous pathway, which functions similarly (or analogous) to the ribulose monophosphate (RuMP) pathway in methanotrophs is also present in “non-methanotrophic” prokaryotes (Orita et al., 2006), wherein formaldehyde is fixed with ribulose 5-phosphate to form hexulose-6-phosphate (catalyzed via hexulose-6-phosphate synthase (HPS)) and then isomerized to fructose-6-phosphate (catalyzed via hexulose phosphate isomerase (PHI)), which is an intermediate of a central metabolic pathway. Thus, in certain preferred embodiments, a “non-methanotrophic” host microorganism of the invention comprises an endogenous RuMP pathway or an endogenous pathway analogous to the RuMP pathway. As defined herein, a pathway analogous to the RuMP pathway comprises at least a gene, polynucleotide or ORF encoding an enzyme having hexulose-6-phosphate synthase (HPS) activity from enzyme class EC 4.1.2.43 and at least a gene, polynucleotide or ORF encoding a an enzyme having hexulose phosphate isomerase (PHI) activity from enzyme class 5.3.1.27.

In other embodiments, wherein a “non-methanotrophic” host microorganism genome does not encode endogenous enzymes having HPS and PHI activity, the non-methanotroph host microorganism is genetically modified to express HPS and PHI enzymes. Thus, in certain embodiments, a gene, polynucleotide or ORF encoding a hexulose-6-phosphate synthase (HPS) is provided, wherein the gene, polynucleotide or ORF encodes a HPS polypeptide of enzyme class EC 4.1.2.43. In other embodiments, a gene, polynucleotide or ORF encoding a hexulose-6-phosphate synthase (HPS) is provided, wherein the gene, polynucleotide or ORF encodes a HPS polypeptide having at least 90% sequence homology to a M. capsulatus (Bath) HPS polypeptide of SEQ ID NO:173. In other embodiments, a gene, polynucleotide or ORF encoding a hexulose phosphate isomerase (PHI) is provided, wherein the gene, polynucleotide or ORF encodes a PHI polypeptide of enzyme class EC 5.3.1.27. In other embodiments, a gene, polynucleotide or ORF encoding a hexulose phosphate isomerase (PHI) is provided, wherein the gene, polynucleotide or ORF encodes a M. capsulatus (Bath) PHI polypeptide having at least 90% sequence homology to a PHI (also referred to as a sugar isomerase (SIS) domain) polypeptide of SEQ ID NO:175.

Once the formaldehyde has been assimilated into a central metabolic pathway of the methanotroph or non-methanotroph host organism (as described above), the fourth and final step for producing multi-carbon compounds from a methane substrate as described in steps (1)-(3) above, is the introduction of one or more nucleic acids into the host microorganism, wherein the one or more nucleic acids introduced encode one or more enzymes of a relevant multi-carbon compound pathway. Independent of the compound to be produced according to the present invention (e.g., isobutanol, 1-butanol, ethanol, fatty alcohols, fatty acid methyl esters, 2,3-butanediol and the like), any multi-carbon pathway introduced into a host microorganism must utilize a central metabolic molecule (e.g., pyruvate, acetyl-CoA, methionine and oxobutyrate) previously assimilated and introduced into the metabolic pathway through steps (1)-(3) described above. Stated another way, a salient feature of the present invention is the ability of the host microorganism to utilize methane (as a sole carbon source for growth and energy) and to produce multi-carbon compounds (via engineered metabolic pathways introduced therein), without the need for additional or supplemental carbon sources such as carbohydrates.

As defined herein, a relevant “multi-carbon compound pathway”, includes, but is not limited to, a 1-butanol pathway (which includes, but is not limited to, a fermentative 1-butanol pathway, a thiobutanoate pathway, a ketoacid pathway and a methylmalate pathway), an isobutanol pathway, a fatty alcohol pathway, a fatty acid methyl ester pathway and a 2,3-butanediol pathway. A “multi-carbon compound pathway” as further defined herein, may include one specific enzyme from the pathway, multiple enzymes from the pathway or all of the enzymes of the pathway. It will be understood by a person of skill in the art, that the selection of one or more specific pathway enzymes (and nucleic acids encoding the same) may be dependent on the host microorganism (e.g., certain methanotroph hosts or “non-methanotroph” hosts may endogenously encode and express one or more enzymes of a given pathway).

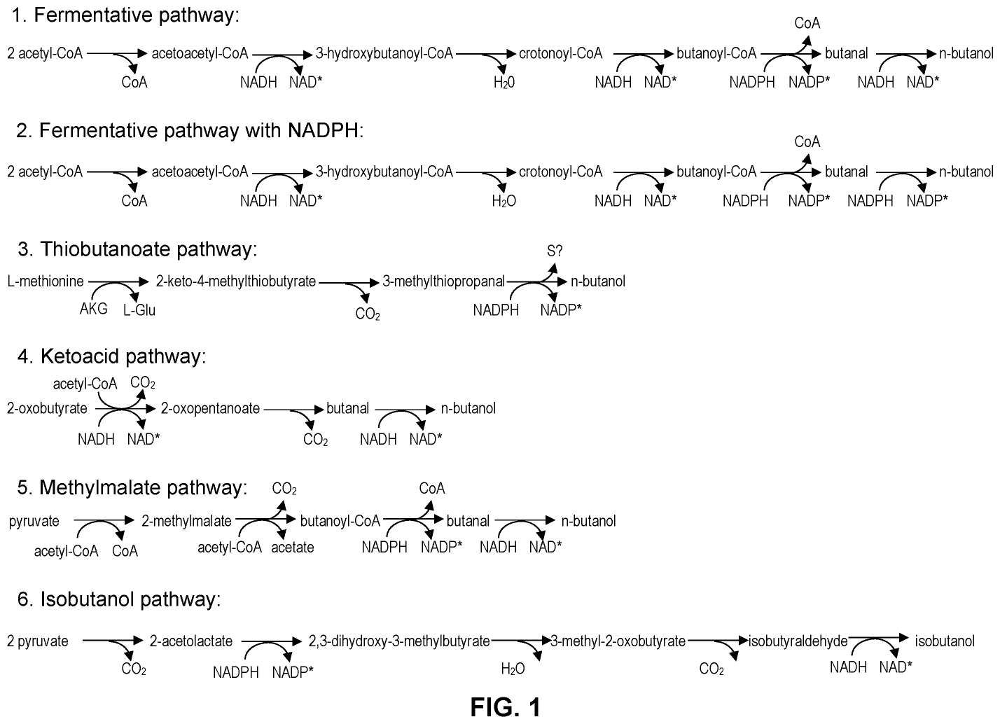

For example, FIG. 1 depicts five representative 1-butanol (i.e., n-butanol) pathways (pathways 1-5), wherein one or more nucleic acids encoding one or more enzymes of any of these pathways may be introduced into a methanotroph (or non-methanotroph) host microorganism and be expressed (or over-expressed) therein to yield 1-butanol. Similarly, FIG. 1 depicts an isobutanol pathway (pathway 6), wherein one or more nucleic acids encoding one or more enzymes of the isobutanol pathway may be introduced into a methanotroph (or non-methanotroph) host microorganism and expressed (or over expressed) therein to yield isobutanol. Further contemplated herein, is the introduction into a methanotroph (or non-methanotroph) host microorganism a combination of nucleic acids encoding one or more enzymes from a 1-butanol pathway and one or more enzymes from an isobutanol pathway.

As depicted in FIG. 1, at least five pathways are known to exist for converting one or more of these metabolic precursors into n-butanol (i.e., 1-butanol). The first synthesis pathway is the classical fermentative n-butanol pathway. Beginning with acetyl-CoA, this six step pathway requires three NADH and one NADHPH, but loses no carbon atoms to by-products formed. The second n-butanol synthesis pathway is the fermentative pathway, but using NADPH instead of NADH as the electron donor for the final conversion of butanal to n-butanol. The third potential n-butanol pathway is the thiobutanoate pathway, which begins with L-methionine, which is subsequently deaminated and then converted to n-butanol in two additional steps that involve loss of carbon dioxide (CO₂) and a sulfur(S) atom by an unknown mechanism. The fourth n-butanol pathway is the ketoacid pathway, which starting from L-threonine, n-butanol is synthesized in four steps, involving both reduction of NAD⁺ and oxidation of NADH, while losing two CO₂. The fifth n-butanol synthesis pathway is the methylmalate pathway, which begins by combining pyruvate with acetyl-CoA to form citramalate (methylmalate), a reaction known to be catalyzed by LeuA in many bacteria, followed by conversion to butanoyl-CoA, which is then converted to n-butanol using the final two reactions of the fermentative pathway. Likewise, as depicted in FIG. 1, at least one isobutanol pathway is known in the art for synthesizing isobutanol from pyruvate, wherein the five-step pathway loses two carbon atoms as CO₂ per molecule of isobutanol synthesized.

Thus, in certain embodiments, the present invention is directed to a method for producing isobutanol from a methane substrate comprising the steps of (a) providing a methanotrophic host microorganism that metabolizes methane (CH₄) to methanol (CH₃OH) and methanol to formaldehyde (H₂C═O); (b) introducing into the methanotroph host and expressing at least one polynucleotide open reading frame (ORF), under the control of suitable regulatory sequences, wherein the at least one polynucleotide ORF encodes a polypeptide that catalyzes a reaction in an isobutanol pathway; (c) feeding the methanotroph host of step (b) a methane substrate under suitable growth conditions, wherein the host metabolizes methane to formaldehyde as set forth in step (a), wherein the formaldehyde is converted to pyruvate by means of an endogenous RuMP pathway or a serine pathway and the host metabolizes pyruvate to produce isobutanol, and (d) optionally recovering the isobutanol produced.

In one particular embodiment, the one or more polynucleotide ORFs introduced in step (b) encode an isobutanol pathway polypeptide thereof selected from an Enzyme Class (EC) comprising EC 2.2.1.6, EC 1.1.1.86, EC 4.2.1.9, EC 4.1.1.72 and EC 1.1.1.1. In other embodiments, the one or more polynucleotide ORFs introduced in step (b) encode an isobutanol pathway polypeptide selected from the group consisting of acetolactate synthase (ALS), ketol-acid reductoisomerase (KARI), dihydroxy-acid dehydratase (DHAD), ketoacid decarboxylase (KDC) and alcohol dehydrogenase (ADH). In certain embodiments, the ALS polypeptide catalyzes the substrate to product conversion of pyruvate to acetolactate; the KARI polypeptide catalyzes the substrate to product conversion of acetolactate to 2,3-dihydroxyisovalerate; the DHAD polypeptide catalyzes the substrate to product conversion of 2,3-dihydroxyisovalerate to ketoisovalerate; the KDC polypeptide catalyzes the substrate to product conversion of ketoisovalerate to isobutryaldehyde and ADH polypeptide catalyzes the substrate to product conversion of isobutyraldehyde to isobutanol. In other embodiments, the ALS polypeptide comprises an amino acid sequence comprising at least 90% sequence homology to SEQ ID NO:2, the KARI polypeptide comprises an amino acid sequence comprising at least 90% sequence homology to SEQ ID NO:4, the DHAD polypeptide comprises an amino acid sequence comprising at least 90% sequence homology to SEQ ID NO:6, the KDC polypeptide comprises an amino acid sequence comprising at least 90% sequence homology to SEQ ID NO: 8 and the ADH polypeptide comprises an amino acid sequence comprising at least 90% sequence homology to SEQ ID NO:10. In yet other embodiments, the one or more polynucleotide ORFs introduced in step (b) encode the complete isobutanol pathway comprising an ALS polypeptide, a KARI polypeptide, a DHAD polypeptide, a KDC polypeptide and an ADH polypeptide. In certain embodiments, the ORFs encoding the complete isobutanol pathway are comprised in one operon, two operons or three operons, wherein each operon may comprise the same promoter or a different promoter, wherein the same or different promoters may be constitutive or inducible.

In certain embodiments, a methanotroph host microorganism is modified or genetically engineered to express one or more enzymes of a metabolic pathway capable of producing n-butanol, isobutanol, fatty (or aliphatic long chain) alcohols, fatty acid methyl esters and the like. In particular embodiments, a methanotroph of the invention is selected from genera consisting of Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis and Methyloacidophilum. In other embodiments, the methanotroph is from the phylum Verrucomicrobia. Previously published work has shown that several species within these taxa can be genetically transformed by introducing DNA constructs on plasmid vectors (Stafford et al., 2003), or by integrating them into the bacterial chromosome (Welander & Summons, 2012). Thus, a vector construct of the invention will typically comprise the pathway genes or polynucleotide ORFs, which are initially constructed and cloned into E. coli to generate sufficient quantities of the vector, and then the vectors are subsequently transformed into the host microorganism for expression.

In other embodiments, the invention is directed to a method for producing isobutanol from a methane substrate comprising the steps of (a) providing a “non-methanotroph” host microorganism which has been genetically engineered to express a methane monooxygenase (MMO) (and optionally a methanol dehydrogenase (MDH)) and wherein the non-methanotroph host comprises either an endogenous RuMP pathway or an endogenous serine pathway, (b) introducing into the host and expressing at least one polynucleotide open reading frame (ORF), under the control of suitable regulatory sequences, wherein the at least one polynucleotide ORF encodes a polypeptide that catalyzes a reaction in an isobutanol pathway; (c) feeding the host of step (b) a methane substrate under suitable growth conditions, wherein the MMO polypeptide catalyzes the substrate to product conversion of methane to methanol, an endogenous MDH polypeptide catalyzes the substrate to product conversion of methanol to formaldehyde, the formaldehyde produced is converted to pyruvate through an endogenous RuMP or serine pathway and the host metabolizes pyruvate to produce isobutanol, and (d) optionally recovering the isobutanol produced. Methods for heterologous expression of pMMO genes have been described in Gou et al. (2006). Methods for heterologous expression of sMMO genes have been described in Lloyd et al. (1999). Suitable microbial hosts for heterologous expression include microorganisms that have the ability to process methanol and formaldehyde, that have multiple heterotrophic growth modes, and/or that can assemble complex membranes and metalloprotein complexes. Such organisms include methylotrophic yeasts (e.g., Pichia pastoris) as well as bacteria such as Pseudomonas putida, Cupriavidus metallidurans and Rhodobacter sphaeroides.

In certain embodiments, the one or more polynucleotide ORFs introduced in step (b) above, encode an isobutanol pathway polypeptide selected from an Enzyme Class (EC) comprising EC 2.2.1.6, EC 1.1.1.86, EC 4.2.1.9, EC 4.1.1.72 and EC 1.1.1.1. In other embodiments, the one or more polynucleotide ORFs introduced in step (b) encode an isobutanol pathway polypeptide selected from the group consisting of acetolactate synthase (ALS), ketol-acid reductoisomerase (KARI), dihydroxy-acid dehydratase (DHAD), ketoacid decarboxylase (KDC) and alcohol dehydrogenase (ADH). In yet other embodiments, the ALS polypeptide catalyzes the substrate to product conversion of pyruvate to acetolactate; the KARI polypeptide catalyzes the substrate to product conversion of acetolactate to 2,3-dihydroxyisovalerate; the DHAD polypeptide catalyzes the substrate to product conversion of 2,3-dihydroxyisovalerate to ketoisovalerate; the KDC polypeptide catalyzes the substrate to product conversion of ketoisovalerate to isobutryaldehyde and ADH polypeptide catalyzes the substrate to product conversion of isobutyraldehyde to isobutanol.

In one particular embodiment, the ALS polypeptide comprises an amino acid sequence comprising at least 90% sequence homology to SEQ ID NO:2, the KARI polypeptide comprises an amino acid sequence comprising at least 90% sequence homology to SEQ ID NO:4, the DHAD polypeptide comprises an amino acid sequence comprising at least 90% sequence homology to SEQ ID NO:6, the KDC polypeptide comprises an amino acid sequence comprising at least 90% sequence homology to SEQ ID NO: 8 and the ADH polypeptide comprises an amino acid sequence comprising at least 90% sequence homology to SEQ ID NO:10. In certain other embodiments, the one or more polynucleotide ORFs introduced in step (b) encode the complete isobutanol pathway comprising an ALS polypeptide, a KARI polypeptide, a DHAD polypeptide, a KDC polypeptide and an ADH polypeptide. In another embodiment, the methane monooxygenase (MMO) is a soluble MMO of Enzyme Class EC 1.14.13.25 or a particulate MMO of Enzyme Class 1.14.18.3. In other embodiments, the MMO comprises an amino acid sequence having at least 90% sequence homology to a particulate methane monooxygenase (pMMO) of operon 1 comprising pmoC1 subunit 1 (SEQ ID NO:12), pmoA subunit 1 (SEQ ID NO:14), pmoB subunit 1 (SEQ ID NO: 16) or a pMMO of operon 2 comprising pmoC subunit 2 (SEQ ID NO:18), pmoA subunit 2 (SEQ ID NO:20), pmoB subunit 2 (SEQ ID NO:22). In other embodiments, the MMO comprises an amino acid sequence having at least 90% sequence homology to a soluble methane monooxygenase (sMMO) selected from mmoX (SEQ ID NO:24), mmoY (SEQ ID NO:26), mmoB (SEQ ID NO: 28), mmoZ (SEQ ID NO:30), mmoD (SEQ ID NO:32) or mmoC (SEQ ID NO:34).

In certain embodiments where an exogenous methanol dehydrogenase (MDH) is optionally provided and expressed in a host microorganism, the MDH is a polypeptide from Enzyme Class 1.14.18.3. In certain other embodiments, the MDH comprises an amino acid sequence comprising at least 90% sequence homology to mxaF (SEQ ID NO:36), mxaJ (SEQ ID NO:38), mxaG (SEQ ID NO:40), mxal (SEQ ID NO:42), mxaR (SEQ ID NO:44), mxaA (SEQ ID NO:46), mxaC (SEQ ID NO:48), mxaK (SEQ ID NO:50), mxaL (SEQ ID NO: 52) or mcaD (SEQ ID NO:54).

In other embodiments, the invention is directed to a method for producing 1-butanol from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing at least one polynucleotide open reading frame (ORF), under the control of suitable regulatory sequences, wherein the at least one polynucleotide ORF encodes a polypeptide that catalyzes a reaction in a 1-butanol pathway; (c) feeding the methanotroph host of step (b) a methane substrate under suitable growth conditions, wherein the host metabolizes methane to formaldehyde as set forth in step (a), wherein the formaldehyde is converted to pyruvate by means of an endogenous type I RuMP pathway or a type II serine pathway and the host metabolizes pyruvate to produce 1-butanol, and (d) optionally recovering the 1-butanol produced. In certain embodiments, the one or more polynucleotide ORFs introduced in step (b) encode a 1-butanol pathway polypeptide selected from an Enzyme Class (EC) comprising EC 4.3.1.19, EC 2.3.3.6, EC 4.2.1.33, EC 1.1.1.85, EC 4.1.1.72, and EC 1.1.1.1. In another embodiment, the one or more polynucleotide ORFs introduced in step (b) encode a 1-butanol pathway polypeptide selected from the group consisting of L-threonine ammonia-lyase, 2-ethylmalate synthase (or 2-isopropylmalate synthase), isopropylmalate isomerase (or 3-isopropylmalate dehydratase), 3-isopropylmalate dehydrogenase, 2-ketoacid decarboxylase (KDC) and alcohol dehydrogenase (ADH). In certain other embodiments, L-threonine ammonia-lyase catalyzes the substrate to product conversion of L-threonine to 2-oxybutanoate (2-ketobutyrate) and ammonia; the 2-ethylmalate synthase catalyzes the substrate to product conversion of 2-oxybutanoate and acetyl-CoA to 2-ethylmalate; the isopropylmalate isomerase catalyzes the substrate to product conversion of 2-ethylmalate to 3-ethylmalate; the 3-isopropylmalate dehydrogenase catalyzes the substrate to product conversion of 3-ethylmalate to 2-ketovalerate, CO₂ and NADH; the KDC catalyzes the substrate to product conversion of 2-ketovalerate to butryaldehyde and the ADH catalyzes the substrate to product conversion of butyraldehyde to 1-butanol.

In certain embodiments, a L-threonine ammonia-lyase comprises an amino acid sequence having at least 90% sequence homology to SEQ ID NO:56, a 2-ethylmalate synthase comprises an amino acid sequence having at least 90% sequence homology to SEQ ID NO:58, a isopropylmalate isomerase comprises an amino acid sequence having at least 90% sequence homology to SEQ ID NO:60 and SEQ ID NO:62, a 3-isopropylmalate dehydrogenase comprises an amino acid sequence having at least 90% sequence homology to SEQ ID NO:64, the KDC comprises an amino acid sequence having at least 90% sequence homology to SEQ ID NO:8 and the ADH comprises an amino acid sequence having at least 90% sequence homology to SEQ ID NO: 10. In one particular embodiment, the one or more polynucleotide ORFs introduced in step (b) encode the complete 1-butanol pathway comprising an L-threonine ammonia-lyase, a 2-ethylmalate synthase, an isopropylmalate isomerase, a 3-isopropylmalate dehydrogenase, a KDC and an ADH.

In other embodiments, the invention is directed to a method for producing 1-butanol from a methane substrate comprising the steps of (a) providing a “non-methanotroph” host microorganism which has been genetically engineered to express a methane monooxygenase (MMO) (and optionally a methanol dehydrogenase (MDH)) and wherein the non-methanotroph host comprises either an endogenous RuMP pathway or an endogenous serine pathway, (b) introducing into the host and expressing at least one polynucleotide open reading frame (ORF), under the control of suitable regulatory sequences, wherein the at least one polynucleotide ORF encodes a polypeptide that catalyzes a reaction in a 1-butanol pathway; (c) feeding the host of step (b) a methane substrate under suitable growth conditions, wherein the MMO polypeptide catalyzes the substrate to product conversion of methane to methanol, an endogenous MDH polypeptide catalyzes the substrate to product conversion of methanol to formaldehyde, the formaldehyde produced is converted to pyruvate through an endogenous RuMP or serine pathway and the host metabolizes pyruvate to produce 1-butanol, and (d) optionally recovering the 1-butanol produced.

In certain embodiments, the non-methanotroph host microorganism is genetically modified to express an exogenous methane monooxygenase (MMO). In one embodiment, the methane monooxygenase is a soluble MMO (sMMO) of Enzyme Class EC 1.14.13.25 or a particulate MMO (pMMO) of Enzyme Class 1.14.18.3. In other embodiments, the MMO comprises an amino acid sequence having at least 90% sequence homology to a particulate methane monooxygenase (pMMO) of operon 1 comprising pmoC1 subunit 1 (SEQ ID NO: 12), pmoA subunit 1 (SEQ ID NO:14), pmoB subunit 1 (SEQ ID NO: 16) or a pMMO of operon 2 comprising pmoC subunit 2 (SEQ ID NO: 18), pmoA subunit 2 (SEQ ID NO: 20), pmoB subunit 2 (SEQ ID NO:22). In other embodiments, the MMO comprises an amino acid sequence having at least 90% sequence homology to a soluble methane monooxygenase (sMMO) selected from mmoX (SEQ ID NO:24), mmoY (SEQ ID NO:26), mmoB (SEQ ID NO:28), mmoZ (SEQ ID NO:30), mmoD (SEQ ID NO:32) or mmoC (SEQ ID NO: 34).

In certain embodiments where an exogenous methanol dehydrogenase (MDH) is optionally provided and expressed in a host microorganism, the MDH is a polypeptide from Enzyme Class 1.14.18.3. In certain other embodiments, the MDH comprises an amino acid sequence comprising at least 90% sequence homology to mxaF (SEQ ID NO:36), mxaJ (SEQ ID NO:38), mxaG (SEQ ID NO:40), mxal (SEQ ID NO:42), mxaR (SEQ ID NO:44), mxaA (SEQ ID NO:46), mxaC (SEQ ID NO:48), mxaK (SEQ ID NO:50), mxaL (SEQ ID NO: 52) or mcaD (SEQ ID NO:54).

In one particular embodiment, the one or more polynucleotide ORFs introduced in step (b) encode a 1-butanol pathway polypeptide selected from an Enzyme Class (EC) comprising EC 4.3.1.19, EC 2.3.3.6, EC 4.2.1.33, EC 1.1.1.85, EC 4.1.1.72, and EC 1.1.1.1. In another embodiment, the one or more polynucleotide ORFs introduced in step (b) encode a 1-butanol pathway polypeptide selected from the group consisting of L-threonine ammonia-lyase, 2-ethylmalate synthase (or 2-isopropylmalate synthase), isopropylmalate isomerase (or 3-isopropylmalate dehydratase), 3-isopropylmalate dehydrogenase, 2-ketoacid decarboxylase (KDC) and alcohol dehydrogenase (ADH). In certain other embodiments, L-threonine ammonia-lyase catalyzes the substrate to product conversion of L-threonine to 2-oxybutanoate (2-ketobutyrate) and ammonia; the 2-ethylmalate synthase catalyzes the substrate to product conversion of 2-oxybutanoate and acetyl-CoA to 2-ethylmalate; the isopropylmalate isomerase catalyzes the substrate to product conversion of 2-ethylmalate to 3-ethylmalate; the 3-isopropylmalate dehydrogenase catalyzes the substrate to product conversion of 3-ethylmalate to 2-ketovalerate, CO₂ and NADH; the KDC catalyzes the substrate to product conversion of 2-ketovalerate to butryaldehyde and the ADH catalyzes the substrate to product conversion of butyraldehyde to 1-butanol.

In certain embodiments, a L-threonine ammonia-lyase comprises an amino acid sequence having at least 90% sequence homology to SEQ ID NO:56, a 2-ethylmalate synthase comprises an amino acid sequence having at least 90% sequence homology to SEQ ID NO:58, a isopropylmalate isomerase comprises an amino acid sequence having at least 90% sequence homology to SEQ ID NO:60 and SEQ ID NO:62, a 3-isopropylmalate dehydrogenase comprises an amino acid sequence having at least 90% sequence homology to SEQ ID NO:64, the KDC comprises an amino acid sequence having at least 90% sequence homology to SEQ ID NO:8 and the ADH comprises an amino acid sequence having at least 90% sequence homology to SEQ ID NO:10. In one particular embodiment, the one or more polynucleotide ORFs introduced in step (b) encode the complete 1-butanol pathway comprising an L-threonine ammonia-lyase, a 2-ethylmalate synthase, an isopropylmalate isomerase, a 3-isopropylmalate dehydrogenase, a KDC and an ADH.

In certain other embodiments, the invention is directed to a method for producing fatty alcohols from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a fatty-acyl-CoA reductase (FAR); (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes acetyl-CoA to produce a fatty alcohol, and (d) recovering the fatty alcohol produced. In certain embodiments, the FAR polypeptide is further defined as a polypeptide from Enzyme Class EC 1.2.1.50. In yet other embodiments, the FAR polypeptide catalyzes the substrate to product conversion of fatty acetyl-CoA to a fatty alcohol. In another embodiment, a FAR polypeptide comprises an amino acid sequence having at least 90% sequence homology to SEQ ID NO:66.

In still other embodiments, the invention is directed to a method for producing a fatty alcohol from a methane substrate comprising the steps of (a) providing a “non-methanotroph” host microorganism which has been genetically engineered to express a methane monooxygenase (MMO) (and optionally a methanol dehydrogenase (MDH)) and wherein the non-methanotroph host comprises either an endogenous RuMP pathway or an endogenous serine pathway, (b) introducing into the host microorganism and expressing a polynucleotide open reading frame (ORF), under the control of suitable regulatory sequences, wherein the polynucleotide ORF encodes a fatty-acyl-CoA reductase (FAR), (c) feeding the host microorganism of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes acetyl-CoA to produce a fatty alcohol, and (d) optionally recovering the fatty alcohol produced.

In certain embodiments, the non-methanotroph host microorganism is genetically modified to express an exogenous methane monooxygenase (MMO). In one embodiment, the methane monooxygenase is a soluble MMO (sMMO) of Enzyme Class EC 1.14.13.25 or a particulate MMO (pMMO) of Enzyme Class 1.14.18.3. In other embodiments, the MMO comprises an amino acid sequence having at least 90% sequence homology to a particulate methane monooxygenase (pMMO) of operon 1 comprising pmoC1 subunit 1 (SEQ ID NO:12), pmoA subunit 1 (SEQ ID NO:14), pmoB subunit 1 (SEQ ID NO: 16) or a pMMO of operon 2 comprising pmoC subunit 2 (SEQ ID NO:18), pmoA subunit 2 (SEQ ID NO: 20), pmoB subunit 2 (SEQ ID NO:22). In other embodiments, the MMO comprises an amino acid sequence having at least 90% sequence homology to a soluble methane monooxygenase (sMMO) selected from mmoX (SEQ ID NO:24), mmoY (SEQ ID NO:26), mmoB (SEQ ID NO:28), mmoZ (SEQ ID NO:30), mmoD (SEQ ID NO:32) or mmoC (SEQ ID NO: 34).

In certain embodiments, where an exogenous methanol dehydrogenase (MDH) is optionally provided and expressed in a host microorganism, the MDH is a polypeptide from Enzyme Class 1.14.18.3. In certain other embodiments, the MDH comprises an amino acid sequence comprising at least 90% sequence homology to mxaF (SEQ ID NO:36), mxaJ (SEQ ID NO:38), mxaG (SEQ ID NO:40), mxal (SEQ ID NO:42), mxaR (SEQ ID NO:44), mxaA (SEQ ID NO:46), mxaC (SEQ ID NO:48), mxaK (SEQ ID NO:50), mxaL (SEQ ID NO: 52) or mcaD (SEQ ID NO:54).

In another embodiment, the invention is directed to a method for producing a fatty acid ester from a methane substrate comprising the steps of (a) providing a methanotrophic host microorganism that metabolizes methane (CH₄) to methanol (CH₃OH) and methanol to formaldehyde (H₂C═O), (b) introducing into the methanotroph host and expressing a polynucleotide open reading frame (ORF), under the control of suitable regulatory sequences, wherein the polynucleotide ORF encodes a wax ester synthase (WES); (c) feeding the methanotroph host of step (b) a methane substrate under suitable growth conditions, wherein the host metabolizes methane to formaldehyde as set forth in step (a), wherein the formaldehyde is converted to acetyl-CoA by means of an endogenous type I RuMP pathway or a type II serine pathway and the host metabolizes fatty-acyl-CoA and alcohols to produce a fatty acid ester, and (d) recovering the fatty acid ester produced. In one particular embodiment, the WES polypeptide is further defined as a polypeptide from Enzyme Class EC 2.3.1.75. In another embodiment, the WES polypeptide catalyzes the substrate to product conversion of a fatty acid to a fatty acid esters. In another embodiment, the WES polypeptide catalyzes the substrate to product conversion of fatty alcohol and acyl-CoA to fatty acid esters. In one particular embodiment, the WES polypeptide comprises an amino acid sequence having at least 90% sequence homology to a WES polypeptide selected from SEQ ID NO:68, SEQ ID NO: 70, SEQ ID NO: 72, SEQ ID NO: 74, SEQ ID NO: 76 and SEQ ID NO: 78.

In another embodiment, the invention is directed to a method for producing a fatty acid ester from a methane substrate comprising the steps of (a) providing a “non-methanotroph” host microorganism which has been genetically engineered to express a methane monooxygenase (MMO) (and optionally a methanol dehydrogenase (MDH)) and wherein the non-methanotroph host comprises either an endogenous RuMP pathway or an endogenous serine pathway, (b) introducing into the host microorganism and expressing at least one polynucleotide open reading frame (ORF), under the control of suitable regulatory sequences, wherein the at least one polynucleotide ORF encodes a wax ester synthase; (c) feeding the host of step (b) a methane substrate under suitable growth conditions, wherein the MMO polypeptide catalyzes the substrate to product conversion of methane to methanol, an endogenous MDH polypeptide catalyzes the substrate to product conversion of methanol to formaldehyde, wherein the formaldehyde is converted to acetyl-CoA by means of an endogenous RuMP or serine pathway and the host metabolizes fatty-acyl-CoA and alcohols to produce a fatty acid ester, and (d) recovering the fatty acid ester produced.

In one particular embodiment, the WES polypeptide is further defined as a polypeptide from Enzyme Class EC 2.3.1.75. In another embodiment, the WES polypeptide catalyzes the substrate to product conversion of a fatty acid to a fatty acid ester. In another embodiment, the WES polypeptide catalyzes the substrate to product conversion of fatty alcohol and acyl-CoA to fatty acid esters. In one particular embodiment, the WES polypeptide comprises an amino acid sequence having at least 90% sequence homology to a WES polypeptide selected from SEQ ID NO:68, SEQ ID NO: 70, SEQ ID NO: 72, SEQ ID NO: 74, SEQ ID NO: 76 and SEQ ID NO: 78.

In certain embodiments, the non-methanotroph host microorganism is genetically modified to express an exogenous methane monooxygenase (MMO). In one embodiment, the methane monooxygenase is a soluble MMO (sMMO) of Enzyme Class EC 1.14.13.25 or a particulate MMO (pMMO) of Enzyme Class 1.14.18.3. In other embodiments, the MMO comprises an amino acid sequence having at least 90% sequence homology to a particulate methane monooxygenase (pMMO) of operon 1 comprising pmoC1 subunit 1 (SEQ ID NO:12), pmoA subunit 1 (SEQ ID NO:14), pmoB subunit 1 (SEQ ID NO: 16) or a pMMO of operon 2 comprising pmoC subunit 2 (SEQ ID NO: 18), pmoA subunit 2 (SEQ ID NO: 20), pmoB subunit 2 (SEQ ID NO:22). In other embodiments, the MMO comprises an amino acid sequence having at least 90% sequence homology to a soluble methane monooxygenase (sMMO) selected from mmoX (SEQ ID NO:24), mmoY (SEQ ID NO:26), mmoB (SEQ ID NO:28), mmoZ (SEQ ID NO:30), mmoD (SEQ ID NO:32) or mmoC (SEQ ID NO: 34).

In certain embodiments, where an exogenous methanol dehydrogenase (MDH) is optionally provided and expressed in a host microorganism, the MDH is a polypeptide from Enzyme Class 1.14.18.3. In certain other embodiments, the MDH comprises an amino acid sequence comprising at least 90% sequence homology to mxaF (SEQ ID NO:36), mxaJ (SEQ ID NO:38), mxaG (SEQ ID NO:40), mxal (SEQ ID NO:42), mxaR (SEQ ID NO:44), mxaA (SEQ ID NO:46), mxaC (SEQ ID NO:48), mxaK (SEQ ID NO:50), mxaL (SEQ ID NO: 52) or mcaD (SEQ ID NO:54).

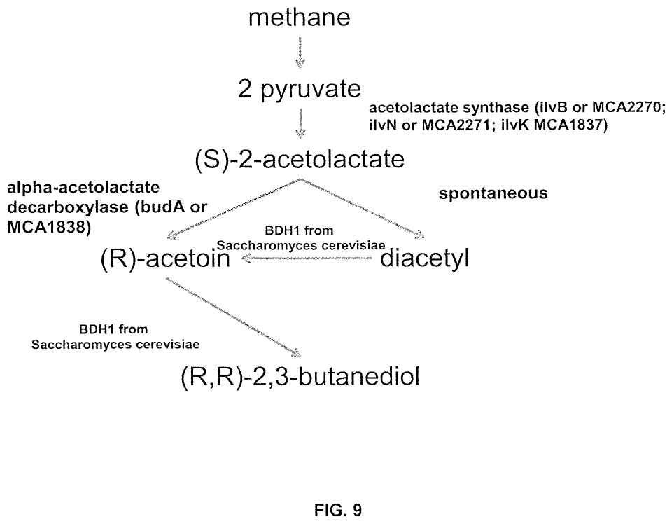

In certain other embodiments, the invention is directed to methods of producing 2,3-butanediol from a methane substrate. The compound 2,3-butanediol (a four-carbon diol) is an important intermediate for the chemical industry. At the commercial scale, 2,3-butanediol is mainly produced or generated from petroleum, where it serves as a precursor for the production of various commodity and specialty chemicals, such as the solvent methyl ethyl ketone (MEK), gamma-butyrolactone (GBL) and 1,3-butadiene. The biological production of 2,3-butanediol from methane requires engineering the native (or endogenous) metabolism of methanotrophs to take advantage of their endogenous production of (R)-acetoin (FIG. 9). The compound (R)-acetoin is produced in methanotrophs from two molecules of pyruvate, which are ultimately derived from methane. By introducing and expressing a single gene (SEQ ID NO:156) encoding a (2R,3R)-2,3-butanediol dehydrogenase (BDH1) from Saccharomyces cerevisiae in a suitable microbial expression host (such as M. capsulatus (Bath)), the (R)-acetoin is converted into 2,3-butanediol. Thus, in certain embodiments, a host microorganism of the invention is genetically modified to express an exogenous (2R,3R)-2,3-butanediol dehydrogenase (BDH1) having at least 90% sequence homology to a BDH1 polypeptide of SEQ ID NO:157.

General methods for gene synthesis and DNA cloning, as well as vector and plasmid construction, are well known in the art, and are described in a number of publications (Lipps, 2008; Peccoud, 2012; Ausubel et al., 2002). More specifically, techniques such as digestion and ligation-based cloning, as well as in vitro and in vivo recombination methods, can be used to assemble DNA fragments encoding a polypeptide that catalyzes a substrate to product conversion into a suitable vector. These methods include restriction digest cloning, sequence- and ligation-independent Cloning (SLIC) (Li & Elledge, 2012), Golden Gate cloning (Engler et al., 2009), Gibson assembly (Gibson et al., 2009), and the like (Merryman & Gibson, 2012; Wang et al., 2012). Some of these methods can be automated and miniaturized for high-throughput applications (Yehezkel et al., 2011; Ma et al., 2012).

In certain embodiments, the cloning procedures use in vitro homologous recombination, to insert DNA fragments into a vector (e.g., the In-Fusion kit from Clontech Laboratories, Inc. (Mountain View, CA)). For example, (1) the recipient vector is linearized by a restriction digest and purified; (2) PCR primers that are complementary to the fragment to be cloned and that are complementary (with 15-base pair extensions) to the ends of the linearized vector are used to amplify the insert, using high-fidelity polymerase; (3) the size of the PCR amplicon is verified by agarose gel electrophoresis; (4) the PCR product is purified by a spin-column; (5) the In-Fusion reaction is run according to the manufacturer's instructions; (6) competent E. coli cells are transformed with 2.5 μL of the reaction products; (7) positive transformants are selected from colonies grown on antibiotic selection medium and transferred to individual liquid cultures with the appropriate antibiotic; (8) the cells are harvested after overnight growth at 37° C. with 200 rpm shaking and (9) the plasmid DNA is extracted and analyzed for the correct insert.

The plasmid vector is chosen so that it will be capable of replicating in both an E. coli host (for cloning and amplification) and a methanotrophic or non-methanotrophic host microorganism (for metabolic pathway expression). The plasmid can be transferred from the E. coli donor cell to the recipient cell via bacterial conjugation. In addition, the vector contains a promoter sequence upstream of the one or more polynucleotide ORFs that are to be expressed. The promoter sequence can be included as part of the insert so that it can be adjusted and tested for each new construct. Broad-host-range (bhr) vectors for different gram-negative bacterial hosts have been described in the literature (Marx & Lidstrom, 2001). These vectors typically contain the following components: (1) an origin of replication that is functional in E. coli (colE1); (2) an oriV/IncP origin of replication for the non-E. coli host; (3) an oriT/IncP origin of transfer, which is needed for transferring a bacterial plasmid from a bacterial host such as E. coli to the recipient during bacterial conjugation; (4) a traJ′ gene, which codes for a transcriptional activator that initiates production of the proteins needed for conjugative transfer; and (5) a trfA, the replication initiation protein gene of plasmid RK2 which binds to a activates oriV.

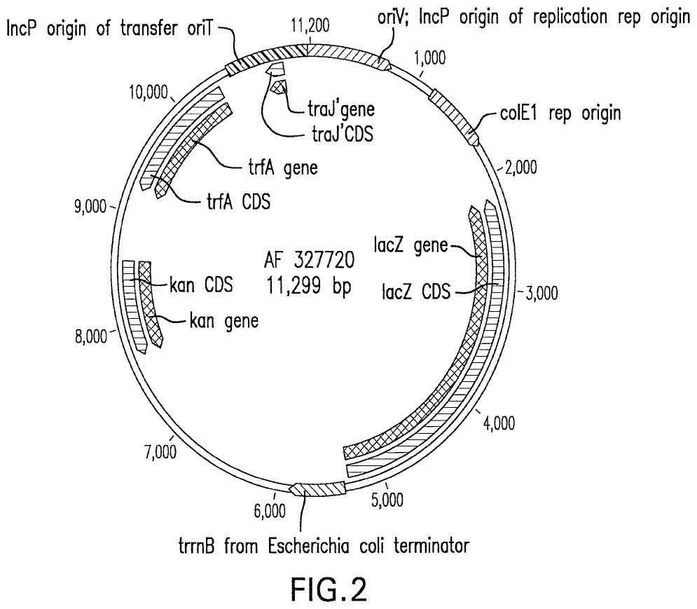

In one embodiment, the conjugative bhr plasmid is based on pCM132 (GenBank Accession No. AF327720, SEQ ID NO:79) (Marx & Lidstrom, 2001), which has been engineered to contain a kanamycin resistance gene for plasmid selection and a lacZ (beta-galactosidase) gene for identifying plasmids with DNA inserts based on colony color using indolyl-galactoside-based substrates. Genes (or polynucleotide ORFs thereof) of interest can be inserted into the polylinker region that lies between the rrnB transcription terminator and the 5′-end of the lacZ gene (e.g., see, FIG. 2).

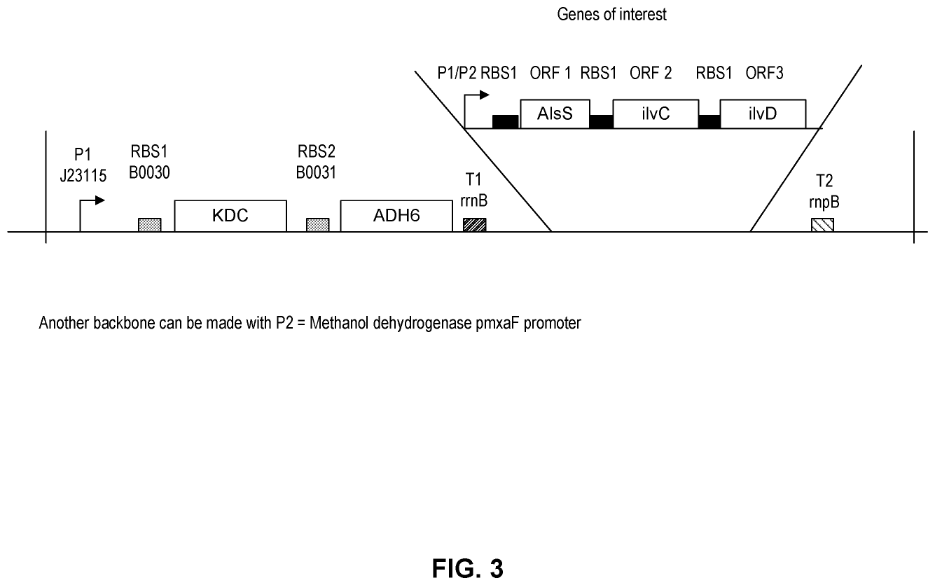

Typical gene cassettes for expressing an engineered metabolic pathway in a host microorganism such as a methanotroph are shown in FIG. 3. The cassette comprises one or more open reading frames (ORFs) which encode the enzymes of the introduced pathway, a promoter for directing transcription of the downstream ORF(s) within the operon, ribosome binding sites for directing translation of the mRNAs encoded by the individual ORF(s), and a transcriptional terminator sequence. Due to the modular nature of the various components of the expression cassette, one can create combinatorial permutations of these arrangements by substituting different components at one or more of the positions. One can also reverse the orientation of one or more of the ORFs to determine whether any of these alternate orientations improve the product yield.

In one embodiment, the plasmids generated as part of the present invention are based on the broad-host-range expression vector pCM132 (Marx & Lidstrom, 2001). In this embodiment, the use of the Clontech (catalog no. 639647) InFusion HD Cloning System kit is one example of how to construct plasmids, but is not meant to limit or exclude other methods that are known in the art, including Gibson assembly, yeast in vivo recombination, PCR Splicing by Overlap Extension, or any combination of these with standard molecular biology techniques.

In certain embodiments of the invention, the plasmids of interest are generated in a modular fashion such that various modules, including suitable regulatory sequences, can be easily assembled or replaced as needed and are amenable to scaled-up, high-throughput assembly. The plasmids are designed to consist of multiple linear modules: a vector backbone and one or more vector inserts. The 5′ and 3′ ends of individual modules have overlapping sequence homology to the ends of adjacent modules within the designed plasmid. The overlapping homology between the modules allows them to be assembled into a circular plasmid using the Clontech InFusion HD Cloning System kit or other assembly method known in the art. Primers were designed to introduce homologous ends to the PCR-amplified products to facilitate assembly.

Vector backbones of the invention contain the components of the plasmid that will remain constant. In certain embodiments, the broad-host range vector pCM132 is modified to produce vector backbones for the plasmids (vectors) of the invention. The pCM132 vector, further described below in the Examples section, consists of the following components: trrnB terminator, kanamycin resistance gene, trfA, IncP oriT, IncP oriV, colE1 ori, and lacZ. This parental vector was modified to replace lacZ with a vector insert that contains promoter sequence(s) to produce plasmids pJSvec (SEQ ID NO: 80) and pMZT3 (SEQ ID NO: 81). In certain embodiments of the invention, vector backbones were PCR-amplified with the NEB Phusion master mix (M0531L) according to the manufacturer's instructions, unless specified otherwise.

The general rationale or procedure for selecting the appropriate ORFs for a given pathway was to examine a list of pathway-relevant genes as specified in the literature. Using this set of pathway-relevant genes as a target, BLAST searches were run, looking for genes in three groups: (1) similar genes found in microbial hosts that are phylogenetically close to the ones already listed in the literature, (2) similar genes found in microbes that are phylogenetically distant from the microbial host of the targeted gene, and (3) homologs that are similar to the target gene but that are found in the wild-type methanotroph or non-methanotroph organism that is to be used as the expression host. An example of the above strategy would be to target the kivD gene (encoding alpha-ketoisovalerate decarboxylase) from Lactococcus lactis: the first group would contain genes from species similar to L. lactis, including Lactococcus itself; the second group would be genes similar to kivD, but found in organisms phylogenetically distant from L. lactis; and finally the last group would include a kivD gene in a microbe of interest, specifically, Methylococcus capsulatus (Bath). Thus, in certain embodiments of the invention, the exemplary polynucleotide and polypeptide sequences set forth in Table 1 are used to identify similar or homologous polynucleotide, genes, ORFs and polypeptides found in microbial hosts that are (1) phylogenetically close to the ones already listed, (2) found in microbes that are phylogenetically distant from the microbial host of the targeted sequence, and (3) homologs that are similar to the target gene but that are found in the wild-type methanotroph or non-methanotroph organism that is to be used as the expression host.

For example, genes encoding similar proteins or polypeptides to those of the invention may isolated directly by using all or a portion of a nucleic acid (e.g., see Table 1, below) or a primer sequence (e.g., see Table 2, below) as DNA hybridization probes to screen libraries from any desired microorgansim using methodology well known to those skilled in the art. Specific oligonucleotide probes based upon these nucleic acid sequences can be designed and synthesized by methods known in the art (Sambrook et al., 1989; Ausubel et al., 1987). Moreover, the entire sequence can be used directly to synthesize DNA probes by methods known to the skilled artisan such as random primers, DNA labeling, nick translation, or end-labeling techniques, or RNA probes using available in vitro transcription systems. In addition, specific primers can be designed and used to amplify a part of or the full-length of the instant sequence. The resulting amplification products can be labeled directly during amplification reactions or labeled after amplification reactions, and used as probes to isolate full-length DNA fragments under conditions of appropriate stringency.

Alternatively a nucleic acid sequence of the invention may be employed as a hybridization reagent for the identification of homologs. The basic components of a nucleic acid hybridization test include a probe, a sample suspected of containing the gene or gene fragment of interest, and a specific hybridization method. Probes are typically single stranded nucleic acid sequences which are complementary to the nucleic acid sequences to be detected. Probes are “hybridizable” to the nucleic acid sequence to be detected. The probe length can vary from 5 bases to tens of thousands of bases, and will depend upon the specific test to be done. Typically a probe length of about 15 bases to about 30 bases is suitable. Only part of the probe molecule need be complementary to the nucleic acid sequence to be detected. In addition, the complementarity between the probe and the target sequence need not be perfect. base. Hybridization methods are well defined and know in the art.

An important component of these engineered operons is the promoter sequence. The promoter must be chosen based on its compatibility with the transcriptional machinery of the host organism, as well as its ability to tune the desired level of gene expression (e.g., high or low). For example, one may introduce the strong pmxaF or pmmoX promoters from a methanotroph to generate high expression levels in a methanotrophic or non-methanotroph host. Alternatively, one can introduce a promoter from the Anderson promoter collection, which is a library of constitutive sigma70 bacterial promoters (http://partsregistry.org/Promoters/Catalog/Anderson; Registry of Standard Biological Parts), such as J23100 (strong) or J23115 (weak), to modulate expression of different ORFs or combinations of ORFs. Inducible promoters, whose activity is controlled by the addition of exogenous small molecule activators, such as IPTG, arabinose or salicylate, can also be used to provide temporal control of gene expression. However, regardless of the choice of promoter, its effect on host expression must be empirically tested in vivo to be certain of its effectiveness for achieving the desired level of expression.

These different combinatorial permutations of the cassette can be synthesized, cloned and expressed in the target host organism (via chemical transformation, electroporation, or conjugation of the DNA) so that the production of a multi-carbon product can be compared. The best candidate or candidates can then be further engineered to provide additional improvements in product yield by repeating the design-build-test cycle.

In one embodiment, the host microorganism for expressing the plasmid is a methanotroph, and plasmid vector(s) containing the metabolic pathway expression cassettes are readily mobilized into these organisms via conjugation. Various methods for bacterial conjugation are known in the art, and one of the most widely used methods takes advantage of a strain of E. coli S17-1, which has an RP4 plasmid (with the RK2 tra genes for transfer of genetic material) inserted into the chromosome for mobilizing oriT (RP4)-carrying plasmids (Simon et al. 1983; Simon, 1984).

The transfer of plasmid containing RP4-mob from E. coli to methanotrophs, as further described in the Examples section, was based on the conjugation methods described previously (Martin & Murrell, 1995; Ali, 2006). A 10 ml overnight E. coli S17-1λ pir culture, containing RP4-mob plasmid, was collected on a 0.2 μm pore-size nitrocellulose filter (Millipore). The E. coli donor strain was washed twice with 50 ml NMS. A 50 ml methanotroph culture grown to mid exponential phase (A₅₄₀ of 0.2-0.5) was also collected on the same filter and washed again with 50 mL NMS medium. The filter was placed on an NMS agar plate containing 0.02% (w/v) proteose peptone and incubated for 24 hours at 30° C. with methane except for M. capsulatus, which was incubated at 37° C. for 24 hours.

Following incubation, the cells were washed with 10 ml NMS and collected by centrifugation (7,000×g for 10 min) before re-suspending the cells in 1 ml NMS. Aliquots (50-100 μl) of the cells were spread onto NMS plates containing selective antibiotics and incubated at the appropriate temperature. Colonies typically formed on the plates after 8-12 days. (Note: the E. coli S17-1λ pir strain has chromosomally integrated conjugal transfer functions, thus allowing transfer of plasmid to occur by means of a bi-parental mating without a helper plasmid). Transconjugants can also be purified by serial cultivation in liquid medium containing the appropriate antibiotics for selection, followed by plating onto selective NMS agar plates to obtain single colonies.

In an alternative method for expressing metabolic pathway genes in a microbial host, the biosynthetic pathway genes are inserted directly into the chromosome. Methods for chromosomal modification include both non-targeted and targeted deletions and insertions. For example, non-targeted insertions can be achieved by using transposon mutagenesis to make insertion mutants or gene “knockouts” in vitro using the EZ-Tn5 <KAN-2> Insertion Kit (Epicentre). Briefly, the procedure is as follows, according to the manufacturer: Preparation: prepare 0.2 μg of recombinant DNA for the EZ-Tn5<KAN-2> insertion reaction. Day 1: perform the 2-hour in vitro EZ-Tn5<KAN-2> insertion reaction; transform competent recA-E. coli with 1 μl of the reaction mix and select for kanamycin-resistant transposon insertion clones on kanamycin plates overnight. Day 2: prepare DNA from kanamycin-resistant colonies, (and optionally map the EZ-Tn5<KAN-2> Transposon insertion sites and optionally (DNA) sequence chosen clones bi-directionally using the unlabeled forward and reverse transposon-specific primers supplied in the kit.

For targeted modifications, various methods have been developed based on RecA-dependent homologous recombination (Hamilton et al., 1989; Link et al., 1997; Posfai et al., 1999). However, using antibiotic resistance markers for deletion/insertion is limited by the number of different antibiotics that can be used in a given target organism. For this reason, markerless insertion methods have been developed. For example, Yu et al. (2008) describe a deletion procedure in which expression of the A-Red recombinase genes (gam, bet and exo) and the I-SceI endonuclease gene are controlled by tightly regulated promoters ParaB and PrhaB. Arabinose and rhamnose added to cultures to induce ParaB and PrhaB are used and depleted by the bacteria. Thus, by changing the carbon source in the medium from arabinose to rhamnose in bacteria that contain the pREDI plasmid, one can replace a targeted genomic region with a markerless deletion cassette and subsequently delete the selection markers that were introduced.

Sun et al. (2008) also describe methods for sequence-specific insertion or deletion of genes within a bacterial genome. This method permits multiple markerless insertions and scarless deletions in the targeted genome. In the Sun et al. method, a target gene can be deleted in two steps. In the first step, a linear DNA fragment is created that carries the cat (chloramphenicol resistance) gene and sacB (a levansucrase gene that confers sensitivity to sucrose). The fragment is flanked by long (500 bp) regions of DNA that are homologous to the regions that flank the targeted deletion site. The DNA fragment is electroporated into the host cell, which already contains plasmid pKD46, a vector containing the genes for λ Red recombination. Homologous recombination then directs the replacement of the targeted gene. Medium containing chloramphenicol is used to select for cells that contain the desired insertion or deletion. In the second step, a second DNA fragment that contains the desired deletion or insertion is electroporated into host cells that contain the pKD46 plasmid. By plating the resulting cells on medium containing sucrose, one can select for transformants in which the cat-sacB cassette has been replaced. These candidates are also screened for sensitivity to chloramphenicol, and the deletion can be confirmed by PCR and sequencing. By repeating the process, multiple deletions and/or insertions can be achieved. The pKD46 plasmid can then be removed by culturing the cells at 37 C. Thus, by using various genes encoding the isobutanol, butanol, fatty alcohol and fatty acid ester biosynthetic pathways, these pathways can be inserted into the genome of a methanotroph (or non-methanotroph), and unwanted genes (e.g., genes that encode for enzymes that produce competing products) can be removed.

U.S. Patent Publication No. 2006/0057726 describes using sacB gene and the pGP704 suicide vector to engineer markerless insertions into single carbon (C1) metabolizing bacteria. Yomantas et al. (2010) describes methods for markerless substitutions in the genome of the methylotrophic bacterium Methylophilus methylotrophus.

Several methanotroph strains were evaluated according to the present invention as potential hosts for pathway engineering. Of the well characterized methanotroph strains, Methylosinus trichosporium OB3b (NCIMB 11131) and Methylococcus capsulatus str. Bath (NCIMB 11853) were examined for their case of transformability (via conjugation), growth rate, and suitability for industrial fermentation. Both strains can be cultivated in liquid or agar containing Nitrate Mineral Salts (NMS) medium (Whittenbury et al., 1970; Bowman, 2000). Although both strains were found to transform with approximately equal efficiency, Methylococcus capsulatus (Bath) has the advantage of growing about twice as fast as M. trichosporium (ca. 24-30 to reach saturation in shake flask growth). In addition, the ability of M. capsulatus (Bath) to grow more readily at 45° C. is an advantage in industrial cultivation, since this relatively high temperature will impede the growth of other potentially contaminating microorganisms. Furthermore, the complete genome sequence of M. capsulatus (Bath) has been published (Ward et al., 2004), and as such, manipulation of its genome via genetic engineering is readily available to one of skill in the art. Thus, in certain embodiments, M. capsulatus (Bath) is used as a model organism for further development of genetically modified host microorganisms.

Following conjugation, positive methanotroph trans-conjugants were purified on NMS agar containing the appropriate antibiotic selection (e.g., 15 μg/ml kanamycin for selecting the plasmid and counter-selecting the untransformed methanotroph host cells, and 10 μg/ml for counter-selecting the E. coli donor cells). Alternatively, transconjugants can be purified by serial cultivation in liquid medium containing the appropriate antibiotics for selection, followed by plating onto selective NMS agar plates to obtain single colonies. Colonies were used to inoculate small (5-10 ml) starter cultures in liquid NMS medium containing, for example, 15 μg/ml kanamycin in 125-ml flasks. The flasks were stoppered with tight-fitting Suba Seals to create a closed atmosphere inside the flasks. A volume of gas corresponding to 20% of the total volume of the flask and composed of 95% methane and 5% carbon dioxide was injected via a sterile syringe and 23-gauge needle into each flask. Flasks were shaken at 200 rpm and 45° C. When these cultures achieved an optical density of A₅₄₀>0.5 (after about 24 hours), a 1:100 dilution of these cells was used to inoculate 125 ml (or larger volume) cultures, and the same growth protocol was followed. Growth in shake flasks is most robust when the liquid volume is maintained at about 5-10% of the nominal volume of the flask so that good aeration of the liquid is achieved. These flasks were then used for the subsequent assays of product formation. In certain examples related to 2-KIV feeding experiments, only the ketoacid intermediate was added along with the methane and CO₂ at the zero time point.

After approximately 72 hours of growth, the cultures were harvested for analysis by gas chromatography. The sealed flasks were first chilled for at least 1 hour on ice, to concentrate any volatile organic compounds from the vapor phase into the liquid phase. After opening the flasks, an aliquot of the culture was diluted 1:2 with ethyl acetate in a clean 50 ml tube to extract and concentrate the isobutanol, butanol, fatty alcohols or fatty acid esters. After vortexing or shaking (and centrifugation to separate the phases), a small volume of the organic layer (approximately 1 ml) was filtered through a 0.2 μm PTFE filter, and 1 μl of the purified extract was then injected into an Agilent 7890A GC equipped with a Leap Technologies (Carrboro, NC) CombiPAL autosampler for analysis. Appropriate purified standards were included to generate a standard curve and determine the concentration of the targeted product. Each measurement included a positive control and a negative control (e.g., a wild-type sample or other appropriate background control) with each sample set. Additional details of the methods used for the specific products are given in the Examples section. Strains with the highest levels of production were designated for further scale-up in 1-10 liter fermentors.

During the analysis of the engineered host strains, unexpectedly high levels of isobutanol and butanol consumption (up to 30 mM after 72 hours of growth) was observed even in wild-type cultures of M. capsulatus (Bath), and therefore it was important to find mutant strains that can produce these products at a rate that is greater than their inherent rate of consumption. In certain embodiments of the invention, the competing alcohol dehydrogenase and alcohol oxidase activities are identified, and reduced or eliminated by gene knockouts, as described above.

For initial fermentation scale-up in the 1-10 liter range, methods similar to those described in Theisen et al. (2005) and U.S. Pat. No. 4,594,324 can be used, with specific modifications for M. capsulatus (Bath). A fermentation system such as the Sartorius-Stedim Biostat A plus system (Goettingen, Germany) can be used, or other equivalent fermentation systems and methods for methanotroph fermentation (e.g., see Jiang et al., 2010). An Applikon ADI 1030 Bio Controller and ADI 1035 BioConsole (Applikon Biotechnology Inc., Foster City, CA) can also be used for the 10 liter vessel.

The starting inoculum is created by inoculating a large colony of M. capsulatus (Bath) containing the desired plasmid from a plate culture into 10 ml of sterile NMS medium containing kanamycin, as described above. After 24 to 48 hours, when the optical density (A₅₄₀) of the culture is greater than 0.5, five starter flasks of NMS medium are inoculated at 1:100 dilution. The liquid volumes in these starter inocula can range in size from 20 ml each for a 1 liter fermentor to 200 ml each for a 10 liter fermentor (i.e., about a 10% inoculum).

After autoclaving the NMS medium in the fermentor vessel, the phosphate salts portion of the NMS medium and the kanamycin (both sterilized) are added to the vessel. The same inlet can be used to inject the starter cultures. Air is supplied as oil-free compressor air, and the methane carbon source is supplied from a pre-mixed tank (Airgas) containing 95% methane and 5% CO₂. The air and methane are mixed to 15-20% methane using equipment that is rated intrinsically safe or explosion proof to eliminate the possibility of sparking or static electricity, which could lead to an explosion. The gas flow rate depends on the fermentor size and culture density, but a value of 0.75 liters per minute for 10 liters is typical. The gas mixture is fed into the fermentor, and the entire culture is mixed with an impeller rotating at approximately 200 rpm for agitation, the rate of which may be increased during growth. For maintenance of the culture pH at 6.8, 0.1 M HCl or 1 M NaOH is added as needed. The temperature is maintained at 45° C. by a thermostatic jacket. The effluent gas is fed through a water-jacketed condenser to reduce liquid loss at 45° C., and vented to a fume hood.

The fermentation is monitored (via pH and dissolved oxygen probes) and controlled using Sartorius BioPAT MFCS bioprocess control software (Sartorius Corp, Bohemia, NY). A dissolved oxygen concentration below 1% saturation with air (typically 0.2-0.3%) is desirable to avoid wasting methane. Periodically, small samples of the fermentation broth are removed by sterile transfer and used to measure the optical density of the culture. These samples can also be used to monitor product formation using the methods described above and in the Examples section. Purity of the culture can also be checked by plating a small sample onto R2A agar, which allows most organisms other than methanotrophs to grow. Cultures achieve an optical density (A₅₄₀) of greater than 9 after about 48 hours. For M. capsulatus (Bath), 1 ml of culture with A₅₄₀ equal to 1 corresponds to about 0.23-0.25 mg of dry weight of biomass. When the maximum cell density or product concentration is achieved, the culture can be harvested and analyzed.

For large-scale commercial fermentation, a system based on the fermentor design employed by Norferm (Norefem, AS; Stavanger, Norway) for production of single-cell protein can be used (Bothe et al., 2002; EP 1419234; U.S. Publication No. 2009/0263877). The largest system has a total volume of 300 m³ (300,000 liters) and an annual production capacity of 10,000 tons of biomass (van Laere et al., 2005). Publications such as EP 1419234, U.S. Publication No. 2009/0263877 and Villadsen (2012), and references therein, describe a loop reactor and bioprocess methods for culturing methanotrophs at the commercial scale. The advantage of this design is that nutrient gases such as methane and oxygen are supplied to the system in such a way that exposure of the cells to nutrient-depleted culture medium or to unduly high concentrations of nutrient gases is minimized.

However, when using “wet” natural gas as a nutrient feedstock, the problem of acetate and propionate toxicity (resulting from the oxidation of ethane and propane, respectively) may need to be addressed (Bothe et al., 2002; Eiteman & Altman, 2006). A genetic approach is to eliminate (knock-out) or knock-down the ethanol and propanol dehydrogenases and acetaldehyde/propionaldehyde dehydrogenases that convert the ethanol and propanol to the corresponding acids. Another approach is to introduce the genes for acetate assimilation from an organism that can use it as a carbon source, such as E. coli (Wolfe, 2005). For example, AMP-ACS (acetate: CoA ligase [AMP forming]; EC 6.2.1.1) catalyzes the conversion of acetate and ATP to an enzyme-bound acetyladenylate (acetyl-AMP) and pyrophosphate. In a subsequent step, it reacts the acetyl-AMP with CoASH (CoenzymeA-SH) to acetyl-CoA and free AMP. Similarly, AMP-ACS can activate and assimilate propionate (Wolfe, 2005). In this way, the two potentially harmful organic acids are converted into the useful intermediate, acetyl-CoA. These genes can be cloned and expressed in a methanotroph host by the methods described above.

Another aspect of the commercial production of multicarbon compounds from methane using the present invention involves recovering and purifying the desired product from the fermentation broth. The method to be used depends on the physico-chemical properties of the product and the nature and composition of the fermentation medium and cells. For example, U.S. Pat. No. 8,101,808 describes methods for recovering C3-C6 alcohols from fermentation broth using continuous flash evaporation and phase separation processing. Thus, the biologically produced multi-carbon compounds of the invention may be isolated from the fermentation medium using methods known in the art for Acetone-butanol-ethanol (ABE) fermentations For example, solids may be removed from the fermentation medium by centrifugation, filtration, decantation, wherein the multi-carbon compounds of the invention may be isolated from the fermentation medium using methods such as distillation, azeotropic distillation, liquid-liquid extraction, adsorption, gas stripping, membrane evaporation, or pervaporation.

In certain embodiments the invention, the fermentation process produces greater than about 7% (v/v) concentration of the desired multi-carbon product in the fermentation broth, and the product is separated from the rest of the medium using membrane separation technology to achieve about a 12% or greater concentration of the product, at which point relatively small molecules (such as isobutanol) can be further purified by phase separation in an integrated system (Hickey & Slater, 1990; Neel, 1995; Hägg, 1998; Liu et al., 2011). Continuous recovery of the product from the fermentation medium has the advantage of possibly reducing the toxicity effects of the multi-carbon products.

For longer-chain alcohols, such as fatty alcohols, U.S. Pat. No. 8,268,599 describes methods for separating these components from the aqueous phase of the fermentation by bi-phasic separation, whereby the immiscibility of the product compounds with the fermentation broth allows the organic phase to be collected and removed. This separation can also reduce the toxic effects of the product on the host microbial cells.

U.S. Publication No. 2007/0251141 describes methods for recovering fatty acid methyl esters (FAMEs) from a liquid suspension by adding urea and creating a phase separation whereby the saturated and unsaturated FAMEs can be recovered separately. Membrane separation methods can also be applied to purifying fatty acid ester products such as biodiesel (Saleh, 2011).

In certain embodiments, a methane substrate of the invention is provided or obtained from a natural gas source, wherein the natural gas is “wet” natural gas or “dry” natural gas. Natural gas is referred to as “dry” natural gas when it is almost pure methane, having had most of the other commonly associated hydrocarbons removed. When other hydrocarbons are present, the natural gas is referred to as “wet”. Wet natural gas typically comprises about 70-90% methane, about 0-20% ethane, propane and butane (combined total), about 0-8% CO₂, about 0-5% N2, about 0-5% H₂S and trace amounts of oxygen, helium, argon, neon and xenon. In certain other embodiments, a methane substrate of the invention is provided or obtained from methane emissions, or methane off-gases, which are generated by a variety of natural and human-influenced processes, including anaerobic decomposition in solid waste landfills, enteric fermentation in ruminant animals, organic solids decomposition in digesters and wastewater treatment operations, and methane leakage in fossil fuel recovery, transport, and processing systems.

Table 1 below, provides exemplary polynucleotide and polypeptide sequences for implementing various embodiments of the present invention. These sequences are not meant to limit or exclude the use of other polynucleotide sequences encoding polypeptides or enzymes useful for producing multi-carbon compounds according to the present invention. For example, one of skill in the art can search gene sequence databases (or genome databases) and/or protein sequence databases (e.g., via BLAST or other sequence search algorithms) to identify homologous polynucleotides encoding one or more enzyme activities based on the reference sequences set forth in Table 1. Alternatively, a homologous polynucleotide may be isolated directly by using all or a portion of a nucleic acid sequence set forth in Table 1 (or a primer sequence set forth below in Table 2) as DNA hybridization probes to screen libraries from any desired microorgansim and/or PCR amplify a desired polynucleotide sequence using methodology well known to those skilled in the art.

TABLE 1
Exemplary Nucleic Acid and Polypeptide Sequences Described in the Invention

| Pathway or | | Nucleic acid | | Polypeptide | |
|---|---|---|---|---|---|
| Reaction | Gene Name | SEQ | Enzyme Name | SEQ ID | Organism |
| isobutanol | MCA1837 | SEQ ID NO: 1 | ALS | SEQ ID NO: 2 | M. capsulatus, Bath |
| isobutanol | MCA2272 | SEQ ID NO: 3 | KARI | SEQ ID NO: 4 | M. capsulatus, Bath |
| isobutanol | MCA2082 | SEQ ID NO: 5 | DHAD | SEQ ID NO: 6 | M. capsulatus, Bath |
| isobutanol | MCA0996 | SEQ ID NO: 7 | KDC | SEQ ID NO: 8 | M. capsulatus, Bath |
| isobutanol | YMR318C | SEQ ID NO: 9 | ADH | SEQ ID NO: 10 | S. cerevisiae |
| isobutanol | MtKDC | SEQ ID NO: 82 | KDC | SEQ ID NO: 162 | M. trichosporium |
| isobutanol | MtADH | SEQ ID NO: 83 | ADH | SEQ ID NO: 163 | M. trichosporium |
| isobutanol | McADH-2a | SEQ ID NO: 84 | ADH | SEQ ID NO: 164 | M. capsulatus, Bath |
| isobutanol | McADH-2b | SEQ ID NO: 85 | ADH | SEQ ID NO: 165 | M. capsulatus, Bath |
| Isobutanol | LlkivD | SEQ ID NO: 86 | KDC | SEQ ID NO: 166 | L. lactis |
| Isobutanol | ScPDC6 | SEQ ID NO: 87 | KDC | SEQ ID NO: 167 | S. cerevisiae |
| Isobutanol | ScARO10 | SEQ ID NO: 88 | KDC | SEQ ID NO: 168 | S. cerevisiae |
| Isobutanol | ScADH2 | SEQ ID NO: 89 | ADH | SEQ ID NO: 169 | S. cerevisiae |
| Isobutanol | ScPDC1 | SEQ ID NO: 90 | KDC | SEQ ID NO: 170 | S. cerevisiae |
| isobutanol | CaPDC | SEQ ID NO: 91 | KDC | SEQ ID NO: 171 | C. acetobutylicum |
| CH₄ to CH₃OH | MCA1798 | SEQ ID NO: 11 | pmoC subunit 1 | SEQ ID NO: 12 | M. capsulatus, Bath |
| CH₄ to CH₃OH | MCA1797 | SEQ ID NO: 13 | pmoA subunit 1 | SEQ ID NO: 14 | M. capsulatus, Bath |
| CH₄ to CH₃OH | MCA1796 | SEQ ID NO: 15 | pmoB subunit 1 | SEQ ID NO: 16 | M. capsulatus, Bath |
| CH₄ to CH₃OH | MCA2855 | SEQ ID NO: 17 | pmoC subunit 2 | SEQ ID NO: 18 | M. capsulatus, Bath |
| CH₄ to CH₃OH | MCA2854 | SEQ ID NO: 19 | pmoA subunit 2 | SEQ ID NO: 20 | M. capsulatus, Bath |
| CH₄ to CH₃OH | MCA2853 | SEQ ID NO: 21 | pmoB subunit 2 | SEQ ID NO: 22 | M. capsulatus, Bath |
| CH₄ to CH₃OH | MCA1194 | SEQ ID NO: 23 | mmoX | SEQ ID NO: 24 | M. capsulatus, Bath |
| CH₄ to CH₃OH | MCA1195 | SEQ ID NO: 25 | mmoY | SEQ ID NO: 26 | M. capsulatus, Bath |
| CH₄ to CH₃OH | MCA1196 | SEQ ID NO: 27 | mmoB | SEQ ID NO: 28 | M. capsulatus, Bath |
| CH₄ to CH₃OH | MCA1198 | SEQ ID NO: 29 | mmoZ | SEQ ID NO: 30 | M. capsulatus, Bath |
| CH₄ to CH₃OH | MCA1199 | SEQ ID NO: 31 | mmoD | SEQ ID NO: 32 | M. capsulatus, Bath |
| CH₄ to CH₃OH | MCA1200 | SEQ ID NO: 33 | mmoC | SEQ ID NO: 34 | M. capsulatus, Bath |
| CH₃OH to H₂CO | MCA0779 | SEQ ID NO: 35 | mxaF | SEQ ID NO: 36 | M. capsulatus, Bath |
| CH₃OH to H₂CO | MCA0780 | SEQ ID NO: 37 | mxaJ | SEQ ID NO: 38 | M. capsulatus, Bath |
| CH₃OH to H₂CO | MCA0781 | SEQ ID NO: 39 | mxaG | SEQ ID NO: 40 | M. capsulatus, Bath |
| CH₃OH to H₂CO | MCA0782 | SEQ ID NO: 41 | mxaI | SEQ ID NO: 42 | M. capsulatus, Bath |
| CH₃OH to H₂CO | MCA0783 | SEQ ID NO: 43 | mxaR | SEQ ID NO: 44 | M. capsulatus, Bath |
| CH₃OH to H₂CO | MCA0785 | SEQ ID NO: 45 | mxaA | SEQ ID NO: 46 | M. capsulatus, Bath |
| CH₃OH to H₂CO | MCA0786 | SEQ ID NO: 47 | mxaC | SEQ ID NO: 48 | M. capsulatus, Bath |
| CH₃OH to H₂CO | MCA0787 | SEQ ID NO: 49 | mxaK | SEQ ID NO: 50 | M. capsulatus, Bath |
| CH₃OH to H₂CO | MCA0788 | SEQ ID NO: 51 | mxaL | SEQ ID NO: 52 | M. capsulatus, Bath |
| CH₃OH to H₂CO | MCA0789 | SEQ ID NO: 53 | mxaD | SEQ ID NO: 54 | M. capsulatus, Bath |
| 1-butanol | MCA0354 | SEQ ID NO: 55 | Threonine- | SEQ ID NO: 56 | M. capsulatus, Bath |
| | | | ammonia-lyase | | |
| 1-butanol | MCA2275 | SEQ ID NO: 57 | 2-ethylmalate | SEQ ID NO: 58 | M. capsulatus, Bath |
| | | | synthase | | |
| 1-butanol | MCA2065 | SEQ ID NO: 59 | Isopropyl malate | SEQ ID NO: 60 | M. capsulatus, Bath |
| | | | dehydratase, | | |
| | | | large subunit | | |
| 1-butanol | MCA2064 | SEQ ID NO: 61 | Isopropyl malate | SEQ ID NO: 62 | M. capsulatus, Bath |
| | | | dehydratase, | | |
| | | | small subunit | | |
| 1-butanol | VIMSS17191 | SEQ ID NO: 160 | tdcB | SEQ ID NO: 161 | E. coli |
| 1-butanol | MCA0996 | SEQ ID NO: 7 | KDC | SEQ ID NO: 8 | M. capsulatus, Bath |
| 1-butanol | YMR318C | SEQ ID NO: 9 | ADH | SEQ ID NO: 10 | S. cerevisiae |
| 1-butanol | MtKDC | SEQ ID NO: 82 | KDC | SEQ ID NO: 162 | M. trichosporium |
| 1-butanol | MtADH | SEQ ID NO: 83 | ADH | SEQ ID NO: 163 | M. trichosporium |
| 1-butanol | McADH-2a | SEQ ID NO: 84 | ADH | SEQ ID NO: 164 | M. capsulatus, Bath |
| 1-butanol | McADH-2b | SEQ ID NO: 85 | ADH | SEQ ID NO: 165 | M. capsulatus, Bath |
| 1-butanol | LlkivD | SEQ ID NO: 86 | KDC | SEQ ID NO: 166 | L. lactis |
| 1-butanol | ScPDC6 | SEQ ID NO: 87 | KDC | SEQ ID NO: 167 | S. cerevisiae |
| 1-butanol | ScARO10 | SEQ ID NO: 88 | KDC | SEQ ID NO: 168 | S. cerevisiae |
| 1-butanol | ScADH2 | SEQ ID NO: 89 | ADH | SEQ ID NO: 169 | S. cerevisiae |
| 1-butanol | ScPDC1 | SEQ ID NO: 90 | KDC | SEQ ID NO: 170 | S. cerevisiae |
| 1-butanol | CaPDC | SEQ ID NO: 91 | KDC | SEQ ID NO: 171 | C. acetobutylicum |
| Fatty alcohol | FAR | SEQ ID NO: 65 | FAR | SEQ ID NO: 66 | M. algicola |
| Fatty acid ester | Ab-wax-dgaT | SEQ ID NO: 67 | wax-dgaT | SEQ ID NO: 68 | A. baylyi |
| Fatty acid ester | Psyc_0223 | SEQ ID NO: 69 | PaWES | SEQ ID NO: 70 | P. arcticus |
| Fatty acid ester | ROP_02100 | SEQ ID NO: 71 | RoWES1 | SEQ ID NO: 72 | R. opacus |
| Fatty acid ester | ROP_13050 | SEQ ID NO: 73 | RoWES2 | SEQ ID NO: 74 | R. opacus |
| Fatty acid ester | ROP_54550 | SEQ ID NO: 75 | RoWES3 | SEQ ID NO: 76 | R. opacus |
| Fatty acid ester | ROP_26950 | SEQ ID NO: 77 | RoWES4 | SEQ ID NO: 78 | R. opacus |
| 2,3-butanediol | YAL060W | SEQ ID NO: 156 | Bdh1 | SEQ ID NO: 157 | S. cerevisiae |
| RuMP | MCA3049 | SEQ ID NO: 160 | HPS | SEQ ID NO: 161 | M. capsulatus, Bath |
| RuMP | MCA3050 | SEQ ID NO: 162 | HPS/PHI | SEQ ID NO: 163 | M. capsulatus, Bath |

TABLE 2
Plasmid, Primer, Promoter and Gene Fragment
Sequences Described in the Invention

| Name | Nucleic acid SEQ ID |
|---|---|
| pCM132 | SEQ ID NO: 79 |
| pJSvec | SEQ ID NO: 80 |
| pMZT3 | SEQ ID NO: 81 |
| JPS00082 | SEQ ID NO: 92 |
| JPS00031 | SEQ ID NO: 93 |
| JPS00032 | SEQ ID NO: 94 |
| GMV257 | SEQ ID NO: 95 |
| JPS00118 | SEQ ID NO: 96 |
| JPS00119 | SEQ ID NO: 97 |
| ESG00087 | SEQ ID NO: 98 |
| GMV251 | SEQ ID NO: 99 |
| rnpB | SEQ ID NO: 100 |
| JPS00161 | SEQ ID NO: 101 |
| JPS00162 | SEQ ID NO: 102 |
| JPS00163 | SEQ ID NO: 103 |
| JPS00164 | SEQ ID NO: 104 |
| JPS00172 | SEQ ID NO: 105 |
| JPS00173 | SEQ ID NO: 106 |
| JPS00174 | SEQ ID NO: 107 |
| JPS00176 | SEQ ID NO: 108 |
| JPS00177 | SEQ ID NO: 109 |
| JPS00157 | SEQ ID NO: 110 |
| JPS00178 | SEQ ID NO: 111 |
| Me-AM1 PmxaF | SEQ ID NO: 112 |
| JPS00169 | SEQ ID NO: 113 |
| GMV00251 | SEQ ID NO: 114 |
| JPS00170 | SEQ ID NO: 115 |
| JPS00171 | SEQ ID NO: 116 |
| JPS00153 | SEQ ID NO: 117 |
| JPS00151 | SEQ ID NO: 118 |
| JPS00154 | SEQ ID NO: 119 |
| JPS00183 | SEQ ID NO: 120 |
| JPS00185 | SEQ ID NO: 121 |
| J23100 | SEQ ID NO: 122 |
| J23100 hybrid | SEQ ID NO: 123 |
| J23115 | SEQ ID NO: 124 |
| GMV00233 | SEQ ID NO: 125 |
| GMV00235 | SEQ ID NO: 126 |
| GMV00433 | SEQ ID NO: 127 |
| GMV00434 | SEQ ID NO: 128 |
| GMV00435 | SEQ ID NO: 129 |
| GMV00436 | SEQ ID NO: 130 |
| GMV00437 | SEQ ID NO: 131 |
| GMV00438 | SEQ ID NO: 132 |
| GMV00439 | SEQ ID NO: 133 |
| GMV00440 | SEQ ID NO: 134 |
| GMV00441 | SEQ ID NO: 135 |
| GMV00442 | SEQ ID NO: 136 |
| ESG00084 | SEQ ID NO: 137 |
| ESG00088 | SEQ ID NO: 138 |
| pMZT37 | SEQ ID NO: 139 |
| MaFAR-g1 | SEQ ID NO: 140 |
| MaFAR-g2 | SEQ ID NO: 141 |
| MaFAR-g3 | SEQ ID NO: 142 |
| MaFAR-g4 | SEQ ID NO: 143 |
| GMV410 | SEQ ID NO: 144 |
| GMV411 | SEQ ID NO: 145 |
| GMV412 | SEQ ID NO: 146 |
| GMV413 | SEQ ID NO: 147 |
| GMV414 | SEQ ID NO: 148 |
| GMV415 | SEQ ID NO: 149 |
| GMV416 | SEQ ID NO: 150 |
| GMV417 | SEQ ID NO: 151 |
| GMV418 | SEQ ID NO: 152 |
| GMV419 | SEQ ID NO: 153 |
| GMV420 | SEQ ID NO: 154 |
| GMV421 | SEQ ID NO: 155 |
| GMV422 | SEQ ID NO: 158 |
| GMV423 | SEQ ID NO: 159 |

### EXAMPLES

The present invention is further defined in the following Examples. It should be understood that these examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### Biosynthetic Production of Isobutanol from Methane

Initial experiments were performed to confirm and validate enzymatic activity of isobutanol pathway enzymes at the relatively high temperatures (i.e., 45° C.) requisite for growth of one preferred methanotroph host organism, Methylococcus capsulatus (Bath). Thus, in this example, the methanotroph M. capsulatus was engineered in the first series of experiments to overexpress two isobutanol pathway enzymes, ketoacid decarboxylase (KDC) and alcohol dehydrogenase (ADH), prior to introducing the full complement of five isobutanol pathway enzymes (Atsumi et al., 2010) into M. capsulatus. Following the functional validation of KDC and ADH activity in M. capsulatus (set forth below), the complete five-gene isobutanol pathway was introduced into M. capsulatus, the results of which are set forth below.

### Gene Selection, Synthesis and Cloning

For the two-gene (isobutanol) pathway experiments (and for the downstream section of the five-gene isobutanol pathway set forth below), ketoacid decarboxylase (KDC) and alcohol dehydrogenase (ADH) genes were amplified by colony PCR from Methylosinus trichosporium (strain: OB3b, National Collection of Industrial, Food and Marine Bacteria (NCIMB) Accession No: 11131) and Methylococcus capsulatus (Bath). The Methylosinus trichosporium gene, MtKDC, encoding KDC is set forth in SEQ ID NO: 82, Methylosinus trichosporium gene, MtADH, encoding ADH is set forth in SEQ ID NO: 83. The Methylococcus capsulatus (Bath) gene, McKDC, encoding KDC is set forth in SEQ ID NO:7, the Methylococcus capsulatus (Bath) genes, McADH-2a and McADH-2b, encoding two ADH2 homologs, are set forth in SEQ ID NO:84 and SEQ ID NO:85, respectively.

Other KDC and ADH genes such as L1KIVD: Lactococcus lactis KDC (SEQ ID NO: 86); ScPDC6: Saccharomyces cerevisiae PDC6 (SEQ ID NO:87); ScARO10: S. cerevisiae ARO10 (SEQ ID NO:88); ScADH2: S. cerevisiae ADH2 (SEQ ID NO:89); ScPDC1: S. cerevisiae PDC1 (SEQ ID NO:90); CaPDC: Clostridium acetobutylicum PDC (SEQ ID NO:91) were codon optimized for expression in M. capsulatus and de novo synthesized by GenScript (Piscataway, NJ). Various KDC and ADH combinations were cloned with a constitutive promoter (J23115) or inducible (Ptrc) promoter into plasmid pCM132 (Accession No. AF327720; SEQ ID NO:79) with the Clontech In-Fusion kit (Mountain View, CA). A gene for the ds-Red protein was used as a control. Plasmids were transformed into E. coli S17-1 for conjugation.

### Vector Inserts

Vector inserts contain the DNA fragments that are to be carried in the plasmid. The vector inserts were designed as exchangeable parts to the vector backbone described above. In one embodiment of the 2-gene pathway example, the plasmids were designed to contain two inserts made up of Methylococcus capsulatus KDC (MCA0996; SEQ ID NO:7) and Saccharomyces cerevisiae ADH6 (YMR318C; SEQ ID NO:9) genes. Both genes were amplified from genomic DNA of their respective hosts, with the primers described above in Tables 2 and below in Table 3.

TABLE 3
Plasmid insert modules, templates and primers

| | | Modules | | | |
|---|---|---|---|---|---|
| Plasmid | PCR rxn: | Vector Backbone | Insert 1 | Insert 2 | Insert 3 |
| pJS0025 | template | pJSvec | MCA0996 (M. | YMR318C (S. | — |
| | | | capsulatus DNA) | cerevisiae DNA) | |
| | primer 1 | JPS0082 | JPS0032 | JPS00118 | — |
| | primer 2 | JPS0031 | GMV00257 | JPS00119 | — |
| pGMV145 | template | pMZT3 | MCA0996 (M. | YMR318C (S. | — |
| | | | capsulatus DNA) | cerevisiae DNA) | |
| | primer 1 | JPS0082 | GMV00251 | JPS00118 | — |
| | primer 2 | ESG00087 | GMV00257 | JPS00119 | — |
| pJS034 | template | pGMV145 | IDT gBlock | — | — |
| | | | synthesized | | |
| | | | rnpB DNA | | |
| | primer 1 | JPS00161 | JPS00163 | — | — |
| | primer 2 | JPS00162 | JPS00164 | — | — |
| pJS041 | template | pJS034 | MCA1837 (M. | MCA2272 (M. | MCA2082 |
| pJS041n | | | capsulatus DNA) | capsulatus DNA) | (M. |
| | | | | | capsulatus |
| | | | | | DNA) |
| | primer 1 | JPS00162 | JPS00173 | JPS00176 | JPS00157 |
| | primer 2 | JPS00172 | JPS00174 | JPS00177 | JPS00178 |
| pJS048 | template | pJS034 | IDT gBlock | — | — |
| | | | synthesized Me-AM1 | | |
| | | | PmxaF DNA | | |
| | primer 1 | JPS00169 | JPS00170 | — | — |
| | primer 2 | GMV251 | JPS00171 | — | — |
| pJS038 | template | pGMV145 | MCA1837 (M. | — | — |
| | | | capsulatus DNA) | | |
| | primer 1 | JPS00153 | JPS00151 | — | — |
| | primer 2 | GMV251 | JPS00154 | — | — |
| pJS042 | template | pJS048 | pJS038 | — | — |
| pJS042n | primer 1 | JPS00162 | JPS00173 | — | — |
| | primer 2 | JPS00172 | JPS00178 | — | — |
| pJS050 | template | pJS041n | pJS041n | — | — |
| | primer 1 | JPS00183 | JPS00174 | — | — |
| | primer 2 | JPS00176 | JPS00185 | — | — |

The modular parts (i.e., vector backbone and vector inserts) were PCR amplified (as listed in Table 3) with NEB Phusion master mix (New England Biolabs; Ipswich, MA) according to the manufacturer's instructions and in vitro assembled with the Clontech InFusion HD Cloning System kit (Clontech; Mountain View, CA) according to the manufacturer's instructions to generate circular plasmid listed below.

The in vitro assembled plasmids (2 μl of the InFusion reaction) were transformed into chemically competent NEB Turbo E. coli cells, screened by colony PCR, purified, and subsequently sequence verified.

The plasmid pJSvec (SEQ ID NO:80) served as the template for the vector backbone with an inducible promoter and consisted of the pCM132 cloning vector (SEQ ID NO: 79), lacIq, and the IPTG-inducible pTrc promoter.

The plasmid pMZT3 (SEQ ID NO:81) served as the template for the vector backbone with a constitutive promoter and consisted of the pCM132 (SEQ ID NO:79) cloning vector and E. coli J23115 promoter (SEQ ID NO:124).

The plasmid pJS0025 was designed to express M. capsulatus KDC (MCA0996; SEQ ID NO:7) and S. cerevisiae ADH6 (YMR 318C; SEQ ID NO:9) from the inducible promoter.

The plasmid pGMV145 was designed to express M. capsulatus KDC (MCA0996; SEQ ID NO:7) and S. cerevisiae ADH6 (YMR 318C; SEQ ID NO:9) from the constitutive promoter.

The plasmid pJS034 introduced a second terminator sequence into pGMV145. The pGMV145 vector backbone was PCR amplified with primers JPS00161 (SEQ ID NO: 101)/JPS00162 (SEQ ID NO:102) and KOD mastermix. The insert contained DNA sequence for rnpB (SEQ ID NO:100) synthesized as a gBlock from Integrated DNA Technologies (Coralville, IA) and amplified with JPS00163 (SEQ ID NO:103)/JPS00164 (SEQ ID NO:104) primers.

### Expression of the Full Five-Gene Pathway for Methane-to-Isobutanol Conversion

In order to synthesize isobutanol from methane (i.e., via pyruvate), without the need to exogenously supply a ketoacid intermediate, the pJS041 and pJS04In plasmids were designed to express all five isobutanol pathway genes: (1) M. capsulatus KDC (MCA0996; SEQ ID NO:7) and (2) S. cerevisiae ADH6 (YMR318C; SEQ ID NO:9) from the J23115 constitutive promoter (SEQ ID NO:124), and (3) M. capsulatus ilvK (MCA1837; SEQ ID NO: 1), (4) M. capsulatus ilvC (MCA2272; SEQ ID NO:3), and (5) M. capsulatus ilvD (MCA2082; SEQ ID NO:5) from the J23100 constitutive promoter (see, FIG. 3). Plasmid pJS041n contains the canonical J23100 promoter sequence (5′-TTGACGGCTAGCTCAGTCCTAGGTACAGTGCTAGC-3′; SEQ ID NO:122), and plasmid pJS041 contains a modified J23100 promoter sequence (5′-TTGACGGCTAGCTCAGCCCTTGGTACAATGCTAGC-3′; SEQ ID NO:123), which represents a hybrid fusion of the J23100 and J23115 (SEQ ID NO:124) promoters that arose during the process of cloning and generating the plasmid in E. coli (Table 3). This mutated construct was retained and tested to see whether the promoter mutations might impart improved production of isobutanol in the microbial expression host (e.g., M. capsulatus (Bath)).

TABLE 4
Sequence comparison between the “hybrid”
promoters in plasmids pJS041 and pJS042 and
the canonical promoters J23115 and J23100

| | |
|---|---|
| J23115 | TTTATAGCTAGCTCAGCC |
| (SEQ ID NO: 124) | CTTGGTACAATGCTAGC |
| pJS041-hybrid | TTGACGGCTAGCTCAGCC |
| (SEQ ID NO: 123) | CTTGGTACAATGCTAGC |
| J23100 | TTGACGGCTAGCTCAGTC |
| (SEQ ID NO: 122) | CTAGGTACAGTGCTAGC |

The pJS048 plasmid replaced the J23100 promoter with the MxaF promoter (SEQ ID NO: 112) from Methylobacterium extorquens AM-1 in pJS034.

The pJS050 plasmid was designed to express five genes: M. capsulatus KDC (MCA0996; SEQ ID NO:7) and S. cerevisiae ADH6 (YMR318C; SEQ ID NO:9) from the J23115 constitutive promoter and M. capsulatus ilvK (MCA1837; SEQ ID NO:1), M. capsulatus ilvC (MCA2272; SEQ ID NO:3), and M. capsulatus ilvD (MCA2082; SEQ ID NO: 5) from the J23115 constitutive promoter.

### Conjugations

The method for conjugal transfer of RP4-mob-containing plasmids into M. trichosporium and M. capsulatus (Bath) was based on the method described previously (Martin & Murrell, 1995; Stafford et. al., 2003). Briefly, 10 ml of a 16 hour culture of E. coli S17-1 carrying the plasmid was collected on a sterile 47 mm, 0.2 μm pore-size, nitrocellulose filter (Millipore). The cells were washed with 50 ml NMS medium without antibiotic. A fresh 50 ml culture of the M. trichosporium or M. capsulatus (Bath) recipient grown to an optical density (A₅₄₀) of 0.2-0.4 (mid-exponential phase of growth) was collected on the same filter as the E. coli S17-1 host cells. The cells were washed with 50 ml NMS and the filter was placed on an NMS agar plate supplemented with 0.02% (w/v) Proteose Peptone (Difco Laboratories, Detroit, MI) and incubated for 24 hours at 30° C. (for M. trichosporium) or 37° C. (M. capsulatus (Bath)) in the presence of 20-25% methane (CH₄) (v/v) in air. After incubation, the cells from the conjugation plate were washed from the filter with 10 ml of NMS, pelleted by centrifugation at 7,000×g, and re-suspended in 1 ml of NMS. 150 μl aliquots were spread onto selective NMS plates containing 10 μg/ml nalidixic acid to select against E. coli and 15 μg/ml kanamycin for plasmid selection and incubated at 30° C. or 45° C. for M. trichosporium or M. capsulatus, respectively. The remaining cells were grown in NMS liquid containing 10 μg/ml nalidixic acid and 15 μg/ml kanamycin (Sigma, St. Louis, MO) as a secondary selection process. Cells grown in liquid selection were serially passaged three times, before spreading onto selective NMS plates for clone isolation.

M. capsulatus Growth Conditions

From a saturated starter culture, M. capsulatus (Bath) cells were diluted 1:100 into 10 ml of fresh NMS containing 15 μg/ml kanamycin in a 125-ml shake flask. For ketoacid feeding experiments, cultures were treated with 1 g/L 2-ketovalerate (CAS #1821 Feb. 9) or 8 g/L 2-ketoisovalerate (CAS #3715-19-5) with or without the inducer, 0.1 mM isopropylthiogalactoside (IPTG). The flasks were closed with Suba-seals, injected with 20-25% CH₄ (v/v) in air, and incubated at 45° C. for 0-120 hours.

Extraction of Alcohols from the Growth Medium

1. Isobutanol production: The shake-flask samples were prepared for extraction by cooling them on ice for 1 hour, which ensures that the volatile organic compounds (VOC's) in the vapor phase were not lost to the atmosphere after the Suba-seal is opened.

2. If extracting from a 9-10 ml culture, all of the culture was transferred to a 50 ml tube. For samples with high isobutanol productions (e.g., pGMV 145), 10 ml of ethyl acetate was added for extraction. For samples with low isobutanol production, only 3 ml of ethyl acetate was used. Once ethyl acetate was added to the cultures, they were subjected to either vortexing (1-2 minutes) or shaking at room temperature (for 1 hour) for efficient extraction.

3. The tubes were then centrifuged at 4000 rpm for 20 minutes in an Eppendorf 5810 centrifuge equipped with an A-4-81 rotor.

4. One (1) ml of the organic layer was then filtered (0.2 μm PTFE membrane) and transferred to 2 ml glass Agilent gas chromatography vials for analysis.

### GC-FID Analysis for Isobutanol

The extracted alcohol compounds were quantified with the Agilent 7890A gas chromatograph (GC) with flame ionization detector and PAL auto-sampler. An HP InnoWax capillary column (30 m, 0.32-mm internal diameter, 0.25-mm film thickness; Agilent Technologies, Santa Clara, CA) was used to separate the alcohols. The GC oven temperature was initially set at 35° C. for 1 minute and ramped at rate of 10° C./minute until 85° C. was reached and held for 1 minute. A second temperature ramp of 80° C./minute up to 240° C. was performed and held for 2 minutes. Hydrogen gas was the carrier gas used with 9.3 psi constant inlet pressure. The inlet and detector were maintained at 240° C. A 1 μl sample was injected in split injection mode with a 25:1 split ratio.

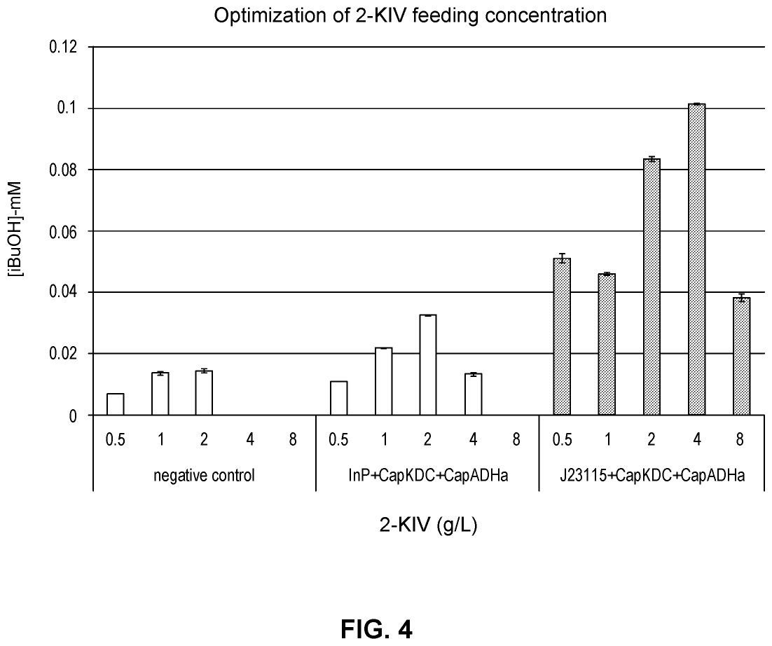

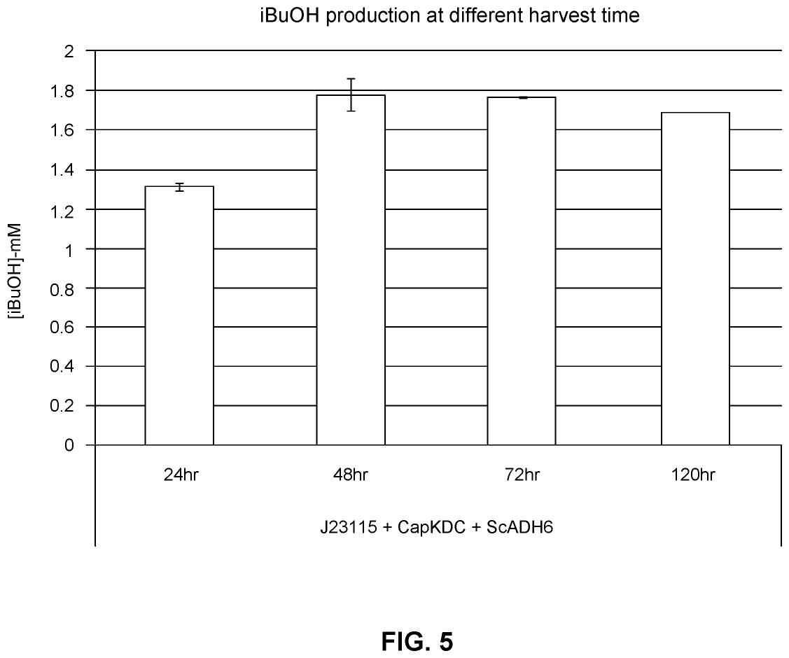

When the two-gene KDC/ADH pathway was expressed in M. capsulatus and the isobutanol production was measured (using exogenous 2-KIV feeding), the following results were observed. A concentration of 2-KIV greater than about 4 g/L had a toxic effect on growth, wherein a 2-KIV concentration of about 2 g/L yielded the best results (FIG. 4). Peak isobutanol production occurred about 48-72 hours after 2-KIV feeding (FIG. 5). E. coli promoters function in M. capsulatus, but not equally well. Constitutive promoters yielded better results than inducible promoters, but the optimal constitutive promoter will typically depend on the individual construct to be used. For example, J23115 was observed to work best for M. capsulatus KDC and M. capsulatus ADH (data not shown). Lastly, different host strains require slightly different concentrations of 2-KIV to maximize isobutanol production.

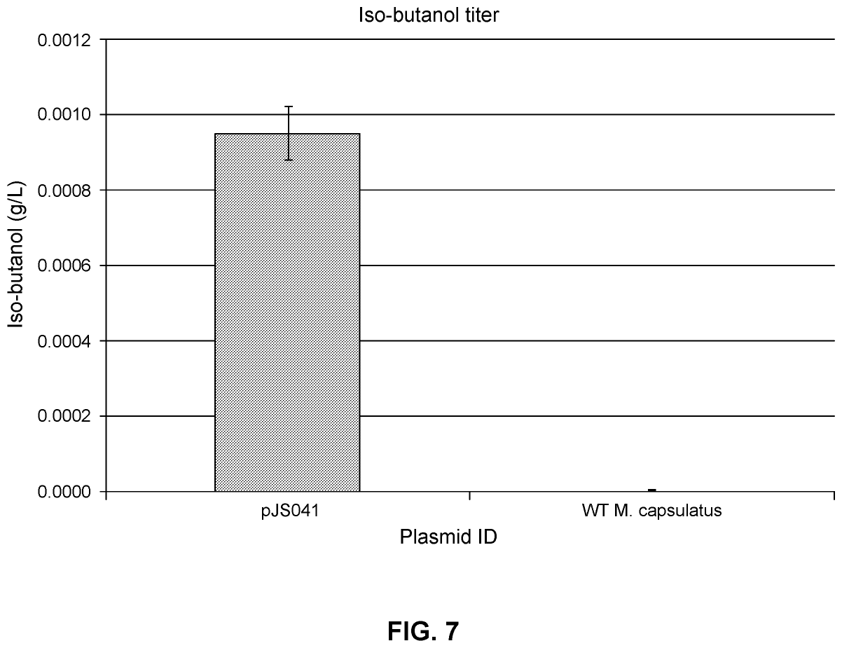

The best two-gene combination with a constitutive promoter (J23115; SEQ ID NO: 124) was M. capsulatus KDC and S. cerevisiae ADH6 (plasmid pGMV145), wherein harvesting after 48-72 hours produced the most isobutanol (FIG. 7). The vector construct using pGMV145, having constitutive promoter J23115, a CapKDC gene (MCA0996; SEQ ID NO: 7), and a ScADH6 gene (YMR318C; SEQ ID NO:9), produced the most isobutanol after 2-KIV feeding, which was about 3 mM (or about 0.22 g/L).

When the complete five-gene isobutanol pathway was introduced into a host strain, plasmid pJS041 yielded the highest levels of isobutanol production, with a measured titer of about 0.001 g/liter (FIG. 7), compared to no detectable production in the wild-type strain.

In certain embodiments, the production of isobutanol from methane substrate in a host strain (i.e., expressing the five-gene isobutanol pathway, e.g. via plasmid pJS041) is further optimized by genetic manipulations described above, as well as by cultivating the host strain in a fermentor culture with continuous CH₄ perfusion, instead of batch addition of CH₄ to the culture medium (as was done for the shake flasks experiments). In other embodiments, the production of increased isobutanol titers from methane in a host strain is further optimized via manipulations to the fermentation process (batch fed or perfusion), such as feeding additional media components as they are depleted (phosphate, nitrate, etc.) and maintaining the pH by continuously adding acid or base.

### Example 2

### Biosynthetic Production of 1-Butanol from Methane

A ketoacid pathway analogous to that described in Example 1, but designed to produce 1-butanol (n-butanol) is engineered in a single carbon (C1) metabolizing microbial host, such as M. capsulatus (Bath). In this example, L-threonine (which is ultimately generated from methane via phospoenolpyruvate) is first de-aminated to 2-ketobutyrate (2-oxobutanoate) by the action of threonine dehydratase (also referred to in the art as threonine ammonia-lyase (EC 4.3.1.19) encoded by the genes ilvA or tdcB) (Shen & Liao, 2008). The tdcB gene product has the biotechnological advantage that the enzyme is a catabolic enzyme, and is not feedback inhibited by L-valine or L-isoleucine (Guillouet et al., 1999).

In the second reaction step, the reaction catalyzed by leuA (encoding isopropylmalate synthase/2-ethylmalate synthase (EC 2.3.3.6)) combines 2-ketobutyrate, acetyl-CoA, and H₂O to create (R)-2-ethylmalate. In the third reaction step, the gene product of leuC and leuD (encoding the two subunits of isopropylmalate isomerase) converts 2-ethylmalate into 3-ethylmalate. In the fourth reaction step, the gene product of leuB (encoding the enzyme 3-isopropylmalate dehydrogenase) converts 3-ethylmalate into 2-ketovalerate). At this stage, the same two enzymes used in the previous example, KDC (acting as a 2-ketovalerate decarboxylase) and ADH2 (alcohol dehydrogenase), are used to convert 2-ketovalerate into 1-butanol.

An alternate pathway (the citramalate pathway) from phosphoenolpyruvate and pyruvate to 2-ketobutyrate has also been described for making 1-butanol (Atsumi & Liao, 2008).

As described, above, the plasmids generated in this study are based on the broad-host-range pCM132 (Accession No. AF327720, SEQ ID NO:79) cloning vector described by Marx & Lidstrom (2001). In this embodiment, the use of the Clontech (catalog no. 639647) InFusion HD Cloning System kit is one example of how to construct plasmids, but is not meant to limit or exclude other methods that are known in the art.

### Vector Backbones

Vector backbones contain the components of the plasmid that will remain constant. The broad-host range pCM132 vector was modified to produce vector backbones for the plasmids in this study. The pCM132 vector consisted of the following components: trrnB terminator, kanamycin resistance gene, trfA, IncP oriT, IncP oriV, colE1 ori, and lacZ. This parental vector was modified to replace lacZ with a vector insert that contains promoter sequence to produce plasmids pMZT3 (SEQ ID NO:81) and pMZT37 (SEQ ID NO: 139).

### Vector Inserts

Vector inserts contain DNA to be added to a vector backbone. The inserts were designed as exchangeable (modular) parts to the vector and in this example consist of Methylococcus capsulatus KDC (MCA0996; SEQ ID NO:7), leuA (MCA2275; SEQ ID NO: 57), leuCDB (MCA2063; SEQ ID NO:63, MCA2064; SEQ ID NO:61 and MCA2065; SEQ ID NO:59), Saccharomyces cerevisiae ADH6 (YMR318C; SEQ ID NO:9), and M. capsulatus ilvA (MCA0354; SEQ ID NO:55) or E. coli tdcB (SEQ ID NO:160) genes. The genes were amplified from genomic DNA of their respective hosts with the primers described in Table 5.

The modular parts (vector backbone and vector insert) were PCR amplified as listed in Table 4 with NEB Phusion master mix according to the manufacturer's instructions and in vitro assembled with the Clontech InFusion HD Cloning System kit according to the manufacturer's instructions to generate circular plasmid. The in vitro assembled plasmids (2 μl of the InFusion reaction) were transformed into chemically competent NEB Turbo E. coli cells, screened for by colony PCR, purified, and subsequently sequence verified.

The pGMV145 plasmid was designed to express M. capsulatus KDC (MCA0996; SEQ ID NO:7) and S. cerevisiae ADH6 (YMR318C; SEQ ID NO:9) from the constitutive promoter.

The pJS034 plasmid introduced a second terminator sequence into pGMV145. The pGMV145 vector backbone was PCR amplified with primers JPS00161 (SEQ ID NO: 101)/JPS00162 (SEQ ID NO:102) and KOD mastermix. The insert was rnpB DNA synthesized as a gBlock from IDT and amplified with JPS00163 (SEQ ID NO: 103)/JPS00164 (SEQ ID NO:104) primers.

The pGMV165 plasmid was designed to express 3 genes: M. capsulatus ilvA (MCA0354; SEQ ID NO:55), M. capsulatus KDC (MCA0996; SEQ ID NO:7) and S. cerevisiae ADH6 (YMR318C; SEQ ID NO:9) from the J23115 (SEQ ID NO:124) constitutive promoter.

The pGMV166 plasmid was designed to express 3 genes: E. coli tdcB (SEQ ID NO: 160), M. capsulatus KDC (MCA0996; SEQ ID NO:7) and S. cerevisiae ADH6 (YMR318C; SEQ ID NO:9) from the J23115 (SEQ ID NO:124) constitutive promoter.

The pGMV167 plasmid was designed to express 7 genes: M. capsulatus ilvA (MCA0354; SEQ ID NO:55), M. capsulatus KDC (MCA0996; SEQ ID NO:7) and S. cerevisiae ADH6 (YMR318C; SEQ ID NO:9) from the J23115 (SEQ ID NO:124) constitutive promoter and M. capsulatus leuCDB (MCA2063; SEQ ID NO:63, MCA2064; SEQ ID NO:61 and MCA2065; SEQ ID NO:59) and M. capsulatus leuA (MCA2275; SEQ ID NO: 57) from second J23115 (SEQ ID NO:124) constitutive promoter.

The pGMV168 plasmid was designed to express 7 genes: E. coli tdcB (SEQ ID NO: 160), M. capsulatus KDC (MCA0996; SEQ ID NO:7) and S. cerevisiae ADH6 (YMR318C; SEQ ID NO:9) from the J23115 constitutive promoter and M. capsulatus leuCDB (MCA2063; SEQ ID NO:63, MCA2064; SEQ ID NO:61 and MCA2065; SEQ ID NO: 59) and leuA (MCA2275; SEQ ID NO:57) from a second J23115 constitutive promoter.

Host strains modified with these plasmids were grown on methane as described in the examples above, harvested, extracted, and analyzed for 1-butanol production.

TABLE 5
Insert Modules, Templates and Primers for 1-Butanol Production

| | | Modules | | | |
|---|---|---|---|---|---|
| | PCR | Vector | | | |
| Plasmid | rxn: | backbone | Insert 1 | Insert 2 | Insert 3 |
| pGMV145 | template | pMZT3 | MCA0996 (M. | YMR318C (S. | — |
| | | | capsulatus DNA) | cerevisiae DNA) | |
| | primer 1 | JPS0082 | GMV00251 | JPS00118 | — |
| | primer 2 | ESG00087 | GMV00257 | JPS00119 | — |
| pJS034 | template | pGMV145 | IDT gBlock | — | — |
| | | | synthesized rnpB | | |
| | | | DNA | | |
| | primer 1 | JPS00161 | JPS00163 | — | — |
| | primer 2 | JPS00162 | JPS00164 | — | — |
| pGMV165 | template | pJS034 | pJS034 | MCA0354 (M. | — |
| | | | | capsulatus DNA) | |
| | primer 1 | GMV435 | GMV433 | GMV431 | — |
| | primer 2 | ESG000087 | GMV434 | GMV432 | — |
| pGMV166 | template | pJS034 | pJS034 | tdcB (E. coli DNA) | — |
| | primer 1 | GMV435 | GMV433 | GMV436 | — |
| | primer 2 | ESG000087 | GMV434 | GMV437 | — |
| pGMV167 | template | pGMV165 | pGMV165 | MCA2063-2065 (M. | MCA2275 (M. |
| | | | | capsulatus DNA) | capsulatus |
| | | | | | DNA) |
| | primer 1 | JPS163 | GMV235 | GMV439 | GMV441 |
| | primer 2 | GMV233 | GMV438 | GMV440 | GMV442 |
| pGMV168 | template | pGMV166 | pGMV166 | MCA2063-2065 (M. | MCA2275 (M. |
| | | | | capsulatus DNA) | capsulatus |
| | | | | | DNA) |
| | primer 1 | JPS163 | GMV235 | GMV439 | GMV441 |
| | primer 2 | GMV233 | GMV438 | GMV440 | GMV442 |

### Example 3

### Biosynthetic Production of Fatty Alcohols from Methane

Conversion of methane to diesel components requires engineering the native metabolism of methanotrophs. The two principal native pathways that can be engineered for increased production of diesel components are the fatty acid pathway and isoprenoid pathway. In the current example, the invention describes the use of the fatty acid pathway for synthesis of diesel (wax ester) components.

Fatty acids are an important source of energy and adenosine triphosphate (ATP) for many cellular organisms. Excess fatty acids, glucose, and other nutrients can be stored efficiently as fat. All cell membranes are built up of phospholipids, each of which contains fatty acids. Fatty acids are also used for protein modification. Fatty acid synthesis is the creation of fatty acids from acetyl-CoA and malonyl-CoA precursors through action of enzymes called fatty acid synthases. Fatty acid chain length and degree of saturation depends on the host microorganism. With regard to M. capsulatus (Bath), the primary fatty acids are C16 with saturated or mono unsaturated carbon chains.

The conversion of methane to diesel components requires the over-expression of specific heterologous (exogenous) enzymes within a methanotroph (or non-methanotroph) host microorganism, wherein the over-expression of specific heterologous (exogenous) enzymes can divert the flux from native fatty acid synthesis to compounds of interest. Key intermediates of the fatty acid pathway are the fatty acyl-ACP molecules. Thus, the over-expression of specific heterologous enzymes in a host microorganism divert the flux from acyl-ACP to diesel components such as fatty acids, fatty alcohols, fatty esters and derivatives thereof. Thus, in certain embodiments, a host microorganism has been engineered to over-express specific enzymes such as a fatty acyl ACP reductase (FAR), a fatty acyl CoA reductase (CAR) and wax ester synthases (WES) for diverting flux from native compounds to compounds of interest. Active expression of these enzymes results in the conversion of methane to diesel components via FARs, CARs and WES enzymes cloned and expressed in a host microorganism (e.g., M. capsulatus (Bath)).

A biosynthetic pathway analogous to that described in Example 1, but designed to produce fatty alcohols can be engineered in a (C1) metabolizing host microorganism, such as M. capsulatus. In this example, fatty acyl-CoA (which is ultimately generated from methane via pyruvate) is converted directly into fatty alcohols by the heterologous overexpression of a fatty-acyl-CoA reductase (FAR).

### Construction of Methanotroph Plasmids for Fatty Alcohol Production

As described, above, the plasmids generated in this study are based on the broad-host-range pCM132 (Accession No. AF327720) cloning vector (SEQ ID NO:79) described by Marx & Lidstrom (2001). In this embodiment, the use of the Clontech (catalog no. 639647) InFusion HD Cloning System kit is one example of how to construct plasmids, but is not meant to limit or exclude other methods that are known in the art.

### Vector Backbones

Vector backbones contain the components of the plasmid that will remain constant. The broad-host range pCM132 vector was modified to produce vector backbones for the plasmids in this study. The pCM132 vector consisted of the following components: trrnB terminator, kanamycin resistance gene, trfA, IncP oriT, IncP oriV, colE1 ori, and lacZ. This parental vector was modified to replace lacZ with a vector insert that contains promoter sequence to produce plasmids pMZT3 (SEQ ID NO:81) and pMZT37 (SEQ ID NO: 139).

### Vector Inserts

Vector inserts contain DNA to be added to the vector backbone. The inserts were designed as exchangeable (modular) parts to the vector and in this embodiment consist of the following components. In this example, the plasmids were designed to contain one insert: Marinobacter algicola fatty acid reductase (MaFAR; SEQ ID NO:65), also known as a fatty acyl-CoA reductase. The MaFAR gene was codon optimized and synthesized as a series of 4 gBlocks from Integrated DNA Technologies (Coralville, IA). The synthesized DNA was designed to include pivot regions to allow proper assembly by InFusion.

### Assembly of the Constructs

The modular parts (vector backbone and vector insert) were PCR amplified as listed in Table 4 with NEB Phusion master mix according to the manufacturer's instructions and in vitro assembled with the Clontech InFusion HD Cloning System kit according to the manufacturer's instructions to generate circular plasmid. The in vitro assembled plasmids (2 μl of the InFusion reaction) were transformed into chemically competent NEB Turbo E. coli cells, screened for by colony PCR, purified, and subsequently sequence verified.

Plasmid pMZT3 (SEQ ID NO:81) served as the template for the vector backbone with a constitutive promoter and consisted of the pCM132 cloning vector, E. coli J23115 promoter. The vector backbone was PCR amplified from the pMZT3 template with primers ESG00084 (SEQ ID NO:137)/ESG00087 (SEQ ID NO:98).

Plasmid pMZT37 (SEQ ID NO:139) served as the template for the vector backbone with a constitutive promoter and consisted of the pCM132 cloning vector, E. coli J23100 promoter. The vector backbone was PCR amplified from the pMZT3 template with primers ESG00084 (SEQ ID NO:137)/ESG00088 (SEQ ID NO:138).

The pGMV147 plasmid was designed to express M. algicola FAR gene (SEQ ID NO: 65) from the J23115 constitutive promoter (SEQ ID NO:124). The modules of this plasmid included the PCR amplified pMZT3 vector backbone and four synthesized DNA gene fragments from IDT (MaFAR-g1; SEQ ID NO:140, MaFAR-g2; SEQ ID NO:141, MaFAR-g3; SEQ ID NO:142 and MaFAR-g4; SEQ ID NO:143).

The pGMV148 plasmid was designed to express M. algicola FAR gene (SEQ ID NO: 65) from the J23110 constitutive promoter (SEQ ID NO:122). The modules of this plasmid included the PCR amplified pMZT37 vector backbone and four synthesized DNA gene fragments from IDT (MaFAR-g1; SEQ ID NO:140, MaFAR-g2; SEQ ID NO:141, MaFAR-g3; SEQ ID NO:142 and MaFAR-g4; SEQ ID NO:143).

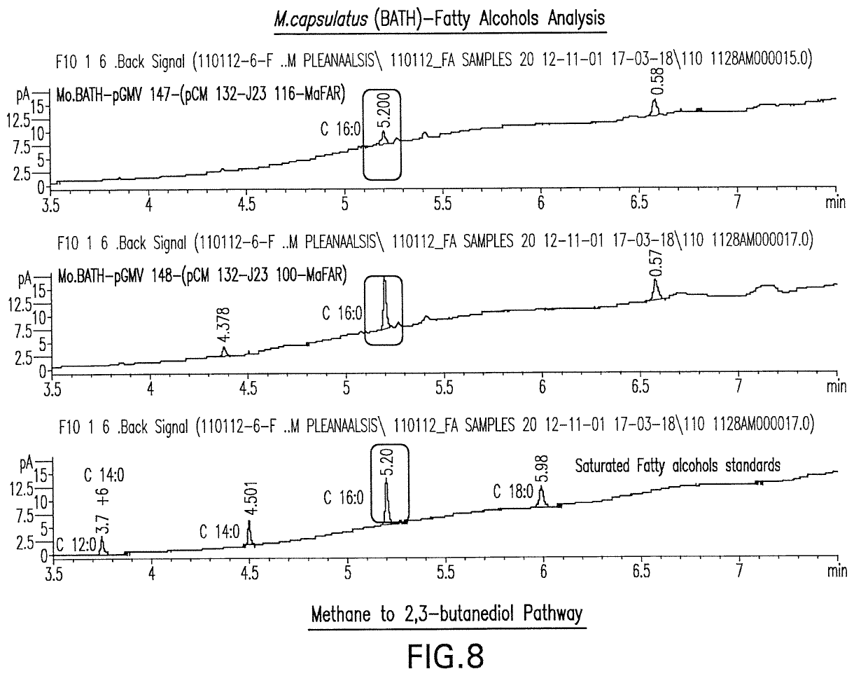

Gas chromatography results after various host strains were grown on methane in shake flasks, extracted, and analyzed as described above, are set forth in FIG. 8. The results indicate that the host strain containing plasmid pGMV148 produced C16:0 alcohol (a fatty alcohol) when grown on methane. The host strain containing plasmid pGMV147 produced only a trace amount of fatty alcohol.

TABLE 6
Insert Modules, Templates and Primers for Fatty Alcohol Production

| | | Modules | | | | |
|---|---|---|---|---|---|---|
| Plasmid | PCR rxn: | Vector backbone | Insert 1 | Insert 2 | Insert 3 | Insert 4 |
| pGMV147 | template | pMZT3 | MaFAR-g1 | MaFAR-g2 | MaFAR-g3 | MaFAR-g4 |
| | primer 1 | ESG00084 | — | — | — | — |
| | primer 2 | ESG00087 | — | — | — | — |
| pGMV148 | template | pMZT37 | MaFAR-g1 | MaFAR-g2 | MaFAR-g3 | MaFAR-g4 |
| | primer 1 | ESG00084 | — | — | — | — |
| | primer 2 | ESG00088 | — | — | — | — |

### Example 4

### Biosynthetic Production of Fatty Acid Methyl Esters from Methane

### Construction of Methanotroph Plasmids for Fatty Acid Ester (Wax Ester) Production

The plasmids generated in this example are based on the broad-host-range pCM132 (Accession no. AF327720, SEQ ID NO: 79) cloning vector described by Marx & Lidstrom (2001). In this embodiment, the use of the Clontech (catalogue no. 639647) InFusion HD Cloning System kit is one example of how to construct plasmids, but is not meant to limit or exclude other methods that are known in the art.

### Vector Backbones

Vector backbones contain the components of the plasmid that will remain constant. The broad-host range pCM132 vector was modified to produce vector backbones for the plasmids in this study. The pCM132 vector consisted of the following components: trrnB terminator, kanamycin resistance gene, trfA, IncP oriT, IncP oriV, colE1 ori, and lacZ. This parental vector was modified to replace lacZ with a vector insert that contains promoter sequence to produce plasmids and pMZT3 and pMZT37.

### Vector Inserts

Vector inserts contain DNA to be added to a vector backbone. The inserts were designed as exchangeable (modular) parts to the vector and in this embodiment consist of a wax ester synthase (WES) derived from Acinetobacter sp. ADP1 (SEQ ID NO:67), Psychrobacter arcticum 273-4 (SEQ ID NO:69) or Rhodococcus opcaus B4 (SEQ ID NO: 71, SEQ ID NO:73, SEQ ID NO:75 or SEQ ID NO:77). The WES genes were codon-optimized and synthesized by GenScript.

### Assembly of the Constructs

The modular parts (vector backbone and vector insert) were PCR amplified as listed in Table 7 with NEB Phusion master mix according to the manufacturer's instructions and in vitro assembled with the Clontech InFusion HD Cloning System kit according to the manufacturer's instructions to generate circular plasmid. The in vitro assembled plasmids (2 μl of the InFusion reaction) were transformed into chemically competent NEB Turbo E. coli cells, screened for by colony PCR, purified, and subsequently sequence verified.

Plasmid pMZT3 (SEQ ID NO:81) served as the template for the vector backbone with a constitutive promoter and consisted of the pCM132 cloning vector, E. coli J23115 promoter. The vector backbone was PCR amplified from the pMZT3 template with primers ESG00084 (SEQ ID NO:137)/ESG00087 (SEQ ID NO:98).

Plasmid pMZT37 (SEQ ID NO:139) served as the template for the vector backbone with a constitutive promoter and consisted of the pCM132 cloning vector, E. coli J23100 promoter. The vector backbone was PCR amplified from the pMZT3 template with primers ESG00084 (SEQ ID NO:137)/ESG00088 (SEQ ID NO:138).

The pGMV153 plasmid was designed to express Acinetobacter sp. ADP1 WES gene (wax-dgaT; SEQ ID NO:67) from the J23115 constitutive promoter. The modules of this plasmid included the PCR amplified pMZT3 vector backbone and the codon-optimized synthesized gene.

The pGMV154 plasmid was designed to express Psychrobacter arcticum 273-4 WES gene (Psyc_0223; SEQ ID NO:69) from the J23115 constitutive promoter. The modules of this plasmid included the PCR amplified pMZT3 vector backbone and the codon-optimized synthesized gene.

The pGMV155 plasmid was designed to express Rhodococcus opcaus B4 WES gene (ROP_02100; SEQ ID NO:71) from the J23115 constitutive promoter. The modules of this plasmid included the PCR amplified pMZT3 vector backbone and the codon-optimized synthesized gene.

The pGMV156 plasmid was designed to express Rhodococcus opcaus B4 WES gene (ROP_13050; SEQ ID NO:73) from the J23115 constitutive promoter. The modules of this plasmid included the PCR amplified pMZT3 vector backbone and the codon-optimized synthesized gene.

The pGMV157 plasmid was designed to express Rhodococcus opcaus B4 WS gene (ROP_26950; SEQ ID NO:77) from the J23115 constitutive promoter. The modules of this plasmid included the PCR amplified pMZT3 vector backbone and the codon-optimized synthesized gene.

The pGMV158 plasmid was designed to express Rhodococcus opcaus B4 WES gene (ROP_54550; SEQ ID NO:75) from the J23115 constitutive promoter. The modules of this plasmid included the PCR amplified pMZT3 vector backbone and the codon-optimized synthesized gene.

The pGMV159 plasmid was designed to express Acinetobacter sp. ADP1 WES gene (wax-dgaT; SEQ ID NO:67) from the J23100 constitutive promoter. The modules of this plasmid included the PCR amplified pMZT3 vector backbone and the codon-optimized synthesized gene.

The pGMV160 plasmid was designed to express Psychrobacter arcticum 273-4 WES gene (Psyc_0223; SEQ ID NO:69) from the J23100 constitutive promoter. The modules of this plasmid included the PCR amplified pMZT3 vector backbone and the codon-optimized synthesized gene.

The pGMV161 plasmid was designed to express Rhodococcus opcaus B4 WES gene (ROP_02100; SEQ ID NO:71) from the J23100 constitutive promoter. The modules of this plasmid included the PCR amplified pMZT3 vector backbone and the codon-optimized synthesized gene.

The pGMV162 plasmid was designed to express Rhodococcus opcaus B4 WES gene (ROP_13050; SEQ ID NO:73) from the J23100 constitutive promoter. The modules of this plasmid included the PCR amplified pMZT3 vector backbone and the codon-optimized synthesized gene.

The pGMV163 plasmid was designed to express Rhodococcus opcaus B4 WES gene (ROP_26950; SEQ ID NO:77) from the J23100 constitutive promoter. The modules of this plasmid included the PCR amplified pMZT3 vector backbone and the codon-optimized synthesized gene.

The pGMV164 plasmid was designed to express Rhodococcus opcaus B4 WES gene (ROP_54550; SEQ ID NO:75) from the J23100 constitutive promoter. The modules of this plasmid included the PCR amplified pMZT3 vector backbone and the codon-optimized synthesized gene.

Strains modified with these plasmids are grown on methane as described in the examples above, harvested, extracted, and analyzed for fatty acid ester production.

TABLE 7
Insert Modules, Templates and Primers for Fatty Acid Ester Production

| | | Modules | |
|---|---|---|---|
| | | Vector | |
| Plasmid | PCR rxn: | backbone | Insert |
| | template | pMZT3 | pUC57-AbWS (WS-dgaT) |
| pGMV153 | primer 1 | ESG00084 | GMV410 |
| | primer 2 | ESG00087 | GMV416 |
| pGMV154 | template | pMZT3 | pUC57-PaWS (Psyc_0223) |
| | primer 1 | ESG00084 | GMV411 |
| | primer 2 | ESG00087 | GMV417 |
| | template | pMZT3 | pUC57-RoWS (ROP_02100) |
| pGMV155 | primer 1 | ESG00084 | GMV412 |
| | primer 2 | ESG00087 | GMV418 |
| pGMV156 | template | pMZT3 | pUC57-RoWS (ROP_13050) |
| | primer 1 | ESG00084 | GMV413 |
| | primer 2 | ESG00087 | GMV419 |
| | template | pMZT3 | pUC57-RoWS (ROP_26950) |
| pGMV157 | primer 1 | ESG00084 | GMV414 |
| | primer 2 | ESG00087 | GMV420 |
| pGMV158 | template | pMZT3 | pUC57-RoWS (ROP_54550) |
| | primer 1 | ESG00084 | GMV415 |
| | primer 2 | ESG00087 | GMV421 |
| | template | pMZT37 | pUC57-AbWS (WS-dgaT) |
| pGMV159 | primer 1 | ESG00084 | GMV410 |
| | primer 2 | ESG00088 | GMV416 |
| pGMV160 | template | pMZT37 | pUC57-PaWS (Psyc_0223) |
| | primer 1 | ESG00084 | GMV411 |
| | primer 2 | ESG00088 | GMV417 |
| | template | pMZT37 | pUC57-RoWS (ROP_02100) |
| pGMV161 | primer 1 | ESG00084 | GMV412 |
| | primer 2 | ESG00088 | GMV418 |
| pGMV162 | template | pMZT37 | pUC57-RoWS (ROP_13050) |
| | primer 1 | ESG00084 | GMV413 |
| | primer 2 | ESG00088 | GMV419 |
| | template | pMZT37 | pUC57-RoWS (ROP_26950) |
| pGMV163 | primer 1 | ESG00084 | GMV414 |
| | primer 2 | ESG00088 | GMV420 |
| pGMV164 | template | pMZT37 | pUC57-RoWS (ROP_54550) |
| | primer 1 | ESG00084 | GMV415 |
| | primer 2 | ESG00088 | GMV421 |

### Example 5

### Biosynthetic Production of 2,3-Butanediol from Methane

The four-carbon (C4) diol 2,3-butanediol is an important intermediate for the chemical industry. At the commercial scale, it is mostly generated from petroleum. It serves as a precursor for the production of various commodity and specialty chemicals, such as the solvent methyl ethyl ketone (MEK), gamma-butyrolactone (GBL), and 1,3-butadiene. The potential production of these downstream commercial products amounts to about 32 million tons per year, with a value of about $43 billion (Köpke et al., 2011).

Biological production of 2,3-butanediol from methane requires engineering the native (or endogenous) metabolism of methanotrophs to take advantage of their endogenous production of (R)-acetoin (FIG. 9). (R)-acetoin is produced in methanotrophs from two molecules of pyruvate, which are ultimately derived from methane. By introducing and expressing the gene (SEQ ID NO: 156) encoding (2R,3R)-2,3-butanediol dehydrogenase (BDH1) from Saccharomyces cerevisiae in a suitable microbial expression host (such as M. capsulatus (Bath)), (R)-acetoin is converted into 2,3-butanediol.

### Construction of Methanotroph Plasmids for 2,3-Butanediol Production

As described, above, the plasmids generated in this study are based on the broad-host-range pCM132 (Accession no. AF327720, SEQ ID NO: 79) cloning vector described by Marx & Lidstrom (2001). In this embodiment, the use of the Clontech (catalogue 639647) InFusion HD Cloning System kit is one example of how to construct plasmids, but is not meant to limit or exclude other methods that are known in the art. Sequences for the ORF and PCR primers are presented below in Table 1.

### Vector Backbones

Vector backbones contain the components of the plasmid that will remain constant. The broad-host range pCM132 vector was modified to produce vector backbones for the plasmids in this example. The pCM132 vector consists of the following components: trrnB terminator, kanamycin resistance gene, trfA, IncP oriT, IncP oriV, colE1 ori, and lacZ. This parental vector has been modified to replace lacZ with a vector insert that contains promoter sequence to produce plasmid pMZT3, which was used for this example.

### Vector Inserts

Vector inserts contain DNA to be added to the vector backbone. The inserts were designed as exchangeable (modular) parts to the vector, and in this embodiment consists of the components listed in Table 1 and Table 8. In this example, the plasmids were designed to contain one insert: Saccharomyces cerevisiae (R,R)-butanediol dehydrogenase (Standard name: Bdh1p (EC 1.1.1.4); SEQ ID NO:156; Systematic gene name: YAL060W).

The BDH1 gene (SEQ ID NO:156) was codon optimized and synthesized by Integrated DNA Technologies (Coralville, IA).

### Assembly of the Constructs

The modular parts (vector backbone and vector insert) were PCR amplified as listed in Table 8 with NEB Phusion master mix according to the manufacturer's instructions and in vitro assembled with the Clontech InFusion HD Cloning System kit according to the manufacturer's instructions to generate circular plasmid. The in vitro assembled plasmids (2 μl of the InFusion reaction) were transformed into chemically competent NEB Turbo E. coli cells, screened for by colony PCR, purified, and subsequently sequence verified.

Plasmid pMZT3 served as the template for the vector backbone with a constitutive promoter and consisted of the pCM132 cloning vector, E. coli J23115 promoter. The vector backbone was PCR amplified from the pMZT3 template with primers ESG00084 (SEQ ID NO: 137)/ESG00087 (SEQ ID NO:98).

The pGMV111 plasmid was designed to express the S. cerevisiae BDH1 gene (SEQ ID NO: 156) from the J23115 constitutive promoter. The modules of this plasmid included the PCR amplified pMZT3 vector backbone and the ScBDH1 insert amplified from the shuttle vector pUC57-ScBDH1 template using primers GMV268 (SEQ ID NO: 158)/GMV271 (SEQ ID NO:159). The plasmid was conjugated from E. coli donor strain S17-1 into the M. capsulatus (Bath) recipient as described above Example 1. The transconjugant strain was purified by repeated rounds of antibiotic selection using kanamycin and naladixic acid to remove the parent cells, as described in Example 1 above.

Cells expressing the pGMV111 plasmid were cultivated in liquid NMS medium in sealed shake flasks in the presence of 20% methane at 45° C. as described above in Example 1, for about 72 hours with 200 rpm shaking. For UPLC analysis, proteins and other debris were separated from the 2,3-butanediol in the growth medium using 2% (wt/vol.) 5-sulfosalicylic acid and centrifugation as described in Köpke et al. (2011). Extracted samples can be analyzed using a BioRad (Hercules, CA) Fast Acid column on a Waters (Milford, MA) Acquity H-class UPLC equipped with a #2414 Refractive Index Detector. Other conditions are as follows: the mobile phase is 5 mM H₂SO4, the flow rate is 0.4 ml/min, the column is maintained at 40 C, and the product is detected at 410 nm.

Methods for the processing of biologically produced 1,3-propanediol and 2,3-butanediol are further described by Xiu & Zeng, 2008.

For GC analysis, the 2,3-butanediol can be extracted from the culture medium with ethyl acetate, as described in Xiao et al., (2012). The extracted sample is analyzed on an Agilent (Santa Clara, CA) 7890A GC equipped with a Leap Technologies CombiPAL autosampler and a flame ionization detector. Either an Agilent HP-INNOWax or HP-5 MS GC column can be used to separate the components according to the method of Xiao et al. (2012). Alternatively, the samples can be analyzed on a Waters Acquity H-Class UPLC equipped with a Waters 2414 Refractive Index detector using a method similar to that of Köpke et al. (2011). A BioRad (Hercules, CA) Fast Acid Column operated at 40° C. with a flow rate of 0.4 ml/minute and a 5 mM H₂SO4 mobile phase can be used to perform the separation. Samples for either GC or UPLC can be quantitated against a series of known concentrations of purified (D-(−)-, L-(+)-, and meso-)2,3-butanediol standards (Sigma, St. Louis, MO).

At the industrial fermentation scale, the 2,3-butanediol product can be extracted from the fermentation medium using one of the following methods: steam stripping, solvent extraction, aqueous two-phase extraction, reactive extraction, and pervaporation. These methods are described in Xiu & Zeng (2008).

TABLE 8

| | | Modules | |
|---|---|---|---|
| | | Vector | |
| Plasmid | PCR reaction: | backbone | Insert |
| pGMV111 | template | pMZT3 | pUC57- |
| | | | ScBDH1 |
| | primer 1 | ESG00084 | GMV268 |
| | primer 2 | ESG00087 | GMV271 |

Following is a list of citations for application.
U.S. Pat. No. 4,594,324
U.S. Pat. No. 4,982,023
U.S. Pat. No. 6,576,449
U.S. Pat. No. 6,660,507
U.S. Pat. No. 6,767,744
U.S. Pat. No. 6,818,424
U.S. Pat. No. 6,969,595
U.S. Pat. No. 7,026,464
U.S. Pat. No. 7,851,188
U.S. Pat. No. 7,910,342
U.S. Pat. No. 7,943,362
U.S. Pat. No. 7,977,084
U.S. Pat. No. 7,993,889
U.S. Pat. No. 8,017,375
U.S. Pat. No. 8,030,021
U.S. Pat. No. 8,101,808
U.S. Pat. No. 8,158,404
U.S. Pat. No. 8,232,089
U.S. Pat. No. 8,263,373
U.S. Pat. No. 8,268,599
U.S. Pat. No. 8,283,143
U.S. Pat. No. 8,349,587
U.S. Publication No. 2006/0057726
U.S. Publication No. 2007/0251141
U.S. Publication No. 2009/0263877
U.S. Publication No. 2010/0274033
U.S. Publication No. 2011/0301388
U.S. Publication No. 2012/0009640
European Patent No. EP 1419234
European Patent No. EP 1328639
European Patent No. EP 1416808
European Patent No. EP 1625204
European Patent No. EP 1694854
European Patent No. EP 2464722
European Patent No. EP 306466
European Patent No. EP 418187
PCT Publication No. WO 2001/60974
Alayon et al., “Catalytic Conversion of Methane to Methanol Using Cu-Zeolites”, Chimia, 66:668-674, 2012.
Ali, H., “Development of Genetic Tools in Methanotrophs and the Molecular Regulation of Methane Monooxygenase”, Ph.D. Thesis, Univ. of Warwick, Coventry, U. K., 2006.
Anthony, C. and Williams, P., “The structure and mechanism of methanol dehydrogenase”, Biochim. Biophys. Acta., 1647:18-23, 2003.
Arakawa et al., “Catalysis research of relevance to carbon management: progress, challenges, and opportunities”, Chem. Rev., 101:953-996, 2001.
Atsumi, S. et al., “Engineering the isobutanol biosynthetic pathway in Escherichia coli by comparison of three aldehyde reductase/alcohol dehydrogenase genes”, Appl. Microbiol. Biotechnology, 85:651-657, 2010.
Ausubel et al., “Current Protocols in Molecular Biology”, pub. by Greene Publishing Assoc. and Wiley-Interscience, 1987.
Ausubel, F. M., Brent, R., Kingston, R. E., Moore, D. D., Seidman, J. G., Smith, J. A., and Struhl, K., eds., Short Protocols in Molecular Biology, Fifth Edition. Wiley, 2002.
Avalos et al., “Compartmentalization of Metabolic Pathways in Yeast Mitochondria Improves the Production of Branched-Chain Alcohols”, Nature Biotechnology, Advanced Online Publication, Feb. 17, 2013, pages 1-7, doi: 10.1038/nbt.2509.
Bothe et al., “Heterotrophic bacteria growing in association with Methylococcus capsulatus (Bath) in a single cell protein production process”, Appl. Microbiol. Biotechnol., 59:33-39, 2002.
Bothe, H., Jensen, K. M., Mergel, A., Larsen, J., Jorgensen, C., Bothe, H. and Jorgensen, L., “Heterotrophic bacteria growing in association with Methylococcus capsulatus (Bath) in a single cell protein production process”, Appl. Microbiol. Biotechnol. 59:33-39, 2002.
Bowman, J., “The methanotrophs—the families Methylococcaceae and Methylocystaceae”, In: The Prokaryotes (Dworkin, M., ed.). Springer Verlag, New York, 2000 (http://link.springer-ny.com/link/service/books/10125).
Chistoserdova et al., “A genomic view of methane oxidation by aerobic bacteria and anaerobic archaea”, Genome Biol., 6:208, 2005.
Chistoserdova et al., “The Expanding World of Methylotrophic Metabolism”, Annu. Rev. Microbiol., 63:477-499, 2009.
Chistoserdova, L., “Modularity of methylotrophy, revisited”, Environ. Microbiol., 13:2603-2622, 2011.
Culpepper, M. A. and Rosenzweig, A. C., “Architecture and active site of particulate methane monooxygenase”, Crit. Rev. Biochem. Mol. Biol., 47:483-492, 2012.
Dunfield et al., “Methane oxidation by an extremely acidophilic bacterium of the phylum Verrucomicrobia”, Nature 450:879-882, 2007.
Dunfield et al., “Methylocella silvestris sp. nov., a novel methanotroph isolated from an acidic forest cambisol” Int. J. Syst. Evol. Microbiol., 53:1231-1239, 2003.
Eiteman, M. A. and Altman, E., “Overcoming acetate in Escherichia coli recombinant protein fermentations”, Trends Biotechnol. 24:53-536, 2006.
Engler et al., “Golden Gate Shuffling: A One-Pot DNA Shuffling Method Based on Type IIs Restriction Enzymes”, PLOS One 4: e5553, 2009.
Gellissen et al., “New yeast expression platforms based on methylotrophic Hansenula polymorpha and Pichia pastoris and on dimorphic Arxula adeninivorans and Yarrowia lipolytica—a comparison”, FEMS Yeast Res., 5:1079-1096, 2005.
Gibson et al., “Enzymatic assembly of DNA molecules up to several hundred kilobases”, Nature Methods 6:343-345, 2009.
Gou et al., “Functional expression of the particulate methane mono-oxygenase gene in recombinant Rhodococcus erythropolis”, FEMS Microbiol. Lett. 263:136-141, 2006.
Guillouet et al., “Expression of the Escherichia coli Catabolic Threonine Dehydratase in Corynebacterium glutamicum and Its Effect on Isoleucine Production”, Appl. Environ. Microbiol. 65:3100-3107, 1999.
Hägg, M. B., “Membranes in Chemical Processing—A Review of Applications and Novel Developments, Separation and Purification Methods”, Separ. Purif. Meth. 27:51-168, 1998.
Hakemian A. S. and Rosenzweig, A. C, “The biochemistry of methane oxidation”, Annu. Rev. Biochem. 76:223-241, 2007.
Hamilton, C. M., Aldea, M., Washburn, B. K., Babitzke, P. & Kushner, S. R., “New method for generating deletions and gene replacements in E. coli”, J. Bacteriol. 171:4617-4622, 1989. Hanson, R. S. and Hanson, T. E., “Methanotrophic bacteria”, Microbiol. Rev., 60:439-471, 1996.
Hickey, P. J. and Slater, C. S., “The selective recovery of alcohols from fermentation broths by pervaporation”, Separ. Purif. Meth. 19:93-115, 1990.
Jaeger, W. K. and Egelkraut, T. M., “Biofuel Economics in a Setting of Multiple Objectives and Unintended Consequences”, Renewable and Sustainable Energy Reviews, 15 (9): 4320, 2011. Jang, Y. S. et al., “Enhanced Butanol Production Obtained by Reinforcing the Direct Butanol-Forming Route in Clostridium acetobutylicum”, mBio, 3 (5): 1-9, 2012.
Jiang, H., Chen, Y., Jiang, P., Zhang, C., Smith, T. J., Xing, X.-H. and Murrell, J. C., “Methanotrophs: Multifunctional bacteria with promising applications in environmental bioengineering”, Biochem. Eng. J. 49:277-288, 2010.
Kidnay et al., “Fundamentals of Natural Gas Processing”, Second Edition, 2011 (Dekker Mechanical Engineering). CRC Press, Boca Raton.
Kim, S., Baek, S. H. and Hahn, J. S., “Cellulosic ethanol production using a yeast consortium displaying a minicellulosome and beta-glucosidase”, Microb Cell Fact., 12 (1): 14, 2013.
Kopke et. al., “2,3-Butanediol Production by Acetogenic Bacteria, an Alternative Route to Chemical Synthesis, Using Industrial Waste Gas”, Appl. Environ. Microbiol., 77:5467-5475, 2011.
Li, M. Z. and Elledge, S. J., “SLIC: a method for sequence- and ligation-independent cloning”, Methods Mol. Biol. 852:51-59, 2012.
Link, A. J., Phillips, D. and Church, G. M., “Methods for generating precise deletions and insertions in the genome wild-type Escherichia coli: applications to open reading frame characterization”, J. Bacteriol. 179:6228-6237, 1997.
Lipps, G., ed. “Plasmids: Current Research and Future Trends”, Caister Academic Press, Norfolk, England, U. K., 2008.
Liu, G., Hou D., Wei, W., Xiangli, F. and Jin, W., “Pervaporation separation of butanol-water mixtures using polydimethylsiloxane/ceramic composite membrane’, Chin. J. Chem. Eng. 19:40-44, 2011.
Lloyd et al., “Heterologous expression of soluble methane monooxygenase genes in methanotrophs containing only particulate methane monooxygenase”, Arch. Microbiol. 171:364-370, 1999.
Ma et al., “DNA synthesis, assembly and applications in synthetic biology”, Curr. Opin. Chem. Biol. 16:1-8, 2012.
Martin, H. and Murrell, J. C., “Methane monooxygenase mutants of Methylosinus trichosporium constructed by marker-exchange mutagenesis”, FEMS Microbiol. Lett. 127:243-248, 1995.
Marx, C. J. & Lidstrom, M. E., “Development of improved versatile broad-host-range vectors for use in methylotrophs and other Gram-negative bacteria”, Microbiology 147:2065-2075, 2001.
Merryman, C. and Gibson, D. G., “Methods and applications for assembling large DNA constructs”, Metabol. Eng. 14:196-204, 2012.
Murrel et al., “Molecular biology and regulation of methane monooxygenase”, Arch. Microbiol., 173:325-332, 2000.
Neel, J., “Pervaporation” In: Membrane Science and Technology, 1995 (Noble, R. D. & Stern, S. A., eds.) Elsevier Science, Amsterdam, The Netherlands.
Orita et al., “The Ribulose Monophosphate Pathway Substitutes for the Missing Pentose Phosphate Pathway in the Archaeon Thermococcus kodakaraensis”, J. Bacteriology, 188 (13): 4698-4704, 2006.
Peccoud, J., ed. “Gene Synthesis: Methods and Protocols” (Methods in Molecular Biology, Vol. 852). Humana Press, New York, 2012.
Phillips, R. B., Jameel, H, and Chang, H. M., “Integration of pulp and paper technology with bioethanol production”, Biotechnol Biofuels, 6 (1): 13, 2013.
Posfai, G., Kolisnychenko, V., Bereczki, Z. and Blattner, F. R., “Markerless gene replacement in Escherichia coli stimulated by a double-strand break in the chromosome”, Nucleic Acids Res. 27:4409-4415, 1999.
Rosenzweig, A. C. and Ragsdale, S. W. “Methods in Methane Metabolism”, Part B: Methanotrophy. Methods Enzymol., 495:1-309, 2011 (b).
Rosenzweig, A. C. and Ragsdale, S. W., “Methods in Methane Metabolism”, Part A. Methods Enzymol., 494:1-373, 2011 (a).
Rudolf, A., Karhumaa, K. and Hahn-Hagerdal, B., “Ethanol Production from Traditional and Emerging Raw Materials”, Yeast Biotechnology: Diversity and Applications, Chapter 23, pages 489-513, 2009.
Saka, S. and Kusdiana, D., “Biodiesel fuel from rapeseed oil as prepared in supercritical methanol”, Fuel, 80:225, 2001.
Saleh, J., “A Membrane Separation Process for Biodiesel Purification’, Ph.D. Thesis, 2011, University of Ottawa, Canada.
Sambrook et al., Molecular Cloning: A Laboratory Manual, Cold Spring Harbor Laboratory Press, 1989.
Sambrook, J., Fritsch, E. F. and Maniatis, T., “Molecular Cloning: A Laboratory Manual”; Cold Spring Harbor Laboratory Press, Cold Spring Harbor, 1989.
Schrader et al., “Methanol-based industrial biotechnology: current status and future perspectives of methylotrophic bacteria”, Trends Biotechnol., 27:107-115, 2009.
Semrau et al., “Facultative methanotrophy: false leads, true results, and suggestions for future research”, FEMS Microbiol. Lett., 323:1-12, 2011.
Shen, C. R. and Liao, J. C., “Metabolic engineering of Escherichia coli for 1-butanol and 1-propanol production via the keto-acid pathways”, Metab. Eng. 10:312-320, 2008.
Silhavy, et al., “Experiments with Gene Fusions”, Cold Spring Harbor Laboratory, Cold Spring Harbor, N.Y. 1984,
Simon R. “High frequency mobilization of Gram-negative bacterial replicons by the in vitro constructed Tn5-mob transposons”, Mol. Gen. Genet. 196:413-420, 1984.
Simon, R., Priefer, U. and Puhler, A. “A Broad Host Range Mobilization System for In Vivo Genetic Engineering: Transposon Mutagenesis in Gram Negative Bacteria”, Nature Biotechnology 1:784-791, 1983.
Stafford et al., “rpoN, mmoR and mmoG, genes involved in regulating the expression of soluble methane monooxygenase in Methylosinus trichosporium OB3b”, Microbiol. 149:1771-1784, 2003.
Stanley, S. H. and Dalton, H., “Role of ribulose-1,5-biphosphate carboxylase/oxygenase in Methylococcus capsulatus”, J. Gen. Microbiol., 128:2927-2935, 1982.
Sun, W., Wang, S. and Curtis III, R., “Highly efficient method for introducing successive multiple scarless gene deletions and markerless gene insertions into the Yersinia pestis chromosome”, Appl. Environ. Microbiol. 74:4241-4245, 2008.
Theisen et al., “Regulation of methane oxidation in the facultative methanotroph Methylocella silvestris BL2”, Mol. Microbiol. 58:682-692, 2005.
Tinberg, C. E. and Lippard, S. J., “Dioxygen activation in soluble methane monooxygenase”, Acc. Chem. Res., 44:280-288, 2007.
Trotsenko, Y. A. and Murrell, J. C., “Metabolic Aspects of Aerobic Obligate Methanotrophy”, Adv. Appl. Microbiol., 63:183-229, 2008.
U.S. Department of the Interior, U.S. Geological Survey World Petroleum Assessment, 2000, U.S. Geological Survey Digital Data Series-DDS-60 (URL: http://pubs. usgs.gov/dds/dds-060/)
van Laere, V., van Batenburg, O. & Huizing, H. J., “InnoFisk1: Feasibility study into a new concept for sustainable aquaculture on board of a ship”, Innovation Network Rural Areas and Agricultural Systems, 2005.
Veazey, M. W., “GTL Tech Converts Methane to Ethylene without Fischer Tropsch”, Rigzone. Apr. 10, 2012 (http://www.rigzone.com/news/article.asp?a_id=116784)
Wang et al., “Available methods for assembling expression cassettes for synthetic biology”, Appl. Microbiol. Biotechnol. 93:1853-1863, 2012.
Ward, N., Larsen, Ø., Sakwa, J., et al., PLOS Biol. 2: e303, 2004.
Welander, P. V. and Summons, R. E., “Discovery, taxonomic distribution, and phenotypic characterization of a gene required for 3-methylhopanoid production”, Proc. Natl. Acad. Sci., 109:12905-12910, 2012.
Whittenbury, R., Phillips, K. C. and Wilkinson, J. F., “Enrichment, isolation and some properties of methane-utilizing bacteria”, J. Gen. Microbiol. 61:205-218, 1970.
Wolfe, A. J., “The acetate switch”, Microbiol. Mol. Biol. Rev. 69:12-50, 2005.
Wright, C. K. and Wimberly, M. C., “Recent Land Use Change in the Western Corn Belt Threatens Grasslands and Wetlands”, Proceedings of the National Academy of Sciences—Early Edition, February 19, pg. 1-6, 2013; DOI: 10.1073/pnas.1215404110.
Xiao et al., “Thermophilic fermentation of acetoin and 2,3-butanediol by a novel Geobacillus strain”, Biotechnol. for Biofuels 5:88, 2012.
Xingye et al., “In vitro Reconstitution and Steady-State analysis of the Fatty Acid Synthase from Escherichia coli”, Proceedings of the National Academy of Sciences, 10 (8): 18643-18648, 2011.
Xiu, Z.-L. and Zeng, A.-P., “Present state and perspective of downstream processing of biologically produced 1,3-propanediol and 2,3-butanediol”, Appl. Microbiol. Biotechnol. 78:917-926, 2008.
Yangkai et al., “De novo Biosynthesis of Biodiesel by Escherichia coli in Optimized Fed-Batch Cultivation”, PLOS ONE, 6 (5): 1-7, 2011.
Yehezkel et al., “Computer-aided high-throughput cloning of bacteria in liquid medium”, Biotechniques 50:124-127, 2011.
Yomantas, Y. A., Tokmakova, I. L., Gorshkova, N. V., Abalakina, E. G., Kazakova, S. M., Gak, E. R. and Mashko, S. V., “Aromatic amino acid auxotrophs constructed by recombinant marker exchange in Methylophilus methylotrophus AS1 cells expressing the aroP-encoded transporter of Escherichia coli”, Appl. Environ. Microbiol. 76:75-83, 2010.
Yu, B. J., Kang, K. H., Lee, J. H., Sung, B. H., Kim, M. S. and Kim, S. C., “Rapid and efficient construction of markerless deletions in the Escherichia coli genome”, Nucleic Acids Res. 36: e84, 2008.
Yurimoto, H., Katoh, N. and Sakai, Y., “Assimilation, dissimilation, and detoxification of formaldehyde, a central metabolic intermediate of methylotrophic metabolism”, Chem Rec., 5:367-375, 2005.
Yurimoto, H., Katoh, N. and Sakai, Y., “Genomic organization and biochemistry of the ribulose monophosphate pathway and its application in biotechnology”, Appl Microbiol Biotechnol., 84:407-416, 2009.

| Sequence Listing | | | |
|---|---|---|---|
| 1 | Sequence Listing Information | | |
| 1-1 | File Name | 35016-001C3.xml | |
| 1-2 | DTD Version | V1_3 | |
| 1-3 | Software Name | WIPO Sequence | |
| 1-4 | Software Version | 2.3.0 | |
| 1-5 | Production Date | 2024 May 24 | |
| 1-6 | Original free text language | | |
| | code | | |
| 1-7 | Non English free text | | |
| | language code | | |
| 2 | General Information | | |
| 2-1 | Current application: | US | |
| | IP Office | | |
| 2-2 | Current application: | | |
| | Application number | | |
| 2-3 | Current application: | | |
| | Filing date | | |
| 2-4 | Current application: | 35016-001C3 | |
| | Applicant file reference | | |
| 2-5 | Earliest priority | US | |
| | application: IP Office | | |
| 2-6 | Earliest priority | 61/782,830 | |
| | application: | | |
| | Application number | | |
| 2-7 | Earliest priority | 2013 Mar. 14 | |
| | application: | | |
| | Filing date | | |
| 2-8en | Applicant name | Coleman, William J | |
| 2-8 | Applicant name: Name Latin | | |
| 2-9 | Inventor name | | |
| 2-9 | Inventor name: Name Latin | | |
| 2-10en | Invention title | Biological Production of Multi- | |
| | | Carbon Compounds From Methane | |
| 2-11 | Sequence Total Quantity | 175 | |
| 3-1 | Sequences | | |
| 3-1-1 | Sequence Number [ID] | 1 | |
| 3-1-2 | Molecule Type | DNA | |
| 3-1-3 | Length | 1662 | |
| | | source 1 . . . 1662 | |
| 3-1-4-1 | Features Location/ | mol_type = other DNA | |
| | Qualifiers | organism = Methylococcus capsulatus | |
| | NonEnglishQualifier Value | | |
| 3-1-5 | Residues | | |
| | atgcgtgaaa cgatacctcc ccgcaccggc gccgacctgc tggtcgactc cctccaggcg | | 60 |
| | ctgggcgtcg aatacgtctt cggcgtgccc ggcggcgcga tactcccgat cctgaacgtg | | 120 |
| | ctggccgacc gcggcccgcg cttcatcgtt tgccgggacg aaaccggcgc cgccttcatg | | 180 |
| | gcccagtcct ggggccggat caccggccgg cccggcgtgg tgctcaccac ctccggcccc | | 240 |
| | ggcctcatca acgccgtctg tggcgtcgct accgccacag aggaccgcga cccgctggtc | | 300 |
| | gtcatcaccg gccaggtgcc gcgggccgtg caattcaagc agagccacat gaacctggat | | 360 |
| | tcggtcggcc tgttcgcgcc gatcaccaaa tggagcgtcg aggtcgagga accgaatact | | 420 |
| | gtatcggaaa tcctggtcaa cgccttccgc accgcgcaga cgccgtgcgc cggagccgtc | | 480 |
| | cacgtctcgg taccgaacga catgctcacc gcgccggtca ccgcgcaggc cctggcgccg | | 540 |
| | gccgaacccg ccgtctgggg aacggccccg gccgccgtcg tcgaacgcgc ggcgtccctg | | 600 |
| | ctgaacgatg ccaaagcccc ggccatcctg ctcggattgc gggccagcac acctggagcg | | 660 |
| | gcggcggcgg tccggcgttt cctggagcgg catccgctgc cggtggcgat gaccttcgaa | | 720 |
| | gccgccggca ccctgtcccg cgatctggtc gatcagttcg tcggccgggt cggctacgtg | | 780 |
| | ctcaaccagc cgggcgacga ggtgctgcgc caagccgatc tggtactcac gatcggctac | | 840 |
| | gacccgatcg aatacgaacc ttccgcctgg atctcaccgc agtcgcaggc gatccacctg | | 900 |
| | gatgccctgc ccgccgccgt cgaccgggcc taccaccctg ccgccgaact ggtcggcgac | | 960 |
| | atcgccgcca acctggccgc gctcggcagc ctgctccgaa tcgaggatcg agccggacgc | | 1020 |
| | cccgccgtcg ccgcggcgcg gcggcgtctg ctggaggagc aagcccgcgg cgcagcactg | | 1080 |
| | accggtatgc cgatccaccc cttgcgcttc attcacgacc ttcgggccac gctggacgac | | 1140 |
| | gaggcgacgg tgacctgcga cgtcggcgcc cacgagatct ggatggcccg ctacttcttc | | 1200 |
| | tgctacgccc cgcgtcacct gctgttcagc atgggccacc agaccatggg cgtcgccctg | | 1260 |
| | ccctgggcca tcggcgcggc cctggcccgg cccggcaaga aagtggtttc ggtatccggc | | 1320 |
| | gacggctcct tcctcatgac ctgcatggaa ctggaaaccg cggtgcgcct caaactgccg | | 1380 |
| | atcgtgcaca tcgtctggaa agacggcggc tacaacctga tccacagcct gcagatgcgc | | 1440 |
| | gactatgggc gcagcttcgg cgccgagttc ggccccaccg acttcgtcaa actggcggag | | 1500 |
| | gccttcggcg cgatcgggta ccggatcgag tccgcggacg ggatcgtccc tgtgctgaac | | 1560 |
| | cgggcgctcg cggccgacgc gccggtgctg atcgaagtgc ccatcgacta cagcgacaac | | 1620 |
| | gtccacctgg tcgaggcgat cgacgcctcg gcgcagcact ga | | 1662 |
| 3-2 | Sequences | | |
| 3-2-1 | Sequence Number [ID] | 2 | |
| 3-2-2 | Molecule Type | AA | |
| 3-2-3 | Length | 553 | |
| | | source 1 . . . 553 | |
| 3-2-4-1 | Features Location/ | mol_type = protein | |
| | Qualifiers | organism = Methylococcus capsulatus | |
| | NonEnglishQualifer Value | | |
| 3-2-5 | Residues | | |
| | MRETIPPRTG ADLLVDSLQA LGVEYVFGVP GGAILPILNV LADRGPRFIV CRDETGAAFM | | 60 |
| | AQSWGRITGR PGVVLTTSGP GLINAVCGVA TATEDRDPLV VITGQVPRAV QFKQSHMNLD | | 120 |
| | SVGLFAPITK WSVEVEEPNT VSEILVNAFR TAQTPCAGAV HVSVPNDMLT APVTAQALAP | | 180 |
| | AEPAVWGTAP AAVVERAASL LNDAKAPAIL LGLRASTPGA AAAVRRFLER HPLPVAMTFE | | 240 |
| | AAGTLSRDLV DQFVGRVGYV LNQPGDEVLR QADLVLTIGY DPIEYEPSAW ISPQSQAIHL | | 300 |
| | DALPAAVDRA YHPAAELVGD IAANLAALGS LLRIEDRAGR PAVAAARRRL LEEQARGAAL | | 360 |
| | TGMPIHPLRF IHDLRATLDD EATVTCDVGA HEIWMARYFF CYAPRHLLFS MGHQTMGVAL | | 420 |
| | PWAIGAALAR PGKKVVSVSG DGSFLMTCME LETAVRLKLP IVHIVWKDGG YNLIHSLQMR | | 480 |
| | DYGRSFGAEF GPTDFVKLAE AFGAIGYRIE SADGIVPVLN RALAADAPVL IEVPIDYSDN | | 540 |
| | VHLVEAIDAS AQH | | 553 |
| 3-3 | Sequences | | |
| 3-3-1 | Sequence Number [ID] | 3 | |
| 3-3-2 | Molecule Type | DNA | |
| 3-3-3 | Length | 1017 | |
| | | source 1 . . . 1017 | |
| 3-3-4-1 | Features Location/ | mol_type = other DNA | |
| | Qualifiers | organism = Methylococcus capsulatus | |
| | NonEnglishQualifer Value | | |
| 3-3-5 | Residues | | |
| | atgcagattt actacgacaa agacgccgac ctttccatca tccagggaaa gaaggttgcc | | 60 |
| | atcatcggct acggctcgca gggccacgcc cacgccaaca acctcaagga ttccggagtg | | 120 |
| | caggtcgtgg tggggctgcg tccgggttcg gcttccgcca agaaggccga gaacgccggc | | 180 |
| | ctcgcggtcg cctcggtcga ggatgcggtc aaacaggcgg acgtcatcat gatcctggcg | | 240 |
| | ccggacgagc atcaggcccg cctctacaat gaacagatcg cgccgaacat caagcagggc | | 300 |
| | gccgccctcg ccttcgccca cggcttcaac atccacttcg agcagatcac cccgcgcgcc | | 360 |
| | gacctcgacg tgatcatgat cgcgcccaag ggtcccggcc atctggtacg ttccacctac | | 420 |
| | acccagggcg gcggcgtgcc ctcgctgatc gccgtgtacc agaatgccag cgggcgcgcc | | 480 |
| | aaggaactcg cgctgtccta tgcttcggcc aatggcggcg gtcgggctgg tatcatcgag | | 540 |
| | accaccttcc gcgaagagac cgaaaccgat ctgttcggcg aacaggccgt cctgtgtggc | | 600 |
| | ggcgccaccg cactggtgca ggcgggtttc gagacgctgg tcgaagccgg ttatgcgccc | | 660 |
| | gagatggcct atttcgagtg tctgcacgaa ctcaagctga tcgtcgacct gatgtacgaa | | 720 |
| | ggcggcatcg ccaacatgcg ttattcgatc tccaatacgg cagagtacgg cgacctgacc | | 780 |
| | cgtggtccgc gcatcgtcac cgagcagacc aagcaggaaa tgaagaaaat cctgcgcgag | | 840 |
| | atccagaccg gcgaattcgc ccgtgagttc attttggaaa accaggccgg agccgccacc | | 900 |
| | ctgaaagcga aacgccgtct cggccgagag catctcatcg agagcgtggg cgccaggctg | | 960 |
| | cgcgacatga tgccgtggat caaggccaac cgcattgtgg acacgagcaa gaactga | | 1017 |
| 3-4 | Sequences | | |
| 3-4-1 | Sequence Number [ID] | 4 | |
| 3-4-2 | Molecule Type | AA | |
| 3-4-3 | Length | 338 | |
| | | source 1 . . . 338 | |
| 3-4-4-1 | Features Location/ | mol_type = protein | |
| | Qualifiers | organism = Methylococcus capsulatus | |
| | NonEnglishQualifier Value | | |
| 3-4-5 | Residues | | |
| | MQIYYDKDAD LSIIQGKKVA IIGYGSQGHA HANNLKDSGV QVVVGLRPGS ASAKKAENAG | | 60 |
| | LAVASVEDAV KQADVIMILA PDEHQARLYN EQIAPNIKQG AALAFAHGFN IHFEQITPRA | | 120 |
| | DLDVIMIAPK GPGHLVRSTY TQGGGVPSLI AVYQNASGRA KELALSYASA NGGGRAGIIE | | 180 |
| | TTFREETETD LFGEQAVLCG GATALVQAGF ETLVEAGYAP EMAYFECLHE LKLIVDLMYE | | 240 |
| | GGIANMRYSI SNTAEYGDLT RGPRIVTEQT KQEMKKILRE IQTGEFAREF ILENQAGAAT | | 300 |
| | LKAKRRLGRE HLIESVGARL RDMMPWIKAN RIVDTSKN | | 338 |
| 3-5 | Sequences | | |
| 3-5-1 | Sequence Number [ID] | 5 | |
| 3-5-2 | Molecule Type | DNA | |
| 3-5-3 | Length | 1689 | |
| | | source 1 . . . 1689 | |
| 3-5-4-1 | Features Location/ | mol_type = other DNA | |
| | Qualifiers | organism = Methylococcus capsulatus | |
| | NonEnglishQualifier Value | | |
| 3-5-5 | Residues | | |
| | atgaccgaca agcacccccg tccccattcg tcccaggtcg tcgacggcat ggagcgcgcc | | 60 |
| | ccgagccgcg cgatgctgca cgccgtcggc ttcgccgatg ccgacttcgc caaaccgcag | | 120 |
| | atcggcatcg cttccacctg ggcgatggtg acgccgtgca acatgcacat caacaagctc | | 180 |
| | gccgaggacg cagcacgcgg cgtcgacggc ggcggcggca aggcagtgat cttcaacacc | | 240 |
| | atcaccattt ccgacggcat ctcgatgggc accgaaggaa tgaaatactc cctcgtgtcg | | 300 |
| | cgggaagtca tcgccgactc gatcgaaacc gtggtggcct gtcagggtta tgacggcgtg | | 360 |
| | gtcgccatcg gcggctgcga caagaacatg cccggctgcc tgatcgccct cgcccgcctc | | 420 |
| | aaccgtccgg cggtgttcgt ctatggcggc accatcctgc cgggctgcca cgacggcaag | | 480 |
| | aagctggacg tggtgtcggt gttcgaagcg gtcggcgccc gcgccaacca ccgcatcgac | | 540 |
| | gatgccgaac tgcacgccat cgaatccaat gccatccccg gtccgggctc ctgcggtggc | | 600 |
| | atgtataccg ccaacaccat ggcctccgcc atcgaggcat tagggatgag cctgccgggc | | 660 |
| | agttcggccc aggtggccat ttcccgcgcc aaggaactgg attgcgagcg ggccggcgcg | | 720 |
| | caggtcctca agctcctgga cctggggctc aaaccccgcg acatcatgac caagaaggcg | | 780 |
| | ttcgagaacg ccatcacggt ggtgatcgcc ctgggcggct ccaccaacgc cgtgctgcac | | 840 |
| | ctcctggcca tggccaacgc ctgcggcgtc gacctgaagc tcgacgattt cacccgcatc | | 900 |
| | gggcgcaaag tgccgatgct ggcggatctg aaacccagcg gcagatactc catggccgaa | | 960 |
| | ctggtggaaa tcggcggcat ccagccgctg atgaagacct tgctggacgc gggactcctg | | 1020 |
| | cacggcgact gcatgaccgt aaccggcaag accctggaag aaaacctggc cgacgcgccc | | 1080 |
| | gactacccgg ccggacaaga catgatccgg tcgctggaca accccatcaa aaaggacagc | | 1140 |
| | catctggtga tcctcaaggg caacctggcg ccggaaggcg cggtcgccaa gatcaccggc | | 1200 |
| | aaggaaggac tgagcttcac cggcaccgcc cgcgtattcg actgcgagga agcggcgctc | | 1260 |
| | acggccatcc tcgacggcac gatcgtgaaa ggcgacgtca tcgtcatccg ctatgaaggc | | 1320 |
| | cccaagggcg gccccggcat gcgcgagatg ctctcgccga cctcggcggt catgggcaag | | 1380 |
| | ggattgggca aggaggtcgc cctcatcacc gacggccgct tttccggcgg cacccacggc | | 1440 |
| | ttcgtggtcg gccacatcac gccggaagcc tacaccggcg gccccctggc gatcgtccgg | | 1500 |
| | gacggcgata ccatcaccat cgacgccgag acccgcgaat tgagcctgca cgtcaccgac | | 1560 |
| | gatgaaatcg gccggcgcct ggcgcagtgg actcaaccgg cgccgcgcta caccaagggc | | 1620 |
| | gtgctggcca aatacgccag gttggtgagc ccggcctcgg aaggcgccgt caccgacgac | | 1680 |
| | ggcctctga | | 1689 |
| 3-6 | Sequences | | |
| 3-6-1 | Sequence Number [ID] | 6 | |
| 3-6-2 | Molecule Type | AA | |
| 3-6-3 | Length | 562 | |
| | | source 1 . . . 562 | |
| 3-6-4-1 | Features Location/ | mol_type = protein | |
| | Qualifiers | organism = Methylococcus capsulatus | |
| | NonEnglishQualifier Value | | |
| 3-6-5 | Residues | | |
| | MTDKHPRPHS SQVVDGMERA PSRAMLHAVG FADADFAKPQ IGIASTWAMV TPCNMHINKL | | 60 |
| | AEDAARGVDG GGGKAVIFNT ITISDGISMG TEGMKYSLVS REVIADSIET VVACQGYDGV | | 120 |
| | VAIGGCDKNM PGCLIALARL NRPAVFVYGG TILPGCHDGK KLDVVSVFEA VGARANHRID | | 180 |
| | DAELHAIESN AIPGPGSCGG MYTANTMASA IEALGMSLPG SSAQVAISRA KELDCERAGA | | 240 |
| | QVLKLLDLGL KPRDIMTKKA FENAITVVIA LGGSTNAVLH LLAMANACGV DLKLDDFTRI | | 300 |
| | GRKVPMLADL KPSGRYSMAE LVEIGGIQPL MKTLLDAGLL HGDCMTVTGK TLEENLADAP | | 360 |
| | DYPAGQDMIR SLDNPIKKDS HLVILKGNLA PEGAVAKITG KEGLSFTGTA RVFDCEEAAL | | 420 |
| | TAILDGTIVK GDVIVIRYEG PKGGPGMREM LSPTSAVMGK GLGKEVALIT DGRFSGGTHG | | 480 |
| | FVVGHITPEA YTGGPLAIVR DGDTITIDAE TRELSLHVTD DEIGRRLAQW TQPAPRYTKG | | 540 |
| | VLAKYARLVS PASEGAVTDD GL | | 562 |
| 3-7 | Sequences | | |
| 3-7-1 | Sequence Number [ID] | 7 | |
| 3-7-2 | Molecule Type | DNA | |
| 3-7-3 | Length | 1650 | |
| | | source 1 . . . 1650 | |
| 3-7-4-1 | Features Location/ | mol_type = other DNA | |
| | Qualifiers | organism = Methylococcus capsulatus | |
| | NonEnglishQualifier Value | | |
| 3-7-5 | Residues | | |
| | atgggcacgg ttgagcctgg cgctatcgga caacatctgc tcgcctgcct ttaccaggcg | | 60 |
| | ggcgtcgggc acatcttcgg cgttcccggc gattacgtgc tgggcttcta tgatctgatg | | 120 |
| | gccaaaggtc ccgtccggca tatcgggacc acgcgggagg acaccgccgc cttcgccgcc | | 180 |
| | gacggctatg cccgctgccg gggcatgggc gcgctggcgg tgacttacgg ggtcggtgcg | | 240 |
| | ctcaacaccg tcaacgccgt cgccggcgcc tatgcggaat cctcgccggt ggtggtcatc | | 300 |
| | agcggtgcgc cgggggtgcg cgagcaaagg gaagacccgt tgatccacca ccgcttcggg | | 360 |
| | ccgttccggt tccagcgcga gatattcgaa cggatcacct gcgccgccgt ggtgctggac | | 420 |
| | gatccggtga tcgccttccg gcaggtggag cgtgcgctcg cggccgcccg tcagcactgc | | 480 |
| | aagccggtgt acatcgagat tcccgccgac cgggtgatgg cgccgggata tccgattcca | | 540 |
| | caggaaaccc cggaaacgcc ttccagcgac gattcggccc tggcggaggc ggtcgccgag | | 600 |
| | gccgcggagc tcctgggccg tgcggtgtcg ccggtgatcc ttgcaggcgt cgagttgcac | | 660 |
| | cggcgagggc tccaggacgc cctcgtcggc ctcgtcgagc aggcgcgcct gccggtggcg | | 720 |
| | gcgaccttga ccggcaagtc ggtgttcgcc gagcgccatc ccgcctatct gggggtgtac | | 780 |
| | gagggtgcga tgagcacgga aaacgcgcgc tacatggtcg agcagtccga cctcctgctg | | 840 |
| | atgctcgggg tcacgctgaa cgatgtcgac acgggcatct acacggcgcg tctcgatccg | | 900 |
| | cagcgcatcg tccgcgcagc ccagaacgag gtcgtgattc gccatcaccg ctatccccgc | | 960 |
| | gtcctgctcg cggacttcgt cacggccctg gcgcggtccg tcaaggcccg gggcgaggcg | | 1020 |
| | tttccgatgc cggcggggcc ggaaccgtgg gactttcccg cgccggaccg gccgatgacg | | 1080 |
| | atcgcccggc tggtggagcg gctcgaccgc gcgctgacct ccgacatgat cgtagtgtgc | | 1140 |
| | gacgtcggcg actgcctgtt cgcagccacc gacctgcgcg tgcacgagcg cagcgaattc | | 1200 |
| | ctggcgtccg ccttctatac ctcgatgggg ttcgcggtgc ccgccgccct cggggcccag | | 1260 |
| | atcgcccgtc cggaccaccg ggcgctgatc ctggtcggcg acggtgcctt ccagatgacc | | 1320 |
| | ggaacggagc tgtcgaccca tgcccgtctc ggcctggcgc ccatcgtggt ggtgctcgac | | 1380 |
| | aatcgcggtt acagcaccga gcgcttcatc ctcgacggag ccttcaacga catcgccgac | | 1440 |
| | tggcgcttcc accggctggg cgaggtgttc ggccccctac agggctacga cgcgcccgac | | 1500 |
| | gaagcggcgt tcgaaaacgc gctcagcgaa gcgctggtca accgaaacat gccgagcctc | | 1560 |
| | atcaacgtcc gtctttcccc cggcgatgcc tcgatagcca tgaagcgtct cgccgggcat | | 1620 |
| | ctgcagtgcc gggtcaaggg cgagggctga | | 1650 |
| 3-8 | Sequences | | |
| 3-8-1 | Sequence Number [ID] | 8 | |
| 3-8-2 | Molecule Type | AA | |
| 3-8-3 | Length | 549 | |
| 3-8-4-1 | | source 1 . . . 549 | |
| | Features Location/ | mol_type = protein | |
| | Qualifiers | organism = Methylococcus capsulatus | |
| | NonEnglishQualifier Value | | |
| 3-8-5 | Residues | | |
| | MGTVEPGAIG QHLLACLYQA GVGHIFGVPG DYVLGFYDLM AKGPVRHIGT TREDTAAFAA | | 60 |
| | DGYARCRGMG ALAVTYGVGA LNTVNAVAGA YAESSPVVVI SGAPGVREQR EDPLIHHRFG | | 120 |
| | PFRFQREIFE RITCAAVVLD DPVIAFRQVE RALAAARQHC KPVYIEIPAD RVMAPGYPIP | | 180 |
| | QETPETPSSD DSALAEAVAE AAELLGRAVS PVILAGVELH RRGLQDALVG LVEQARLPVA | | 240 |
| | ATLTGKSVFA ERHPAYLGVY EGAMSTENAR YMVEQSDLLL MLGVTLNDVD TGIYTARLDP | | 300 |
| | QRIVRAAQNE VVIRHHRYPR VLLADFVTAL ARSVKARGEA FPMPAGPEPW DFPAPDRPMT | | 360 |
| | IARLVERLDR ALTSDMIVVC DVGDCLFAAT DLRVHERSEF LASAFYTSMG FAVPAALGAQ | | 420 |
| | IARPDHRALI LVGDGAFQMT GTELSTHARL GLAPIVVVLD NRGYSTERFI LDGAFNDIAD | | 480 |
| | WRFHRLGEVF GPLQGYDAPD EAAFENALSE ALVNRNMPSL INVRLSPGDA SIAMKRLAGH | | 540 |
| | LQCRVKGEG | | 549 |
| 3-9 | Sequences | | |
| 3-9-1 | Sequence Number [ID] | 9 | |
| 3-9-2 | Molecule Type | DNA | |
| 3-9-3 | Length | 1083 | |
| 3-9-4-1 | | source 1 . . . 1083 | |
| | Features Location/ | mol_type = other DNA | |
| | Qualifiers | organism = Saccharomyces cerevisiae | |
| | NonEnglishQualifier Value | | |
| 3-9-5 | Residues | | |
| | atgtcttatc ctgagaaatt tgaaggtatc gctattcaat cacacgaaga ttggaaaaac | | 60 |
| | ccaaagaaga caaagtatga cccaaaacca ttttacgatc atgacattga cattaagatc | | 120 |
| | gaagcatgtg gtgtctgcgg tagtgatatt cattgtgcag ctggtcattg gggcaatatg | | 180 |
| | aagatgccgc tagtcgttgg tcatgaaatc gttggtaaag ttgtcaagct agggcccaag | | 240 |
| | tcaaacagtg ggttgaaagt cggtcaacgt gttggtgtag gtgctcaagt cttttcatgc | | 300 |
| | ttggaatgtg accgttgtaa gaatgataat gaaccatact gcaccaagtt tgttaccaca | | 360 |
| | tacagtcagc cttatgaaga cggctatgtg tcgcagggtg gctatgcaaa ctacgtcaga | | 420 |
| | gttcatgaac attttgtggt gcctatccca gagaatattc catcacattt ggctgctcca | | 480 |
| | ctattatgtg gtggtttgac tgtgtactct ccattggttc gtaacggttg cggtccaggt | | 540 |
| | aaaaaagttg gtatagttgg tcttggtggt atcggcagta tgggtacatt gatttccaaa | | 600 |
| | gccatggggg cagagacgta tgttatttct cgttcttcga gaaaaagaga agatgcaatg | | 660 |
| | aagatgggcg ccgatcacta cattgctaca ttagaagaag gtgattgggg tgaaaagtac | | 720 |
| | tttgacacct tcgacctgat tgtagtctgt gcttcctccc ttaccgacat tgacttcaac | | 780 |
| | attatgccaa aggctatgaa ggttggtggt agaattgtct caatctctat accagaacaa | | 840 |
| | cacgaaatgt tatcgctaaa gccatatggc ttaaaggctg tctccatttc ttacagtgct | | 900 |
| | ttaggttcca tcaaagaatt gaaccaactc ttgaaattag tctctgaaaa agatatcaaa | | 960 |
| | atttgggtgg aaacattacc tgttggtgaa gccggcgtcc atgaagcctt cgaaaggatg | | 1020 |
| | gaaaagggtg acgttagata tagatttacc ttagtcggct acgacaaaga attttcagac | | 1080 |
| | tag | | 1083 |
| 3-10 | Sequences | | |
| 3-10-1 | Sequence Number [ID] | 10 | |
| 3-10-2 | Molecule Type | AA | |
| 3-10-3 | Length | 360 | |
| | | source 1 . . . 360 | |
| 3-10-4-1 | Features Location/ | mol_type = protein | |
| | Qualifiers | organism = Saccharomyces cerevisiae | |
| | NonEnglishQualifier Value | | |
| 3-10-5 | Residues | | |
| | MSYPEKFEGI AIQSHEDWKN PKKTKYDPKP FYDHDIDIKI EACGVCGSDI HCAAGHWGNM | | 60 |
| | KMPLVVGHEI VGKVVKLGPK SNSGLKVGQR VGVGAQVFSC LECDRCKNDN EPYCTKFVTT | | 120 |
| | YSQPYEDGYV SQGGYANYVR VHEHFVVPIP ENIPSHLAAP LLCGGLTVYS PLVRNGCGPG | | 180 |
| | KKVGIVGLGG IGSMGTLISK AMGAETYVIS RSSRKREDAM KMGADHYIAT LEEGDWGEKY | | 240 |
| | FDTFDLIVVC ASSLTDIDFN IMPKAMKVGG RIVSISIPEQ HEMLSLKPYG LKAVSISYSA | | 300 |
| | LGSIKELNQL LKLVSEKDIK IWVETLPVGE AGVHEAFERM EKGDVRYRFT LVGYDKEFSD | | 360 |
| 3-11 | Sequences | | |
| 3-11-1 | Sequence Number [ID] | 11 | |
| 3-11-2 | Molecule Type | DNA | |
| 3-11-3 | Length | 783 | |
| | | source 1 . . . 783 | |
| 3-11-4-1 | Features Location/ | mol_type = other DNA | |
| | Qualifiers | organism = Methylococcus capsulatus | |
| | NonEnglishQualifier Value | | |
| 3-11-5 | Residues | | |
| | atggcagcaa caaccattgg tggtgcagct gcggcggaag cgccgctgct ggacaagaag | | 60 |
| | tggctcacgt tcgcactggc gatttacacc gtgttctacc tgtgggtgcg gtggtacgaa | | 120 |
| | ggtgtctatg gctggtccgc cggactggac tcgttcgcgc cggagttcga gacctactgg | | 180 |
| | atgaatttcc tgtacaccga gatcgtcctg gagatcgtga cggcttcgat cctgtggggc | | 240 |
| | tatctctgga agacccgcga ccgcaacctg gccgcgctga ccccgcgtga agagctgcgc | | 300 |
| | cgcaacttca cccacctggt gtggctggtg gcctacgcct gggccatcta ctggggcgca | | 360 |
| | tcctacttca ccgagcagga cggcacctgg catcagacga tcgtgcgcga caccgacttc | | 420 |
| | acgccgtcgc acatcatcga gttctatctg agctacccga tctacatcat caccggtttt | | 480 |
| | gcggcgttca tctacgccaa gacgcgtctg ccgttcttcg cgaagggcat ctcgctgccg | | 540 |
| | tacctggtgc tggtggtggg tccgttcatg attctgccga acgtgggtct gaacgaatgg | | 600 |
| | ggccacacct tctggttcat ggaagagctg ttcgtggcgc cgctgcacta cggcttcgtg | | 660 |
| | atcttcggct ggctggcact ggccgtcatg ggcaccctga cccagacctt ctacagcttc | | 720 |
| | gctcagggcg ggctggggca gtcgctctgt gaagccgtgg acgaaggctt gatcgcgaaa | | 780 |
| | taa | | 783 |
| 3-12 | Sequences | | |
| 3-12-1 | Sequence Number [ID] | 12 | |
| 3-12-2 | Molecule Type | AA | |
| 3-12-3 | Length | 260 | |
| | | source 1 . . . 260 | |
| 3-12-4-1 | Features Location/ | mol_type = protein | |
| | Qualifiers | organism = Methylococcus capsulatus | |
| | NonEnglishQualifier Value | | |
| 3-12-5 | Residues | | |
| | MAATTIGGAA AAEAPLLDKK WLTFALAIYT VFYLWVRWYE GVYGWSAGLD SFAPEFETYW | | 60 |
| | MNFLYTEIVL EIVTASILWG YLWKTRDRNL AALTPREELR RNFTHLVWLV AYAWAIYWGA | | 120 |
| | SYFTEQDGTW HQTIVRDTDF TPSHIIEFYL SYPIYIITGF AAFIYAKTRL PFFAKGISLP | | 180 |
| | YLVLVVGPFM ILPNVGLNEW GHTFWFMEEL FVAPLHYGFV IFGWLALAVM GTLTQTFYSF | | 240 |
| | AQGGLGQSLC EAVDEGLIAK | | 260 |
| 3-13 | Sequences | | |
| 3-13-1 | Sequence Number [ID] | 13 | |
| 3-13-2 | Molecule Type | DNA | |
| 3-13-3 | Length | 744 | |
| | | source 1 . . . 744 | |
| 3-13-4-1 | Features Location/ | mol_type = other DNA | |
| | Qualifiers | organism = Methylococcus capsulatus | |
| | NonEnglishQualifier Value | | |
| 3-13-5 | Residues | | |
| | atgagtgctg cgcaatctgc ggttcgttcg cacgccgaag cggtccaggt atcccggacc | | 60 |
| | atcgactgga tggcgttgtt cgtggtgttt ttcgtgatcg tgggctcgta ccacattcat | | 120 |
| | gccatgctca ccatgggtga ctgggacttc tggtcggact ggaaagaccg tcgactgtgg | | 180 |
| | gtcacggtga ccccgatcgt actggtcacc ttcccggcgg ccgtacaatc ctacctgtgg | | 240 |
| | gagcggtatc gtctgccctg gggagccacc gtgtgcgtcc tgggtctgct gctgggcgag | | 300 |
| | tggatcaacc gttatttcaa cttctggggc tggacctact tcccgatcaa cttcgtgttc | | 360 |
| | cctgcctcgc tggtgccggg cgccatcatc ctggacaccg tgctgatgct gtcgggcagc | | 420 |
| | tacctgttca ccgcgatcgt cggtgcgatg ggctggggtc tgatcttcta cccgggcaac | | 480 |
| | tggccgatca tcgcgccgct gcacgtgccg gtggaataca acggcatgct gatgtcgatc | | 540 |
| | gccgacatcc agggttacaa ctatgtgcgt acgggtacgc ctgagtacat ccgcatggta | | 600 |
| | gagaagggca ccctgcgtac cttcggtaag gacgtggcgc cggtatcggc attcttctcc | | 660 |
| | gcgttcatgt cgatcctgat ctacttcatg tggcacttca tcggtcgctg gttctccaac | | 720 |
| | gaacggttcc tgcagagcac ctga | | 744 |
| 3-14 | Sequences | | |
| 3-14-1 | Sequence Numer [ID] | 14 | |
| 3-14-2 | Molecule Type | AA | |
| 3-14-3 | Length | 247 | |
| | | source 1 . . . 247 | |
| 3-14-4-1 | Features Location/ | mol_type = protein | |
| | Qualifiers | organism = Methylococcus capsulatus | |
| | NonEnglishQualifier Value | | |
| 3-14-5 | Residues | | |
| | MSAAQSAVRS HAEAVQVSRT IDWMALFVVF FVIVGSYHIH AMLTMGDWDF WSDWKDRRLW | | 60 |
| | VTVTPIVLVT FPAAVQSYLW ERYRLPWGAT VCVLGLLLGE WINRYFNFWG WTYFPINFVF | | 120 |
| | PASLVPGAII LDTVLMLSGS YLFTAIVGAM GWGLIFYPGN WPIIAPLHVP VEYNGMLMSI | | 180 |
| | ADIQGYNYVR TGTPEYIRMV EKGTLRTFGK DVAPVSAFFS AFMSILIYFM WHFIGRWFSN | | 240 |
| | ERFLQST | | 247 |
| 3-15 | Sequences | | |
| 3-15-1 | Sequence Numer [ID] | 15 | |
| 3-15-2 | Molecule Type | DNA | |
| 3-15-3 | Length | 1245 | |
| | | source 1 . . . 1245 | |
| 3-15-4-1 | Features Location/ | mol_type = other DNA | |
| | Qualifiers | organism = Methylococcus capsulatus | |
| | NonEnglishQualifier Value | | |
| 3-15-5 | Residues | | |
| | atgaaaacaa taaaggaccg gattgcaaaa tggtctgcaa tcggactgct gtccgccgtg | | 60 |
| | gcagcgaccg ccttctatgc gccgagcgcc agcgcccacg gtgagaaatc gcaggccgcg | | 120 |
| | ttcatgcgta tgcgtaccat ccactggtac gacctgagct ggtcgaaaga gaaagtcaag | | 180 |
| | atcaacgaga ccgtggaaat caaaggcaag ttccacgtgt tcgaaggctg gccggaaacg | | 240 |
| | gtcgacgaac cggatgtggc gttcctgaac gtcggcatgc cgggtccggt gttcatccgc | | 300 |
| | aaggaatcgt acatcggcgg tcagctggtg ccgcgttccg tacgtctgga aatcggcaag | | 360 |
| | acctatgact tccgggttgt cctcaaagcc cgtcgtccgg gtgactggca cgttcacacc | | 420 |
| | atgatgaacg tccagggcgg tggaccgatc atcggtcccg gcaaatggat caccgtggaa | | 480 |
| | ggctccatga gtgaattccg caaccccgtc accaccctga ccggtcagac ggtggacctg | | 540 |
| | gagaactaca acgaaggcaa cacctatttc tggcacgcct tctggttcgc catcggagtt | | 600 |
| | gcctggatcg gctactggtc gcgtcgaccg atcttcatcc cccgtctgct gatggtggat | | 660 |
| | gccggtcgtg cggatgaact ggtgtccgcc accgaccgca aggtggcgat gggcttcctg | | 720 |
| | gccgccacca tcctgatcgt ggtcatggcc atgtccagcg ccaacagcaa gtacccgatc | | 780 |
| | accatcccgc tgcaggccgg caccatgcgt ggcatgaagc cgctggaact gccggcgccg | | 840 |
| | acggtatcgg tgaaagtgga agacgccacc taccgggtac cgggccgcgc catgcggatg | | 900 |
| | aagctgacca tcaccaacca cggcaacagc ccgatccggc tgggtgagtt ctacaccgcc | | 960 |
| | tcggtgcgtt tcctggattc cgacgtgtac aaggacacca ccggctatcc ggaagacctg | | 1020 |
| | ctggccgaag acggcctgag cgtcagcgac aacagcccgc tggctccggg tgagacgcgc | | 1080 |
| | acggtcgacg tgacggcgtc cgacgcggcg tgggaagtgt accgtctgtc cgacatcatc | | 1140 |
| | tacgatccgg acagccgttt cgccggtctg ctgttcttct tcgacgccac tggcaaccgc | | 1200 |
| | caggtcgtcc agatcgacgc accgctgatc ccgtcgttca tgtaa | | 1245 |
| 3-16 | Sequences | | |
| 3-16-1 | Sequence Number [ID] | 16 | |
| 3-16-2 | Molecule Type | AA | |
| 3-16-3 | Length | 414 | |
| | | source 1 . . . 414 | |
| 3-16-4-1 | Features Location/ | mol_type = protein | |
| | Qualifiers | organism = Methylococcus capsulatus | |
| | NonEnglishQualifier Value | | |
| 3-16-5 | Residues | | |
| | MKTIKDRIAK WSAIGLLSAV AATAFYAPSA SAHGEKSQAA FMRMRTIHWY DLSWSKEKVK | | 60 |
| | INETVEIKGK FHVFEGWPET VDEPDVAFLN VGMPGPVFIR KESYIGGQLV PRSVRLEIGK | | 120 |
| | TYDFRVVLKA RRPGDWHVHT MMNVQGGGPI IGPGKWITVE GSMSEFRNPV TTLTGQTVDL | | 180 |
| | ENYNEGNTYF WHAFWFAIGV AWIGYWSRRP IFIPRLLMVD AGRADELVSA TDRKVAMGFL | | 240 |
| | AATILIVVMA MSSANSKYPI TIPLQAGTMR GMKPLELPAP TVSVKVEDAT YRVPGRAMRM | | 300 |
| | KLTITNHGNS PIRLGEFYTA SVRFLDSDVY KDTTGYPEDL LAEDGLSVSD NSPLAPGETR | | 360 |
| | TVDVTASDAA WEVYRLSDII YDPDSRFAGL LFFFDATGNR QVVQIDAPLI PSFM | | 414 |
| 3-17 | Sequences | | |
| 3-17-1 | Sequence Number [ID] | 17 | |
| 3-17-2 | Molecule Type | DNA | |
| 3-17-3 | Length | 783 | |
| | | source 1 . . . 783 | |
| 3-17-4-1 | Features Location/ | mol_type = other DNA | |
| | Qualifiers | organism = Methylococcus capsulatus | |
| | NonEnglishQualifier Value | | |
| 3-17-5 | Residues | | |
| | atggcagcaa caaccattgg tggtgcagct gcggcggaag cgccgctgct ggacaagaag | | 60 |
| | tggctcacgt tcgcactggc gatttacacc gtgttctacc tgtgggtgcg gtggtacgaa | | 120 |
| | ggtgtctatg gctggtccgc cggactggac tcgttcgcgc cggagttcga gacctactgg | | 180 |
| | atgaatttcc tgtacaccga gatcgtcctg gagatcgtga cggcttcgat cctgtggggc | | 240 |
| | tatctctgga agacccgcga ccgcaacctg gccgcgctga ccccgcgtga agagctgcgc | | 300 |
| | cgcaacttca cccacctggt gtggctggtg gcctacgcct gggccatcta ctggggcgca | | 360 |
| | tcctacttca ccgagcagga cggcacctgg catcagacga tcgtgcgcga caccgacttc | | 420 |
| | acgccgtcgc acatcatcga gttctatctg agctacccga tctacatcat caccggtttt | | 480 |
| | gcggcgttca tctacgccaa gacgcgtctg ccgttcttcg cgaagggcat ctcgctgccg | | 540 |
| | tacctggtgc tggtggtggg tccgttcatg attctgccga acgtgggtct gaacgaatgg | | 600 |
| | ggccacacct tctggttcat ggaagagctg ttcgtggcgc cgctgcacta cggcttcgtg | | 660 |
| | atcttcggct ggctggcact ggccgtcatg ggcaccctga cccagacctt ctacagcttc | | 720 |
| | gctcagggcg ggctggggca gtcgctctgt gaagccgtgg acgaaggctt gatcgcgaaa | | 780 |
| | taa | | 783 |
| 3-18 | Sequences | | |
| 3-18-1 | Sequence Number [ID] | 18 | |
| 3-18-2 | Molecule Type | AA | |
| 3-18-3 | Length | 260 | |
| | | source 1 . . . 260 | |
| 3-18-4-1 | Features Location/ | mol_type = protein | |
| | Qualifiers | organism = Methylococcus capsulatus | |
| | NonEnglishQualifier Value | | |
| 3-18-5 | Residues | | |
| | MAATTIGGAA AAEAPLLDKK WLTFALAIYT VFYLWVRWYE GVYGWSAGLD SFAPEFETYW | | 60 |
| | MNFLYTEIVL EIVTASILWG YLWKTRDRNL AALTPREELR RNFTHLVWLV AYAWAIYWGA | | 120 |
| | SYFTEQDGTW HQTIVRDTDF TPSHIIEFYL SYPIYIITGF AAFIYAKTRL PFFAKGISLP | | 180 |
| | YLVLVVGPFM ILPNVGLNEW GHTFWFMEEL FVAPLHYGFV IFGWLALAVM GTLTQTFYSF | | 240 |
| | AQGGLGQSLC EAVDEGLIAK | | 260 |
| 3-19 | Sequences | | |
| 3-19-1 | Sequence Number [ID] | 19 | |
| 3-19-2 | Molecule Type | DNA | |
| 3-19-3 | Length | 744 | |
| | | source 1 . . . 744 | |
| 3-19-4-1 | Features Location/ | mol_type = other DNA | |
| | Qualifiers | organism = Methylococcus capsulatus | |
| | NonEnglishQualifier Value | | |
| 3-19-5 | Residues | | |
| | atgagtgctg cgcaatctgc ggttcgttcg cacgccgaag cggtccaggt atcccggacc | | 60 |
| | atcgactgga tggcgttgtt cgtggtgttt ttcgtgatcg tgggctcgta ccacattcac | | 120 |
| | gccatgctca ccatgggtga ctgggacttc tggtcggact ggaaagaccg tcgactgtgg | | 180 |
| | gtcacggtga ccccgatcgt actggtcacc ttcccggcgg ccgtacaatc ctacctgtgg | | 240 |
| | gagcggtatc gtctgccctg gggagccacc gtgtgcgtcc tgggtctgct gctgggcgag | | 300 |
| | tggatcaacc gttatttcaa cttctggggc tggacctact tcccgatcaa cttcgtgttc | | 360 |
| | cctgcctcgc tggtgccggg cgccatcatc ctggacaccg tgctgatgct gtcgggcagc | | 420 |
| | tacctgttca ccgcgatcgt cggtgcgatg ggctggggtc tgatcttcta cccgggcaac | | 480 |
| | tggccgatca tcgcgccgct gcacgtgccg gtggaataca acggcatgct gatgtcgatc | | 540 |
| | gccgacatcc agggttacaa ctatgtgcgt acgggtacgc ctgagtacat ccgcatggta | | 600 |
| | gagaagggca ccctgcgtac cttcggtaag gacgtggcgc cggtatcggc attcttctcc | | 660 |
| | gcgttcatgt cgatcctgat ctacttcatg tggcacttca tcggtcgctg gttctccaac | | 720 |
| | gaacggttcc tgcagagcac ctga | | 744 |
| 3-20 | Sequences | | |
| 3-20-1 | Sequence Number [ID] | 20 | |
| 3-20-2 | Molecule Type | AA | |
| 3-20-3 | Length | 247 | |
| | | source 1 . . . 247 | |
| 3-20-4-1 | Features Location/ | mol_type = protein | |
| | Qualifiers | organism = Methylococcus capsulatus | |
| | NonEnglishQualifier Value | | |
| 3-20-5 | Residues | | |
| | MSAAQSAVRS HAEAVQVSRT IDWMALFVVF FVIVGSYHIH AMLTMGDWDF WSDWKDRRLW | | 60 |
| | VTVTPIVLVT FPAAVQSYLW ERYRLPWGAT VCVLGLLLGE WINRYFNFWG WTYFPINFVE | | 120 |
| | PASLVPGAII LDTVLMLSGS YLFTAIVGAM GWGLIFYPGN WPIIAPLHVP VEYNGMLMSI | | 180 |
| | ADIQGYNYVR TGTPEYIRMV EKGTLRTFGK DVAPVSAFFS AFMSILIYFM WHFIGRWFSN | | 240 |
| | ERFLQST | | 247 |
| 3-21 | Sequences | | |
| 3-21-1 | Sequence Number [ID] | 21 | |
| 3-21-2 | Molecule Type | DNA | |
| 3-21-3 | Length | 1245 | |
| | | source 1 . . . 1245 | |
| 3-21-4-1 | Features Location/ | mol_type = other DNA | |
| | Qualifiers | organism = Methylococcus capsulatus | |
| | NonEnglishQualifier Value | | |
| 3-21-5 | Residues | | |
| | atgaaaacaa taaaggaccg gattgcaaaa tggtctgcaa tcggactgct gtccgccgtg | | 60 |
| | gcagcgaccg ccttctatgc gccgagcgcc agcgcccacg gtgagaaatc gcaggccgcg | | 120 |
| | ttcatgcgta tgcgtaccat ccactggtac gacctgagct ggtcgaaaga gaaagtcaag | | 180 |
| | atcaacgaga ccgtggaaat caaaggcaag ttccacgtgt tcgaaggctg gccggaaacg | | 240 |
| | gtcgacgaac cggatgtggc gttcctgaac gtcggcatgc cgggtccggt gttcatccgc | | 300 |
| | aaggaatcgt acatcggcgg tcagctggtg ccgcgttccg tacgtctgga aatcggcaag | | 360 |
| | acctatgact tccgggttgt cctcaaagcc cgtcgtccgg gtgactggca cgttcacacc | | 420 |
| | atgatgaacg tccagggcgg tggaccgatc atcggtcccg gcaaatggat caccgtggaa | | 480 |
| | ggctccatga gtgaattccg caaccccgtc accaccctga ccggtcagac ggtggacctg | | 540 |
| | gagaactaca acgaaggcaa cacctatttc tggcacgcct tctggttcgc catcggagtt | | 600 |
| | gcctggatcg gctactggtc gcgtcgaccg atcttcatcc cccgtctgct gatggtggat | | 660 |
| | gccggtcgtg cggacgaact ggtgtccgcc accgaccgca aggtggcgat gggcttcctg | | 720 |
| | gccgccacca tcctgatcgt ggtcatggcc atgtccagcg ccaacagcaa gtacccgatc | | 780 |
| | accatcccgc tgcaggccgg caccatgcgt ggcatgaagc cgctggaact gccggcgccg | | 840 |
| | acggtatcgg tgaaagtgga agacgccacc taccgggtac cgggccgcgc catgcggatg | | 900 |
| | aagctgacca tcaccaacca cggcaacagc ccgatccggc tgggtgagtt ctacaccgcc | | 960 |
| | tcggtgcgtt tcctggattc cgacgtgtac aaggacacca ccggctatcc ggaagacctg | | 1020 |
| | ctggccgaag acggcctgag cgtcagcgac aacagcccgc tggctccggg tgagacccgc | | 1080 |
| | acggtcgacg tgacggcgtc cgacgcggcg tgggaagtgt accgtctgtc cgacatcatc | | 1140 |
| | tacgatccgg acagccgttt cgccggtctg ctgttcttct tcgacgccac tggcaaccgc | | 1200 |
| | caggtcgtcc agatcgacgc accgctgatc ccgtcgttca tgtaa | | 1245 |
| 3-22 | Sequences | | |
| 3-22-1 | Sequence Number [ID] | 22 | |
| 3-22-2 | Molecule Type | AA | |
| 3-22-3 | Length | 414 | |
| | | source 1 . . . 414 | |
| 3-22-4-1 | Features Location/ | mol_type = protein | |
| | Qualifiers | organism = Methylococcus capsulatus | |
| | NonEnglishQualifier Value | | |
| 3-22-5 | Residues | | |
| | MKTIKDRIAK WSAIGLLSAV AATAFYAPSA SAHGEKSQAA FMRMRTIHWY DLSWSKEKVK | | 60 |
| | INETVEIKGK FHVFEGWPET VDEPDVAFLN VGMPGPVFIR KESYIGGQLV PRSVRLEIGK | | 120 |
| | TYDFRVVLKA RRPGDWHVHT MMNVQGGGPI IGPGKWITVE GSMSEFRNPV TTLTGQTVDL | | 180 |
| | ENYNEGNTYF WHAFWFAIGV AWIGYWSRRP IFIPRLLMVD AGRADELVSA TDRKVAMGFL | | 240 |
| | AATILIVVMA MSSANSKYPI TIPLQAGTMR GMKPLELPAP TVSVKVEDAT YRVPGRAMRM | | 300 |
| | KLTITNHGNS PIRLGEFYTA SVRFLDSDVY KDTTGYPEDL LAEDGLSVSD NSPLAPGETR | | 360 |
| | TVDVTASDAA WEVYRLSDII YDPDSRFAGL LFFFDATGNR QVVQIDAPLI PSFM | | 414 |
| 3-23 | Sequences | | |
| 3-23-1 | Sequence Number [ID] | 23 | |
| 3-23-2 | Molecule Type | DNA | |
| 3-23-3 | Length | 1584 | |
| | | source 1 . . . 1584 | |
| 3-23-4-1 | Features Location/ | mol_type = other DNA | |
| | Qualifiers | organism = Methylococcus capsulatus | |
| | NonEnglishQualifier Value | | |
| 3-23-5 | Residues | | |
| | atggcactta gcaccgcaac caaggccgcg acggacgcgc tggctgccaa tcgggcaccc | | 60 |
| | accagcgtga atgcacagga agtgcaccgt tggctccaga gcttcaactg ggatttcaag | | 120 |
| | aacaaccgga ccaagtacgc caccaagtac aagatggcga acgagaccaa ggaacagttc | | 180 |
| | aagctgatcg ccaaggaata tgcgcgcatg gaggcagtca aggacgaaag gcagttcggt | | 240 |
| | agcctgcagg atgcgctgac ccgcctcaac gccggtgttc gcgttcatcc gaagtggaac | | 300 |
| | gagaccatga aagtggtttc gaacttcctg gaagtgggcg aatacaacgc catcgccgct | | 360 |
| | accgggatgc tgtgggattc cgcccaggcg gcggaacaga agaacggcta tctggcccag | | 420 |
| | gtgttggatg aaatccgcca cacccaccag tgtgcctacg tcaactacta cttcgcgaag | | 480 |
| | aacggccagg acccggccgg tcacaacgat gctcgccgca cccgtaccat cggtccgctg | | 540 |
| | tggaagggca tgaagcgcgt gttttccgac ggcttcattt ccggcgacgc cgtggaatgc | | 600 |
| | tccctcaacc tgcagctggt gggtgaggcc tgcttcacca atccgctgat cgtcgcagtg | | 660 |
| | accgaatggg ctgccgccaa cggcgatgaa atcaccccga cggtgttcct gtcgatcgag | | 720 |
| | accgacgaac tgcgccacat ggccaacggt taccagaccg tcgtttccat cgccaacgat | | 780 |
| | ccggcttccg ccaagtatct caacacggac ctgaacaacg ccttctggac ccagcagaag | | 840 |
| | tacttcacgc cggtgttggg catgctgttc gagtatggct ccaagttcaa ggtcgagccg | | 900 |
| | tgggtcaaga cgtggaaccg ctgggtgtac gaggactggg gcggcatctg gatcggccgt | | 960 |
| | ctgggcaagt acggggtgga gtcgccgcgc agcctcaagg acgccaagca ggacgcttac | | 1020 |
| | tgggctcacc acgacctgta tctgctggct tatgcgctgt ggccgaccgg cttcttccgt | | 1080 |
| | ctggcgctgc cggatcagga agaaatggag tggttcgagg ccaactaccc cggctggtac | | 1140 |
| | gaccactacg gcaagatcta cgaggaatgg cgcgcccgcg gttgcgagga tccgtcctcg | | 1200 |
| | ggcttcatcc cgctgatgtg gttcatcgaa aacaaccatc ccatctacat cgatcgcgtg | | 1260 |
| | tcgcaagtgc cgttctgccc gagcttggcc aagggcgcca gcaccctgcg cgtgcacgag | | 1320 |
| | tacaacggcc agatgcacac cttcagcgac cagtggggcg agcgcatgtg gctggccgag | | 1380 |
| | ccggagcgct acgagtgcca gaacatcttc gaacagtacg aaggacgcga actgtcggaa | | 1440 |
| | gtgatcgccg aactgcacgg gctgcgcagt gatggcaaga ccctgatcgc ccagccgcat | | 1500 |
| | gtccgtggcg acaagctgtg gacgttggac gatatcaaac gcctgaactg cgtcttcaag | | 1560 |
| | aacccggtga aggcattcaa ttga | | 1584 |
| 3-24 | Sequences | | |
| 3-24-1 | Sequence Number [ID] | 24 | |
| 3-24-2 | Molecule Type | AA | |
| 3-24-3 | Length | 527 | |
| | | source 1 . . . 527 | |
| 3-24-4-1 | Features Location/ | mol_type = protein | |
| | Qualifiers | organism = Methylococcus capsulatus | |
| | NonEnglishQualifier Value | | |
| 3-24-5 | Residues | | |
| | MALSTATKAA TDALAANRAP TSVNAQEVHR WLQSFNWDFK NNRTKYATKY KMANETKEQF | | 60 |
| | KLIAKEYARM EAVKDERQFG SLQDALTRLN AGVRVHPKWN ETMKVVSNFL EVGEYNAIAA | | 120 |
| | TGMLWDSAQA AEQKNGYLAQ VLDEIRHTHQ CAYVNYYFAK NGQDPAGHND ARRTRTIGPL | | 180 |
| | WKGMKRVFSD GFISGDAVEC SLNLQLVGEA CFTNPLIVAV TEWAAANGDE ITPTVFLSIE | | 240 |
| | TDELRHMANG YQTVVSIAND PASAKYLNTD LNNAFWTQQK YFTPVLGMLF EYGSKFKVEP | | 300 |
| | WVKTWNRWVY EDWGGIWIGR LGKYGVESPR SLKDAKQDAY WAHHDLYLLA YALWPTGFFR | | 360 |
| | LALPDQEEME WFEANYPGWY DHYGKIYEEW RARGCEDPSS GFIPLMWFIE NNHPIYIDRV | | 420 |
| | SQVPFCPSLA KGASTLRVHE YNGQMHTFSD QWGERMWLAE PERYECQNIF EQYEGRELSE | | 480 |
| | VIAELHGLRS DGKTLIAQPH VRGDKLWTLD DIKRLNCVFK NPVKAFN | | 527 |
| 3-25 | Sequences | | |
| 3-25-1 | Sequence Number [ID] | 25 | |
| 3-25-2 | Molecule Type | DNA | |
| 3-25-3 | Length | 1170 | |
| | | source 1 . . . 1170 | |
| 3-25-4-1 | Features Location/ | mol_type = other DNA | |
| | Qualifiers | organism = Methylococcus capsulatus | |
| | NonEnglishQualifier Value | | |
| 3-25-5 | Residues | | |
| | atgagcatgt taggagaaag acgccgcggt ctgaccgatc cggaaatggc ggccgtcatt | | 60 |
| | ttgaaggcgc ttcctgaagc tccgctggac ggcaacaaca agatgggtta tttcgtcacc | | 120 |
| | ccccgctgga aacgcttgac ggaatatgaa gccctgaccg tttatgcgca gcccaacgcc | | 180 |
| | gactggatcg ccggcggcct ggactggggc gactggaccc agaaattcca cggcggccgc | | 240 |
| | ccttcctggg gcaacgagac cacggagctg cgcaccgtcg actggttcaa gcaccgtgac | | 300 |
| | ccgctccgcc gttggcatgc gccgtacgtc aaggacaagg ccgaggaatg gcgctacacc | | 360 |
| | gaccgcttcc tgcagggtta ctccgccgac ggtcagatcc gggcgatgaa cccgacctgg | | 420 |
| | cgggacgagt tcatcaaccg gtattggggc gccttcctgt tcaacgaata cggattgttc | | 480 |
| | aacgctcatt cgcagggcgc ccgggaggcg ctgtcggacg taacccgcgt cagcctggct | | 540 |
| | ttctggggct tcgacaagat cgacatcgcc cagatgatcc aactcgaacg gggtttcctc | | 600 |
| | gccaagatcg tacccggttt cgacgagtcc acagcggtgc cgaaggccga atggacgaac | | 660 |
| | ggggaggtct acaagagcgc ccgtctggcc gtggaagggc tgtggcagga ggtgttcgac | | 720 |
| | tggaacgaga gcgctttctc ggtgcacgcc gtctatgacg cgctgttcgg tcagttcgtc | | 780 |
| | cgccgcgagt tctttcagcg gctggctccc cgcttcggcg acaatctgac gccattcttc | | 840 |
| | atcaaccagg cccagacata cttccagatc gccaagcagg gcgtacagga tctgtattac | | 900 |
| | aactgtctgg gtgacgatcc ggagttcagc gattacaacc gtaccgtgat gcgcaactgg | | 960 |
| | accggcaagt ggctggagcc cacgatcgcc gctctgcgcg acttcatggg gctgtttgcg | | 1020 |
| | aagctgccgg cgggcaccac tgacaaggaa gaaatcaccg cgtccctgta ccgggtggtc | | 1080 |
| | gacgactgga tcgaggacta cgccagcagg atcgacttca aggcggaccg cgatcagatc | | 1140 |
| | gttaaagcgg ttctggcagg attgaaataa | | 1170 |
| 3-26 | Sequences | | |
| 3-26-1 | Sequence Number [ID] | 26 | |
| 3-26-2 | Molecule Type | AA | |
| 3-26-3 | Length | 389 | |
| | | source 1 . . . 389 | |
| 3-26-4-1 | Features Location/ | mol_type = protein | |
| | Qualifiers | organism = Methylococcus capsulatus | |
| | NonEnglishQualifier Value | | |
| 3-26-5 | Residues | | |
| | MSMLGERRRG LTDPEMAAVI LKALPEAPLD GNNKMGYFVT PRWKRLTEYE ALTVYAQPNA | | 60 |
| | DWIAGGLDWG DWTQKFHGGR PSWGNETTEL RTVDWFKHRD PLRRWHAPYV KDKAEEWRYT | | 120 |
| | DRFLQGYSAD GQIRAMNPTW RDEFINRYWG AFLFNEYGLF NAHSQGAREA LSDVTRVSLA | | 180 |
| | FWGFDKIDIA QMIQLERGFL AKIVPGFDES TAVPKAEWTN GEVYKSARLA VEGLWQEVFD | | 240 |
| | WNESAFSVHA VYDALFGQFV RREFFQRLAP RFGDNLTPFF INQAQTYFQI AKQGVQDLYY | | 300 |
| | NCLGDDPEFS DYNRTVMRNW TGKWLEPTIA ALRDFMGLFA KLPAGTTDKE EITASLYRVV | | 360 |
| | DDWIEDYASR IDFKADRDQI VKAVLAGLK | | 389 |
| 3-27 | Sequences | | |
| 3-27-1 | Sequence Number [ID] | 27 | |
| 3-27-2 | Molecule Type | DNA | |
| 3-27-3 | Length | 426 | |
| | | source 1 . . . 426 | |
| 3-27-4-1 | Features Location/ | mol_type = other DNA | |
| | Qualifiers | organism = Methylococcus capsulatus | |
| | NonEnglishQualifier Value | | |
| | Residues | | |
| 3-27-5 | atgagcgtaa acagcaacgc atacgacgcc ggcatcatgg gcctgaaagg caaggacttc | | 60 |
| | gccgatcagt tctttgccga cgaaaaccaa gtggtccatg aaagcgacac ggtcgttctg | | 120 |
| | gtcctcaaga agtcggacga gatcaatacc tttatcgagg agatccttct gacggactac | | 180 |
| | aagaagaacg tcaatccgac ggtaaacgtg gaagaccgcg cgggttactg gtggatcaag | | 240 |
| | gccaacggca agatcgaggt cgattgcgac gagatttccg agctgttggg gcggcagttc | | 300 |
| | aacgtctacg acttcctcgt cgacgtttcc tccaccatcg gccgggccta taccctgggc | | 360 |
| | aacaagttca ccattaccag tgagctgatg ggcctggacc gcaagctcga agactatcac | | 420 |
| | gcttaa | | 426 |
| 3-28 | Sequences | | |
| 3-28-1 | Sequence Number [ID] | 28 | |
| 3-28-2 | Molecule Type | AA | |
| 3-28-3 | Length | 138 | |
| | | source 1 . . . 138 | |
| 3-28-4-1 | Features Location/ | mol_type = protein | |
| | Qualifiers | organism = Methylococcus capsulatus | |
| | NonEnglishQualifier Value | | |
| 3-28-5 | Residues | | |
| | MSVNSNAYDA GIMGLKGKDF ADQFFADENQ VVHESDTVVL VLKKSDEINT FIEEILLTDY | | 60 |
| | KKNVNPTVNV EDRAGYWWIK ANGKIEVDCD EISELLGRQF NVYDFLVDVS STIGRAYTLG | | 120 |
| | NKFTITSELM GLDRKLED | | 138 |
| 3-29 | Sequences | | |
| 3-29-1 | Sequence Number [ID] | 29 | |
| 3-29-2 | Molecule Type | DNA | |
| 3-29-3 | Length | 513 | |
| | | source 1 . . . 513 | |
| 3-29-4-1 | Features Location/ | mol_type = other DNA | |
| | Qualifiers | organism = Methylococcus capsulatus | |
| | NonEnglishQualifier Value | | |
| 3-29-5 | Residues | | |
| | atggcgaaac tgggtataca cagcaacgac acccgcgacg cctgggtgaa caagatcgcg | | 60 |
| | cagctcaaca ccctggaaaa agcggccgag atgctgaagc agttccggat ggaccacacc | | 120 |
| | acgccgttcc gcaacagcta cgaactggac aacgactacc tctggatcga ggccaagctc | | 180 |
| | gaagagaagg tcgccgtcct caaggcacgc gccttcaacg aggtggactt ccgtcataag | | 240 |
| | accgctttcg gcgaggatgc caagtccgtt ctggacggca ccgtcgcgaa gatgaacgcg | | 300 |
| | gccaaggaca agtgggaggc ggagaagatc catatcggtt tccgccaggc ctacaagccg | | 360 |
| | ccgatcatgc cggtgaacta tttcctggac ggcgagcgtc agttggggac ccggctgatg | | 420 |
| | gaactgcgca acctcaacta ctacgacacg ccgctggaag aactgcgcaa acagcgcggt | | 480 |
| | gtgcgggtgg tgcatctgca gtcgccgcac tga | | 513 |
| 3-30 | Sequences | | |
| 3-30-1 | Sequence Number [ID] | 30 | |
| 3-30-2 | Molecule Type | AA | |
| 3-30-3 | Length | 170 | |
| | | source 1 .. 170 | |
| 3-30-4-1 | Features Location/ | mol_type = protein | |
| | Qualifiers | organism = Methylococcus capsulatus | |
| | NonEnglishQualifier Value | | |
| 3-30-5 | Residues | | |
| | MAKLGIHSND TRDAWVNKIA QLNTLEKAAE MLKQFRMDHT TPFRNSYELD NDYLWIEAKL | | 60 |
| | EEKVAVLKAR AFNEVDFRHK TAFGEDAKSV LDGTVAKMNA AKDKWEAEKI HIGFRQAYKP | | 120 |
| | PIMPVNYFLD GERQLGTRLM ELRNLNYYDT PLEELRKQRG VRVVHLQSPH | | 170 |
| 3-31 | Sequences | | |
| 3-31-1 | Sequence Number [ID] | 31 | |
| 3-31-2 | Molecule Type | DNA | |
| 3-31-3 | Length | 312 | |
| | | source 1 . . . 312 | |
| 3-31-4-1 | Features Location/ | mol_type = other DNA | |
| | Qualifiers | organism = Methylococcus capsulatus | |
| | NonEnglishQualifier Value | | |
| 3-31-5 | Residues | | |
| | atggtcgaat cggcatttca gccattttcg ggcgacgcag acgaatggtt cgaggaacca | | 60 |
| | cggccccagg ccggtttctt cccttccgcg gactggcatc tgctcaaacg ggacgagacc | | 120 |
| | tacgcagcct atgccaagga tctcgatttc atgtggcggt gggtcatcgt ccgggaagaa | | 180 |
| | aggatcgtcc aggagggttg ctcgatcagc ctggagtcgt cgatccgcgc cgtgacgcac | | 240 |
| | gtactgaatt attttggtat gaccgaacaa cgcgccccgg cagaggaccg gaccggcgga | | 300 |
| | gttcaacatt ga | | 312 |
| 3-32 | Sequences | | |
| 3-32-1 | Sequence Number [ID] | 32 | |
| 3-32-2 | Molecule Type | AA | |
| 3-32-3 | Length | 103 | |
| | | source 1 . . . 103 | |
| 3-32-4-1 | Features Location/ | mol_type = protein | |
| | Qualifiers | organism = Methylococcus capsulatus | |
| | NonEnglishQualifier Value | | |
| 3-32-5 | Residues | | |
| | MVESAFQPFS GDADEWFEEP RPQAGFFPSA DWHLLKRDET YAAYAKDLDF MWRWVIVREE | | 60 |
| | RIVQEGCSIS LESSIRAVTH VLNYFGMTEQ RAPAEDRTGG VQH | | 103 |
| 3-33 | Sequences | | |
| 3-33-1 | Sequence Number [ID] | 33 | |
| 3-33-2 | Molecule Type | DNA | |
| 3-33-3 | Length | 1047 | |
| | | source 1 . . . 1047 | |
| 3-33-4-1 | Features Location/ | mol_type = other DNA | |
| | Qualifiers | organism = Methylococcus capsulatus | |
| | NonEnglishQualifier Value | | |
| 3-33-5 | Residues | | |
| | atgcagcgag ttcacactat cacggcggtg acggaggatg gcgaatcgct ccgcttcgaa | | 60 |
| | tgccgttcgg acgaggacgt catcaccgcc gccctgcgcc agaacatctt tctgatgtcg | | 120 |
| | tcctgccggg agggcggctg tgcgacctgc aaggccttgt gcagcgaagg ggactacgac | | 180 |
| | ctcaagggct gcagcgttca ggcgctgccg ccggaagagg aggaggaagg gttggtgttg | | 240 |
| | ttgtgccgga cctacccgaa gaccgacctg gaaatcgaac tgccctatac ccattgccgc | | 300 |
| | atcagttttg gtgaggtcgg cagtttcgag gcggaggtcg tcggcctcaa ctgggtttcg | | 360 |
| | agcaacaccg tccagtttct tttgcagaag cggcccgacg agtgcggcaa ccgtggcgtg | | 420 |
| | aaattcgaac ccggtcagtt catggacctg accatccccg gcaccgatgt ctcccgctcc | | 480 |
| | tactcgccgg cgaaccttcc taatcccgaa ggccgcctgg agttcctgat ccgcgtgtta | | 540 |
| | ccggagggac ggttttcgga ctacctgcgc aatgacgcgc gtgtcggaca ggtcctctcg | | 600 |
| | gtcaaagggc cactgggcgt gttcggtctc aaggagcggg gcatggcgcc gcgctatttc | | 660 |
| | gtggccggcg gcaccgggtt ggcgccggtg gtctcgatgg tgcggcagat gcaggagtgg | | 720 |
| | accgcgccga acgagacccg catctatttc ggtgtgaaca ccgagccgga attgttctac | | 780 |
| | atcgacgagc tcaaatccct ggaacgatcg atgcgcaatc tcaccgtgaa ggcctgtgtc | | 840 |
| | tggcacccga gcggggactg ggaaggcgag cagggctcgc ccatcgatgc gttgcgggaa | | 900 |
| | gacctggagt cctccgacgc caacccggac atttatttgt gcggtccgcc gggcatgatc | | 960 |
| | gatgccgcct gcgagctggt acgcagccgc ggtatccccg gcgaacaggt cttcttcgaa | | 1020 |
| | aaattcctgc cgtccggggc ggcctga | | 1047 |
| 3-34 | Sequences | | |
| 3-34-1 | Sequence Number [ID] | 34 | |
| 3-34-2 | Molecule Type | AA | |
| 3-34-3 | Length | 348 | |
| | | source 1 . . . 348 | |
| 3-34-4-1 | Features Location/ | mol_type = protein | |
| | Qualifiers | organism = Methylococcus capsulatus | |
| | NonEnglishQualifier Value | | |
| 3-34-5 | Residues | | |
| | MQRVHTITAV TEDGESLRFE CRSDEDVITA ALRQNIFLMS SCREGGCATC KALCSEGDYD | | 60 |
| | LKGCSVQALP PEEEEEGLVL LCRTYPKTDL EIELPYTHCR ISFGEVGSFE AEVVGLNWVS | | 120 |
| | SNTVQFLLQK RPDECGNRGV KFEPGQFMDL TIPGTDVSRS YSPANLPNPE GRLEFLIRVL | | 180 |
| | PEGRFSDYLR NDARVGQVLS VKGPLGVFGL KERGMAPRYF VAGGTGLAPV VSMVRQMQEW | | 240 |
| | TAPNETRIYF GVNTEPELFY IDELKSLERS MRNLTVKACV WHPSGDWEGE QGSPIDALRE | | 300 |
| | DLESSDANPD IYLCGPPGMI DAACELVRSR GIPGEQVFFE KFLPSGAA | | 348 |
| 3-35 | Sequences | | |
| 3-35-1 | Sequence Number [ID] | 35 | |
| 3-35-2 | Molecule Type | DNA | |
| 3-35-3 | Length | 1806 | |
| | | source 1 . . . 1806 | |
| 3-35-4-1 | Features Location/ | mol_type = other DNA | |
| | Qualifiers | organism = Methylococcus capsulatus | |
| | NonEnglishQualifier Value | | |
| 3-35-5 | Residues | | |
| | atgcaaattt gcaaactggc aagtggctgc ggcgggtcga tgctggcgat ggccgccgtg | | 60 |
| | ctagccgcgc aatccacgca cgccaattcg gagctggacc ggctgtcgaa ggacgaccgg | | 120 |
| | aactgggtca tgcagaccaa ggactacagc gccacccact tcagccggct gacggaaatc | | 180 |
| | aatagccaca acgtcaagaa cctgaaggtg gcctggaccc tgtccaccgg cacgttgcat | | 240 |
| | ggccacgaag gtgcgccgtt ggtggtggac ggcatcatgt acatccacac gccgttcccc | | 300 |
| | aacaacgtct atgcagtcga cctgaacgac acccgcaaga tgctgtggca gtacaagccc | | 360 |
| | aagcagaatc cggcggcccg cgcggtggct tgctgcgacg tggtcaaccg cggtctggcc | | 420 |
| | tacgtgccgg ccggcgagca cggtccggcg aagatcttcc tcaaccagct tgacggccac | | 480 |
| | atcgtcgcac tcaacgccaa gaccggcgaa gagatatgga agatggaaaa ttccgacatc | | 540 |
| | gccatgggct ccaccctcac cggcgcgcct ttcgtggtga aggacaaggt actggtaggt | | 600 |
| | tcggccgggg ccgagctggg cgtgcgtggc tacgtcacgg cctataacat caaggacggc | | 660 |
| | aagcaggagt ggcgggccta tgccaccggt cccgacgaag acttgttgct ggacaaggac | | 720 |
| | ttcaacaagg acaacccgca ttacggtcag ttcggcctgg ggctctcaac ctgggagggt | | 780 |
| | gatgcctgga agatcggcgg cggcaccaat tggggctggt atgcctatga tcccaagttg | | 840 |
| | gacatgatct actacggttc cggcaatccg gcaccctgga acgagaccat gcggcccggc | | 900 |
| | gacaacaaat ggaccatgac catctggggc cgcgacgccg acaccggccg cgccaagttc | | 960 |
| | ggctaccaga agacgccgca cgacgagtgg gattacgccg gtgtcaacta catgggtctg | | 1020 |
| | tccgaacagg aagtggacgg caagctgacg ccgctgctga cccatcccga ccgcaacggt | | 1080 |
| | ctggtgtata cgctgaaccg ggaaaccggc gccctggtca atgccttcaa gatcgatgac | | 1140 |
| | accgtcaact gggtgaaaaa ggtcgatctg aagaccggcc tgccgatccg cgatccggag | | 1200 |
| | tacagcaccc gcatggacca caatgccaaa ggcatctgtc cctcggccat gggctatcac | | 1260 |
| | aaccagggca tcgagtccta cgatccggac aagaagctgt tcttcatggg cgtgaaccac | | 1320 |
| | atctgcatgg actgggagcc gttcatgctg ccctaccgcg ccggccagtt ctttgtgggg | | 1380 |
| | gcgaccctca acatgtatcc gggacccaag gggatgctgg gtcaggtcaa ggcgatgaac | | 1440 |
| | gcggtcaccg gcaagatgga atgggaagtg ccggagaagt ttgcggtctg gggtggcacc | | 1500 |
| | ttggcgaccg ccggcgacct cgtgttctac ggtaccctcg acggcttcat caaggcccgc | | 1560 |
| | gacacccgta ccggcgagct gaagtggcag ttccagttgc cctccggcgt gatcggccat | | 1620 |
| | cccatcacct atcagcacaa cggcaagcaa tacattgcca tctactccgg cgtcggcggc | | 1680 |
| | tggccaggag tagggctggt attcgacctg aaggacccga ccgcaggtct gggagctgtg | | 1740 |
| | ggtgcgttca gggaactggc gcattacacc cagatgggtg gatcggtgtt cgtgttctcg | | 1800 |
| | ctttga | | 1806 |
| 3-36 | Sequences | | |
| 3-36-1 | Sequence Number [ID] | 36 | |
| 3-36-2 | Molecule Type | AA | |
| 3-36-3 | Length | 601 | |
| | | source 1 . . . 601 | |
| 3-36-4-1 | Features Location/ | mol_type = protein | |
| | Qualifiers | organism = Methylococcus capsulatus | |
| | NonEnglishQualifier Value | | |
| 3-36-5 | Residues | | |
| | MQICKLASGC GGSMLAMAAV LAAQSTHANS ELDRLSKDDR NWVMQTKDYS ATHFSRLTEI | | 60 |
| | NSHNVKNLKV AWTLSTGTLH GHEGAPLVVD GIMYIHTPFP NNVYAVDLND TRKMLWQYKP | | 120 |
| | KQNPAARAVA CCDVVNRGLA YVPAGEHGPA KIFLNQLDGH IVALNAKTGE EIWKMENSDI | | 180 |
| | AMGSTLTGAP FVVKDKVLVG SAGAELGVRG YVTAYNIKDG KQEWRAYATG PDEDLLLDKD | | 240 |
| | FNKDNPHYGQ FGLGLSTWEG DAWKIGGGTN WGWYAYDPKL DMIYYGSGNP APWNETMRPG | | 300 |
| | DNKWTMTIWG RDADTGRAKF GYQKTPHDEW DYAGVNYMGL SEQEVDGKLT PLLTHPDRNG | | 360 |
| | LVYTLNRETG ALVNAFKIDD TVNWVKKVDL KTGLPIRDPE YSTRMDHNAK GICPSAMGYH | | 420 |
| | NQGIESYDPD KKLFFMGVNH ICMDWEPFML PYRAGQFFVG ATLNMYPGPK GMLGQVKAMN | | 480 |
| | AVTGKMEWEV PEKFAVWGGT LATAGDLVFY GTLDGFIKAR DTRTGELKWQ FQLPSGVIGH | | 540 |
| | PITYQHNGKQ YIAIYSGVGG WPGVGLVFDL KDPTAGLGAV GAFRELAHYT QMGGSVFVFS | | 600 |
| | L | | 601 |
| 3-37 | Sequences | | |
| 3-37-1 | Sequence Number [ID] | 37 | |
| 3-37-2 | Molecule Type | DNA | |
| 3-37-3 | Length | 573 | |
| | | source 1 . . . 573 | |
| 3-37-4-1 | Features Location/ | mol_type = other DNA | |
| | Qualifiers | organism = Methylococcus capsulatus | |
| | NonEnglishQualifier Value | | |
| 3-37-5 | Residues | | |
| | atgctcacca gcagtcctta ttaccggtcc ggctacgtat tcgtctaccg caaggacacg | | 60 |
| | ggactgagca tccaagattg gaacagcgcg gcactgaaga ccgtgaagcg gatcgcattc | | 120 |
| | atgccggata ccccggctga gacgatgatc cgcaccatcg gccgctacaa cgacatgttc | | 180 |
| | aactacatgc actctctggt cggtttcaag tcgcggcgta accagtacgt gcgctacgac | | 240 |
| | ccggccaagc tggtggcgga agtcgccgac ggcaacgcgg aagtcgcggt gttgtggggg | | 300 |
| | ccggcggcgg cgcgctatgt cagaggggcg gggctggcca tgaccgtcat ccccgacgac | | 360 |
| | aaccggcggt ccgacggcga gaaagtgccc caccactatt cgacttccgt cggcgtgcgc | | 420 |
| | aagggcgagg aggccctgct caagcagatc gaccaggttc tggcccgctt cggcaaggaa | | 480 |
| | gtgaatgcgg tgctggaggc ggaaggcatt ccgctgttgc ccatggatga aaaaccggcc | | 540 |
| | aggacggctt cccatgatcg aaggaaaggc tag | | 573 |
| 3-38 | Sequences | | |
| 3-38-1 | Sequence Number [ID] | 38 | |
| 3-38-2 | Molecule Type | AA | |
| 3-38-3 | Length | 190 | |
| | | source 1 . . . 190 | |
| 3-38-4-1 | Features Location/ | mol_type = protein | |
| | Qualifiers | organism = Methylococcus capsulatus | |
| | NonEnglishQualifier Value | | |
| 3-38-5 | Residues | | |
| | MLTSSPYYRS GYVFVYRKDT GLSIQDWNSA ALKTVKRIAF MPDTPAETMI RTIGRYNDMF | | 60 |
| | NYMHSLVGFK SRRNQYVRYD PAKLVAEVAD GNAEVAVLWG PAAARYVRGA GLAMTVIPDD | | 120 |
| | NRRSDGEKVP HHYSTSVGVR KGEEALLKQI DQVLARFGKE VNAVLEAEGI PLLPMDEKPA | | 180 |
| | RTASHDRRKG | | 190 |
| 3-39 | Sequences | | |
| 3-39-1 | Sequence Number [ID] | 39 | |
| 3-39-2 | Molecule Type | DNA | |
| 3-39-3 | Length | 480 | |
| | | source 1 . . . 480 | |
| 3-39-4-1 | Features Location/ | mol_type = other DNA | |
| | Qualifiers | organism = Methylococcus capsulatus | |
| | NonEnglishQualifier Value | | |
| 3-39-5 | Residues | | |
| | atgaagctga agaatgcgag gttcgacgtg gctggcatgt gtgtcgccgg gttgttggcg | | 60 |
| | ctgcccgcgc aggccgacat taccctgcgg catgccgtca ccggcgagac gctggagttg | | 120 |
| | tcctacgcca aggcgggcgg cgacacgcaa gccgtcaagc agttcctgca gaccggcaag | | 180 |
| | aacccttaca acggcaacaa ggaggtagtg gaacagggac atagtctgta tctgtcagcc | | 240 |
| | tgttccggct gccacggcca tgaggccgaa ggcaagctcg gtccgggatt ggcggacgac | | 300 |
| | tattggacct atccccgcgc ggccaccgac gtcggtttgt tcgaaatcct gttcggcggc | | 360 |
| | gcgcagggca tgatggggcc gcagtacgtc aacctcaaca atgacgaaat gctcaagatc | | 420 |
| | atggcctgga tccgcagcct ttaccggggc gatccagcca aggccgaatg gctgaaatga | | 480 |
| 3-40 | Sequences | | |
| 3-40-1 | Sequence Number [ID] | 40 | |
| 3-40-2 | Molecule Type | AA | |
| 3-40-3 | Length | 159 | |
| | | source 1 . . . 159 | |
| 3-40-4-1 | Features Location/ | mol_type = protein | |
| | Qualifiers | organism = Methylococcus capsulatus | |
| | NonEnglishQualifier Value | | |
| 3-40-5 | Residues | | |
| | MKLKNARFDV AGMCVAGLLA LPAQADITLR HAVTGETLEL SYAKAGGDTQ AVKQFLQTGK | | 60 |
| | NPYNGNKEVV EQGHSLYLSA CSGCHGHEAE GKLGPGLADD YWTYPRAATD VGLFEILFGG | | 120 |
| | AQGMMGPQYV NLNNDEMLKI MAWIRSLYRG DPAKAEWLK | | 159 |
| 3-41 | Sequences | | |
| 3-41-1 | Sequence Number [ID] | 41 | |
| 3-41-2 | Molecule Type | DNA | |
| 3-41-3 | Length | 285 | |
| | | source 1 . . . 285 | |
| 3-41-4-1 | Features Location/ | mol_type = other DNA | |
| | Qualifiers | organism = Methylococcus capsulatus | |
| | NonEnglishQualifier Value | | |
| 3-41-5 | Residues | | |
| | atgatgcaga aaacgagttt cgtcgcggcc gccatggccg tttcgttcgc ggcgggtgtc | | 60 |
| | caggcctatg acggtaccca ctgcaaggcg cccggaaact gctgggagcc caagcccggt | | 120 |
| | tatccggaca aggtcgccgg cagcaagtac gaccccaagc atgacccgaa cgagctcaac | | 180 |
| | aagcaggcgg agtcgatcaa ggcgatggaa gcccgcaacc agaagcgcgt ggagaactac | | 240 |
| | gccaagaccg gcaagttcgt ctacaaggtc gaagacatca aatga | | 285 |
| 3-42 | Sequences | | |
| 3-42-1 | Sequence Number [ID] | 42 | |
| 3-42-2 | Molecule Type | AA | |
| 3-42-3 | Length | 94 | |
| | | source 1 . . . 94 | |
| 3-42-4-1 | Features Location/ | mol_type = protein | |
| | Qualifiers | organism = Methylococcus capsulatus | |
| | NonEnglishQualifier Value | | |
| 3-42-5 | Residues | | |
| | MMQKTSFVAA AMAVSFAAGV QAYDGTHCKA PGNCWEPKPG YPDKVAGSKY DPKHDPNELN | | 60 |
| | KQAESIKAME ARNQKRVENY AKTGKFVYKV EDIK | | 94 |
| 3-43 | Sequences | | |
| 3-43-1 | Sequence Number [ID] | 43 | |
| 3-43-2 | Molecule Type | DNA | |
| 3-43-3 | Length | 1020 | |
| | | source 1 . . . 1020 | |
| 3-43-4-1 | Features Location/ | mol_type = other DNA | |
| | Qualifiers | organism = Methylococcus capsulatus | |
| | NonEnglishQualifier Value | | |
| 3-43-5 | Residues | | |
| | atgaatctag acacccccct tgccgacggc ctggaacgcg ccaaacgctt cgaacagggg | | 60 |
| | ctgcagcaag tcgtgctcgg gcaggagcgc ccgatccgcc tgctgaccct ggccgtgttc | | 120 |
| | gcccgcggtc atgcgctgct cgaaggcggc gtcggcgtcg ggaagaccac cttgctccgt | | 180 |
| | gcggtggcgc gcggcatcgg cggcgattac gagcggatcg agggcaccat cgacctgatg | | 240 |
| | ccgaacgatc tggtctatta cacctacctg gacgagcaag gtaggccggg cgtcgcgccg | | 300 |
| | gggcctttgc tcaagcacgg ggagcagctt tccatttttt tcttcaacga gatcaaccgc | | 360 |
| | gcccggcccc aggtgcattc cctcctgcta cgggtcatgg ccgagcgcag cgtgtcggct | | 420 |
| | ttcaaccgcg agtaccggtt tccgtacctg caggtgttcg ccgaccgcaa ccgggtggaa | | 480 |
| | aaggaggaga ctttcgaatt gcccgcggcg gcgcgcgacc gcttcatgct cgaaatcgcc | | 540 |
| | atcgagccgc cggccgatcc tgcgcatatc gaccaaatcc tgttcgaccc gcgtttctac | | 600 |
| | gatcccgacc ggctggtcgc gtccgcgccg gccgatacgc tctcgttccg tgaactcaac | | 660 |
| | ggcattgccg aagccctgca aggcggcatc cacgtcagcg cccgtctcag atcctatgtc | | 720 |
| | caggatctgt ggcgcgcgac ccggcggccg gaggatttcg gcatcgctct ccacgaggcg | | 780 |
| | gattccggcg acatgatcga ggccggttcc agtccccgcg gcatgagcta cttggtccgg | | 840 |
| | ctggcgcggg tgcaggcgtg gctcagtggc cgggaccggg tcgagccgga ggacgttcaa | | 900 |
| | tacgtgttcg ctccggcggt cggccaccgc atcttcctca agccggtcta cgaataccgc | | 960 |
| | cgcgccgagc tgatcccgga gctggtcggc aagctgatcc gccggatcgc ggcgccatga | | 1020 |
| 3-44 | Sequences | | |
| 3-44-1 | Sequence Number [ID] | 44 | |
| 3-44-2 | Molecule Type | AA | |
| 3-44-3 | Length | 339 | |
| | | source 1 . . . 339 | |
| 3-44-4-1 | Features Location/ | mol_type = protein | |
| | Qualifiers | organism = Methylococcus capsulatus | |
| | NonEnglishQualifier Value | | |
| 3-44-5 | Residues | | |
| | MNLDTPLADG LERAKRFEQG LQQVVLGQER PIRLLTLAVF ARGHALLEGG VGVGKTTLLR | | 60 |
| | AVARGIGGDY ERIEGTIDLM PNDLVYYTYL DEQGRPGVAP GPLLKHGEQL SIFFFNEINR | | 120 |
| | ARPQVHSLLL RVMAERSVSA FNREYRFPYL QVFADRNRVE KEETFELPAA ARDRFMLEIA | | 180 |
| | IEPPADPAHI DQILFDPRFY DPDRLVASAP ADTLSFRELN GIAEALQGGI HVSARLRSYV | | 240 |
| | QDLWRATRRP EDFGIALHEA DSGDMIEAGS SPRGMSYLVR LARVQAWLSG RDRVEPEDVQ | | 300 |
| | YVFAPAVGHR IFLKPVYEYR RAELIPELVG KLIRRIAAP | | 339 |
| 3-45 | Sequences | | |
| 3-45-1 | Sequence Number [ID] | 45 | |
| 3-45-2 | Molecule Type | DNA | |
| 3-45-3 | Length | 873 | |
| | | source 1 . . . 873 | |
| 3-45-4-1 | Features Location/ | mol_type = other DNA | |
| | Qualifiers | organism = Methylococcus capsulatus | |
| | NonEnglishQualifier Value | | |
| 3-45-5 | Residues | | |
| | gtggtttggt ctctcctgcc ggtcgcggcc ttggtatcgg ttccacttca tggcgccact | | 60 |
| | tcgctctcgt tcgacacgcc ccgcgccttc ggctacgtca tcggtgatct catccgccac | | 120 |
| | gaggttcggg tcgaaaccga tgcggggcag ggaatagagg ctgcgtccct gcccaaggaa | | 180 |
| | ggctggatca accgctggct gctgctgcgg cgggtcgaag tccgccgcga gggcaggcac | | 240 |
| | cggatactga cgctggaata ccagactttc tacgccccgt tggaagtgaa gaacctcacg | | 300 |
| | attcccggct tcgagctgca actggccggt tcgggcgaac ggttggcggt cccggactgg | | 360 |
| | actttcacca ccgcgccgat ccgggagctg tcggtgctgc gcgccgaagg cccgtcgatg | | 420 |
| | cgtccggacg ccgcaccggc gccgctgccg actctcggcc ccgccgccgc gagcgtcggt | | 480 |
| | tccggcctcg cagccacggg cgcgctggcc tggtgggcct atctgagcgc ctggctgccg | | 540 |
| | ttcgtgtcgc gcggccgtca tttcgccgag gcccgccggg tgctgcggga tctgcgcggc | | 600 |
| | ctgggagaca gccgggaggc attgcgcaga ggtttttcct gtctgcacca ggctttcaat | | 660 |
| | cggacttcgg gtgagccgct gttcatcgaa gggctggacg agttcttccg gagccatccg | | 720 |
| | gcctacgatc tcttgcggga cgagatccag gacttcttcc tggcctcgta tgaagtcttt | | 780 |
| | ttcggagagg gcgcaccggc gccgtcgttc gacctggcgc gcatggaggc gttggcccgt | | 840 |
| | tcgtgccagc ttgccgaaag gaggcggcca tga | | 873 |
| 3-46 | Sequences | | |
| 3-46-1 | Sequence Number [ID] | 46 | |
| 3-46-2 | Molecule Type | AA | |
| 3-46-3 | Length | 290 | |
| | | source 1 . . . 290 | |
| 3-46-4-1 | Features Location/ | mol_type = protein | |
| | Qualifiers | organism = Methylococcus capsulatus | |
| | NonEnglishQualifier Value | | |
| 3-46-5 | Residues | | |
| | VVWSLLPVAA LVSVPLHGAT SLSFDTPRAF GYVIGDLIRH EVRVETDAGQ GIEAASLPKE | | 60 |
| | GWINRWLLLR RVEVRREGRH RILTLEYQTF YAPLEVKNLT IPGFELQLAG SGERLAVPDW | | 120 |
| | TFTTAPIREL SVLRAEGPSM RPDAAPAPLP TLGPAAASVG SGLAATGALA WWAYLSAWLP | | 180 |
| | FVSRGRHFAE ARRVLRDLRG LGDSREALRR GFSCLHQAFN RTSGEPLFIE GLDEFFRSHP | | 240 |
| | AYDLLRDEIQ DFFLASYEVF FGEGAPAPSF DLARMEALAR SCQLAERRRP | | 290 |
| 3-47 | Sequences | | |
| 3-47-1 | Sequence Number [ID] | 47 | |
| 3-47-2 | Molecule Type | DNA | |
| 3-47-3 | Length | 984 | |
| | | source 1 . .. 984 | |
| 3-47-4-1 | Features Location/ | mol_type = other DNA | |
| | Qualifiers | organism = Methylococcus capsulatus | |
| | NonEnglishQualifier Value | | |
| 3-47-5 | Residues | | |
| | atgaccgatt gggcgctgga cacgccgtac ttgctgtggg gcctgccgct ggcgctgctt | | 60 |
| | ccgttgtggc ggttgccgct gcgccctgcc ccgtgttcct ggcatgcatt gttgcccgcc | | 120 |
| | gatactgcgt cgcgggccgt cgacctgagt ctgcgcctcg ccggtgccgg cgccatcctg | | 180 |
| | gcgctgctgc tgggcagtgc cggtctgcat cggcgcgagt acaccgtcga acgcaccggc | | 240 |
| | tacggcgccc acatggtgct gctgctggac cgcagccgca gcatggatga cagcttcgca | | 300 |
| | gggcgtactc ccacgggcgg cgaggaatcc aagtccgccg cggcggagcg cctcctgagc | | 360 |
| | ggtttcgtct cgagcggacg caacgatctg gtcggggtcg ccgccttcag cacctccccg | | 420 |
| | ttgttcgtgc tgccgctgac cgacaacaag gctgcggtgc tggcggcggt ccacgccatg | | 480 |
| | aagctgccgg gtctggcgca gacgcatgtg agcaaggggc tggcgatggc gctttcgtat | | 540 |
| | ttcggcgacg attcgaccgc gggttcgcgt atcgtcctgc tggtgtccga cggtgccgcc | | 600 |
| | gaggtggacc cggacagcga gctgaagctg cgccgctggt tcaaggagaa gggcgtacgg | | 660 |
| | ctgtactgga tattcctgcg caccgcgggc agccacggta tcttcgaaac tccggacaac | | 720 |
| | ccggaggaag acaacgccca ggcgcggccc gagcgctatc tgcatctgtt tttcaacagt | | 780 |
| | ctgggcatcc cctaccgcgc ctacgaggcg gaagacgccg acgccctcaa gcgcgccatc | | 840 |
| | gccgacgtcg accgcgagga gcagcggccg ctgcgctatg ccgagcgggt gccgcggcgg | | 900 |
| | gatctgcaag ccttttgtta tctggcggcg gcgctggctc tggcctggct ggtcgccgcg | | 960 |
| | aagggcatgg aggtggcgcg atga | | 984 |
| 3-48 | Sequences | | |
| 3-48-1 | Sequence Number [ID] | 48 | |
| 3-48-2 | Molecule Type | AA | |
| 3-48-3 | Length | 327 | |
| | | source 1 . . . 327 | |
| 3-48-4-1 | Features Location/ | mol_type = protein | |
| | Qualifiers | organism = Methylococcus capsulatus | |
| | NonEnglishQualifier Value | | |
| 3-48-5 | Residues | | |
| | MTDWALDTPY LLWGLPLALL PLWRLPLRPA PCSWHALLPA DTASRAVDLS LRLAGAGAIL | | 60 |
| | ALLLGSAGLH RREYTVERTG YGAHMVLLLD RSRSMDDSFA GRTPTGGEES KSAAAERLLS | | 120 |
| | GFVSSGRNDL VGVAAFSTSP LFVLPLTDNK AAVLAAVHAM KLPGLAQTHV SKGLAMALSY | | 180 |
| | FGDDSTAGSR IVLLVSDGAA EVDPDSELKL RRWFKEKGVR LYWIFLRTAG SHGIFETPDN | | 240 |
| | PEEDNAQARP ERYLHLFFNS LGIPYRAYEA EDADALKRAI ADVDREEQRP LRYAERVPRR | | 300 |
| | DLQAFCYLAA ALALAWLVAA KGMEVAR | | 327 |
| 3-49 | Sequences | | |
| 3-49-1 | Sequence Number [ID] | 49 | |
| 3-49-2 | Molecule Type | DNA | |
| 3-49-3 | Length | 519 | |
| | | source 1 . . . 519 | |
| 3-49-4-1 | Features Location/ | mol_type = other DNA | |
| | Qualifiers | organism = Methylococcus capsulatus | |
| | NonEnglishQualifier Value | | |
| 3-49-5 | Residues | | |
| | atgctggcct tgtcggcgtt gctggagctc aggcaatggc ggaaggccgc ggcggccaat | | 60 |
| | gccgatatcg ccgagctgct gggggggcac gacatcgccc cggaacggct ggcggcggca | | 120 |
| | tcgccccaag tcctgttggc gcgggccgtg tatttcgtgc ggcacgagcg ctacggcgac | | 180 |
| | gcgctggagc tgctgaacct gctggagacc cggggcgatg gcgccttccg cgccgacgtg | | 240 |
| | tattacaacc agggcaatct gcagcttgcc caggctctgg accgcgtcga aaaatcggaa | | 300 |
| | atggaccagg cccgggtctt cgccgaactg gccaaggaag cctaccggcg tgccttgtcg | | 360 |
| | ctggcacccg gccactggga cgccaaatac aacctggaag tggccatgcg cctcatgccc | | 420 |
| | gaaatggacc gggtcagccc tgccgatgac gaggcgcccg cggctgaatc caaacggctg | | 480 |
| | tggacaggtt tgcccggact cccgcgaggc ctgccttga | | 519 |
| 3-50 | Sequences | | |
| 3-50-1 | Sequence Number [ID] | 50 | |
| 3-50-2 | Molecule Type | AA | |
| 3-50-3 | Length | 172 | |
| | | source 1 . . . 172 | |
| 3-50-4-1 | Features Location/ | mol_type = protein | |
| | Qualifiers | organism = Methylococcus capsulatus | |
| | NonEnglishQualifier Value | | |
| 3-50-5 | Residues | | |
| | MLALSALLEL RQWRKAAAAN ADIAELLGGH DIAPERLAAA SPQVLLARAV YFVRHERYGD | | 60 |
| | ALELLNLLET RGDGAFRADV YYNQGNLQLA QALDRVEKSE MDQARVFAEL AKEAYRRALS | | 120 |
| | LAPGHWDAKY NLEVAMRLMP EMDRVSPADD EAPAAESKRL WTGLPGLPRG LP | | 172 |
| 3-51 | Sequences | | |
| 3-51-1 | Sequence Number [ID] | 51 | |
| 3-51-2 | Molecule Type | DNA | |
| 3-51-3 | Length | 972 | |
| | | source 1 . . . 972 | |
| 3-51-4-1 | Features Location/ | mol_type = other DNA | |
| | Qualifiers | organism = Methylococcus capsulatus | |
| | NonEnglishQualifier Value | | |
| 3-51-5 | Residues | | |
| | ttgagcatct ggcggcagcg cgttgccgat ccggtttttg ccggcctgat tgtagccctc | | 60 |
| | cttctggcgg tagccgcctg tttcccgctc cggctggtgc tggagcggct ggtgttcagc | | 120 |
| | cacatcgtcg tcgtcgacat cacccgcagc atgaacgtcg aggactaccg gcgaggcgcg | | 180 |
| | cgcgccgtgt cgcggctgga attcgtcagg cagagcctga tcggcgccgt ggccgacctg | | 240 |
| | ccctgcggct ccgctgtggg ggtgggcgtt ttcaccgaac gcgagccggc gctactgttc | | 300 |
| | gagccgatcg aaacctgcgc cggcttttcc gccatcagcg ccgccatcga acagctcgac | | 360 |
| | tggcgcatgg cctgggctgc cgacagtctg atcgccgcag gtctgcacaa caccctggat | | 420 |
| | ttgctggggc gcggcgatgc ggacgtgatt ttcgtcaccg acggccatga ggcgccgcca | | 480 |
| | ctcaatcccc gctactgccc ggacttcagc gacctcagag gcaaggtccg ggggctgatc | | 540 |
| | gtcggagtgg gaggactgag cctctcgccc atccccaagt acgacgagtc ggggcggcgt | | 600 |
| | tcgggcgttt atggcgagga cgaagtcccg cagcgctcga gcttcggcct gtcggagctg | | 660 |
| | ccgcccgagc agatcgaggg ctaccacgcc cgcaacgctc ccttcggcag cgagagagcc | | 720 |
| | gggggcacgg aacatctgtc ccagctcaag gaaggatatt tgcgccagct cgccgaagcc | | 780 |
| | gccggcctgg gctaccaccg cctggaatcg cccgaaggac tgggccgcgc tctcacggca | | 840 |
| | ccggccttgg cgcggcgcca gcggatcgcc acagacgtcc gctggattcc cgccgccctg | | 900 |
| | gcgctcgccg tactgatggc ggtgtatctg cgggtgctgc tgccgcgtcc tggattttca | | 960 |
| | acctcaaact ga | | 972 |
| 3-52 | Sequences | | |
| 3-52-1 | Sequence Number [ID] | 52 | |
| 3-52-2 | Molecule Type | AA | |
| 3-52-3 | Length | 323 | |
| | | source 1 . . . 323 | |
| 3-52-4-1 | Features Location/ | mol_type = protein | |
| | Qualifiers | organism = Methylococcus capsulatus | |
| | NonEnglishQualifier Value | | |
| 3-52-5 | Residues | | |
| | LSIWRQRVAD PVFAGLIVAL LLAVAACFPL RLVLERLVFS HIVVVDITRS MNVEDYRRGA | | 60 |
| | RAVSRLEFVR QSLIGAVADL PCGSAVGVGV FTEREPALLF EPIETCAGFS AISAAIEQLD | | 120 |
| | WRMAWAADSL IAAGLHNTLD LLGRGDADVI FVTDGHEAPP LNPRYCPDFS DLRGKVRGLI | | 180 |
| | VGVGGLSLSP IPKYDESGRR SGVYGEDEVP QRSSFGLSEL PPEQIEGYHA RNAPFGSERA | | 240 |
| | GGTEHLSQLK EGYLRQLAEA AGLGYHRLES PEGLGRALTA PALARRQRIA TDVRWIPAAL | | 300 |
| | ALAVLMAVYL RVLLPRPGFS TSN | | 323 |
| 3-53 | Sequences | | |
| 3-53-1 | Sequence Number [ID] | 53 | |
| 3-53-2 | Molecule Type | DNA | |
| 3-53-3 | Length | 525 | |
| | | source 1 . . . 525 | |
| 3-53-4-1 | Features Location/ | mol_type = other DNA | |
| | Qualifiers | organism = Methylococcus capsulatus | |
| | NonEnglishQualifier Value | | |
| 3-53-5 | Residues | | |
| | atgaaaccga tgctcatcct gaccgcgttg ctgttcgcct ccgtttcgtt ggcgcacgga | | 60 |
| | cccacccccc aaaaggtcgt cgagaccgtg gagatcgcgg ctcccgtgga ccgggtctgg | | 120 |
| | aacgccgtga aggatttcgg tgccatcgcg cagtggaatc ccgctctggc caagagcgaa | | 180 |
| | agcaccggcg gcaacaccac cggcgagaag cgcatcctcc attttcccaa cggcgagcag | | 240 |
| | ctcaccgagg aactcgatgc ctacgacccg gcagcccacg aatacaccta ccggctgggc | | 300 |
| | aaggacaacg tcaaggcgct gccggccagt tcctactccg ccgtgctcaa ggtcaaggcc | | 360 |
| | accgagacgg gcagccagat cgaatggaag agtcggctct atcgcggcga taccggaaac | | 420 |
| | ttcccgccgg acgagctgaa cgacgaggcc gccgttgcgg cgatgcagag gtttttccgc | | 480 |
| | gccgggctgg acaatctcaa gaaaagtctt gggcccctcg aatga | | 525 |
| 3-54 | Sequences | | |
| 3-54-1 | Sequence Number [ID] | 54 | |
| 3-54-2 | Molecule Type | AA | |
| 3-54-3 | Length | 174 | |
| | | source 1 . . . 174 | |
| 3-54-4-1 | Features Location/ | mol_type = protein | |
| | Qualifiers | organism = Methylococcus capsulatus | |
| | NonEnglishQualifier Value | | |
| 3-54-5 | Residues | | |
| | MKPMLILTAL LFASVSLAHG PTPQKVVETV EIAAPVDRVW NAVKDFGAIA QWNPALAKSE | | 60 |
| | STGGNTTGEK RILHFPNGEQ LTEELDAYDP AAHEYTYRLG KDNVKALPAS SYSAVLKVKA | | 120 |
| | TETGSQIEWK SRLYRGDTGN FPPDELNDEA AVAAMQRFFR AGLDNLKKSL GPLE | | 174 |
| 3-55 | Sequences | | |
| 3-55-1 | Sequence Number [ID] | 55 | |
| 3-55-2 | Molecule Type | DNA | |
| 3-55-3 | Length | 1530 | |
| | | source 1 . . . 1530 | |
| 3-55-4-1 | Features Location/ | mol_type = other DNA | |
| | Qualifiers | organism = Methylococcus capsulatus | |
| | NonEnglishQualifier Value | | |
| 3-55-5 | Residues | | |
| | atgctgcaaa aatacataga gaagattctg cgcgcccgtg tctacgacgt tgcccaggag | | 60 |
| | accccgctgg acccggcgcc cggcctgtcg cggcggctgg acaacacggt gctgatcaag | | 120 |
| | cgcgaggacc tgcagccggt gttctcgttc aagctgcgcg gcgcctacaa caagatcgcc | | 180 |
| | tcgctcacac ccgaggcgcg cgcggccggc gtgatcgcgg cctccgccgg caaccacgcc | | 240 |
| | cagggcgtgg cactggcggc gcagcggctg ggcatccgcg ccgtgatcgt gatgccttgc | | 300 |
| | accaccccgc atatcaaggt cgatgcggtg cgcaaccgag gcggtgaggt cgtactgcat | | 360 |
| | ggcgacgcct atgacgaagc ctacgaacat gcgctggaac tggcccgcga ccagtgcctg | | 420 |
| | accttcgtcc acccctacga cgatccggaa gtcatcgccg ggcaaggcac catcggcatg | | 480 |
| | gaaatcctgc gccagcacca ggacgccatc cacgccatct tcgtgcctgt gggcggcggc | | 540 |
| | ggattgatcg ccggcatcgc cgcctacgtc aagttcgtgc gcccggacat ccgcgtcatc | | 600 |
| | ggcgtggaac cagtggactc cgactgcctg caccgggcgc tgaaagccaa gcggcgggtg | | 660 |
| | atcctgaagc aggtgggcct gttcgccgac ggcgtcgcgg tgaagcaggt cggcaaggaa | | 720 |
| | ccgttccatc tcgcccacca gtgggtggac gaggtcgtga ccgtcgacac cgacgaaatc | | 780 |
| | tgcgccgcca tcaaggacat cttcgacgac acccgctcca tcgccgagcc ggcgggcgcg | | 840 |
| | ctgggcatcg ccgggctcaa gaaatacgtg gccgaaacag gaatcaagaa cgcgtgcctg | | 900 |
| | gtggcgatcg aaagcggcgc caacatcaac ttcgaccggc tgcgccacgt cgctgagcgc | | 960 |
| | gccgagatcg gcgaaaagcg cgaactgctg ctggcagtga cgatccccga gcggcccggc | | 1020 |
| | agcttcctcg aattctgccg ggtgctgggc cgccgcaaca tcaccgaatt caactaccgc | | 1080 |
| | ttcttcgacg aaaaggccgc ccaggtgttc gtcggcctcc cggtggcgag cggcgcgatc | | 1140 |
| | gaccgcgaaa gcctggtccg cgaattcgaa cgccagggtt tcggcgtgct cgacctgacc | | 1200 |
| | ggcaacgaac tcgccatcga acacatccgc tacatggtcg gcggccacgc gccgaaactg | | 1260 |
| | ctggacgaac aggtctacag cttcgaattc cccgagcgac ccggcgcgct gctgcgcttc | | 1320 |
| | ctgtccatca tgggcgggcg ctggaacatc agcctgttcc attaccgcaa ccacggcgcc | | 1380 |
| | gccttcggcc gggtactgat gggcatccag gtgccgaaac cggaacgcaa ggccttccgg | | 1440 |
| | gaattcctcg aagccatcgg ctacgccttc aaggaggaaa cccaaaatcc cgcctaccgg | | 1500 |
| | ctgttcgcgg ggggcagcga gcgggggtga | | 1530 |
| 3-56 | Sequences | | |
| 3-56-1 | Sequence Number [ID] | 56 | |
| 3-56-2 | Molecule Type | AA | |
| 3-56-3 | Length | 509 | |
| | | source 1 . . . 509 | |
| 3-56-4-1 | Features Location/ | mol_type = protein | |
| | Qualifiers | organism = Methylococcus capsulatus | |
| | NonEnglishQualifier Value | | |
| 3-56-5 | Residues | | |
| | MLQKYIEKIL RARVYDVAQE TPLDPAPGLS RRLDNTVLIK REDLQPVFSF KLRGAYNKIA | | 60 |
| | SLTPEARAAG VIAASAGNHA QGVALAAQRL GIRAVIVMPC TTPHIKVDAV RNRGGEVVLH | | 120 |
| | GDAYDEAYEH ALELARDQCL TFVHPYDDPE VIAGQGTIGM EILRQHQDAI HAIFVPVGGG | | 180 |
| | GLIAGIAAYV KFVRPDIRVI GVEPVDSDCL HRALKAKRRV ILKQVGLFAD GVAVKQVGKE | | 240 |
| | PFHLAHQWVD EVVTVDTDEI CAAIKDIFDD TRSIAEPAGA LGIAGLKKYV AETGIKNACL | | 300 |
| | VAIESGANIN FDRLRHVAER AEIGEKRELL LAVTIPERPG SFLEFCRVLG RRNITEFNYR | | 360 |
| | FFDEKAAQVF VGLPVASGAI DRESLVREFE RQGFGVLDLT GNELAIEHIR YMVGGHAPKL | | 420 |
| | LDEQVYSFEF PERPGALLRF LSIMGGRWNI SLFHYRNHGA AFGRVLMGIQ VPKPERKAFR | | 480 |
| | EFLEAIGYAF KEETQNPAYR LFAGGSERG | | 509 |
| 3-57 | Sequences | | |
| 3-57-1 | Sequence Number [ID] | 57 | |
| 3-57-2 | Molecule Type | DNA | |
| 3-57-3 | Length | 1545 | |
| | | source 1 . . . 545 | |
| 3-57-4-1 | Features Location/ | mol_type = other DNA | |
| | Qualifiers | organism = Methylococcus capsulatus | |
| | NonEnglishQualifier Value | | |
| 3-57-5 | Residues | | |
| | atgcacgaca gactgatcat tttcgacacg accttgcgcg acggagagca gagccccggc | | 60 |
| | gcgtccatga cccgcgatga aaaggtccgc atcgcccggg cgctggagcg tctgaaggtc | | 120 |
| | gacgtcatcg aggcgggctt tcccgccgcc agccccggcg atttcgaggc cgtccaggcc | | 180 |
| | gtggcccgga ccatcaagga cagcagggtc tgcggcctgg cccgcgccct cgaccgcgac | | 240 |
| | atcgaccgcg ccggcgaagc cctcaaggac gcccagcgcg cccgcatcca caccttcatc | | 300 |
| | gccacctcgc ccatccacat gcggcacaag ctgcagatgt cgcccgacca ggtggtggaa | | 360 |
| | tacgcggtca aggccgtcaa gcgggcccgc cagtacaccg acgacgtgga attctcgccc | | 420 |
| | gaggacgccg gacgctccga ggaggatttc ctctgccgca tcctggaagc cgtgatcgat | | 480 |
| | gcgggggcga ccacgctgaa catccccgac accgtcggct acgccttccc ggaacagttc | | 540 |
| | gggcacatga tcggccggct gatcgagcgg attccgaact ccgacaaggc cgtgttctcg | | 600 |
| | gttcactgcc acaacgacct gggactggcg gtcgccaatt cgctggccgc cgtgctgcac | | 660 |
| | ggcgcgcgcc aggtggaatg caccatcaac gggctgggcg agcgggccgg caacgccgcg | | 720 |
| | ctggaagaga tcgtcatggc ggtgcgcacc cgtaaagaca tcttcccctg ccacaccgac | | 780 |
| | atcgagacac gggaaatcgt ggcctgctcc aaactggtct ccagcatcac cggtttcccg | | 840 |
| | atccagccca acaaggccat cgtcggcgcc aacgccttcg cccacgagtc gggcatccac | | 900 |
| | caggacggtg tgctcaagag ccgggaaacc tacgagatca tgagcgccga ggacgtgggg | | 960 |
| | tggagcacca accgcatggt gctgggcaaa cattccggcc gcaacgcgtt ccgtacccgg | | 1020 |
| | atgcaggaac tcggcatcga gttcgcctcg gaagaggaac tgaactcggt gttccagcgc | | 1080 |
| | ttcaaggtgc tggccgacaa gaagcacgag atcttcgacg aggacctcca ggccctcatc | | 1140 |
| | accgaagccg gcgcagaagc cgaagacgaa cgggtcaagc tggtcgcgct gcgggtctgc | | 1200 |
| | tcggaaacgg gcgagattcc ccacgcccag gtcaccatca aggtggacaa cgaggaacgc | | 1260 |
| | accggcacat cgagcggcgg cggcgccgtg gacgccagcc tcaaggccat cgaatcgctg | | 1320 |
| | ctgcacacgg acaccgcgct gacgctgtac tcggtcaaca acatcaccag cggcaccgac | | 1380 |
| | gcccagggcg aggtcaccgt gcggctcgag aaaggcgggc gcatcgtcaa cggccagggc | | 1440 |
| | gccgataccg acatcgtgat cgcctcggcc aaggcctacg tcaacgccgt gaacaagctg | | 1500 |
| | ctggcgccca tccagcgcac ccacccgcaa gtcggggatg tgtga | | 1545 |
| 3-58 | Sequences | | |
| 3-58-1 | Sequences Number [ID] | 58 | |
| 3-58-2 | Molecule Type | AA | |
| 3-58-3 | Length | 514 | |
| | | source 1 . . . 514 | |
| 3-58-4-1 | Features Location/ | mol_type = protein | |
| | Qualifiers | organism = Methylococcus capsulatus | |
| | NonEnglishQualifier Value | | |
| 3-58-5 | Residues | | |
| | MHDRLIIFDT TLRDGEQSPG ASMTRDEKVR IARALERLKV DVIEAGFPAA SPGDFEAVQA | | 60 |
| | VARTIKDSRV CGLARALDRD IDRAGEALKD AQRARIHTFI ATSPIHMRHK LQMSPDQVVE | | 120 |
| | YAVKAVKRAR QYTDDVEFSP EDAGRSEEDF LCRILEAVID AGATTLNIPD TVGYAFPEQF | | 180 |
| | GHMIGRLIER IPNSDKAVFS VHCHNDLGLA VANSLAAVLH GARQVECTIN GLGERAGNAA | | 240 |
| | LEEIVMAVRT RKDIFPCHTD IETREIVACS KLVSSITGFP IQPNKAIVGA NAFAHESGIH | | 300 |
| | QDGVLKSRET YEIMSAEDVG WSTNRMVLGK HSGRNAFRTR MQELGIEFAS EEELNSVFQR | | 360 |
| | FKVLADKKHE IFDEDLQALI TEAGAEAEDE RVKLVALRVC SETGEIPHAQ VTIKVDNEER | | 420 |
| | TGTSSGGGAV DASLKAIESL LHTDTALTLY SVNNITSGTD AQGEVTVRLE KGGRIVNGQG | | 480 |
| | ADTDIVIASA KAYVNAVNKL LAPIQRTHPQ VGDV | | 514 |
| 3-59 | Sequences | | |
| 3-59-1 | Sequences Number [ID] | 59 | |
| 3-59-2 | Molecule Type | DNA | |
| 3-59-3 | Length | 1413 | |
| | | source 1 . . . 413 | |
| 3-59-4-1 | Features Location/ | mol_type = other DNA | |
| | Qualifiers | organism = Methylococcus capsulatus | |
| | NonEnglishQualifier Value | | |
| 3-59-5 | Residues | | |
| | atgagcggaa aaacccttta cgacaagctg tgggacgacc acgtcgtgca tgtcgatgcg | | 60 |
| | gacggatcgt gcctgatcta catcgatcgt catctaatcc acgaggtgac ctcgcctcag | | 120 |
| | gcattcgaag ggctgcggat ggcggggcgt gtaccctggc gggtggatgc caatcttgcg | | 180 |
| | gtggccgacc acaacgtccc caccgccgac cgcgacaggg gtatcgccga tccggtgtcg | | 240 |
| | cgcctgcagg tggaaaccct ggacaagaac tgcgccgatt tcggcatcac cgaattcgcg | | 300 |
| | atggacgacg tgcgccaggg tatcgtgcat gtgatcgggc ccgagcaggg cgcgaccctg | | 360 |
| | ccgggcatga ccatcgtttg cggcgattcg catacttcga ctcacggtgc tttcggggcg | | 420 |
| | ctcgccttcg ggatcggcac ttccgaggtc gagcacgtac tggccacgca atgcctggtg | | 480 |
| | cagcgcaagg cgaagaacat gctggtccgc gtccagggca agctggcgcc gggcgtgacg | | 540 |
| | gcgaaagatc tggtactggc ggtcatcggc cgtatcggaa ccgccggcgg caccggctac | | 600 |
| | accatcgaat tcgctggcga agccattcgc ggcctgtcga tggaaggccg gatgacggtc | | 660 |
| | tgcaacatgg cgatcgaggc gggcgcacgt gccggcctgg tggcggtgga cgaagtcacg | | 720 |
| | ctcgactatc tcgagggccg cccgttcgct ccggcgggcg cgttgtggga gcgggcggtc | | 780 |
| | gaggcatgga aagacctgca cagcgatccg gatgcggtat tcgacaaggt cgtcgagatc | | 840 |
| | gatgccgcca gcatcaagcc gcaggtgacc tggggaactt cgccggaaca ggtcgtgccg | | 900 |
| | gtggatgccg aggtgcccga cccggccacg gaagccgatc ccgtgcggcg ggaaagcatg | | 960 |
| | gagcgggcgc tgcagtacat ggatctcctg ccgggcacgc caatcggcgc gatccgggtc | | 1020 |
| | gatcgggtgt tcatcggctc ctgcaccaat gccaggatcg aggatctgcg cgccgcggcg | | 1080 |
| | gaagtcgtcc gggggcacaa gcgcgctgcc agcgtgaagc aggcactggt ggtgcccggc | | 1140 |
| | tcgggtttgg tcaagcggca ggcggagcag gaggggctgg acaaggtgtt cctcgaggcc | | 1200 |
| | ggtttcgaat ggcgcgaccc gggttgttcc atgtgtctgg cgatgaacgc cgaccgcctg | | 1260 |
| | gaacccggcg agcgttgcgc ctcgacctcc aaccggaatt ttgaggggcg ccagggctat | | 1320 |
| | ggcgggcgta cccatctggt gagtccggcc atggcggctg cggcggccat tcacgggcat | | 1380 |
| | ttcgtcgaca tcaccgaagg agggcgcgca tga | | 1413 |
| 3-60 | Sequences | | |
| 3-60-1 | Sequences Number [ID] | 60 | |
| 3-60-2 | Molecule Type | AA | |
| 3-60-3 | Length | 470 | |
| | | source 1 . . . 470 | |
| 3-60-4-1 | Features Location/ | mol_type = protein | |
| | Qualifiers | organism = Methylococcus capsulatus | |
| | NonEnglishQualifier Value | | |
| 3-60-5 | Residues | | |
| | MSGKTLYDKL WDDHVVHVDA DGSCLIYIDR HLIHEVTSPQ AFEGLRMAGR VPWRVDANLA | | 60 |
| | VADHNVPTAD RDRGIADPVS RLQVETLDKN CADFGITEFA MDDVRQGIVH VIGPEGGATL | | 120 |
| | PGMTIVCGDS HTSTHGAFGA LAFGIGTSEV EHVLATQCLV QRKAKNMLVR VQGKIAPGVT | | 180 |
| | AKDLVLAVIG RIGTAGGTGY TIEFAGEAIR GLSMEGRMTV CNMAIEAGAR AGLVAVDEVT | | 240 |
| | LDYLEGRPFA PAGALWERAV EAWKDLHSDP DAVFDKVVEI DAASIKPQVT WGTSPEQVVP | | 300 |
| | VDAEVPDPAT EADPVRRESM ERALQYMDLL PGTPIGAIRV DRVFIGSCTN ARIEDLRAAA | | 360 |
| | EVVRGHKRAA SVKQALVVPG SGLVKRQAEQ EGLDKVFLEA GFEWRDPGCS MCLAMNADRL | | 420 |
| | EPGERCASTS NRNFEGRQGY GGRTHLVSPA MAAAAAIHGH FVDIEGGRA | | 470 |
| 3-61 | Sequences | | |
| 3-61-1 | Sequences Number [ID] | 61 | |
| 3-61-2 | Molecule Type | DNA | |
| 3-61-3 | Length | 639 | |
| | | source 1 . . . 639 | |
| 3-61-4-1 | Features Location/ | mol_type = other DNA | |
| | Qualifiers | organism = Methylococcus capsulatus | |
| | NonEnglishQualifier Value | | |
| 3-61-5 | Residues | | |
| | atgaagcctt tcaagaaatt cacttcgcga gtcgtgccgt tggaccgcgc caatgtcgac | | 60 |
| | accgacgcca tcattcccaa gcagttcctg aagtccatcc gccgcagcgg gttcggtccc | | 120 |
| | tatctgttcg acgagtggcg ttacctggac cgtggcgagc ccgacatgga ttgcagccac | | 180 |
| | cgtccgctca acccggagtt cgtgctcaac ctgccctgtt acgccggcgc caggatattg | | 240 |
| | ctggcccgca agaacttcgg ctgtggctcc tcgcgcgagc atgcgccctg ggcgctggag | | 300 |
| | gattacggct tccgcgccat catcgcgccg agtttcgccg atatcttcta caacaactgc | | 360 |
| | ttcaagaacg gcatcctgcc catcgtgctc gacgaggcca cggtcgaccg gctgtttagc | | 420 |
| | gaggccgggc ccggcttcga gctcaccgtc gacctggagt cgcagaccgt ggcgacgccg | | 480 |
| | ttcggcgaga ccttccattt cgacgtggat gcctcccgca agcatcgtct gctgaacggc | | 540 |
| | ctggacgaca tcggtctgac ccttcagcat gccgatgcca tccgcgccta cgaagccgcc | | 600 |
| | cgcaggaagt ccgcaccctg gctgtttgcc gtcccttga | | 639 |
| 3-62 | Sequences | | |
| 3-62-1 | Sequences Number [ID] | 62 | |
| 3-62-2 | Molecule Type | AA | |
| 3-62-3 | Length | 212 | |
| | | source 1 . . . 212 | |
| 3-62-4-1 | Features Location/ | mol_type = protein | |
| | Qualifiers | organism = Methylococcus capsulatus | |
| | NonEnglishQualifier Value | | |
| 3-62-5 | Residues | | |
| | MKPFKKFTSR VVPLDRANVD TDAIIPKQFL KSIRRSGFGP YLFDEWRYLD RGEPDMDCSH | | 60 |
| | RPLNPEFVLN LPCYAGARIL LARKNFGCGS SREHAPWALE DYGFRAIIAP SFADIFYNNC | | 120 |
| | FKNGILPIVL DEATVDRLFS EAGPGFELTV DLESQTVATP FGETFHFDVD ASRKHRLLNG | | 180 |
| | LDDIGLTLQH ADAIRAYEAA RRKSAPWLFA VP | | 212 |
| 3-63 | Sequences | | |
| 3-63-1 | Sequence Number [ID] | 63 | |
| 3-63-2 | Molecule Type | DNA | |
| 3-63-3 | Length | 1083 | |
| | | source 1 . . . 1083 | |
| 3-63-4-1 | Features Location/ | mol_type = other DNA | |
| | Qualifiers | organism = Methylococcus capsulatus | |
| | NonEnglishQualifier Value | | |
| 3-63-5 | Residues | | |
| | atgactatca aaatcgctgt cttgcccggt gacggcatcg gtcccgaaat cgtcgccgag | | 60 |
| | gccctgaagg ttctggactg cctgcggtcc gacttcggcc tcgcggtcga aaccgaacac | | 120 |
| | gccctgatcg gcggcgcagc ctatgatgcg cacggcacgc cgttccccaa ggaaaccctg | | 180 |
| | gagctgtgcc gggctgccga ttcgatcctg cttggagcgg tcggcggtcc caaatgggag | | 240 |
| | ccgttggatt attcgctgcg gcccgagcgg gggctcctgg gcttgcgttc ggagctggaa | | 300 |
| | ctgttttcca acctgcgccc ggcggtgctc taccctcagc tggtgtcggc ttcgaccctc | | 360 |
| | aagcccgagg tggtcgccgg cctcgacatc atgatcgtgc gggagctgac cggcggcata | | 420 |
| | tatttcggca agccgcgcgg tcgtcgcatc aacgaggacg gagagcggga gggctacaac | | 480 |
| | accctggtat acagcgaatc ggaaatccgc cgcatagccc atagcgcgtt ccagatcgcc | | 540 |
| | cggaagcgta acaggcgcct gtgcagcatc gacaaggcca atgtgctgga atgcacggaa | | 600 |
| | ctgtggcgcg aggtggtgat cgaggtcggc aaggactatc ccgacgtggc gctgagccac | | 660 |
| | atgtacgtgg acaacgccgc gatgcagctg gtccgtaacc cgaagcagtt cgacgtgatg | | 720 |
| | ctgaccgaca acatgttcgg cgacatcctg tccgactgtg ccgccatgct gaccggctcg | | 780 |
| | atcggcatgc tgccttcggc ttccctcgcc gagagcggca aggggatgta cgagcccatc | | 840 |
| | cacggttcgg ccccggatat cgccggccgc ggcatcgcca acccgatcgc caccatcctg | | 900 |
| | tcgctggcca tgatgttgcg ctacagcttc gatgacgcgg tctcggcaga gcggatcggg | | 960 |
| | aaggcggtgc agacggcgct ggatcagggt ttccgcacgg cggacatcgc ctcggaaggc | | 1020 |
| | accgtcgagg tcggtaccgc tgcgatgggc gatgccatcg tcgccgcctt gcgcgccgtc | | 1080 |
| | tga | | 1083 |
| 3-64 | Sequences | | |
| 3-64-1 | Sequence Number [ID] | 64 | |
| 3-64-2 | Molecule Type | AA | |
| 3-64-3 | Length | 360 | |
| | | source 1 . . . 360 | |
| 3-64-4-1 | Features Location/ | mol_type = protein | |
| | Qualifiers | organism = Methylococcus capsulatus | |
| | NonEnglishQualifier Value | | |
| 3-64-5 | Residues | | |
| | MTIKIAVLPG DGIGPEIVAE ALKVLDCLRS DFGLAVETEH ALIGGAAYDA HGTPFPKETL | | 60 |
| | ELCRAADSIL LGAVGGPKWE PLDYSLRPER GLLGLRSELE LFSNLRPAVL YPQLVSASTL | | 120 |
| | KPEVVAGLDI MIVRELTGGI YFGKPRGRRI NEDGEREGYN TLVYSESEIR RIAHSAFQIA | | 180 |
| | RKRNRRLCSI DKANVLECTE LWREVVIEVG KDYPDVALSH MYVDNAAMQL VRNPKQFDVM | | 240 |
| | LTDNMFGDIL SDCAAMLTGS IGMLPSASLA ESGKGMYEPI HGSAPDIAGR GIANPIATIL | | 300 |
| | SLAMMLRYSF DDAVSAERIG KAVQTALDQG FRTADIASEG TVEVGTAAMG DAIVAALRAV | | 360 |
| 3-65 | Sequences | | |
| 3-65-1 | Sequence Number [ID] | 65 | |
| 3-65-2 | Molecule Type | DNA | |
| 3-65-3 | Length | 1477 | |
| | | source 1 . . . 1477 | |
| 3-65-4-1 | Features Location/ | mol_type = other DNA | |
| | Qualifiers | organism = Marinobacter algicola | |
| | NonEnglishQualifier Value | | |
| 3-65-5 | Residues | | |
| | atggcgaccc agcagcagca gaacggcgcc tcggcgagcg gcgtcctgga acagttgcgc | | 60 |
| | gggaagcatg tcctgataac cggtaccacc ggtttccttg gcaaggtagt cctggaaaag | | 120 |
| | ctgatccgca cagtcccgga catcggcggc atccacctcc tgatccgggg caacaagagg | | 180 |
| | catccggccg cccgtgaacg gttcttgaac gagatcgcca gcagttcggt cttcgagcgt | | 240 |
| | ctgcgccacg acgacaacga ggccttcgaa accttcctgg aagaaagggt gcactgtata | | 300 |
| | accggagagg tcaccgagag tcgtttcggc cttaccccgg agcgcttccg cgcgctggcg | | 360 |
| | ggtcaggtgg acgccttcat caattcggcc gcctccgtca acttccgcga ggaactggac | | 420 |
| | aaggcgctga agatcaatac gctgtgcctg gagaatgtcg cggcccttgc tgaactcaac | | 480 |
| | agtgcgatgg cggtcatcca ggtttcgacc tgctacgtta acggcaagaa tagcgggcag | | 540 |
| | atcaccgaat cggtcatcaa gcccgcgggg gagtccatcc cgcgtagcac cgatgggtac | | 600 |
| | tatgaaatcg aagaattggt gcacctgctg caggacaaaa tcagcgatgt gaaggcccga | | 660 |
| | tactccggga aggttctgga aaaaaaattg gtggacctag gcatccggga agccaataac | | 720 |
| | tacgggtgga gcgatacata taccttcacc aagtggctgg gcgaacagct cctcatgaag | | 780 |
| | gccctgagcg gcagatcgct gaccatcgtg cggccgtcga tcatcgagtc ggcattggaa | | 840 |
| | gagcccagcc cggggtggat tgaaggcgtc aaggtcgccg atgccatcat actggcctac | | 900 |
| | gcgagggaga aggtatcgct ctttcctggc aagcggagcg gcatcatcga cgtcatccca | | 960 |
| | gtggatctgg tggccaattc gatcattctg tccctggcgg aggcgctctc cggttcgggc | | 1020 |
| | cagcggcgta tctatcagtg ctgcagcggc ggctcgaacc ccatctccct cgggaagttc | | 1080 |
| | atcgactatc tgatggcgga ggcgaagacc aactacgcgg cctacgatca gctgttctac | | 1140 |
| | cgccgcccca ccaagccgtt cgtggccgtc aaccgcaaac tcttcgacgt cgtcgtgggc | | 1200 |
| | ggcatgcggg tcccgctctc gatcgcgggc aaagccatgc gcctggcggg acaaaaccgc | | 1260 |
| | gaactgaagg tcctgaagaa tctggatacg acccggtccc tggccaccat tttcgggttc | | 1320 |
| | tacaccgctc cggactacat ctttcgcaat gacagcctga tggccctggc ctcgcgcatg | | 1380 |
| | ggcgagctgg accgcgtgtt gttccccgtt gacgcccgtc agatcgactg gcagctgtat | | 1440 |
| | ctgtgcaaaa tccacctcgg cgggctgaat cggtacg | | 1477 |
| 3-66 | Sequences | | |
| 3-66-1 | Sequence Number [ID] | 66 | |
| 3-66-2 | Molecule Type | AA | |
| 3-66-3 | Length | 55 | |
| | | source 1 . . . 511 | |
| 3-66-4-1 | Features Location/ | mol_type = protein | |
| | Qualifiers | organism = Marinobacter algicola | |
| | NonEnglishQualifier Value | | |
| 3-66-5 | Residues | | |
| | MATQQQQNGA SASGVLEQLR GKHVLITGTT GFLGKVVLEK LIRTVPDIGG IHLLIRGNKR | | 60 |
| | HPAARERFLN EIASSSVFER LRHDDNEAFE TFLEERVHCI TGEVTESRFG LTPERFRALA | | 120 |
| | GQVDAFINSA ASVNFREELD KALKINTLCL ENVAALAELN SAMAVIQVST CYVNGKNSGQ | | 180 |
| | ITESVIKPAG ESIPRSTDGY YEIEELVHLL QDKISDVKAR YSGKVLEKKL VDLGIREANN | | 240 |
| | YGWSDTYTFT KWLGEQLLMK ALSGRSLTIV RPSIIESALE EPSPGWIEGV KVADAIILAY | | 300 |
| | AREKVSLFPG KRSGIIDVIP VDLVANSIIL SLAEALSGSG QRRIYQCCSG GSNPISLGKF | | 360 |
| | IDYLMAEAKT NYAAYDQLFY RRPTKPFVAV NRKLFDVVVG GMRVPLSIAG KAMRLAGQNR | | 420 |
| | ELKVLKNLDT TRSLATIFGF YTAPDYIFRN DSLMALASRM GELDRVLFPV DARQIDWQLY | | 480 |
| | LCKIHLGGLN RYALKERKLY SLRAADTRKK A | | 511 |
| 3-67 | Sequences | | |
| 3-67-1 | Sequence Number [ID] | 67 | |
| 3-67-2 | Molecule Type | DNA | |
| 3-67-3 | Length | 1377 | |
| | | source 1 . . . 1377 | |
| 3-67-4-1 | Features Location/ | mol_type = other DNA | |
| | Qualifiers | organism = Acinetobacter baylyi | |
| | NonEnglishQualifier Value | | |
| 3-67-5 | Residues | | |
| | atgcgccccc tgcaccccat cgacttcatc ttcctgagcc tggaaaagcg gcagcagccc | | 60 |
| | atgcacgtcg gcggcctgtt cctgttccag atcccggaca acgcccccga caccttcatc | | 120 |
| | caggacctgg tcaacgacat ccgcatctcc aagagcatcc cggtgccgcc cttcaacaac | | 180 |
| | aagctgaacg gcctgttctg ggacgaagac gaggagttcg acctggacca ccatttccgg | | 240 |
| | cacatcgccc tgccgcatcc cggccgcatc cgggaactgc tgatctacat ctcccaggag | | 300 |
| | cacagcaccc tgctggaccg cgcgaagccg ctgtggacct gcaacatcat cgaaggcatc | | 360 |
| | gagggcaacc ggttcgccat gtatttcaag atccaccatg cgatggtcga cggcgtggcc | | 420 |
| | ggcatgcgcc tgatcgaaaa gtcgctgtcc catgacgtca ccgagaagag catcgtcccg | | 480 |
| | ccctggtgcg tggaaggcaa gcgggcgaag cgcctgcggg agccgaagac cggcaagatc | | 540 |
| | aagaagatca tgtcgggcat caagtcccag ctgcaggcca cccccaccgt catccaggaa | | 600 |
| | ctgtcgcaga ccgtgttcaa ggacatcggc cgcaacccgg accacgtcag ctcgttccag | | 660 |
| | gccccctgct ccatcctgaa ccagcgggtg tccagctcgc gccggttcgc cgcgcagtcg | | 720 |
| | ttcgacctgg accgcttccg gaacatcgcg aagtccctga acgtcaccat caacgacgtc | | 780 |
| | gtgctggccg tgtgcagcgg cgccctgcgc gcgtacctga tgagccacaa ctcgctgccg | | 840 |
| | tccaagcccc tgatcgcgat ggtcccggcg tcgatccgca acgacgacag cgacgtgtcg | | 900 |
| | aaccggatca ccatgatcct ggccaacctg gcgacccata aggacgaccc gctgcagcgc | | 960 |
| | ctggagatca tccgccggag cgtccagaac tcgaagcagc gcttcaagcg gatgacctcc | | 1020 |
| | gaccagatcc tgaactacag cgcggtcgtg tatggcccgg ccggcctgaa catcatcagc | | 1080 |
| | ggcatgatgc ccaagcgcca ggccttcaac ctggtcatct cgaacgtgcc gggcccgcgc | | 1140 |
| | gagccgctgt actggaacgg cgccaagctg gacgcgctgt atcccgcctc catcgtcctg | | 1200 |
| | gacggccagg ccctgaacat caccatgacc agctacctgg acaagctgga ggtcggcctg | | 1260 |
| | atcgcgtgcc gcaacgccct gccgcggatg cagaacctgc tgacccatct ggaggaagag | | 1320 |
| | atccagctgt tcgaaggcgt gatcgcgaag caggaggaca tcaagaccgc caactga | | 1377 |
| 3-68 | Sequences | | |
| 3-68-1 | Sequence Number [ID] | 68 | |
| 3-68-2 | Molecule Type | AA | |
| 3-68-3 | Length | 458 | |
| | | source 1 . . . 458 | |
| 3-68-4-1 | Features Location/ | mol_type = protein | |
| | Qualifiers | organism = Acinetobacter baylyi | |
| | NonEnglishQualifier Value | | |
| | NonEnglishQualifier Value | NonEnglishQualifier Value | |
| 3-68-5 | Residues | | |
| | MRPLHPIDFI FLSLEKRQQP MHVGGLFLFQ IPDNAPDTFI QDLVNDIRIS KSIPVPPFNN | | 60 |
| | KLNGLFWDED EEFDLDHHFR HIALPHPGRI RELLIYISQE HSTLLDRAKP LWTCNIIEGI | | 120 |
| | EGNRFAMYFK IHHAMVDGVA GMRLIEKSLS HDVTEKSIVP PWCVEGKRAK RLREPKTGKI | | 180 |
| | KKIMSGIKSQ LQATPTVIQE LSQTVEKDIG RNPDHVSSFQ APCSILNQRV SSSRRFAAQS | | 240 |
| | FDLDRFRNIA KSLNVTINDV VLAVCSGALR AYLMSHNSLP SKPLIAMVPA SIRNDDSDVS | | 300 |
| | NRITMILANL ATHKDDPLQR LEIIRRSVQN SKQRFKRMTS DQILNYSAVV YGPAGLNIIS | | 360 |
| | GMMPKRQAFN LVISNVPGPR EPLYWNGAKL DALYPASIVL DGQALNITMT SYLDKLEVGL | | 420 |
| | IACRNALPRM QNLLTHLEEE IQLFEGVIAK QEDIKTAN | | 458 |
| 3-69 | Sequences | | |
| 3-69-1 | Sequence Number [ID] | 69 | |
| 3-69-2 | Molecule Type | DNA | |
| 3-69-3 | Length | 1428 | |
| | | source 1 . . . 1428 | |
| 3-69-4-1 | Features Location/ | mol_type = other DNA | |
| | Qualifiers | organism = Psychrobacter articus | |
| | NonEnglishQualifier Value | | |
| 3-69-5 | Residues | | |
| | atgcgcctgc tgaccgccgt cgaccagctg ttcctgctgc tggagtcccg caagcacccg | | 60 |
| | atgcacgtgg gcggcctgtt cctgttcgaa ctgccggaga acgccgacat ctcgttcgtc | | 120 |
| | caccagctgg tgaagcagat gcaggactcc gacgtcccgc ccaccttccc cttcaaccag | | 180 |
| | gtgctggaac acatgatgtt ctggaaggag gacaagaact tcgacgtcga acaccatctg | | 240 |
| | caccatgtgg ccctgccgaa gcccgcgcgc gtccgggagc tgctgatgta cgtgtcccgc | | 300 |
| | gaacacggcc ggctgctgga ccgcgcgatg ccgctgtggg aatgccatgt catcgagggc | | 360 |
| | atccagccgg aaaccgaggg cagccccgag cggttcgccc tgtatttcaa gatccaccat | | 420 |
| | tcgctggtcg acggcatcgc cgcgatgcgc ctggtgaaga agagcctgtc gcagtcgccg | | 480 |
| | aacgaacccg tgaccctgcc gatctggagc ctgatggccc accatcggaa ccagatcgac | | 540 |
| | gcgatcttcc ccaaggagcg gagcgccctg cgcatcctga aggaacaggt ctcgaccatc | | 600 |
| | aagccggtgt tcaccgagct gctgaacaac ttcaagaact acaacgacga ctcgtatgtc | | 660 |
| | tccaccttcg acgcgccccg cagcatcctg aaccgccgga tcagcgcctc gcgccggatc | | 720 |
| | gccgcgcagt cgtacgacat caagcggttc aacgacatcg ccgaacgcat caacatctcc | | 780 |
| | aagaacgacg tcgtgctggc cgtgtgcagc ggcgcgatcc gccgctacct gatcagcatg | | 840 |
| | gacgcgctgc cgagcaagcc cctgatcgcc ttcgtcccga tgtcgctgcg caccgacgac | | 900 |
| | tccatcgcgg gcaaccagct gtcgttcgtg ctggccaacc tgggcaccca cctggacgac | | 960 |
| | cccctgtccc ggatcaagct gatccatcgc tccatgaaca acagcaagcg ccggttccgc | | 1020 |
| | cggatgaacc aggcccaggt catcaactac agcatcgtgt cgtatgcctg ggagggcatc | | 1080 |
| | aacctggcga ccgacctgtt cccgaagaag caggccttca acctgatcat ctcgaacgtg | | 1140 |
| | ccgggcagcg agaagcccct gtactggaac ggcgcgcgcc tggaaagcct gtatccggcc | | 1200 |
| | tcgatcgtgt tcaacggcca ggccatgaac atcaccctgg cgtcctacct ggacaagatg | | 1260 |
| | gagttcggca tcaccgcctg cagcaaggcg ctgccgcacg tccaggacat gctgatgctg | | 1320 |
| | atcgaggaag agctgcagct gctggagtcc gtcagcaagg aactggagtt caacggcatc | | 1380 |
| | accgtgaagg acaagtcgga aaagaagctg aagaagctgg ccccgtga | | 1428 |
| 3-70 | Sequences | | |
| 3-70-1 | Sequence Number [ID] | 70 | |
| 3-70-2 | Molecule Type | AA | |
| 3-70-3 | Length | 475 | |
| | | source 1 . . . 475 | |
| 3-70-4-1 | Features Location/ | mol_type = protein | |
| | Qualifiers | organism = Psychrobacter articus | |
| | NonEnglishQualifier Value | | |
| 3-70-5 | Residues | | |
| | MRLLTAVDQL FLLLESRKHP MHVGGLFLFE LPENADISFV HQLVKQMQDS DVPPTFPFNQ | | 60 |
| | VLEHMMFWKE DKNFDVEHHL HHVALPKPAR VRELLMYVSR EHGRLLDRAM PLWECHVIEG | | 120 |
| | IQPETEGSPE RFALYFKIHH SLVDGIAAMR LVKKSLSQSP NEPVTLPIWS LMAHHRNQID | | 180 |
| | AIFPKERSAL RILKEQVSTI KPVFTELLNN FKNYNDDSYV STFDAPRSIL NRRISASRRI | | 240 |
| | AAQSYDIKRF NDIAERINIS KNDVVLAVCS GAIRRYLISM DALPSKPLIA FVPMSLRTDD | | 300 |
| | SIAGNQLSFV LANLGTHLDD PLSRIKLIHR SMNNSKRRFR RMNQAQVINY SIVSYAWEGI | | 360 |
| | NLATDLFPKK QAFNLIISNV PGSEKPLYWN GARLESLYPA SIVFNGQAMN ITLASYLDKM | | 420 |
| | EFGITACSKA LPHVQDMLML IEEELQLLES VSKELEFNGI TVKDKSEKKL KKLAP | | 475 |
| 3-71 | Sequences | | |
| 3-71-1 | Sequence Number [ID] | 71 | |
| 3-71-2 | Molecule Type | DNA | |
| 3-71-3 | Length | 1386 | |
| | | source 1 . .. 1386 | |
| 3-71-4-1 | Features Location/ | mol_type = other DNA | |
| | Qualifiers | organism = Rhodococcus opacus | |
| | NonEnglishQualifier Value | | |
| 3-71-5 | Residues | | |
| | atgtccgtga tgtccccgac cgaggcgatg ttcgtcctgt tcgagacccc gagccacccg | | 60 |
| | atgcacatgg gcgcgctgga gctgttcgag ccgccgcgcg agtcgggccc ggaccacgcc | | 120 |
| | cgcctgatgt tcgaggcgct gatctcccag gaaggcgcca gcgacacctt ccgccggcgc | | 180 |
| | gccgtccggc cgctgcgcgg cgcgtcgtac ccctggtggt ccgtcgacga ccgggtggac | | 240 |
| | ctgggctatc acgtccgcca taccgccgtg ccgggccggg gccgcatgga ggacctgctg | | 300 |
| | tcgctggtgt cccagatgca cggcatgccc ctggacccgc agcaccccat gtgggagatc | | 360 |
| | catgtcatcg aaggcctggc cgacggccgc accgcggtgt tcagcaagat ccatctgtcg | | 420 |
| | ctgatggacg gcccggccgg cctgcggctg ctgcaccatg cgctgagcac cgacccggac | | 480 |
| | gcccgcgact gccccgcgcc gtggaccccc ggcgtcagcg gcacctcgcg gcgcgaatcg | | 540 |
| | gccctgccgg tcgccgcggt gcgggcgggc gtgcgcgccg cgacctccat cgtcggcgtg | | 600 |
| | ctgcccgccc tggcgaaggt cgcctacgac ggcgtgcggg accagcacct gaccctgccg | | 660 |
| | ctgcagagcc cgcccaccat gctgaacgtc cccgtgggcc gggcccgcaa gctggccgcg | | 720 |
| | cggagctggc cgatccggcg cctggtctcg gtggccgcgg ccgcgcgcac caccatcaac | | 780 |
| | gccgtcgtgc tggcgatgtg ctcgggcgcc ctgcgccact acctggtcga gcagtatgcc | | 840 |
| | ctgccggaag cgcccctgac cgccatgctg cccgtgccgc tggacctggg cggcaccatg | | 900 |
| | atcggcccgc gtggccgcga ccacggcgtc ggcgcgatgg tcgtgggcct ggcgaccgac | | 960 |
| | gaggccgacc ccgccgcgcg gctggcccgc atcagcgagt cggtcgaaca caccaaccgc | | 1020 |
| | gtgttcggcg cgctgtccca tacccagttc caggtcatgt ccgccctggc gatcagcccg | | 1080 |
| | atcctgctgg aacccgtccg gcgcttcgtg gacgacaccc cgcccccgtt caacgtgatg | | 1140 |
| | atctcgtaca tgccgggtcc gtcccggccg cgctattgga acggcgcgcg gctggacgcc | | 1200 |
| | gtctaccccg cgccgaccgt gctgggcggc caggccctga gcatcaccct gacctcccgc | | 1260 |
| | agcggccagc tggacgtcgg cgtcgtgggc gaccggcagg ccgtgccgca cctgcagcgc | | 1320 |
| | atcatcaccc atctggagac ctccctgacc gacctggaaa acgccgtggc cgcgagcggc | | 1380 |
| | acctga | | 1386 |
| 3-72 | Sequences | | |
| 3-72-1 | Sequence Number [ID] | 72 | |
| 3-72-2 | Molecule Type | AA | |
| 3-72-3 | Length | 461 | |
| | | source 1 . . . 461 | |
| 3-72-4-1 | Features Location/ | mol_type = protein | |
| | Qualifiers | organism = Rhodococcus opacus | |
| | NonEnglishQualifier Value | | |
| 3-72-5 | Residues | | |
| | MSVMSPTEAM FVLFETPSHP MHMGALELFE PPRESGPDHA RLMFEALISQ EGASDTFRRR | | 60 |
| | AVRPLRGASY PWWSVDDRVD LGYHVRHTAV PGRGRMEDLL SLVSQMHGMP LDPQHPMWEI | | 120 |
| | HVIEGLADGR TAVFSKIHLS LMDGPAGLRL LHHALSTDPD ARDCPAPWTP GVSGTSRRES | | 180 |
| | ALPVAAVRAG VRAATSIVGV LPALAKVAYD GVRDQHLTLP LQSPPTMLNV PVGRARKLAA | | 240 |
| | RSWPIRRLVS VAAAARTTIN AVVLAMCSGA LRHYLVEQYA LPEAPLTAML PVPLDLGGTM | | 300 |
| | IGPRGRDHGV GAMVVGLATD EADPAARLAR ISESVEHTNR VFGALSHTQF QVMSALAISP | | 360 |
| | ILLEPVRRFV DDTPPPFNVM ISYMPGPSRP RYWNGARLDA VYPAPTVLGG QALSITLTSR | | 420 |
| | SGQLDVGVVG DRQAVPHLQR IITHLETSLT DLENAVAASG T | | 461 |
| 3-73 | Sequences | | |
| 3-73-1 | Sequence Number [ID] | 73 | |
| 3-73-2 | Molecule Type | DNA | |
| 3-73-3 | Length | 1356 | |
| | | source 1 . . . 1356 | |
| 3-73-4-1 | Features Location/ | mol_type = other DNA | |
| | Qualifiers | organism = Rhodococcus opacus | |
| | NonEnglishQualifier Value | | |
| 3-73-5 | Residues | | |
| | atgccggtca ccgactccat cttcctgctg ggcgaaagcc gcgagcaccc gatgcacgtg | | 60 |
| | ggctccctgg aactgttcac ccccccggac gacgccggcc cggactacgt caagtcgatg | | 120 |
| | cacgagaccc tgctgaagca taccgacgtg gaccccacct tccgcaagaa gccggcgggc | | 180 |
| | cccgtcggct cgctgggcaa cgtgtggtgg gccgacgagt ccgacgtcga cctggaatac | | 240 |
| | cacgtgcgcc atagcgcgct gccggccccc tatcgcgtcc gggaactgct gaccctgacc | | 300 |
| | tcgcggctgc acggcaccct gctggaccgc catcggccgc tgtgggagat gtacctgatc | | 360 |
| | gaaggcctga gcgacggccg cttcgccatc tataccaagc tgcaccatag cctgatggac | | 420 |
| | ggcgtctcgg gcctgcgcct gctgatgcgg accctgtcga ccgacccgga cgtgcgcgac | | 480 |
| | gccccgcccc cgtggaacct gccgcggccc gccgcggcca acggcgcggc cccggacctg | | 540 |
| | tggtcggtcg tgaacggcgt ccgccggacc gtcggcgacg tggccggcct ggcgcccgcc | | 600 |
| | tccctgcgca tcgcgcggac cgcgatgggc cagcacgaca tgcgcttccc gtacgaggcg | | 660 |
| | ccccggacca tgctgaacgt gccgatcggc ggcgcccgcc ggttcgcggc ccagtcctgg | | 720 |
| | cccctggaac gcgtccatgc cgtgcggaag gcggccggcg tcagcgtgaa cgacgtcgtg | | 780 |
| | atggccatgt gcgcgggcgc cctgcgcggc tatctggagg aacagaacgc gctgccggac | | 840 |
| | gagcccctga tcgcgatggt cccggtgtcc ctgcgggacg aacagcaggc ggacgccggc | | 900 |
| | ggcaacgccg tcggcgtgac cctgtgcaac ctggcgaccg acgtcgacga ccccgccgag | | 960 |
| | cgcctgaccg cgatcagcgc ctcgatgtcc cagggcaagg aactgttcgg cagcctgacc | | 1020 |
| | tcgatgcagg cgctggcctg gtcggcggtg aacatgtccc cgatcgccct gaccccggtc | | 1080 |
| | cccggcttcg tgcggttcac ccccccgccc ttcaacgtca tcatcagcaa cgtgccgggc | | 1140 |
| | ccccgcaaga ccatgtactg gaacggctcc cggctggacg gcatctatcc gaccagcgtc | | 1200 |
| | gtgctggacg gccaggccct gaacatcacc ctgaccacca acggcggcaa cctggacttc | | 1260 |
| | ggcgtcatcg gctgccgccg gtccgtgccg agcctgcagc gcatcctgtt ctacctggaa | | 1320 |
| | gcggccctgg gcgagctgga agcggccctg ctgtga | | 1356 |
| 3-74 | Sequences | | |
| 3-74-1 | Sequence Number [ID] | 74 | |
| 3-74-2 | Molecule Type | AA | |
| 3-74-3 | Length | 451 | |
| | | source 1 . . . 451 | |
| 3-74-4-1 | Features Location/ | mol_type = protein | |
| | Qualifiers | organism = Rhodococcus opacus | |
| | NonEnglishQualifier Value | | |
| 3-74-5 | Residues | | |
| | MPVTDSIFLL GESREHPMHV GSLELFTPPD DAGPDYVKSM HETLLKHTDV DPTFRKKPAG | | 60 |
| | PVGSLGNVWW ADESDVDLEY HVRHSALPAP YRVRELLTLT SRLHGTLLDR HRPLWEMYLI | | 120 |
| | EGLSDGRFAI YTKLHHSLMD GVSGLRLLMR TLSTDPDVRD APPPWNLPRP AAANGAAPDL | | 180 |
| | WSVVNGVRRT VGDVAGLAPA SLRIARTAMG QHDMRFPYEA PRTMLNVPIG GARRFAAQSW | | 240 |
| | PLERVHAVRK AAGVSVNDVV MAMCAGALRG YLEEQNALPD EPLIAMVPVS LRDEQQADAG | | 300 |
| | GNAVGVTLCN LATDVDDPAE RLTAISASMS QGKELFGSLT SMQALAWSAV NMSPIALTPV | | 360 |
| | PGFVRFTPPP FNVIISNVPG PRKTMYWNGS RLDGIYPTSV VLDGQALNIT LTTNGGNLDF | | 420 |
| | GVIGCRRSVP SLQRILFYLE AALGELEAAL L | | 451 |
| 3-75 | Sequences | 75 | |
| 3-75-1 | Sequence Number [ID] | DNA | |
| 3-75-2 | Molecule Type | 1395 | |
| 3-75-3 | Length | source 1 .. 1395 | |
| 3-75-4-1 | Features Location/ | mol_type = other DNA | |
| | Qualifiers | organism = Rhodococcus opacus | |
| | NonEnglishQualifier Value | | |
| 3-75-5 | Residues | | |
| | atgcccctgc cgatgtcccc cctggactcc atgttcctgc tgggcgaaag ccgcgagcac | | 60 |
| | ccgatgcacg tgggcggcgt cgaaatcttc cagctgcccg agggcgccga cacctacgac | | 120 |
| | atgcgggcga tgctggaccg cgccctggcg gacggcgacg gcatcgtcac cccgcggctg | | 180 |
| | gccaagcgcg cgcgccggtc gttcagctcg ctgggccagt ggtcctggga aaccgtggac | | 240 |
| | gacatcgacc tgggccacca tatccggcac gacgccctgc cggcccctgg cggcgaggcc | | 300 |
| | gaactgatgg cgctgtgctc gcgcctgcac ggctccctgc tggaccgcag ccggccgctg | | 360 |
| | tgggagatgc atctgatcga aggcctgagc gacggccgct tcgccgtcta taccaagatc | | 420 |
| | caccatgccg tcgcggacgg cgtgaccgcc atgaagatgc tgcggaacgc gctgagcgag | | 480 |
| | aactcggacg accgcgacgt gccggccccc tggcagccgc gtggcccgcg gccccagcgc | | 540 |
| | accccctcca gcaagggctt ctccctgagc ggcctggccg gctcgaccct gcggaccgcg | | 600 |
| | cgcgagaccg tcggcgaagt ggccggcctg gtcccggccc tggcgggcac cgtgagccgg | | 660 |
| | gccttccgcg accagggcgg cccgctggcc ctgtcggcgc cgaagacccc cttcaacgtc | | 720 |
| | cccatcaccg gcgcccgcca gttcgccgcg cagtcgtggc cgctggaacg cctgcggctg | | 780 |
| | gtggccaagc tgtcggactc caccatcaac gacgtcgtgc tggccatgtc gtccggcgcg | | 840 |
| | ctgcggtcct acctggagga ccagaacgcc ctgccggcgg accccctgat cgcgatggtc | | 900 |
| | ccggtgtccc tgaagagcca gcgcgaagcc gcgaccggca acaacatcgg cgtcctgatg | | 960 |
| | tgcaacctgg gcacccacct gcgggagccg gccgaccgcc tggaaaccat ccggaccagc | | 1020 |
| | atgcgcgagg gcaaggaagc ctatggctcg atgaccgcga cccagatcct ggccatgtcc | | 1080 |
| | gcgctgggcg ccgcgccgat cggcgccagc atgctgttcg gccataactc gcgcgtccgg | | 1140 |
| | ccgcccttca acctgatcat ctccaacgtg ccgggcccca gctcgccgct gtactggaac | | 1200 |
| | ggcgcccgcc tggacgcgat ctatccgctg agcgtccccg tggacggcca gggcctgaac | | 1260 |
| | atcacctgca cctcgaacga cgacatcatc tccttcggcg tcaccggctg ccggtccgcc | | 1320 |
| | gtgccggacc tgaagagcat ccccgcgcgc ctgggccatg agctgcgggc cctggaacgc | | 1380 |
| | gcggtgggca tctga | | 1395 |
| 3-76 | Sequences | | |
| 3-76-1 | Sequence Number [ID] | 76 | |
| 3-76-2 | Molecule Type | AA | |
| 3-76-3 | Length | 464 | |
| | | source 1 . . . 464 | |
| 3-76-4-1 | Features Location/ | mol_type = protein | |
| | Qualifiers | organism = Rhodococcus opacus | |
| | NonEnglishQualifier Value | | |
| 3-76-5 | Residues | | |
| | MPLPMSPLDS MFLLGESREH PMHVGGVEIF QLPEGADTYD MRAMLDRALA DGDGIVTPRL | | 60 |
| | AKRARRSFSS LGQWSWETVD DIDLGHHIRH DALPAPGGEA ELMALCSRLH GSLLDRSRPL | | 120 |
| | WEMHLIEGLS DGRFAVYTKI HHAVADGVTA MKMLRNALSE NSDDRDVPAP WQPRGPRPQR | | 180 |
| | TPSSKGFSLS GLAGSTLRTA RETVGEVAGL VPALAGTVSR AFRDQGGPLA LSAPKTPFNV | | 240 |
| | PITGARQFAA QSWPLERLRL VAKLSDSTIN DVVLAMSSGA LRSYLEDQNA LPADPLIAMV | | 300 |
| | PVSLKSQREA ATGNNIGVLM CNLGTHLREP ADRLETIRTS MREGKEAYGS MTATQILAMS | | 360 |
| | ALGAAPIGAS MLFGHNSRVR PPFNLIISNV PGPSSPLYWN GARLDAIYPL SVPVDGQGLN | | 420 |
| | ITCTSNDDII SFGVTGCRSA VPDLKSIPAR LGHELRALER AVGI | | 464 |
| 3-77 | Sequences | | |
| 3-77-1 | Sequence Number [ID] | 77 | |
| 3-77-2 | Molecule Type | DNA | |
| 3-77-3 | Length | 1407 | |
| | | source 1 . . . 1407 | |
| 3-77-4-1 | Features Location/ | mol_type = other DNA | |
| | Qualifiers | organism = Rhodococcus opacus | |
| | NonEnglishQualifier Value | | |
| 3-77-5 | Residues | | |
| | atggccccga ccgactccct gttcctgctg ggcgaatccc gcgagcaccc gatgcacgtg | | 60 |
| | ggcggcctgg cggtcttcac cccggcggag ggcagctcgg ccgcggacgt ccgcgccatg | | 120 |
| | ttcgacgccg cgctggtcgg cgaccgggtg gccgcgccgt tccgcaagcg ggcccgccgg | | 180 |
| | agcgtgacct cgctgggcca gtggggctgg gacaccctgc gcgacgacga ggtcgacctg | | 240 |
| | gaacaccatg tgcgccggga cgccctgccg cagccgggtg gcatggcgga actgatgacc | | 300 |
| | ctggtctccc gcctgcatgg caccctgctg gaccgcagcc ggccgctgtg ggagatgcac | | 360 |
| | ctgatcgaag gcctggccga cggccggtac gcggtgtata ccaagatcca ccatgccctg | | 420 |
| | gcggacggcg ccagcgcgat gcgcctgctg cgggactcga tgtccgagga cccgcatcgc | | 480 |
| | cggaacatgc cgaccccctg gcagccgcgc aaccccctgt cggccgtccc ggacgccggc | | 540 |
| | gtcgcggtga cccccggccc cggcagcgcc ctgcccgcga tggcctggga cgccgcgcgc | | 600 |
| | tccgccgcgg gcgaagtcgc cggcctgctg ccggccgcgc tgggcaccgt ggaccgggcc | | 660 |
| | ctgcacggca agggcggcgc cctgtccctg accgcgccgc ataccctgtt caacgtcccc | | 720 |
| | atcagcggcg cccgccacgt ggccgcgcgg tcgttcccga tcgagcgcat ccggctgctg | | 780 |
| | gccaagcatg ccgacgcgac catcaacgac atcgtgctga ccatgtgcgc cggcaccctg | | 840 |
| | cgcgcgtacc tgcacacccg cgacgccctg ccggacaacc ccctgatcgc gatggtcccg | | 900 |
| | gtgagcctgc gcgcccccga aaccggcacc ggcgaccgcg cccctggcgg caaccgggtc | | 960 |
| | ggcgtgctga tgtgcaacct ggccacccac ctgccggacc ccgcgcatcg cctggagacc | | 1020 |
| | gtccggaact gcatgaacga aggcaaggcc gcgctgcagg ccatgtcgcc ggcgcaggtc | | 1080 |
| | ctggccatgt ccgcgctggg cgccgcgccg ctgggcgtgg agatgttcct gggccgccgg | | 1140 |
| | ggccccctgc gcccgccctt caacgtcgtg atgtcgaacg tggcgggccc gcgcaccccc | | 1200 |
| | ctgtactgga acggcgcccg gctggaatcc ctgtatccgc tgagcatccc caccaccggc | | 1260 |
| | caggccctga acatcacctg cacctccagc gacgaccaga tcgtcttcgg cctgaccggc | | 1320 |
| | tgccgccgga ccgtgccgga cctgcacccc atgctggacc agctggacgc ggagctggac | | 1380 |
| | ctgctggaaa ccgcggtcgg cctgtga | | 1407 |
| 3-78 | Sequences | | |
| 3-78-1 | Sequence Number [ID] | 78 | |
| 3-78-2 | Molecule Type | AA | |
| 3-78-3 | Length | 468 | |
| | | source 1 . . . 468 | |
| 3-78-4-1 | Features Location/ | mol_type = protein | |
| | Qualifiers | organism = Rhodococcus opacus | |
| | NonEnglishQualifier Value | | |
| 3-78-5 | Residues | | |
| | MAPTDSLFLL GESREHPMHV GGLAVFTPAE GSSAADVRAM FDAALVGDRV AAPFRKRARR | | 60 |
| | SVTSLGQWGW DTLRDDEVDL EHHVRRDALP QPGGMAELMT LVSRLHGTLL DRSRPLWEMH | | 120 |
| | LIEGLADGRY AVYTKIHHAL ADGASAMRLL RDSMSEDPHR RNMPTPWQPR NPLSAVPDAG | | 180 |
| | VAVTPGPGSA LPAMAWDAAR SAAGEVAGLL PAALGTVDRA LHGKGGALSL TAPHTLFNVP | | 240 |
| | ISGARHVAAR SFPIERIRLL AKHADATIND IVLTMCAGTL RAYLHTRDAL PDNPLIAMVP | | 300 |
| | VSLRAPETGT GDRAPGGNRV GVLMCNLATH LPDPAHRLET VRNCMNEGKA ALQAMSPAQV | | 360 |
| | LAMSALGAAP LGVEMFLGRR GPLRPPFNVV ISNVAGPRTP LYWNGARLES LYPLSIPTTG | | 420 |
| | QALNITCTSS DDQIVFGLTG CRRTVPDLHP MLDQLDAELD LLETAVGL | | 468 |
| 3-79 | Sequences | | |
| 3-79-1 | Sequence Number [ID] | 79 | |
| 3-79-2 | Molecule Type | DNA | |
| 3-79-3 | Length | 11299 | |
| | | misc_feature 1 . . . 11299 | |
| 3-79-4-1 | Features Location/ | note = pCM132 | |
| | Qualifiers | | |
| | NonEnglishQualifier Value | | |
| | | source 1 . .. 11299 | |
| 3-79-4-2 | Features Location/ | mol_type = other DNA | |
| | Qualifiers | organism = synthetic construct | |
| | NonEnglishQualifier Value | | |
| 3-79-5 | Residues | | |
| | gaccctttcc gacgctcacc gggctggttg ccctcgccgc tgggctggcg gccgtctatg | | 60 |
| | gccctgcaaa cgcgccagaa acgccgtcga agccgtgtgc gagacaccgc ggccgccggc | | 120 |
| | gttgtggata cctcgcggaa aacttggccc tcactgacag atgaggggcg gacgttgaca | | 180 |
| | cttgaggggc cgactcaccc ggcgcggcgt tgacagatga ggggcaggct cgatttcggc | | 240 |
| | cggcgacgtg gagctggcca gcctcgcaaa tcggcgaaaa cgcctgattt tacgcgagtt | | 300 |
| | tcccacagat gatgtggaca agcctgggga taagtgccct gcggtattga cacttgaggg | | 360 |
| | gcgcgactac tgacagatga ggggcgcgat ccttgacact tgaggggcag agtgctgaca | | 420 |
| | gatgaggggc gcacctattg acatttgagg ggctgtccac aggcagaaaa tccagcattt | | 480 |
| | gcaagggttt ccgcccgttt ttcggccacc gctaacctgt cttttaacct gcttttaaac | | 540 |
| | caatatttat aaaccttgtt tttaaccagg gctgcgccct gtgcgcgtga ccgcgcacgc | | 600 |
| | cgaagggggg tgccccccct tctcgaaccc tcccggcccg ctaacgcggg cctcccatcc | | 660 |
| | ccccaggggc tgcgcccctc ggccgcgaac ggcctcaccc caaaaatggc agccaagctg | | 720 |
| | accacttctg cgctcggccc ttccggctgg ctggtttatt gctgataaat ctggagccgg | | 780 |
| | tgagcgtggg tctcgcggta tcattgcagc actggggcca gatggtaagc cctcccgtat | | 840 |
| | cgtagttatc tacacgacgg ggagtcaggc aactatggat gaacgaaata gacagatcgc | | 900 |
| | tgagataggt gcctcactga ttaagcattg gtaactgtca gaccaagttt actcatatat | | 960 |
| | actttagatt gatttaaaac ttcattttta atttaaaagg atctaggtga agatcctttt | | 1020 |
| | tgataatctc atgaccaaaa tcccttaacg tgagttttcg ttccactgag cgtcagaccc | | 1080 |
| | cgtagaaaag atcaaaggat cttcttgaga tccttttttt ctgcgcgtaa tctgctgctt | | 1140 |
| | gcaaacaaaa aaaccaccgc taccagcggt ggtttgtttg ccggatcaag agctaccaac | | 1200 |
| | tctttttccg aaggtaactg gcttcagcag agcgcagata ccaaatactg tccttctagt | | 1260 |
| | gtagccgtag ttaggccacc acttcaagaa ctctgtagca ccgcctacat acctcgctct | | 1320 |
| | gctaatcctg ttaccagtgg ctgctgccag tggcgataag tcgtgtctta ccgggttgga | | 1380 |
| | ctcaagacga tagttaccgg ataaggcgca gcggtcgggc tgaacggggg gttcgtgcac | | 1440 |
| | acagcccagc ttggagcgaa cgacctacac cgaactgaga tacctacagc gtgagctatg | | 1500 |
| | agaaagcgcc acgcttcccg aagggagaaa ggcggacagg tatccggtaa gcggcagggt | | 1560 |
| | cggaacagga gagcgcacga gggagcttcc agggggaaac gcctggtatc tttatagtcc | | 1620 |
| | tgtcgggttt cgccacctct gacttgagcg tcgatttttg tgatgctcgt caggggggcg | | 1680 |
| | gagcctatgg aaaaacgcca gcaacgcggc ctttttacgg ttcctggcct tttgctggcc | | 1740 |
| | ttttgctcac atgttctttc ctgcgttatc ccctgattct gtggataacc gtattaccgc | | 1800 |
| | ctttgagtga gctgataccg ctcgccgcag ccgaacgacc gagcgcagcg agtcagtgag | | 1860 |
| | cgaggaagcg gaagagcgcc caatacgcaa accgcctctc cccgcgcgtt ggccgattca | | 1920 |
| | ttaatgcagc tggcacgaca ggtttcccga ctggaaagcg ggcagtgagc gcaacgcaat | | 1980 |
| | taatgtgagt tagctcactc attaggcacc ccaggcttta cactttatgc ttccggctcg | | 2040 |
| | tatgttgtgt ggaattgtga gcggataaca atttcacaca ggaaacagct atgaccatga | | 2100 |
| | ttacgccaag cttgcatgcc tgcaggtcga ctctagagga tcaattcggc ttccaccgga | | 2160 |
| | attagcttga aatagtacat aatggatttc cttacgcgaa atacgggcag acatggcctg | | 2220 |
| | cccggttatt attatttttg acaccagacc aactggtaat ggtagcgacc ggcgctcagc | | 2280 |
| | tgtaattccg ccgatactga cgggctccag gagtcgtcgc caccaatccc catatggaaa | | 2340 |
| | ccgtcgatat tcagccatgt gccttcttcc gcgtgcagca gatggcgatg gctggtttcc | | 2400 |
| | atcagttgct gttgactgta gcggctgatg ttgaactgga agtcgccgcg ccactggtgt | | 2460 |
| | gggccataat tcaattcgcg cgtcccgcag cgcagaccgt tttcgctcgg gaagacgtac | | 2520 |
| | ggggtataca tgtctgacaa tggcagatcc cagcggtcaa aacaggcggc agtaaggcgg | | 2580 |
| | tcgggatagt tttcttgcgg ccctaatccg agccagttta cccgctctgc tacctgcgcc | | 2640 |
| | agctggcagt tcaggccaat ccgcgccgga tgcggtgtat cgctcgccac ttcaacatca | | 2700 |
| | acggtaatcg ccatttgacc actaccatca atccggtagg ttttccggct gataaataag | | 2760 |
| | gttttcccct gatgctgcca cgcgtgagcg gtcgtaatca gcaccgcatc agcaagtgta | | 2820 |
| | tctgccgtgc actgcaacaa cgctgcttcg gcctggtaat ggcccgccgc cttccagcgt | | 2880 |
| | tcgacccagg cgttagggtc aatgcgggtc gcttcactta cgccaatgtc gttatccagc | | 2940 |
| | ggtgcacggg tgaactgatc gcgcagcggc gtcagcagtt gttttttatc gccaatccac | | 3000 |
| | atctgtgaaa gaaagcctga ctggcggtta aattgccaac gcttattacc cagctcgatg | | 3060 |
| | caaaaatcca tttcgctggt ggtcagatgc gggatggcgt gggacgcggc ggggagcgtc | | 3120 |
| | acactgaggt tttccgccag acgccactgc tgccaggcgc tgatgtgccc ggcttctgac | | 3180 |
| | catgcggtcg cgttcggttg cactacgcgt actgtgagcc agagttgccc ggcgctctcc | | 3240 |
| | ggctgcggta gttcaggcag ttcaatcaac tgtttacctt gtggagcgac atccagaggc | | 3300 |
| | acttcaccgc ttgccagcgg cttaccatcc agcgccacca tccagtgcag gagctcgtta | | 3360 |
| | tcgctatgac ggaacaggta ttcgctggtc acttcgatgg tttgcccgga taaacggaac | | 3420 |
| | tggaaaaact gctgctggtg ttttgcttcc gtcagcgctg gatgcggcgt gcggtcggca | | 3480 |
| | aagaccagac cgttcataca gaactggcga tcgttcggcg tatcgccaaa atcaccgccg | | 3540 |
| | taagccgacc acgggttgcc gttttcatca tatttaatca gcgactgatc cacccagtcc | | 3600 |
| | cagacgaagc cgccctgtaa acggggatac tgacgaaacg cctgccagta tttagcgaaa | | 3660 |
| | ccgccaagac tgttacccat cgcgtgggcg tattcgcaaa ggatcagcgg gcgcgtctct | | 3720 |
| | ccaggtagcg aaagccattt tttgatggac catttcggca cagccgggaa gggctggtct | | 3780 |
| | tcatccacgc gcgcgtacat cgggcaaata atatcggtgg ccgtggtgtc ggctccgccg | | 3840 |
| | ccttcatact gcaccgggcg ggaaggatcg acagatttga tccagcgata cagcgcgtcg | | 3900 |
| | tgattagcgc cgtggcctga ttcattcccc agcgaccaga tgatcacact cgggtgatta | | 3960 |
| | cgatcgcgct gcaccattcg cgttacgcgt tcgctcatcg ccggtagcca gcgcggatca | | 4020 |
| | tcggtcagac gattcattgg caccatgccg tgggtttcaa tattggcttc atccaccaca | | 4080 |
| | tacaggccgt agcggtcgca cagcgtgtac cacagcggat ggttcggata atgcgaacag | | 4140 |
| | cgcacggcgt taaagttgtt ctgcttcatc agcaggatat cctgcaccat cgtctgctca | | 4200 |
| | tccatgacct gaccatgcag aggatgatgc tcgtgacggt taacgcctcg aatcagcaac | | 4260 |
| | ggcttgccgt tcagcagcag cagaccattt tcaatccgca cctcgcggaa accgacatcg | | 4320 |
| | caggcttctg cttcaatcag cgtgccgtcg gcggtgtgca gttcaaccac cgcacgatag | | 4380 |
| | agattcggga tttcggcgct ccacagtttc gggttttcga cgttcagacg tagtgtgacg | | 4440 |
| | cgatcggcat aaccaccacg ctcatcgata atttcaccgc cgaaaggcgc ggtgccgctg | | 4500 |
| | gcgacctgcg tttcaccctg ccataaagaa actgttaccc gtaggtagtc acgcaactcg | | 4560 |
| | ccgcacatct gaacttcagc ctccagtaca gcgcggctga aatcatcatt aaagcgagtg | | 4620 |
| | gcaacatgga aatcgctgat ttgtgtagtc ggtttatgca gcaacgagac gtcacggaaa | | 4680 |
| | atgccgctca tccgccacat atcctgatct tccagataac tgccgtcact ccaacgcagc | | 4740 |
| | accatcaccg cgaggcggtt ttctccggcg cgtaaaaatg cgctcaggtc aaattcagac | | 4800 |
| | ggcaaacgac tgtcctggcc gtaaccgacc cagcgcccgt tgcaccacag atgaaacgcc | | 4860 |
| | gagttaacgc catcaaaaat aattcgcgtc tggccttcct gtagccagct ttcatcaaca | | 4920 |
| | ttaaatgtga gcgagtaaca acccgtcgga ttctccgtgg gaacaaacgg cggattgacc | | 4980 |
| | gtaatgggat aggttacgtt ggtgtagatg ggcgcatcgt aaccgtgcat ctgccagttt | | 5040 |
| | gaggggacga cgacagtatc ggcctcagga agatcgcact ccagccagct ttccggcacc | | 5100 |
| | gcttctggtg ccggaaacca ggcaaagcgc cattcgccat tcaggctgcg caactgttgg | | 5160 |
| | gaagggcgat cggtgcgggc ctcttcgcta ttacgccagc tggcgaaagg gggatgtgct | | 5220 |
| | gcaaggcgat taagttgggt aacgccaggg ttttcccggt cgacccgtaa tcttacgtca | | 5280 |
| | gtaacttcca cagtagttca ccaccttttc cctatagatc ttccgtgcag tttaagccga | | 5340 |
| | attgatcccc gggtaccgag ctcgaatcta gaattccctg ctttcctgat gcaaaaacga | | 5400 |
| | ggctagttta ccgtatctgt ggggggatgg cttgtagata tgacgacagg aagagtttgt | | 5460 |
| | agaaacgcaa aaaggccatc cgtcaggatg gccttctgct taatttgatg cctggcagtt | | 5520 |
| | tatggcgggc gtcctgcccg ccaccctccg ggccgttgct tcgcaacgtt caaatccgct | | 5580 |
| | cccggcggat ttgtcctact caggagagcg ttcaccgaca aacaacagat aaaacgaaag | | 5640 |
| | gcccagtctt tcgactgagc ctttcgtttt atttgatgcc tggcagttcc ctactctcgc | | 5700 |
| | atggggagac cccacactac catcggcgct acggcgtttc acttctgagt tcggcatggg | | 5760 |
| | gtcaggtggg accaccgcgc tactgccgcc aggcaaattc tgttttatca gaccgcttct | | 5820 |
| | gcgttctgat ttaatctgta tcaggctgaa aaattcactg gccgtcgttt tacaacgtcg | | 5880 |
| | tgactgggaa aaccctggcg ttacccaact taatcgcctt gcagcacatc cccctttcgc | | 5940 |
| | cagctggcgt aatagcgaag aggcccgcac cgatcgccct tcccaacagt tgcgcagcct | | 6000 |
| | gaatggcgaa tggcgcctga tgcggtattt tctccttacg catctgtgcg gtatttcaca | | 6060 |
| | ccgcatatgg tgcactctca gtacaatctg ctctgatgcc gcatagttaa gccagccccg | | 6120 |
| | acacccgcca acacccgctg acgcgccctg acgggcttgt ctgctcccgg catccgctta | | 6180 |
| | cagacaagct gtgaccgtct ccgggagctg catgtgtcag aggttttcac cgtcatcacc | | 6240 |
| | gaaacgcgcg agacgaaagg gcctcgtgat acgcctattt ttataggtta atgtcatgat | | 6300 |
| | aataatggtt tcttagcacc ctttctcggt ccttcaacgt tcctgacaac gagcctcctt | | 6360 |
| | ttcgccaatc catcgacaat caccgcgagt ccctgctcga acgctgcgtc cggaccggct | | 6420 |
| | tcgtcgaagg cgtctatcgc ggcccgcaac agcggcgaga gcggagcctg ttcaacggtg | | 6480 |
| | ccgccgcgct cgccggcatc gctgtcgccg gcctgctcct caagcacggc cccaacagtg | | 6540 |
| | aagtagctga ttgtcatcag cgcattgacg gcgtccccgg ccgaaaaacc cgcctcgcag | | 6600 |
| | aggaagcgaa gctgcgcgtc ggccgtttcc atctgcggtg cgcccggtcg cgtgccggca | | 6660 |
| | tggatgcgcg cgccatcgcg gtaggcgagc agcgcctgcc tgaagctgcg ggcattcccg | | 6720 |
| | atcagaaatg agcgccagtc gtcgtcggct ctcggcaccg aatgcgtatg attctccgcc | | 6780 |
| | agcatggctt cggccagtgc gtcgagcagc gcccgcttgt tcctgaagtg ccagtaaagc | | 6840 |
| | gccggctgct gaacccccaa ccgttccgcc agtttgcgtg tcgtcagacc gtctacgccg | | 6900 |
| | acctcgttca acaggtccag ggcggcacgg atcactgtat tcggctgcaa ctttgtcatg | | 6960 |
| | attgacactt tatcactgat aaacataata tgtccaccaa cttatcagtg ataaagaatc | | 7020 |
| | cgcgcgttca atcggaccag cggaggctgg tccggaggcc agacgtgaaa cccaacatac | | 7080 |
| | ccctgatcgt aattctgagc actgtcgcgc tcgacgctgt cggcatcggc ctgattatgc | | 7140 |
| | cggtgctgcc gggcctcctg cgcgatctgg ttcactcgaa cgacgtcacc gcccactatg | | 7200 |
| | gcattctgct ggcgctgtat gcgttggtgc aatttgcctg cgcacctgtg ctgggcgcgc | | 7260 |
| | tgtcggatcg tttcgggcgg cggccaatct tgctcgtctc gctggccggc gccactgtcg | | 7320 |
| | actacgccat catggcgaca gcgcctttcc tttgggttct ctatatcggg cggatcgtgg | | 7380 |
| | ccggcatcac cggggcgact ggggcggtag ccggcgctta tattgccgat gacctgcagg | | 7440 |
| | gggggggggg cgctgaggtc tgcctcgtga agaaggtgtt gctgactcat accaggcctg | | 7500 |
| | aatcgcccca tcatccagcc agaaagtgag ggagccacgg ttgatgagag ctttgttgta | | 7560 |
| | ggtggaccag ttggtgattt tgaacttttg ctttgccacg gaacggtctg cgttgtcggg | | 7620 |
| | aagatgcgtg atctgatcct tcaactcagc aaaagttcga tttattcaac aaagccgccg | | 7680 |
| | tcccgtcaag tcagcgtaat gctctgccag tgttacaacc aattaaccaa ttctgattag | | 7740 |
| | aaaaactcat cgagcatcaa atgaaactgc aatttattca tatcaggatt atcaatacca | | 7800 |
| | tatttttgaa aaagccgttt ctgtaatgaa ggagaaaact caccgaggca gttccatagg | | 7860 |
| | atggcaagat cctggtatcg gtctgcgatt ccgactcgtc caacatcaat acaacctatt | | 7920 |
| | aatttcccct cgtcaaaaat aaggttatca agtgagaaat caccatgagt gacgactgaa | | 7980 |
| | tccggtgaga atggcaaaag cttatgcatt tctttccaga cttgttcaac aggccagcca | | 8040 |
| | ttacgctcgt catcaaaatc actcgcatca accaaaccgt tattcattcg tgattgcgcc | | 8100 |
| | tgagcgagac gaaatacgcg atcgctgtta aaaggacaat tacaaacagg aatcgaatgc | | 8160 |
| | aaccggcgca ggaacactgc cagcgcatca acaatatttt cacctgaatc aggatattct | | 8220 |
| | tctaatacct ggaatgctgt tttcccgggg atcgcagtgg tgagtaacca tgcatcatca | | 8280 |
| | ggagtacgga taaaatgctt gatggtcgga agaggcataa attccgtcag ccagtttagt | | 8340 |
| | ctgaccatct catctgtaac atcattggca acgctacctt tgccatgttt cagaaacaac | | 8400 |
| | tctggcgcat cgggcttccc atacaatcga tagattgtcg cacctgattg cccgacatta | | 8460 |
| | tcgcgagccc atttataccc atataaatca gcatccatgt tggaatttaa tcgcggcctc | | 8520 |
| | gagcaagacg tttcccgttg aatatggctc ataacacccc ttgtattact gtttatgtaa | | 8580 |
| | gcagacagtt ttattgttca tgatgatata tttttatctt gtgcaatgta acatcagaga | | 8640 |
| | ttttgagaca caacgtggct ttcccccccc cccctgcagg tccgacacgg ggatggatgg | | 8700 |
| | cgttcccgat catggtcctg cttgcttcgg gtggcatcgg aatgccggcg ctgcaagcaa | | 8760 |
| | tgttgtccag gcaggtggat gaggaacgtc aggggcagct gcaaggctca ctggcggcgc | | 8820 |
| | tcaccagcct gacctcgatc gtcggacccc tcctcttcac ggcgatctat gcggcttcta | | 8880 |
| | taacaacgtg gaacgggtgg gcatggattg caggcgctgc cctctacttg ctctgcctgc | | 8940 |
| | cggcgctgcg tcgcgggctt tggagcggcg cagggcaacg agccgatcgc tgatcgtgga | | 9000 |
| | aacgataggc ctatgccatg cgggtcaagg cgacttccgg caagctatac gcgccctaga | | 9060 |
| | attgtcaatt ttaatcctct gtttatcggc agttcgtaga gcgcgccgtg cgtcccgagc | | 9120 |
| | gatactgagc gaagcaagtg cgtcgagcag tgcccgcttg ttcctgaaat gccagtaaag | | 9180 |
| | cgctggctgc tgaaccccca gccggaactg accccacaag gccctagcgt ttgcaatgca | | 9240 |
| | ccaggtcatc attgacccag gcgtgttcca ccaggccgct gcctcgcaac tcttcgcagg | | 9300 |
| | cttcgccgac ctgctcgcgc cacttcttca cgcgggtgga atccgatccg cacatgaggc | | 9360 |
| | ggaaggtttc cagcttgagc gggtacggct cccggtgcga gctgaaatag tcgaacatcc | | 9420 |
| | gtcgggccgt cggcgacagc ttgcggtact tctcccatat gaatttcgtg tagtggtcgc | | 9480 |
| | cagcaaacag cacgacgatt tcctcgtcga tcaggacctg gcaacgggac gttttcttgc | | 9540 |
| | cacggtccag gacgcggaag cggtgcagca gcgacaccga ttccaggtgc ccaacgcggt | | 9600 |
| | cggacgtgaa gcccatcgcc gtcgcctgta ggcgcgacag gcattcctcg gccttcgtgt | | 9660 |
| | aataccggcc attgatcgac cagcccaggt cctggcaaag ctcgtagaac gtgaaggtga | | 9720 |
| | tcggctcgcc gataggggtg cgcttcgcgt actccaacac ctgctgccac accagttcgt | | 9780 |
| | catcgtcggc ccgcagctcg acgccggtgt aggtgatctt cacgtccttg ttgacgtgga | | 9840 |
| | aaatgacctt gttttgcagc gcctcgcgcg ggattttctt gttgcgcgtg gtgaacaggg | | 9900 |
| | cagagcgggc cgtgtcgttt ggcatcgctc gcatcgtgtc cggccacggc gcaatatcga | | 9960 |
| | acaaggaaag ctgcatttcc ttgatctgct gcttcgtgtg tttcagcaac gcggcctgct | | 10020 |
| | tggcctcgct gacctgtttt gccaggtcct cgccggcggt ttttcgcttc ttggtcgtca | | 10080 |
| | tagttcctcg cgtgtcgatg gtcatcgact tcgccaaacc tgccgcctcc tgttcgagac | | 10140 |
| | gacgcgaacg ctccacggcg gccgatggcg cgggcagggc agggggagcc agttgcacgc | | 10200 |
| | tgtcgcgctc gatcttggcc gtagcttgct ggaccatcga gccgacggac tggaaggttt | | 10260 |
| | cgcggggcgc acgcatgacg gtgcggcttg cgatggtttc ggcatcctcg gcggaaaacc | | 10320 |
| | ccgcgtcgat cagttcttgc ctgtatgcct tccggtcaaa cgtccgattc attcaccctc | | 10380 |
| | cttgcgggat tgccccgact cacgccgggg caatgtgccc ttattcctga tttgacccgc | | 10440 |
| | ctggtgcctt ggtgtccaga taatccacct tatcggcaat gaagtcggtc ccgtagaccg | | 10500 |
| | tctggccgtc cttctcgtac ttggtattcc gaatcttgcc ctgcacgatt accagctccg | | 10560 |
| | cgaagtcgct cttcttgatg gagcgcatgg ggacgtgctt ggcaatcacg cgcacccccc | | 10620 |
| | ggccgtttta gcggctaaaa aagtcatggc tctgccctcg ggcggaccac gcccatcatg | | 10680 |
| | accttgccaa gctcgtcctg cttctcttcg atcttcgcca gcagggcgag gatcgtggca | | 10740 |
| | tcaccgaacc gcgccgtgcg cgggtcgtcg gtgagccaga gtttcagcag gccgcccagg | | 10800 |
| | cggcccaggt cgccattgat gcgggccagc tcgcggacgt gctcatagtc cacgacgccc | | 10860 |
| | gtgattttgt agccctggcc gacggccagc aggtaggcct acaggctcat gccggccgcc | | 10920 |
| | gccgcctttt cctcaatcgc tcttcgttcg tctggaaggc agtacacctt gataggtggg | | 10980 |
| | ctgcccttcc tggttggctt ggtttcatca gccatccgct tgccctcatc tgttacgccg | | 11040 |
| | gcggtagccg gccagcctcg cagagcagga ttcccgttga gcaccgccag gtgcgaataa | | 11100 |
| | gggacagtga agaaggaaca cccgctcgcg ggtgggccta cttcacctat cctgcccggc | | 11160 |
| | tgacgccgtt ggatacacca aggaaagtct acacgaaccc tttggcaaaa tcctgtatat | | 11220 |
| | cgtgcgaaaa aggatggata taccgaaaaa atcgctataa tgaccccgaa gcagggttat | | 11280 |
| | gcagcggaaa agatccgtc | | 11299 |
| 3-80 | Sequences | | |
| 3-80-1 | Sequence Number [ID] | 80 | |
| 3-80-2 | Molecule Type | DNA | |
| 3-80-3 | Length | 7341 | |
| 3-80-4-1 | Features Location/ | misc_feature 1 . . . 7341 | |
| | Qualifiers | note = pJSvec | |
| | NonEnglishQualifier Value | | |
| | | source 1 . . . 7341 | |
| 3-80-4-2 | Features Location/ | mol_type = other DNA | |
| | Qualifiers | organism = synthetic construct | |
| | NonEnglishQualifier Value | | |
| 3-80-5 | Residues | | |
| | gaccctttcc gacgctcacc gggctggttg ccctcgccgc tgggctggcg gccgtctatg | | 60 |
| | gccctgcaaa cgcgccagaa acgccgtcga agccgtgtgc gagacaccgc ggccgccggc | | 120 |
| | gttgtggata cctcgcggaa aacttggccc tcactgacag atgaggggcg gacgttgaca | | 180 |
| | cttgaggggc cgactcaccc ggcgcggcgt tgacagatga ggggcaggct cgatttcggc | | 240 |
| | cggcgacgtg gagctggcca gcctcgcaaa tcggcgaaaa cgcctgattt tacgcgagtt | | 300 |
| | tcccacagat gatgtggaca agcctgggga taagtgccct gcggtattga cacttgaggg | | 360 |
| | gcgcgactac tgacagatga ggggcgcgat ccttgacact tgaggggcag agtgctgaca | | 420 |
| | gatgaggggc gcacctattg acatttgagg ggctgtccac aggcagaaaa tccagcattt | | 480 |
| | gcaagggttt ccgcccgttt ttcggccacc gctaacctgt cttttaacct gcttttaaac | | 540 |
| | caatatttat aaaccttgtt tttaaccagg gctgcgccct gtgcgcgtga ccgcgcacgc | | 600 |
| | cgaagggggg tgccccccct tctcgaaccc tcccggcccg ctaacgcggg cctcccatcc | | 660 |
| | ccccaggggc tgcgcccctc ggccgcgaac ggcctcaccc caaaaatggc agccaagctg | | 720 |
| | accacttctg cgctcggccc ttccggctgg ctggtttatt gctgataaat ctggagccgg | | 780 |
| | tgagcgtggg tctcgcggta tcattgcagc actggggcca gatggtaagc cctcccgtat | | 840 |
| | cgtagttatc tacacgacgg ggagtcaggc aactatggat gaacgaaata gacagatcgc | | 900 |
| | tgagataggt gcctcactga ttaagcattg gtaactgtca gaccaagttt actcatatat | | 960 |
| | actttagatt gatttaaaac ttcattttta atttaaaagg atctaggtga agatcctttt | | 1020 |
| | tgataatctc atgaccaaaa tcccttaacg tgagttttcg ttccactgag ctgcagaccc | | 1080 |
| | cgtagaaaag atcaaaggat cttcttgaga tccttttttt ctgcgcgtaa tctgctgctt | | 1140 |
| | gcaaacaaaa aaaccaccgc taccagcggt ggtttgtttg ccggatcaag agctaccaac | | 1200 |
| | tctttttccg aaggtaactg gcttcagcag agcgcagata ccaaatactg tccttctagt | | 1260 |
| | gtagccgtag ttaggccacc acttcaagaa ctctgtagca ccgcctacat acctcgctct | | 1320 |
| | gctaatcctg ttaccagtgg ctgctgccag tggcgataag tcgtgtctta ccgggttgga | | 1380 |
| | ctcaagacga tagttaccgg ataaggcgca gcggtcgggc tgaacggggg gttcgtgcac | | 1440 |
| | acagcccagc ttggagcgaa cgacctacac cgaactgaga tacctacagc gtgagctatg | | 1500 |
| | agaaagcgcc acgcttcccg aagggagaaa ggcggacagg tatccggtaa gcggcagggt | | 1560 |
| | cggaacagga gagcgcacga gggagcttcc agggggaaac gcctggtatc tttatagtcc | | 1620 |
| | tgtcgggttt cgccacctct gacttgagcg tcgatttttg tgatgctcgt caggggggcg | | 1680 |
| | gagcctatgg aaaaacgcca gcaacgcggc ctttttacgg ttcctggcct tttgctggcc | | 1740 |
| | ttttgctcac atgttctttc ctgcgttatc ccctgattct gtggataacc gtattaccgc | | 1800 |
| | ctttgagtga gctgataccg ctcgccgcag ccgaacgacc gagcgcagcg agtcagtgag | | 1860 |
| | cgaggaagcg gaagagcgcc caatacgcaa accgcctctc cccgcgcgtt ggccgattca | | 1920 |
| | ttaatgcagc tggcaggaag cggcgatggc ggagctgaat tacattccca accgcgtggc | | 1980 |
| | acaacaactg gcgggcaaac agtcgttgct gattggcgtt gccacctcca gtctggccct | | 2040 |
| | gcacgcgccg tcgcaaattg tcgcggcgat taaatctcgc gccgatcaac tgggtgccag | | 2100 |
| | cgtggtggtg tcgatggtag aacgaagcgg cgtcgaagcc tgtaaagcgg cggtgcacaa | | 2160 |
| | tcttctcgcg caacgcgtca gtgggctgat cattaactat ccgctggatg accaggatgc | | 2220 |
| | cattgctgtg gaagctgcct gcactaatgt tccggcgtta tttcttgatg tctctgacca | | 2280 |
| | gacacccatc aacagtatta ttttctccca tgaagacggt acgcgactgg gcgtggagca | | 2340 |
| | tctggtcgca ttgggtcacc agcaaatcgc gctgttagcg ggcccattaa gttctgtctc | | 2400 |
| | ggcgcgtctg cgtctggctg gctggcataa atatctcact cgcaatcaaa ttcagccgat | | 2460 |
| | agcggaacgg gaaggcgact ggagtgccat gtccggtttt caacaaacca tgcaaatgct | | 2520 |
| | gaatgagggc atcgttccca ctgcgatgct ggttgccaac gatcagatgg cgctgggcgc | | 2580 |
| | aatgcgcgcc attaccgagt ccgggctgcg cgttggtgcg gatatctcgg tagtgggata | | 2640 |
| | cgacgatacc gaagacagct catgttatat cccgccgtca accaccatca aacaggattt | | 2700 |
| | tcgcctgctg gggcaaacca gcgtggaccg cttgctgcaa ctctctcagg gccaggcggt | | 2760 |
| | gaagggcaat cagctgttgc ccgtctcact ggtgaaaaga aaaaccaccc tggcgcccaa | | 2820 |
| | tacgcaaacc gcctctcccc gcgcgttggc cgattcatta atgcagctgg cacgacaggt | | 2880 |
| | ttcccgactg gaaagcgggc agtgagcgca acgcaattaa tgtgagttag cgcgaattga | | 2940 |
| | tctggtttga cagcttatca tcgactgcac ggtgcaccaa tgcttctggc gtcaggcagc | | 3000 |
| | catcggaagc tgtggtatgg ctgtgcaggt ctgaaatcac tgcataattc gtgtcgctca | | 3060 |
| | aggcgcactc ccgttctgga taatgttttt tgcgccgaca tcataacggt tctggcaaat | | 3120 |
| | attctgaaat gagctgttga caattaatca tccggctcgt ataatgtgtg gaattgtgag | | 3180 |
| | cggataacaa tttcacacag gaaacagcgc cgctgagaaa aagcgaagcg gcactgctct | | 3240 |
| | ttaacaattt atcagacaat ctgtgtgggc actcgaccgg aattatcgat taactttatt | | 3300 |
| | attaaaaatt aaagaggtat atattaatgt atcgattaaa taaggaggaa taaacccaga | | 3360 |
| | acgcagaagc ggtctgataa aacagaattt gcctggcggc agtagcgcgg tggtcccacc | | 3420 |
| | tgaccccatg ccgaactcag aagtgaaacg ccgtagcgcc gatggtagtg tggggtctcc | | 3480 |
| | ccatgcgaga gtagggaact gccaggcatc aaataaaacg aaaggctcag tcgaaagact | | 3540 |
| | gggcctttcg ttttatctgt tgtttgtcgg tgaacgctct cctgagtagg acaaatcttg | | 3600 |
| | taggtggacc agttggtgat tttgaacttt tgctttgcca cggaacggtc tgcgttgtcg | | 3660 |
| | ggaagatgcg tgatctgatc cttcaactca gcaaaagttc gatttattca acaaagccgc | | 3720 |
| | cgtcccgtca agtcagcgta atgctctgcc agtgttacaa ccaattaacc aattctgatt | | 3780 |
| | agaaaaactc atcgagcatc aaatgaaact gcaatttatt catatcagga ttatcaatac | | 3840 |
| | catatttttg aaaaagccgt ttctgtaatg aaggagaaaa ctcaccgagg cagttccata | | 3900 |
| | ggatggcaag atcctggtat cggtctgcga ttccgactcg tccaacatca atacaaccta | | 3960 |
| | ttaatttccc ctcgtcaaaa ataaggttat caagtgagaa atcaccatga gtgacgactg | | 4020 |
| | aatccggtga gaatggcaaa agcttatgca tttctttcca gacttgttca acaggccagc | | 4080 |
| | cattacgctc gtcatcaaaa tcactcgcat caaccaaacc gttattcatt cgtgattgcg | | 4140 |
| | cctgagcgag acgaaatacg cgatcgctgt taaaaggaca attacaaaca ggaatcgaat | | 4200 |
| | gcaaccggcg caggaacact gccagcgcat caacaatatt ttcacctgaa tcaggatatt | | 4260 |
| | cttctaatac ctggaatgct gttttcccgg ggatcgcagt ggtgagtaac catgcatcat | | 4320 |
| | caggagtacg gataaaatgc ttgatggtcg gaagaggcat aaattccgtc agccagttta | | 4380 |
| | gtctgaccat ctcatctgta acatcattgg caacgctacc tttgccatgt ttcagaaaca | | 4440 |
| | actctggcgc atcgggcttc ccatacaatc gatagattgt cgcacctgat tgcccgacat | | 4500 |
| | tatcgcgagc ccatttatac ccatataaat cagcatccat gttggaattt aatcgcggcc | | 4560 |
| | tcgagcaaga cgtttcccgt tgaatatggc tcataacacc ccttgtatta ctgtttatgt | | 4620 |
| | aagcagacag ttttattgtt catgatgata tatttttatc ttgtgcaatg taacatcaga | | 4680 |
| | gattttgaga cacaacgtgg ctttcccccc cccccctgca ggtccgacac ggggatggat | | 4740 |
| | ggcgttcccg atcatggtcc tgcttgcttc gggtggcatc ggaatgccgg cgctgcaagc | | 4800 |
| | aatgttgtcc aggcaggtgg atgaggaacg tcaggggcag ctgcaaggct cactggcggc | | 4860 |
| | gctcaccagc ctgacctcga tcgtcggacc cctcctcttc acggcgatct atgcggcttc | | 4920 |
| | tataacaacg tggaacgggt gggcatggat tgcaggcgct gccctctact tgctctgcct | | 4980 |
| | gccggcgctg cgtcgcgggc tttggagcgg cgcagggcaa cgagccgatc gctgatcgtg | | 5040 |
| | gaaacgatag gcctatgcca tgcgggtcaa ggcgacttcc ggcaagctat acgcgcccta | | 5100 |
| | gaattgtcaa ttttaatcct ctgtttatcg gcagttcgta gagcgcgccg tgcgtcccga | | 5160 |
| | gcgatactga gcgaagcaag tgcgtcgagc agtgcccgct tgttcctgaa atgccagtaa | | 5220 |
| | agcgctggct gctgaacccc cagccggaac tgaccccaca aggccctagc gtttgcaatg | | 5280 |
| | caccaggtca tcattgaccc aggcgtgttc caccaggccg ctgcctcgca actcttcgca | | 5340 |
| | ggcttcgccg acctgctcgc gccacttctt cacgcgggtg gaatccgatc cgcacatgag | | 5400 |
| | gcggaaggtt tccagcttga gcgggtacgg ctcccggtgc gagctgaaat agtcgaacat | | 5460 |
| | ccgtcgggcc gtcggcgaca gcttgcggta cttctcccat atgaatttcg tgtagtggtc | | 5520 |
| | gccagcaaac agcacgacga tttcctcgtc gatcaggacc tggcaacggg acgttttctt | | 5580 |
| | gccacggtcc aggacgcgga agcggtgcag cagcgacacc gattccaggt gcccaacgcg | | 5640 |
| | gtcggacgtg aagcccatcg ccgtcgcctg taggcgcgac agccattcct cggccttcgt | | 5700 |
| | gtaataccgg ccattgatcg accagcccag gtcctggcaa agctcgtaga acgtgaaggt | | 5760 |
| | gatcggctcg ccgatagggg tgcgcttcgc gtactccaac acctgctgcc acaccagttc | | 5820 |
| | gtcatcgtcg gcccgcagct cgacgccggt gtaggtgatc ttcacgtcct tgttgacgtg | | 5880 |
| | gaaaatgacc ttgttttgca gcgcctcgcg cgggattttc ttgttgcgcg tggtgaacag | | 5940 |
| | ggcagagcgg gccgtgtcgt ttggcatcgc tcgcatcgtg tccggccacg gcgcaatatc | | 6000 |
| | gaacaaggaa agctgcattt ccttgatctg ctgcttcgtg tgtttcagca acgcggcctg | | 6060 |
| | cttggcctcg ctgacctgtt ttgccaggtc ctcgccggcg gtttttcgct tcttggtcgt | | 6120 |
| | catagttcct cgcgtgtcga tggtcatcga cttcgccaaa cctgccgcct cctgttcgag | | 6180 |
| | acgacgcgaa cgctccacgg cggccgatgg cgcgggcagg gcagggggag ccagttgcac | | 6240 |
| | gctgtcgcgc tcgatcttgg ccgtagcttg ctggaccatc tcggcatcct acgtgaaggt | | 6300 |
| | ttcgcggggc gcacgcatga cggtgcggct tgcgatggtt aacgtccgat cggcggaaaa | | 6360 |
| | ccccgcgtcg atcagttctt gcctgtatgc cttccggtca ccttattcct tcattcaccc | | 6420 |
| | tccttgcggg attgccccga ctcacgccgg ggcaatgtgc atgaagtcgg gatttgaccc | | 6480 |
| | gcctggtgcc ttggtgtcca gataatccac cttatcggca ccctgcacga tcccgtagac | | 6540 |
| | cgtctggccg tccttctcgt acttggtatt ccgaatcttg ttggcaatca ataccagctc | | 6600 |
| | cgcgaagtcg ctcttcttga tggagcgcat ggggacgtgc cgggcggacc cgcgcacccc | | 6660 |
| | ccggccgttt tagcggctaa aaaagtcatg gctctgccct cagcagggcg acgcccatca | | 6720 |
| | tgaccttgcc aagctcgtcc tgcttctctt cgatcttcgc gagtttcagc aggatcgtgg | | 6780 |
| | catcaccgaa ccgcgccgtg cgcgggtcgt cggtgagcca gtgctcatgc aggccgccca | | 6840 |
| | ggcggcccag gtcgccattg atgcgggcca gctcgcggac gtgctcatag tccacgacgc | | 6900 |
| | ccgtgatttt gtagccctgg ccgacggcca gcaggtaggc ctacaggctc atgccggccg | | 6960 |
| | ccgccgcctt ttcctcaatc gctcttcgtt cgtctggaag gcagtacacc ttgataggtg | | 7020 |
| | ggctgccctt cctggttggc ttggtttcat cagccatccg cttgccctca tctgttacgc | | 7080 |
| | cggcggtagc cggccagcct cgcagagcag gattcccgtt gagcaccgcc aggtgcgaat | | 7140 |
| | aagggacagt gaagaaggaa cacccgctcg cgggtgggcc tacttcacct atcctgcccg | | 7200 |
| | gctgacgccg ttggatacac caaggaaagt ctacacgaac cctttggcaa aatcctgtat | | 7260 |
| | atcgtgcgaa aaaggatgga tataccgaaa aaatcgctat aatgaccccg aagcagggtt | | 7320 |
| | atgcagcgga aaagatccgt c | | 7341 |
| 3-81 | Sequences | | |
| 3-81-1 | Sequence Number [ID] | 81 | |
| 3-81-2 | Molecule Type | DNA | |
| 3-81-3 | Length | 10705 | |
| 3-81-4-1 | Feature Location/ | misc_feature 1 . . . 10705 | |
| | Qualifiers | note = pMZT3 | |
| | NonEnglishQualifier Value | | |
| | | source 1 . . . 10705 | |
| 3-81-4-2 | Feature Location/ | mol_type = other DNA | |
| | Qualifiers | organism = synthetic construct | |
| | NonEnglishQualifier Value | | |
| 3-81-5 | Residues | | |
| | tcaccctgtc gggcaatgcc gaggcattct ggcagcagcg ccccctggcc tgtagtggat | | 60 |
| | tacgtgccgg tctgttccat cctaccaccg gctattcact gccgctggcg gttgccgtgg | | 120 |
| | ccgaccgcct gagcgcactt gatgtcttta cgtcggcctc aattcaccag gctattaggc | | 180 |
| | attttgcccg cgagcgctgg cagcagcagc gctttttccg catgctgaat cgcatgctgt | | 240 |
| | ttttagccgg acccgccgat tcacgctggc gggttatgca gcgtttttat ggtttacctg | | 300 |
| | aagatttaat tgcccgtttt tatgcgggaa aactcacgct gaccgatcgg ctacgtattc | | 360 |
| | tgagcggcaa gccgcctgtt ccggtattag cagcattgca agccattatg acgactcatc | | 420 |
| | gttaagagac agaacgaagt gtgaccagaa cgcagaagcg gtctgataaa acagaatttg | | 480 |
| | cctggcggca gtagcgcggt ggtcccacct gaccccatgc cgaactcaga agtgaaacgc | | 540 |
| | cgtagcgccg atggtagtgt ggggtctccc catgcgagag tagggaactg ccaggcatca | | 600 |
| | aataaaacga aaggctcagt cgaaagactg ggcctttcgt tttatctgtt gtttgtcggt | | 660 |
| | gaacgctctc ctgagtagga caaatcttgt aggtggacca gttggtgatt ttgaactttt | | 720 |
| | gctttgccac ggaacggtct gcgttgtcgg gaagatgcgt gatctgatcc ttcaactcag | | 780 |
| | caaaagttcg atttattcaa caaagccgcc gtcccgtcaa gtcagcgtaa tgctctgcca | | 840 |
| | gtgttacaac caattaacca attctgatta gaaaaactca tcgagcatca aatgaaactg | | 900 |
| | caatttattc atatcaggat tatcaatacc atatttttga aaaagccgtt tctgtaatga | | 960 |
| | aggagaaaac tcaccgaggc agttccatag gatggcaaga tcctggtatc ggtctgcgat | | 1020 |
| | tccgactcgt ccaacatcaa tacaacctat taatttcccc tcgtcaaaaa taaggttatc | | 1080 |
| | aagtgagaaa tcaccatgag tgacgactga atccggtgag aatggcaaaa gcttatgcat | | 1140 |
| | ttctttccag acttgttcaa caggccagcc attacgctcg tcatcaaaat cactcgcatc | | 1200 |
| | aaccaaaccg ttattcattc gtgattgcgc ctgagcgaga cgaaatacgc gatcgctgtt | | 1260 |
| | aaaaggacaa ttacaaacag gaatcgaatg caaccggcgc aggaacactg ccagcgcatc | | 1320 |
| | aacaatattt tcacctgaat caggatattc ttctaatacc tggaatgctg ttttcccggg | | 1380 |
| | gatcgcagtg gtgagtaacc atgcatcatc aggagtacgg ataaaatgct tgatggtcgg | | 1440 |
| | aagaggcata aattccgtca gccagtttag tctgaccatc tcatctgtaa catcattggc | | 1500 |
| | aacgctacct ttgccatgtt tcagaaacaa ctctggcgca tcgggcttcc catacaatcg | | 1560 |
| | atagattgtc gcacctgatt gcccgacatt atcgcgagcc catttatacc catataaatc | | 1620 |
| | agcatccatg ttggaattta atcgcggcct cgagcaagac gtttcccgtt gaatatggct | | 1680 |
| | cataacaccc cttgtattac tgtttatgta agcagacagt tttattgttc atgatgatat | | 1740 |
| | atttttatct tgtgcaatgt aacatcagag attttgagac acaacgtggc tttccccccc | | 1800 |
| | ccccctgcag gtccgacacg gggatggatg gcgttcccga tcatggtcct gcttgcttcg | | 1860 |
| | ggtggcatcg gaatgccggc gctgcaagca atgttgtcca ggcaggtgga tgaggaacgt | | 1920 |
| | caggggcagc tgcaaggctc actggcggcg ctcaccagcc tgacctcgat cgtcggaccc | | 1980 |
| | ctcctcttca cggcgatcta tgcggcttct ataacaacgt ggaacgggtg ggcatggatt | | 2040 |
| | gcaggcgctg ccctctactt gctctgcctg ccggcgctgc gtcgcgggct ttggagcggc | | 2100 |
| | gcagggcaac gagccgatcg ctgatcgtgg aaacgatagg cctatgccat gcgggtcaag | | 2160 |
| | gcgacttccg gcaagctata cgcgccctag aattgtcaat tttaatcctc tgtttatcgg | | 2220 |
| | cagttcgtag agcgcgccgt gcgtcccgag cgatactgag cgaagcaagt gcgtcgagca | | 2280 |
| | gtgcccgctt gttcctgaaa tgccagtaaa gcgctggctg ctgaaccccc agccggaact | | 2340 |
| | gaccccacaa ggccctagcg tttgcaatgc accaggtcat cattgaccca ggcgtgttcc | | 2400 |
| | accaggccgc tgcctcgcaa ctcttcgcag gcttcgccga cctgctcgcg ccacttcttc | | 2460 |
| | acgcgggtgg aatccgatcc gcacatgagg cggaaggttt ccagcttgag cgggtacggc | | 2520 |
| | tcccggtgcg agctgaaata gtcgaacatc cgtcgggccg tcggcgacag cttgcggtac | | 2580 |
| | ttctcccata tgaatttcgt gtagtggtcg ccagcaaaca gcacgacgat ttcctcgtcg | | 2640 |
| | atcaggacct ggcaacggga cgttttcttg ccacggtcca ggacgcggaa gcggtgcagc | | 2700 |
| | agcgacaccg attccaggtg cccaacgcgg tcggacgtga agcccatcgc cgtcgcctgt | | 2760 |
| | aggcgcgaca ggcattcctc ggccttcgtg taataccggc cattgatcga ccagcccagg | | 2820 |
| | tcctggcaaa gctcgtagaa cgtgaaggtg atcggctcgc cgataggggt gcgcttcgcg | | 2880 |
| | tactccaaca cctgctgcca caccagttcg tcatcgtcgg cccgcagctc gacgccggtg | | 2940 |
| | taggtgatct tcacgtcctt gttgacgtgg aaaatgacct tgttttgcag cgcctcgcgc | | 3000 |
| | gggattttct tgttgcgcgt ggtgaacagg gcagagcggg ccgtgtcgtt tggcatcgct | | 3060 |
| | cgcatcgtgt ccggccacgg cgcaatatcg aacaaggaaa gctgcatttc cttgatctgc | | 3120 |
| | tgcttcgtgt gtttcagcaa cgcggcctgc ttggcctcgc tgacctgttt tgccaggtcc | | 3180 |
| | tcgccggcgg tttttcgctt cttggtcgtc atagttcctc gcgtgtcgat ggtcatcgac | | 3240 |
| | ttcgccaaac ctgccgcctc ctgttcgaga cgacgcgaac gctccacggc ggccgatggc | | 3300 |
| | gcgggcaggg cagggggagc cagttgcacg ctgtcgcgct cgatcttggc cgtagcttgc | | 3360 |
| | tggaccatcg agccgacgga ctggaaggtt tcgcggggcg cacgcatgac ggtgcggctt | | 3420 |
| | gcgatggttt cggcatcctc ggcggaaaac cccgcgtcga tcagttcttg cctgtatgcc | | 3480 |
| | ttccggtcaa acgtccgatt cattcaccct ccttgcggga ttgccccgac tcacgccggg | | 3540 |
| | gcaatgtgcc cttattcctg atttgacccg cctggtgcct tggtgtccag ataatccacc | | 3600 |
| | ttatcggcaa tgaagtcggt cccgtagacc gtctggccgt ccttctcgta cttggtattc | | 3660 |
| | cgaatcttgc cctgcacgaa taccagctcc gcgaagtcgc tcttcttgat ggagcgcatg | | 3720 |
| | gggacgtgct tggcaatcac gcgcaccccc cggccgtttt agcggctaaa aaagtcatgg | | 3780 |
| | ctctgccctc gggcggacca cgcccatcat gaccttgcca agctcgtcct gcttctcttc | | 3840 |
| | gatcttcgcc agcagggcga ggatcgtggc atcaccgaac cgcgccgtgc gcgggtcgtc | | 3900 |
| | ggtgagccag agtttcagca ggccgcccag gcggcccagg tcgccattga tgcgggccag | | 3960 |
| | ctcgcggacg tgctcatagt ccacgacgcc cgtgattttg tagccctggc cgacggccag | | 4020 |
| | caggtaggcc tacaggctca tgccggccgc cgccgccttt tcctcaatcg ctcttcgttc | | 4080 |
| | gtctggaagg cagtacacct tgataggtgg gctgcccttc ctggttggct tggtttcatc | | 4140 |
| | agccatccgc ttgccctcat ctgttacgcc ggcggtagcc ggccagcctc gcagagcagg | | 4200 |
| | attcccgttg agcaccgcca ggtgcgaata agggacagtg aagaaggaac acccgctcgc | | 4260 |
| | gggtgggcct acttcaccta tcctgcccgg ctgacgccgt tggatacacc aaggaaagtc | | 4320 |
| | tacacgaacc ctttggcaaa atcctgtata tcgtgcgaaa aaggatggat ataccgaaaa | | 4380 |
| | aatcgctata atgaccccga agcagggtta tgcagcggaa aagatccgtc gaccctttcc | | 4440 |
| | gacgctcacc gggctggttg ccctcgccgc tgggctggcg gccgtctatg gccctgcaaa | | 4500 |
| | cgcgccagaa acgccgtcga agccgtgtgc gagacaccgc ggccgccggc gttgtggata | | 4560 |
| | cctcgcggaa aacttggccc tcactgacag atgaggggcg gacgttgaca cttgaggggc | | 4620 |
| | cgactcaccc ggcgcggcgt tgacagatga ggggcaggct cgatttcggc cggcgacgtg | | 4680 |
| | gagctggcca gcctcgcaaa tcggcgaaaa cgcctgattt tacgcgagtt tcccacagat | | 4740 |
| | gatgtggaca agcctgggga taagtgccct gcggtattga cacttgaggg gcgcgactac | | 4800 |
| | tgacagatga ggggcgcgat ccttgacact tgaggggcag agtgctgaca gatgaggggc | | 4860 |
| | gcacctattg acatttgagg ggctgtccac aggcagaaaa tccagcattt gcaagggttt | | 4920 |
| | ccgcccgttt ttcggccacc gctaacctgt cttttaacct gcttttaaac caatatttat | | 4980 |
| | aaaccttgtt tttaaccagg gctgcgccct gtgcgcgtga ccgcgcacgc cgaagggggg | | 5040 |
| | tgccccccct tctcgaaccc tcccggcccg ctaacgcggg cctcccatcc ccccaggggc | | 5100 |
| | tgcgcccctc ggccgcgaac ggcctcaccc caaaaatggc agccaagctg accacttctg | | 5160 |
| | cgctcggccc ttccggctgg ctggtttatt gctgataaat ctggagccgg tgagcgtggg | | 5220 |
| | tctcgcggta tcattgcagc actggggcca gatggtaagc cctcccgtat cgtagttatc | | 5280 |
| | tacacgacgg ggagtcaggc aactatggat gaacgaaata gacagatcgc tgagataggt | | 5340 |
| | gcctcactga ttaagcattg gtaactgtca gaccaagttt actcatatat actttagatt | | 5400 |
| | gatttaaaac ttcattttta atttaaaagg atctaggtga agatcctttt tgataatctc | | 5460 |
| | atgaccaaaa tcccttaacg tgagttttcg ttccactgag cgtcagaccc cgtagaaaag | | 5520 |
| | atcaaaggat cttcttgaga tccttttttt ctgcgcgtaa tctgctgctt gcaaacaaaa | | 5580 |
| | aaaccaccgc taccagcggt ggtttgtttg ccggatcaag agctaccaac tctttttccg | | 5640 |
| | aaggtaactg gcttcagcag agcgcagata ccaaatactg tccttctagt gtagccgtag | | 5700 |
| | ttaggccacc acttcaagaa ctctgtagca ccgcctacat acctcgctct gctaatcctg | | 5760 |
| | ttaccagtgg ctgctgccag tggcgataag tcgtgtctta ccgggttgga ctcaagacga | | 5820 |
| | tagttaccgg ataaggcgca gcggtcgggc tgaacggggg gttcgtgcac acagcccagc | | 5880 |
| | ttggagcgaa cgacctacac cgaactgaga tacctacagc gtgagctatg agaaagcgcc | | 5940 |
| | acgcttcccg aagggagaaa ggcggacagg tatccggtaa gcggcagggt cggaacagga | | 6000 |
| | gagcgcacga gggagcttcc agggggaaac gcctggtatc tttatagtcc tgtcgggttt | | 6060 |
| | cgccacctct gacttgagcg tcgatttttg tgatgctcgt caggggggcg gagcctatgg | | 6120 |
| | aaaaacgcca gcaacgcggc ctttttacgg ttcctggcct tttgctggcc ttttgctcac | | 6180 |
| | atgttctttc ctgcgttatc ccctgattct gtggataacc gtattaccgc ctttgagtga | | 6240 |
| | gctgataccg ctcgccgcag ccgaacgacc gagcgcagcg agtcagtgag cgaggaagcg | | 6300 |
| | gaagagcgcc caatacgcaa accgcctctc cccgcgcgtt ggccgattca ttaatgcagc | | 6360 |
| | tggcagccag cgcttcgtta atacagatgt aggtgttcca cagggtagcc agcagcatcc | | 6420 |
| | tgcgatgcag atccggaaca taatggtgca gggcgctgac tttatagcta gctcagccct | | 6480 |
| | tggtacaatg ctagcgagca accaacactt aaagaggaga aaatgtatcc gtttataagg | | 6540 |
| | acagcccgaa tgacggtctg cgcaaaaaaa cacgttcatc tcactcgcga tgctgcggag | | 6600 |
| | cagttactgg ctgatattga tcgacgcctt gatcagttat tgcccgtgga gggagaacgg | | 6660 |
| | gatgttgtgg gtgccgcgat gcgtgaaggt gcgctggcac cgggaaaacg tattcgcccc | | 6720 |
| | atgttgctgt tgctgaccgc ccgcgatctg ggttgcgctg tcagccatga cggattactg | | 6780 |
| | gatttggcct gtgcggtgga aatggtccac gcggcttcgc tgatccttga cgatatgccc | | 6840 |
| | tgcatggacg atgcgaagct gcggcgcgga cgccctacca ttcattctca ttacggagag | | 6900 |
| | catgtggcaa tactggcggc ggttgccttg ctgagtaaag cctttggcgt aattgccgat | | 6960 |
| | gcagatggcc tcacgccgct ggcaaaaaat cgggcggttt ctgaactgtc aaacgccatc | | 7020 |
| | ggcatgcaag gattggttca gggtcagttc aaggatctgt ctgaagggga taagccgcgc | | 7080 |
| | agcgctgaag ctattttgat gacgaatcac tttaaaacca gcagcgtgtt ttgtgcctcc | | 7140 |
| | atgcagatgg cctcgattgt tgcgaatgcc tccagcgaag cgcgtgattg cctgcatcgt | | 7200 |
| | ttttcacttg atcttggtca ggcatttcaa ctgctggacg atttgaccga tggcatgacc | | 7260 |
| | gacaccggta aggatagcaa tcaggacgcc ggtaaatcga cgctggtcaa tctgttaggc | | 7320 |
| | cctagggcgg ttgaagaacg tctgagacaa catcttcatc ttgccagtga gcatctctct | | 7380 |
| | gcggcctgcc aacacgggca cgccactcaa cattttattc aggcctggtt tgacaaaaaa | | 7440 |
| | ctcgctgccg tcagttaaag gtctctagac aggatgtgtc acacaggaaa ccatgaaacc | | 7500 |
| | aactacggta attggtgcag gcttcggtgg cctggcactg gcaattcgtc tacaggctgc | | 7560 |
| | ggggatcccc gtcttactgc ttgaacaacg tgataaaccc ggcggtcggg cttatgtcta | | 7620 |
| | cgaggatcag gggtttacct ttgatgcagg cccgacggtt atcaccgatc ccagtgccat | | 7680 |
| | tgaagaactg tttgcactgg caggaaaaca gttaaaagag tatgtcgaac tgctgccggt | | 7740 |
| | tacgccgttt taccgcctgt gttgggagtc agggaaggtc tttaattacg ataacgatca | | 7800 |
| | aacccggctc gaagcgcaga ttcagcagtt taatccccgc gatgtcgaag gttatcgtca | | 7860 |
| | gtttctggac tattcacgcg cggtgtttaa agaaggctat ctgaagctcg gtactgtccc | | 7920 |
| | ttttttatcg ttcagagaca tgcttcgcgc cgcacctcaa ctggcgaaac tgcaggcatg | | 7980 |
| | gagaagcgtt tacagtaagg ttgccagtta catcgaagat gaacatctgc gccaggcgtt | | 8040 |
| | ttctttccac tcgctgttgg tgggcggcaa tcccttcgcc acctcatcca tttatacgtt | | 8100 |
| | gatacacgcg ctggagcgtg agtggggcgt ctggtttccg cgtggcggca ccggcgcatt | | 8160 |
| | agttcagggg atgataaagc tgtttcagga tctgggtggt gaagtcgtgt taaacgccag | | 8220 |
| | agtcagccat atggaaacga caggaaacaa gattgaagcc gtgcatttag aggacggtcg | | 8280 |
| | caggttcctg acgcaagccg tcgcgtcaaa tgcagatgtg gttcatacct atcgcgacct | | 8340 |
| | gttaagccag caccctgccg cggttaagca gtccaacaaa ctgcagacta agcgtatgag | | 8400 |
| | taactctctg tttgtgctct attttggttt gaatcaccat catgatcagc tcgcgcatca | | 8460 |
| | cacggtttgt ttcggcccgc gttaccgcga actgattgac gagattttta atcatgatgg | | 8520 |
| | cctcgcagaa gacttctcac tttatctgca cgcgccctgt gtcacggatt cgtcactggc | | 8580 |
| | gcctgaaggt tgcggcagtt actatgtgtt ggcgccggtg ccgcatttag gcaccgcgaa | | 8640 |
| | cctcgactgg acggttgagg ggccaaaact acgcgaccgt atttttgagt accttgagca | | 8700 |
| | gcattacatg cctggcttac ggagtcagct ggtcacgcac cagatgttta cgccgtttga | | 8760 |
| | ttttcgcgac cagcttaatg cctatcaggg ctcagccttt tctgtggagc ccgttcttac | | 8820 |
| | ccagagcgcc tggtttcggc cgcataaccg cgataaaacc attactaatc tctacctggt | | 8880 |
| | cggcgcaggc acgcatcccg gcgcaggcat tcctggcgtc atcggctcgg caaaagcgac | | 8940 |
| | agcaggtttg atgctggagg atctgattta agtgatcgtt gagtggtgaa cttaaagagg | | 9000 |
| | agaaaatgaa taatccgtcg ttactcaatc atgcggtcga aacgatggca gttggctcga | | 9060 |
| | aaagttttgc gacagcctca aagttatttg atgcaaaaac ccggcgcagc gtactgatgc | | 9120 |
| | tctacgcctg gtgccgccat tgtgacgatg ttattgacga ccagacgctg ggcttccagg | | 9180 |
| | cccggcagcc tgccttacaa acgcccgaac aacgtctgat gcaacttgag atgaaaacgc | | 9240 |
| | gccaggccta tgcaggatcg cagatgcacg aaccggcgtt tgcggctttt caggaagtgg | | 9300 |
| | ctatggctca tgatatcgcc ccggcttacg cgtttgatca tctggaaggc ttcgccatgg | | 9360 |
| | atgtacgcga agcgcaatac agccaactgg acgatacgct gcgctattgc tatcacgttg | | 9420 |
| | caggcgttgt cggcttgatg atggcgcaaa tcaggggcgt acgggataac gccacgctgg | | 9480 |
| | accgcgcctg tgaccttggg ctggcatttc agttgaccaa tattgctcgc gataggtggg | | 9540 |
| | acgatgcgca tgcgggccgc tgttatctgc cggcaagctg gctggagcat gaaggtctga | | 9600 |
| | acaaagagaa ttatgcggca cctgaaaacc gtcaggcgct gagccgtatc gcccgtcgtt | | 9660 |
| | tggtgcagga agcagaacct tactatttgt ctgccacagc gggcctggct gggttgcccc | | 9720 |
| | tgcgttcggc ctgggcaatc gctacggcga agcaggttta ccggaaaata ggtgtcaaag | | 9780 |
| | ttgaacaggc cggtcagcaa gcctgggatc agcggcagtc aacgaccacg cccgaaaaat | | 9840 |
| | taacgctgct gctggccgcc tctggtcagg cccttacttc ccggatgcgg gctcatcctc | | 9900 |
| | cccgccctgc gcatctctgg cagcgcccgc tctaatcacg tagcaagctg acagtttaaa | | 9960 |
| | gaggagaaaa tgggagcggc tatgcaaccg cattatgatc tgattctcgt gggggctgga | | 10020 |
| | ctcgcgaatg gccttatcgc cctgcgtctt cagcagcagc aacctgatat gcgtattttg | | 10080 |
| | cttatcgacg ccgcacccca ggcgggcggg aatcatacgt ggtcatttca ccacgatgat | | 10140 |
| | ttgactgaga gccaacatcg ttggatagct tcgctggtgg ttcatcactg gcccgactat | | 10200 |
| | caggtacgct ttcccacacg ccgtcgtaag ctgaacagcg gctacttctg tattacttct | | 10260 |
| | cagcgtttcg ctgaggtttt acagcgacag tttggcccgc acttgtggat ggataccgcg | | 10320 |
| | gtcgcagagg ttaatgcgga atctgttcgg ttgaaaaagg gtcaggttat cggtgcccgc | | 10380 |
| | gcggtgattg acgggcgggg ttatgcggca aactcagcac tgagcgtggg cttccaggcg | | 10440 |
| | tttattggcc aggaatggcg attgagccac ccgcatggtt tatcgtctcc cattatcatg | | 10500 |
| | gatgccacgg tcgatcagca aaatggttat cgcttcgtgt acagcctgcc gctctcgccg | | 10560 |
| | accagattgt taattgaaga cacgcactat atcgataatg cgacattaga tcctgaacgc | | 10620 |
| | gcgcggcaaa atatttgcga ctatgccgcg caacagggtt ggcagcttca gacattgctg | | 10680 |
| | cgtgaagaac agggcgcctt accca | | 10705 |
| 3-82 | Sequences | | |
| 3-82-1 | Sequence Number [ID] | 82 | |
| 3-82-2 | Molecular Type | DNA | |
| 3-82-3 | Length | 1773 | |
| | | source 1 . . . 173 | |
| 3-82-4-1 | Features Location/ | mol_type = other DNA | |
| | Qualifiers | organism = Methylosinus trichosporum | |
| | NonEnglishQualifier Value | | |
| 3-82-5 | Residues | | |
| | atggccagaa aaatgaccgg agcggaaatg gtcgtcgaag ccctgaagga tcagggcgtc | | 60 |
| | gagattatct tcggctatcc cggcggcgcc gtgcttccga tctatgacgc gctcttccac | | 120 |
| | caggagaagg tgcagcacat tctcgtgcgc cacgagcagg gcgccgccca tgcggccgag | | 180 |
| | ggctatgcgc gctcctccgg caaggtcggc gtgctgctgg tcacctccgg acccggcgcc | | 240 |
| | accaacacca tcaccggcct caccgatgcg ctgatggact ccattcccgt ggtctgcatc | | 300 |
| | accggccagg tgccgacgca tctcatcggc tcggacgcct ttcaagagtg cgatacggtc | | 360 |
| | ggcatcaccc gtcactgcac caagcataat tatctggtga agagcgtcga cgatctgccg | | 420 |
| | cgcattctgc acgaggcctt ctatgtcgcc tcgagcgggc ggccgggccc tgtggtcatc | | 480 |
| | gacatcccca aggatgtgca attcgccagc ggaacctata ccggcccgcg caacgtccat | | 540 |
| | cacaagacct atcagcccaa gctcgagggc gacacggagt ctatccgccg cgccgtgaag | | 600 |
| | atgatggccg ccgccaagcg gccgatcttc tacaccggcg gcggcgtcat caattccggt | | 660 |
| | cccgcggcct cgacgctgct gcgcgagctg gtgtcgctga ccggctttcc gatcacctcg | | 720 |
| | accttgatgg gcctcggcgc ctatccgggc tccggcccca attggctcgg catgctcggc | | 780 |
| | atgcacggca ccttcgaggc caataatgcg atgcatgatt gcgatctgat gatcgccgtc | | 840 |
| | ggcgcgcgtt tcgacgatcg catcaccgga cggctcgacg ccttctcgcc cggctcgaag | | 900 |
| | aagatccaca tcgatatcga tcgctcctcg atcaataaga atgtgaagat cgatctgccg | | 960 |
| | atcgtcggcg actgcggcca tgtgctggag agtctggtgc gcgtctggcg ctccgaggcg | | 1020 |
| | atgcacgccg agaagcagcc gctcgacggc tggtggaaga cgatcgacca ttggcgcgag | | 1080 |
| | cgcaagtcgc tcgccttccg caattcggac aaggtgatca agccgcaata cgccgtgcag | | 1140 |
| | cggctctatg cgctcaccaa ggatcgcgat ccctacatca cgacggaagt cggccagcat | | 1200 |
| | cagatgtggg ccgcgcagca ttatcatttc gacgagccca atcgctggat gacttccggc | | 1260 |
| | gggctcggca ccatgggcta tggtctgccg gcggcgatcg gagcgcagct cgcgcatccg | | 1320 |
| | aaatcgctgg tcgtcgacat cgccggcgag gcctcgatcc tgatgaacat tcaggagatg | | 1380 |
| | tcgacggcga tccaatatcg gctgccggtg aaggtgttca tcctcaacaa tgaatatatg | | 1440 |
| | ggcatggtgc gccagtggca ggagctgctg cacggcgggc gctactcgca ctcctattcg | | 1500 |
| | gaggcgctgc ccgatttcgt gaagctcgcc gaagccttcg ggggcaaggg catccgctgc | | 1560 |
| | tcggacccgg cggagctcga tagcgcgatt ctcgagatga tcgactatga cgggccggtg | | 1620 |
| | atcttcgatt gtctcgtcga gaaaaacgag aattgcttcc cgatgatccc gtcgggcaag | | 1680 |
| | gcgcataacg acatgctgct cgccgatctc ggcgacgacg ccggcgtcga gctcggctcg | | 1740 |
| | atcatcgacg agaagggcaa gatgctggtg tga | | 1773 |
| 3-83 | Sequences | | |
| 3-83-1 | Sequence Number [ID] | 83 | |
| 3-83-2 | Molecular Type | DNA | |
| 3-83-3 | Length | 1056 | |
| | | source 1 . . . 1056 | |
| 3-83-4-1 | Features Location/ | mol_type = other DNA | |
| | Qualifiers | organism = Methylosinus trichosporum | |
| | NonEnglishQualifier Value | | |
| 3-83-5 | Residues | | |
| | atgtccacca aagcctatgc cgttgcgtcc gccgaggcgc tcttcggccc gctcgcgatc | | 60 |
| | gagcgccgcg cgctcgggcc cgaggatgta gagatcgaca tcctctattg cggcgtctgc | | 120 |
| | cattccgatc tgcacacggc gcgcagtgaa tggccgggca cgcgctaccc atgcgtcccg | | 180 |
| | ggccacgaga ttgtcggccg cgtcaccgct gtcggcgcga aggtgacgaa attttcggtc | | 240 |
| | ggcgatctcg ccgccgtcgg ctgcatggtc gacagctgcc ggcgatgctt gtcctgcgac | | 300 |
| | gacgggctcg aacaatattg cgagcacggt ttcaccgcca cctataacgg cccgatctac | | 360 |
| | ggctcgggcg agaacacctt tggcggctat tcggagaaaa tcgtcgtcga cgcgcatttc | | 420 |
| | gtgctggcga tccaccattc tgagacgcag cttgccggag tcgcgccgct gctctgcgcc | | 480 |
| | ggcatcacca cttggtcgcc gctcaagcat tggggtgtcg gcccgggaaa atcggtcggc | | 540 |
| | atcgtcggca tcggcgggct cggccatatg ggggtcaagc tcgcccatgc gctcggcgcc | | 600 |
| | catgtcgtcg ccttcaccac ctcgccgtca aagcgcgacg cggccctcgc gctcggcgcc | | 660 |
| | gacgaggtcg tcgtctccac agatcctgcc gctatggcgg cgcgggcggg aagcctcgac | | 720 |
| | ttcattctcg atacggtcgc cgtcgcccat gacctcgacg cttatgtgaa tctgttgaag | | 780 |
| | cgcgatggcg ctctggtgct cgtcggcgtg ccggcgacgc cgcatccctc gccatcggcg | | 840 |
| | ggcgggttga tcttcaagcg gcgccaggtc gccggctcgc tgatcggcgg cgtaaaggag | | 900 |
| | acgcaggaga tgctcgactt ctgcgccgag cgcggcattg tcgcggacat agagacgatc | | 960 |
| | gccatgcagc agatcgagac cgcctatgcg cgcatgctga agaatgatgt gaaataccgc | | 1020 |
| | ttcgtcatcg acatggcgac gctgaaggcg gcgtga | | 1056 |
| 3-84 | Sequences | | |
| 3-84-1 | Sequence Number [ID] | 84 | |
| 3-84-2 | Molecular Type | DNA | |
| 3-84-3 | Length | 1029 | |
| | | source 1 . . . 1029 | |
| 3-84-4-1 | Features Location/ | mol_type = other DNA | |
| | Qualifiers | organism = Methylococcus capsulatus | |
| | NonEnglishQualifier Value | | |
| 3-84-5 | Residues | | |
| | atgaaagctt gggtgatcga ccgaatcggc ccgctggact cgtcgcgaac tctgctacgc | | 60 |
| | gccaccgacc tcccggtgcc ggagcccggc cctggcgaaa tcctgctgca ggtggcggtt | | 120 |
| | tgcggcgtct gccacaccga aatcgacgag atcgagggcc gcaccgcgcc gccgcgcctg | | 180 |
| | ccggtcgtgc ccggacacca agcggtcggt cggatcgcgg ctctcggctc cggcgtggcg | | 240 |
| | gaattcgctt tgggcgaccg cgtcggcgtg gcctggatct tttctgcctg cggagaatgc | | 300 |
| | gaattctgcc ggtcgggacg ggagaacctc tgtttcgcat tctgtgccac cgggcgcgat | | 360 |
| | gtcgacggcg gctacgccca gtacatgacc gtcccggcgg cctttgcttt ccgcattccg | | 420 |
| | gagggattca ccgatgccga agcggcgccg cttctgtgcg ccggcgccat cggttaccgt | | 480 |
| | tcgctcaatc tcagcgggct gaaaaacggc cagccgctgg ggctcaccgg gttcggggct | | 540 |
| | tccgcccatc tggtgctgat gatggcccgg taccggtttc ccgattcgga agtctatgtc | | 600 |
| | tttgcgcgtc atcccgagga gcgcgcgttc gcgcctcagc tgggcgcggt ctgggccggc | | 660 |
| | gacaccgcgg acattgctcc cgccccgctg gccgccatca tcgacacgac gccggcgtgg | | 720 |
| | aagccggtgg tcgcagcgct cgccaacctc gctcccggtg gccggctggt cgttaatgcg | | 780 |
| | atccgcaagg cgccggacga tcgcgcctgt ctcgccgaac tcgactatgc ccggcacttg | | 840 |
| | tggatggaac gggaaatcaa gtcggtcgcc aacgtggcgc gcgatgacgt ggccgggttc | | 900 |
| | ctggcgctgg cggcggaaat gggcatccgt cccgagacgg aggagtaccc gttcgaggat | | 960 |
| | gccgaccggg cgctgctcga cctcaagcaa cgccggattc gcggggcgaa ggtgttgcgg | | 1020 |
| | gtgacttga | | 1029 |
| 3-85 | Sequences | | |
| 3-85-1 | Sequence Number [ID] | 85 | |
| 3-85-2 | Molecular Type | DNA | |
| 3-85-3 | Length | 1068 | |
| | | source 1 . . . 1068 | |
| 3-85-4-1 | Features Location/ | mol_type = other DNA | |
| | Qualifiers | organism = Methylococcus capsulatus | |
| | NonEnglishQualifier Value | | |
| 3-85-5 | Residues | | |
| | atgcctacag ccaaagccta tgccgctttt tccgcagact cggcgctggc gccgttcgtc | | 60 |
| | ctgcagcggc gcgacccact gccccaggac atccgcatcg gaatcctgta ctgcggtgtc | | 120 |
| | tgccattccg acctgcacca ggcacgcaat gagtggaatg cgaccacata tccttgtgtg | | 180 |
| | ccaggccatg agatcgtcgg caaggtcctt gaagtcggcc gcagcgtgac gaagttcaag | | 240 |
| | cccggcgaca cggtcgcggt gggctgcatg gtggattcct gccggacctg cccgaactgc | | 300 |
| | gtggacgccc tggaacagca ctgcgagcac ggccccgtct tcacctacaa cagccccgat | | 360 |
| | ccgcacggcg gcggcatgac cttcggtggc tatgccgaga gcatcgtggt cgacgaggcc | | 420 |
| | ttcgtgctgc ggataccgga cggactggac ctcgcggccg ccgccccgct gttgtgcgcc | | 480 |
| | gggattacca cctattcgcc cctgcggcac tggaaagtgg gggcgggtca gcgggtcggg | | 540 |
| | gtcgtcggtc tgggtggact gggacacatg gcgctcaagt tcgcgcatac cttcggcgcc | | 600 |
| | gaaacggtgc tgttcacgac gacgccggac aaggcggagg atgcccgtcg gctgggagcg | | 660 |
| | gacgaggtcg tcgtgtcgag ggatcccgag gccatggcgc ggcaggccgg ccggttcgat | | 720 |
| | ttcatcctcg acaccgtctc ggcgccccat gacatcgatg cctatctgaa cctgctgagg | | 780 |
| | cgggacggca cgctgaccct ggtcggcgta cctccgcaag gggtacaggt catgcccttc | | 840 |
| | agcctgatcg gcgggcgccg gcgactggct ggttcattga tcggcggcat ccgggaaacc | | 900 |
| | caggagatgc tggatttctg cggcgaacac ggcatcgtct gcgacatcga gctgattccg | | 960 |
| | atccaaggaa tcaacgacgc cttcgagcgc atgctcaaaa gcgacgtgaa ataccgtttc | | 1020 |
| | gtgatcgaca tggcgacgct gaacggggag tcgtccggag ggcgatga | | 1068 |
| 3-86 | Sequences | | |
| 3-86-1 | Sequence Number [ID] | 86 | |
| 3-86-2 | Molecular Type | DNA | |
| 3-86-3 | Length | 1647 | |
| | | source 1 . . . 1647 | |
| 3-86-4-1 | Features Location/ | mol_type = other DNA | |
| | Qualifiers | organism = Lactococcus lactis | |
| | NonEnglishQualifier Value | | |
| 3-86-5 | Residues | | |
| | atgtacaccg tgggcgacta tctgctggac cggctgcatg aactgggcat cgaggaaatc | | 60 |
| | ttcggcgtcc ccggcgacta taacctgcag ttcctggacc agatcatcag ccgcaaggac | | 120 |
| | atgaagtggg tgggcaacgc caacgagctg aacgcctcgt acatggcgga cggctatgcc | | 180 |
| | cggaccaaga aggccgcggc cttcctgacc accttcggcg tcggcgaact gagcgccgtg | | 240 |
| | aacggcctgg cgggctcgta cgccgagaac ctgccggtcg tggaaatcgt cggctccccc | | 300 |
| | accagcaagg tgcagaacga gggcaagttc gtccaccata ccctggccga cggcgacttc | | 360 |
| | aagcacttca tgaagatgca tgaaccggtg accgcggccc gcaccctgct gaccgccgag | | 420 |
| | aacgcgaccg tcgaaatcga ccgcgtgctg agcgcgctgc tgaaggagcg gaagccggtc | | 480 |
| | tatatcaacc tgcccgtcga cgtggcggcc gcgaaggccg agaagccgtc cctgcccctg | | 540 |
| | aagaaggaaa accccacctc gaacacctcc gaccaggaga tcctgaacaa gatccaggaa | | 600 |
| | agcctgaaga acgccaagaa gccgatcgtg atcaccggcc acgagatcat ctcgttcggc | | 660 |
| | ctggaaaaca ccgtcaccca gttcatctcc aagaccaagc tgccgatcac caccctgaac | | 720 |
| | ttcggcaaga gctcggtgga cgagaccctg ccctcgttcc tgggcatcta caacggcaag | | 780 |
| | ctgtccgaac cgaacctgaa ggagttcgtg gaaagcgcgg acttcatcct gatgctgggc | | 840 |
| | gtcaagctga ccgactccag caccggcgcc ttcacccacc atctgaacga gaacaagatg | | 900 |
| | atctcgctga acatcgacga gggcaagatc ttcaacgaat ccatccagaa cttcgacttc | | 960 |
| | gaaagcctga tctcgtccct gctggacctg tccggcatcg agtacaaggg caagtatatc | | 1020 |
| | gacaagaagc aggaagactt cgtcccgagc aacgcgctgc tgtcgcagga ccgcctgtgg | | 1080 |
| | caggccgtgg agaacctgac ccagagcaac gagaccatcg tcgcggaaca gggcacctcg | | 1140 |
| | ttcttcggcg ccagctcgat cttcctgaag ccgaagtcgc acttcatcgg ccagcccctg | | 1200 |
| | tggggctcca tcggctacac cttccccgcc gcgctgggct cgcagatcgc ggacaaggaa | | 1260 |
| | tcccggcatc tgctgttcat cggcgacggc agcctgcagc tgaccgtgca ggagctgggc | | 1320 |
| | ctggccatcc gcgaaaagat caacccgatc tgcttcatca tcaacaacga cggctatacc | | 1380 |
| | gtcgagcggg aaatccacgg cccgaaccag tcgtacaacg acatccccat gtggaactat | | 1440 |
| | tccaagctgc cggagagctt cggcgccacc gaggaacgcg tcgtgtccaa gatcgtccgg | | 1500 |
| | accgagaacg agttcgtcag cgtgatgaag gaagcccagg cggaccccaa ccggatgtac | | 1560 |
| | tggatcgagc tggtgctggc gaaggaagac gccccgaagg tcctgaagaa gatgggcaag | | 1620 |
| | ctgttcgccg aacagaacaa gagctga | | 1647 |
| 3-87 | Sequences | | |
| 3-87-1 | Sequence Number [ID] | 87 | |
| 3-87-2 | Molecular Type | DNA | |
| 3-87-3 | Length | 1692 | |
| | | source 1 . . . 1692 | |
| 3-87-4-1 | Features Location/ | mol_type = other DNA | |
| | Qualifiers | organism = Saccharomyces cerevisiae | |
| | NonEnglishQualifier Value | | |
| 3-87-5 | Residues | | |
| | atgtcggaaa tcaccctggg caagtacctg ttcgagcggc tgaagcaggt caacgtcaac | | 60 |
| | accatcttcg gcctgcccgg cgacttcaac ctgagcctgc tggacaagat ctacgaggtc | | 120 |
| | gacggcctgc gctgggccgg caacgcgaac gaactgaacg ccgcgtacgc cgcggacggc | | 180 |
| | tatgcccgga tcaagggcct gtcggtcctg gtgaccacct tcggcgtggg cgagctgtcg | | 240 |
| | gccctgaacg gcatcgccgg ctcctacgcg gaacacgtcg gcgtgctgca tgtcgtgggc | | 300 |
| | gtcccgagca tctcggccca ggcgaagcag ctgctgctgc accataccct gggcaacggc | | 360 |
| | gacttcaccg tgttccaccg catgtccgcc aacatcagcg agaccacctc gatgatcacc | | 420 |
| | gacatcgcca ccgcgccgag cgaaatcgac cgcctgatcc ggaccacctt catcacccag | | 480 |
| | cggccgtcgt acctgggcct gcccgccaac ctggtcgacc tgaaggtgcc gggcagcctg | | 540 |
| | ctggagaagc ccatcgacct gtcgctgaag ccgaacgacc ccgaggccga aaaggaagtc | | 600 |
| | atcgacaccg tgctggaact gatccagaac agcaagaacc tgatcgacct gtccgacgcc | | 660 |
| | tgcgcgagcc gccacaacgt gaagaaggag acccagaagc tgatcgacct gacccagttc | | 720 |
| | ccggccttcg tcacccccct gggcaagggc tccatcgacg agcagcatcc gcggtacggc | | 780 |
| | ggcgtctatg tgggcaccct gagcaagcag gacgtcaagc aggccgtgga aagcgcggac | | 840 |
| | ctgatcctgt cggtgggcgc cctgctgtcc gacttcaaca ccggctcctt cagctactcg | | 900 |
| | tataagacca agaacgtcgt ggagttccat tcggactacg tcaaggtgaa gaacgcgacc | | 960 |
| | ttcctgggcg tccagatgaa gttcgccctg cagaacctgc tgaaggtgat cccggacgtc | | 1020 |
| | gtgaagggct ataagtccgt cccggtgccc accaagaccc ccgccaacaa gggcgtcccg | | 1080 |
| | gcgtcgaccc ccctgaagca ggaatggctg tggaacgagc tgtccaagtt cctgcaggaa | | 1140 |
| | ggcgacgtga tcatctcgga gaccggcacc tccgcgttcg gcatcaacca gaccatcttc | | 1200 |
| | ccgaaggacg cctacggcat cagccaggtc ctgtggggct cgatcggctt caccaccggc | | 1260 |
| | gccaccctgg gcgccgcgtt cgccgcggag gaaatcgacc cgaacaagcg cgtcatcctg | | 1320 |
| | ttcatcggcg acggctccct gcagctgacc gtgcaggaaa tcagcaccat gatccggtgg | | 1380 |
| | ggcctgaagc cctacctgtt cgtgctgaac aacgacggct ataccatcga gaagctgatc | | 1440 |
| | cacggcccgc atgcggaata caacgagatc cagacctggg accacctggc cctgctgccc | | 1500 |
| | gccttcggcg cgaagaagta tgaaaaccat aagatcgcca ccaccggcga gtgggacgcg | | 1560 |
| | ctgaccaccg actccgagtt ccagaagaac agcgtcatcc gcctgatcga gctgaagctg | | 1620 |
| | ccggtgttcg acgcccccga aagcctgatc aagcaggcgc agctgaccgc cgcgaccaac | | 1680 |
| | gccaagcagt ga | | 1692 |
| 3-88 | Sequences | | |
| 3-88-1 | Sequence Number [ID] | 88 | |
| 3-88-2 | Molecule Type | DNA | |
| 3-88-3 | Length | 1908 | |
| | | source 1 . . . 1908 | |
| 3-88-4-1 | Features Location/ | mol_type = other DNA | |
| | Qualifiers | organism = Saccharomyces cerevisiae | |
| | NonEnglishQualifier Value | | |
| 3-88-5 | Residues | | |
| | atggcccccg tcaccatcga gaagttcgtc aaccaggaag agcggcatct ggtgtccaac | | 60 |
| | cggagcgcga ccatcccgtt cggcgagtac atcttcaagc gcctgctgag catcgacacc | | 120 |
| | aagtcggtgt tcggcgtgcc gggcgacttc aacctgagcc tgctggagta cctgtatagc | | 180 |
| | ccctcggtcg aatcggccgg cctgcgctgg gtgggcacct gcaacgaact gaacgccgcg | | 240 |
| | tacgccgcgg acggctactc ccggtatagc aacaagatcg gctgcctgat caccacctat | | 300 |
| | ggcgtcggcg aactgtcggc gctgaacggc atcgcgggct ccttcgccga gaacgtgaag | | 360 |
| | gtcctgcaca tcgtgggcgt cgccaagtcg atcgactccc gcagctcgaa cttctcggac | | 420 |
| | cggaacctgc accatctggt cccgcagctg catgactcca acttcaaggg ccccaaccac | | 480 |
| | aaggtgtacc atgacatggt gaaggaccgc gtcgcgtgct ccgtggccta tctggaggac | | 540 |
| | atcgaaaccg cctgcgacca ggtggacaac gtcatccggg acatctacaa gtatagcaag | | 600 |
| | ccgggttaca tcttcgtccc cgcggacttc gccgacatgt ccgtgacctg cgacaacctg | | 660 |
| | gtgaacgtcc cgcgcatcag ccagcaggac tgcatcgtgt acccctccga aaaccagctg | | 720 |
| | agcgacatca tcaacaagat cacctcgtgg atctactcca gcaagacccc ggccatcctg | | 780 |
| | ggcgacgtcc tgaccgaccg gtatggcgtg agcaacttcc tgaacaagct gatctgcaag | | 840 |
| | accggcatct ggaacttctc gaccgtcatg ggcaagtcgg tgatcgacga atccaacccg | | 900 |
| | acctacatgg gccagtataa cggcaaggaa ggcctgaagc aggtctacga gcacttcgaa | | 960 |
| | ctgtgcgacc tggtcctgca tttcggcgtg gacatcaacg agatcaacaa cggccactac | | 1020 |
| | accttcacct ataagccgaa cgcgaagatc atccagttcc atcccaacta catccgcctg | | 1080 |
| | gtggacaccc ggcagggcaa cgaacagatg ttcaagggca tcaacttcgc cccgatcctg | | 1140 |
| | aaggagctgt ataagcgcat cgacgtcagc aagctgtcgc tgcagtacga cagcaacgtg | | 1200 |
| | acccagtata ccaacgagac catgcggctg gaagacccca ccaacggcca gtcgtccatc | | 1260 |
| | atcacccagg tccacctgca gaagaccatg ccgaagttcc tgaaccccgg cgacgtcgtg | | 1320 |
| | gtctgcgaga ccggctcctt ccagttcagc gtgcgcgact tcgcgttccc gagccagctg | | 1380 |
| | aagtacatct cgcagggctt cttcctgtcc atcggcatgg ccctgcccgc cgcgctgggc | | 1440 |
| | gtcggcatcg cgatgcagga ccactcgaac gcccatatca acggcggcaa cgtgaaggaa | | 1500 |
| | gactacaagc cgcggctgat cctgttcgaa ggcgacggcg ccgcgcagat gaccatccag | | 1560 |
| | gagctgtcca ccatcctgaa gtgcaacatc ccgctggaag tcatcatctg gaacaacaac | | 1620 |
| | ggctacacca tcgagcgcgc catcatgggc cccacccgga gctataacga cgtgatgtcg | | 1680 |
| | tggaagtgga ccaagctgtt cgaagcgttc ggcgacttcg acggcaagta caccaactcc | | 1740 |
| | accctgatcc agtgcccgag caagctggcc ctgaagctgg aggaactgaa gaactcgaac | | 1800 |
| | aagcgctccg gcatcgagct gctggaagtc aagctgggcg agctggactt ccccgaacag | | 1860 |
| | ctgaagtgca tggtggaggc cgcggccctg aagcggaaca agaagtga | | 1908 |
| 3-89 | Sequences | | |
| 3-89-1 | Sequence Number [ID] | 89 | |
| 3-89-2 | Molecule Type | DNA | |
| 3-89-3 | Length | 1047 | |
| | | source 1 . . . 1047 | |
| | Features Location/ | mol_type = other DNA | |
| 3-89-4-1 | Qualifiers | organism = Saccharomyces cerevisiae | |
| | NonEnglishQualifier Value | | |
| 3-89-5 | Residues | | |
| | atgagcatcc ccgagaccca gaaggccatc atcttctacg agagcaacgg caagctggaa | | 60 |
| | cataaggaca tcccggtgcc caagcccaag ccgaacgaac tgctgatcaa cgtgaagtac | | 120 |
| | agcggcgtct gccacaccga cctgcacgcg tggcatggcg actggccgct gcccaccaag | | 180 |
| | ctgcccctgg tgggcggcca tgaaggcgcc ggcgtcgtgg tcggcatggg cgagaacgtc | | 240 |
| | aagggctgga agatcggcga ctacgcgggc atcaagtggc tgaacggcag ctgcatggcc | | 300 |
| | tgcgagtatt gcgaactggg caacgaatcg aactgcccgc acgcggacct gtccggctac | | 360 |
| | acccatgacg gcagcttcca ggagtatgcc accgcggacg ccgtgcaggc cgcgcacatc | | 420 |
| | ccgcagggca ccgacctggc ggaggtggcc cccatcctgt gcgccggcat caccgtctac | | 480 |
| | aaggcgctga agagcgccaa cctgcgcgcg ggccattggg ccgcgatctc gggcgccgcc | | 540 |
| | ggtggcctgg gctccctggc cgtgcagtac gcgaaggcga tgggctaccg cgtcctgggc | | 600 |
| | atcgacggcg gtccgggcaa ggaagagctg ttcacctccc tgggcggcga agtgttcatc | | 660 |
| | gacttcacca aggagaagga catcgtcagc gccgtggtca aggcgaccaa cggcggcgcc | | 720 |
| | cacggcatca tcaacgtgtc ggtctccgaa gccgcgatcg aggcgtcgac ccgctactgc | | 780 |
| | cgggccaacg gcaccgtggt cctggtgggc ctgcccgcgg gcgccaagtg cagctcggac | | 840 |
| | gtcttcaacc atgtggtcaa gagcatctcg atcgtgggct cgtatgtcgg caaccgcgcc | | 900 |
| | gacacccgcg aggccctgga cttcttcgcc cgtggcctgg tcaagtcccc gatcaaggtg | | 960 |
| | gtcggcctgt ccagcctgcc cgagatctac gaaaagatgg agaagggcca gatcgccggc | | 1020 |
| | cgctatgtgg tcgacacctc caagtga | | 1047 |
| 3-90 | Sequences | | |
| 3-90-1 | Sequence Number [ID] | 90 | |
| 3-90-2 | Molecule Type | DNA | |
| 3-90-3 | Length | 1692 | |
| | | source 1 . . . 1692 | |
| 3-90-4-1 | Features Location/ | mol_type = other DNA | |
| | Qualifiers | organism = Saccharomyces cerevisiae | |
| | NonEnglishQualifier Value | | |
| 3-90-5 | Residues | | |
| | atgagcgaga tcaccctggg caagtacctg ttcgagcggc tgaagcaggt caacgtcaac | | 60 |
| | accgtcttcg gcctgcccgg cgacttcaac ctgagcctgc tggacaagat ctacgaggtc | | 120 |
| | gaaggcatgc gctgggcggg caacgccaac gagctgaacg ccgcgtacgc cgcggacggc | | 180 |
| | tatgcccgga tcaagggcat gtcgtgcatc atcaccacct tcggcgtggg cgagctgtcc | | 240 |
| | gccctgaacg gcatcgcggg cagctacgcc gaacacgtcg gcgtgctgca tgtcgtgggc | | 300 |
| | gtcccgagca tctcggccca ggcgaagcag ctgctgctgc accataccct gggcaacggc | | 360 |
| | gacttcaccg tgttccaccg catgtccgcg aacatcagcg agaccaccgc catgatcacc | | 420 |
| | gacatcgcca ccgcgccggc cgaaatcgac cgctgcatcc ggaccaccta cgtcacccag | | 480 |
| | cggcccgtgt atctgggcct gccggccaac ctggtcgacc tgaacgtgcc cgcgaagctg | | 540 |
| | ctgcagaccc cgatcgacat gtcgctgaag cccaacgacg ccgagtccga aaaggaagtc | | 600 |
| | atcgacacca tcctggcgct ggtcaaggac gccaagaacc cggtgatcct ggcggacgcc | | 660 |
| | tgctgctccc gccacgacgt caaggccgag accaagaagc tgatcgacct gacccagttc | | 720 |
| | cccgccttcg tgaccccgat gggcaagggc tccatcgacg aacagcatcc gcggtacggc | | 780 |
| | ggcgtctatg tgggcaccct gagcaagccc gaagtcaagg aagccgtgga aagcgccgac | | 840 |
| | ctgatcctgt cggtcggcgc cctgctgtcc gacttcaaca ccggctcctt cagctactcg | | 900 |
| | tataagacca agaacatcgt ggagttccac agcgaccaca tgaagatccg caacgccacc | | 960 |
| | ttccccggcg tccagatgaa gttcgtgctg cagaagctgc tgaccaccat cgccgacgcc | | 1020 |
| | gcgaagggct acaagccggt cgcggtgccc gcccggaccc cggcgaacgc cgcggtcccc | | 1080 |
| | gcctcgaccc cgctgaagca ggaatggatg tggaaccagc tgggcaactt cctgcaggaa | | 1140 |
| | ggcgacgtcg tgatcgcgga aaccggcacc tccgccttcg gcatcaacca gaccaccttc | | 1200 |
| | ccgaacaaca cctacggcat cagccaggtg ctgtggggct cgatcggctt caccaccggc | | 1260 |
| | gccaccctgg gcgccgcgtt cgccgcggag gaaatcgacc cgaagaagcg cgtcatcctg | | 1320 |
| | ttcatcggcg acggcagcct gcagctgacc gtgcaggaaa tctcgaccat gatccggtgg | | 1380 |
| | ggcctgaagc cctacctgtt cgtcctgaac aacgacggct ataccatcga gaagctgatc | | 1440 |
| | cacggcccga aggcccagta caacgaaatc cagggctggg accatctgtc gctgctgccc | | 1500 |
| | accttcggcg ccaaggacta tgagacccat cgcgtggcga ccaccggcga atgggacaag | | 1560 |
| | ctgacccagg acaagtcgtt caacgacaac tccaagatcc ggatgatcga gatcatgctg | | 1620 |
| | cccgtcttcg acgcgccgca gaacctggtg gaacaggcca agctgaccgc cgcgaccaac | | 1680 |
| | gcgaagcagt ga | | 1692 |
| 3-91 | Sequences | | |
| 3-91-1 | Sequence Number [ID] | 91 | |
| 3-91-2 | Molecule Type | DNA | |
| 3-91-3 | Length | 3147 | |
| | | source 1 . . . 3147 | |
| 3-91-4-1 | Features Location/ | mol_type = other DNA | |
| | Qualifiers | organism = Saccharomyces cerevisiae | |
| | NonEnglishQualifier Value | | |
| 3-91-5 | Residues | | |
| | atgaagtcgg aatacaccat cggccgctat ctgctggacc gcctgagcga gctgggcatc | | 60 |
| | cgccacatct tcggcgtccc cggcgactac aacctgtcgt tcctggacta catcatggag | | 120 |
| | tataagggca tcgactgggt cggcaactgc aacgaactga aacgccgcta cgccgcggac | | 180 |
| | ggctatgccc gcatcaacgg catcggcgcg atcctgacca ccttcggcgt cggcgagctg | | 240 |
| | tccgccatca acgccatcgc gggcgcctac gcggaacagg tgccggtcgt gaagatcacc | | 300 |
| | ggcatcccca ccgccaaggt ccgcgacaac ggcctgtatg tgcaccatac cctgggcgac | | 360 |
| | ggccgcttcg accacttctt cgagatgttc cgggaagtca ccgtggccga ggcgctgctg | | 420 |
| | agcgaggaaa acgccgcgca ggaaatcgac cgcgtgctga tctcgtgctg gcgccagaag | | 480 |
| | cggccggtcc tgatcaacct gcccatcgac gtgtacgaca agccgatcaa caagccgctg | | 540 |
| | aagcccctgc tggactatac catcagctcg aacaaggaag ccgcgtgcga gttcgtcacc | | 600 |
| | gagatcgtgc cgatcatcaa ccgcgccaag aagcccgtca tcctggcgga ctacggcgtg | | 660 |
| | taccggtatc aggtccagca cgtgctgaag aacctggcgg agaagaccgg cttcccggtc | | 720 |
| | gccaccctgt cgatgggcaa gggcgtgttc aacgaagccc atccgcagtt catcggcgtc | | 780 |
| | tacaacggcg acgtgtccag cccctatctg cgccagcggg tcgacgaggc cgactgcatc | | 840 |
| | atctcggtcg gcgtgaagct gaccgactcc accaccggcg gcttctccca cggcttcagc | | 900 |
| | aagcgcaacg tgatccatat cgacccgttc tccatcaagg ccaagggcaa gaagtacgcg | | 960 |
| | cccatcacca tgaaggacgc cctgaccgaa ctgacctcga agatcgagca ccggaacttc | | 1020 |
| | gaagacctgg acatcaagcc gtacaagtcc gacaaccaga agtatttcgc gaaggagaag | | 1080 |
| | cccatcaccc agaagcgctt cttcgaacgg atcgcccatt tcatcaagga gaaggacgtc | | 1140 |
| | ctgctggcgg aacagggcac ctgcttcttc ggcgccagca ccatccagct gccgaaggac | | 1200 |
| | gcgaccttca tcggccagcc cctgtggggc tccatcggct acaccctgcc ggccctgctg | | 1260 |
| | ggcagccagc tggcggacca gaagcgtcgc aacatcctgc tgatcggcga cggcgccttc | | 1320 |
| | cagatgaccg cgcaggagat ctcgaccatg ctgcgcctgc agatcaagcc gatcatcttc | | 1380 |
| | ctgatcaaca acgacggcta caccatcgag cgcgccatcc acggccggga acaggtgtac | | 1440 |
| | aacaacatcc agatgtggcg gtatcataac gtcccgaagg tgctgggccc caaggaatgc | | 1500 |
| | agcctgacct tcaaggtcca gtcggagacc gaactggaga aggccctgct ggtcgccgac | | 1560 |
| | aaggactgcg agcacctgat cttcatcgaa gtcgtgatgg accgctacga caagccggag | | 1620 |
| | cccctggaac gcctgtccaa gcggttcgcc aaccagaaca acggctatgc gcggatcaac | | 1680 |
| | ggcatcggcg ccattttaac caccttcggc gtgggcgagc tgatcgcgat caacgcgatc | | 1740 |
| | gccggcgcct acgcggagca ggtgccggtg gtcaaaatta ccggcatccc caccgcgaag | | 1800 |
| | gtgcgggaca acggcctgta cgtccatcac accctgggcg acggccggtt cgaccatttc | | 1860 |
| | ttcgaaatgt tccgggaggt gaccgtcgcc gaggcgctgc tgtcggaaga gaacgcggcc | | 1920 |
| | caggagatcg accgcgtcct gatcagctgc tggcggcaga agcgccccgt gctgatcaac | | 1980 |
| | ctgccgatcg acgtctatga caagcccatc aacaagcccc tgaagccgct gctggactac | | 2040 |
| | accatctcgt ccaacaagga agccgcctgc gagttcgtca ccgaaatcgt ccccatcatc | | 2100 |
| | aaccgcgcga agaagccggt gatcctggcc gactatggcg tctatcggta tcaggtgcag | | 2160 |
| | catgtcctga agaacctggc cgaaaagacc ggcttccccg tggccaccct gagcatgggc | | 2220 |
| | aagggcgtct tcaacgaggc gcacccccag ttcatcggcg tgtataacgg cgacgtgagc | | 2280 |
| | tcgccgtacc tgcggcagcg cgtggacgaa gccgactgca tcatcagcgt cggcgtcaag | | 2340 |
| | ctgaccgact cgaccaccgg cggcttctcg cacggcttct cgaagcggaa cgtcatccac | | 2400 |
| | atcgacccgt tctcgatcaa ggcgaagggc aagaagtatg ccccgatcac catgaaggac | | 2460 |
| | gcgctgaccg aactgaccag caagatcgaa catcgcaact tcgaggacct ggacatcaag | | 2520 |
| | ccctacaagt cggacaacca gaagtacttc gccaaggaaa agccgattac tcagaagcgc | | 2580 |
| | ttcttcgagc gcatcgcgca cttcatcaag gaaaaggacg tcctgctggc cgagcaaggc | | 2640 |
| | acctgcttct tcggtgcgtc gaccatccag ctgcccaagg acgccacctt catcggccag | | 2700 |
| | ccgctgtggg gctcgatcgg ctataccctg cccgcgctgc tgggctccca gctggccgat | | 2760 |
| | caaaaacgtc gcaatatttt actgatcggc gacggcgcgt tccagatgac cgcccaggag | | 2820 |
| | atcagcacca tgctgcggct gcagatcaag cccattatct tcctgattaa caacgacggc | | 2880 |
| | tataccatcg aacgggcgat ccacggccgc gagcaggtct ataataatat tcaaatgtgg | | 2940 |
| | cggtatcata atgtgcccaa ggtcctgggc ccgaaggaat gctcgctgac cttcaaggtg | | 3000 |
| | cagagcgaaa ccgagctgga aaaggccctg ctggtcgccg ataaggactg cgaacatctg | | 3060 |
| | atcttcatcg aggtggtcat ggaccggtat gacaagcccg aacccctgga acggctgagc | | 3120 |
| | aagcgcttcg cgaaccagaa caactga | | 3147 |
| 3-92 | Sequences | | |
| 3-92-1 | Sequence Number [ID] | 92 | |
| 3-92-2 | Molecule Type | DNA | |
| 3-92-3 | Length | 35 | |
| 3-92-4-1 | Features Location/ | misc_feature 1 . . . 35 | |
| | Qualifiers | note = JPS00082 | |
| | NonEnglishQualifier Value | | |
| | | source 1 . . . 35 | |
| 3-92-4-2 | Features Location/ | mol_type = other DNA | |
| | Qualifiers | organism = synthetic construct | |
| | NonEnglishQualifier Value | | |
| 3-92-5 | Residues | | |
| | tgcaaggtac actgtcagaa cgcagaagcg gtctg | | 35 |
| 3-93 | Sequences | | |
| 3-93-1 | Sequence Number [ID] | 93 | |
| 3-93-2 | Molecule Type | DNA | |
| 3-93-3 | Length | 28 | |
| 3-93-4-1 | Features Location/ | misc_feature 1 . . . 28 | |
| | Qualifiers | note = JPS00031 | |
| | NonEnglishQualifier Value | | |
| | | source 1 . . . 28 | |
| 3-93-4-2 | Features Location/ | mol_type = other DNA | |
| | Qualifiers | organism = synthetic construct | |
| | NonEnglishQualifier Value | | |
| 3-93-5 | Residues | | |
| | ggtttattcc tccttattta atcgatac | | 28 |
| 3-94 | Sequences | | |
| 3-94-1 | Sequence Number [ID] | 94 | |
| 3-94-2 | Molecule Type | DNA | |
| 3-94-3 | Length | 34 | |
| 3-94-4-1 | Features Location/ | misc_feature 1 . . . 34 | |
| | Qualifiers | note = JPS00032 | |
| | NonEnglishQualifier Value | | |
| | | source 1 . . . 34 | |
| | | mol_type = other DNA | |
| 3-94-4-2 | Features Location/ | organism = synthetic construct | |
| | Qualifiers | | |
| | NonEnglishQualifier Value | | |
| 3-94-5 | Residues | | |
| | aaggaggaat aaaccatggg cacggttgag cctg | | 34 |
| 3-95 | Sequences | | |
| 3-95-1 | Sequence Number [ID] | 95 | |
| 3-95-2 | Molecule Type | DNA | |
| 3-95-3 | Length | 33 | |
| 3-95-4-1 | Features Location/ | misc_feature 1 . . . 33 | |
| | Qualifiers | note = GMV257 | |
| | NonEnglishQualifier Value | | |
| | | source 1 . . . 33 | |
| 3-95-4-2 | Features Location/ | mol_type = other DNA | |
| | Qualifiers | organism = synthetic construct | |
| | NonEnglishQualifier Value | | |
| 3-95-5 | Residues | | |
| | cacatcctgt ctagatcagc cctcgccctt gac | | 33 |
| 3-96 | Sequences | | |
| 3-96-1 | Sequence Number [ID] | 96 | |
| 3-96-2 | Molecule Type | DNA | |
| 3-96-3 | Length | 58 | |
| 3-96-4-1 | Features Location/ | misc_feature 1 . .. 58 | |
| | Qualifiers | note = JPS00118) | |
| | NonEnglishQualifier Value | | |
| | | source 1 . . . 58 | |
| 3-96-4-2 | Features Location/ | mol_typ e= other DNA | |
| | Qualifiers | organism = synthetic construct | |
| | NonEnglishQualifier Value | | |
| 3-96-5 | Residues | | |
| | tctagacagg atgtgtcaca caggaaacca tgtcttatcc | | 58 |
| 3-97 | Sequences | | |
| 3-97-1 | Sequence Number [ID] | 97 | |
| 3-97-2 | Molecule Type | DNA | |
| 3-97-3 | Length | 41 | |
| 3-97-4-1 | Features Location/ | misc_feature 1 . . . 41 | |
| | Qualifiers | note = JPS00119 | |
| | NonEnglishQualifier Value | | |
| | | source 1 . . . 41 | |
| 3-97-4-2 | Features Location/ | mol_type = other DNA | |
| | Qualifiers | organism = synthetic construct | |
| | NonEnglishQualifier Value | | |
| 3-97-5 | Residues | | |
| | acagtgtacc ttgcactagt ctgaaaattc tttgtcgtag | | 41 |
| 3-98 | Sequences | | |
| 3-98-1 | Sequence Number [ID] | 98 | |
| 3-98-2 | Molecule Type | DNA | |
| 3-98-3 | Length | 70 | |
| 3-98-4-1 | Features Location/ | misc_feature 1 . . . 70 | |
| | Qualifiers | note = ESG00087 | |
| | NonEnglishQualifier Value | | |
| | | source 1 . . . 70 | |
| 3-98-4-2 | Features Location/ | mol_type = other DNA | |
| | Qualifiers | organism = synthetic construct | |
| | NonEnglishQualifier Value | | |
| 3-98-5 | Residues | | |
| | gtgttggttg ctcgctagca ttgtaccaag ggctgagcta gctataaagt cagcgccctg | | 60 |
| | caccattatg | | 70 |
| 3-99 | Sequences | | |
| 3-99-1 | Sequence Number [ID] | 99 | |
| 3-99-2 | Molecule Type | DNA | |
| 3-99-3 | Length | 46 | |
| 3-99-4-1 | Features Location/ | misc_feature 1 . . . 46 | |
| | Qualifiers | note = GMV251 | |
| | NonEnglishQualifier Value | | |
| | | source 1 . . . 46 | |
| 3-99-4-2 | Features Location/ | mol_type = other DNA | |
| | Qualifiers | organism = synthetic construct | |
| | NonEnglishQualifier Value | | |
| 3-99-5 | Residues | | |
| | gagcaaccaa cacttaaaga ggagaaaatg ggcacggttg agcctg | | 46 |
| 3-100 | Sequences | | |
| 3-100-1 | Sequence Number [ID] | 100 | |
| 3-100-2 | Molecule Type | DNA | |
| 3-100-3 | Length | 149 | |
| 3-100-4-1 | Features Location/ | misc_feature 1 . . . 149 | |
| | Qualifiers | note = IDT gBlock synthesized rnpB | |
| | NonEnglishQualifier Value | | |
| | | source 1 . . . 149 | |
| 3-100-4-2 | Features Location/ | mol_type = other DNA | |
| | Qualifiers | organism = synthetic construct | |
| | NonEnglishQualifier Value | | |
| 3-100-5 | Residues | | |
| | gctagcacta gtgatcacgt gcttaagccg gcttatcggt cagtttcacc tgatttacgt | | 60 |
| | aaaaacccgc ttcggcgggt ttttgctttt ggaggggcag aaagatgaat gactgtccac | | 120 |
| | gacgctatac ccaaaagaaa accggtacc | | 149 |
| 3-101 | Sequences | | |
| 3-101-1 | Sequence Number [ID] | 101 | |
| 3-101-2 | Molecule Type | DNA | |
| 3-101-3 | Length | 35 | |
| 3-101-4-1 | Features Location/ | misc_feature 1 . . . 35 | |
| | Qualifiers | note = JPS00161 | |
| | NonEnglishQualifier Value | | |
| | | source 1 . . . 35 | |
| 3-101-4-2 | Features Location/ | mol_type = other DNA | |
| | Qualifiers | organism = synthetic construct | |
| | NonEnglishQualifier Value | | |
| 3-101-5 | Residues | | |
| | gcctgataca gattattgta ggtggaccag ttggt | | 35 |
| 3-102 | Sequences | | |
| 3-102-1 | Sequence Number [ID] | 102 | |
| 3-102-2 | Molecule Type | DNA | |
| 3-102-3 | Length | 34 | |
| 3-102-4-1 | Features Location/ | misc_feature 1 . . . 34 | |
| | Qualifiers | note = JPS00162 | |
| | NonEnglishQualifier Value | | |
| | | source 1 . . . 34 | |
| 3-102-4-2 | Features Location/ | mol_type = other DNA | |
| | Qualifiers | organism = synthetic construct | |
| | NonEnglishQualifier Value | | |
| 3-102-5 | Residues | | |
| | ggtttattcc tccttgattt gtcctactca ggag | | 34 |
| 3-103 | Sequences | | |
| 3-103-1 | Sequence Number [ID] | 103 | |
| 3-103-2 | Molecule Type | DNA | |
| 3-103-3 | Length | 34 | |
| 3-103-4-1 | Features Location/ | misc_feature 1 . . . 34 | |
| | Qualifiers | note = JPS00163 | |
| | NonEnglishQualifier Value | | |
| | | source 1 . . . 34 | |
| 3-103-4-2 | Features Location/ | mol_type = other DNA | |
| | Qualifiers | organism = synthetic construct | |
| | NonEnglishQualifier Value | | |
| 3-103-5 | Residues | | |
| | aaggaggaat aaaccgctag cactagtgat cacg | | 34 |
| 3-104 | Sequences | | |
| 3-104-1 | Sequence Number [ID] | 104 | |
| 3-104-2 | Molecule Type | DNA | |
| 3-104-3 | Length | 34 | |
| 3-104-4-1 | Features Location/ | misc_feature 1 . . . 34 | |
| | Qualifiers | note = JPS00164 | |
| | NonEnglishQualifier Value | | |
| | | source 1 . . . 34 | |
| 3-104-4-2 | Features Location/ | mol_type = other DNA | |
| | Qualifiers | organism = synthetic construct | |
| | NonEnglishQualifier Value | | |
| 3-104-5 | Residues | | |
| | taatctgtat caggcggtac cggttttctt ttgg | | 34 |
| 3-105 | Sequences | | |
| 3-105-1 | Sequence Number [ID] | 105 | |
| 3-105-2 | Molecule Type | DNA | |
| 3-105-3 | Length | 34 | |
| 3-105-4-1 | Features Location/ | misc_feature 1 . . . 34 | |
| | Qualifiers | note = JPS00172 | |
| | NonEnglishQualifier Value | | |
| | | source 1 . . . 34 | |
| 3-105-4-2 | Features Location/ | mol_type = other DNA | |
| | Qualifiers | organism = synthetic construct | |
| | NonEnglishQualifier Value | | |
| 3-105-5 | Residues | | |
| | atcagactaa gccttgtgct taagccggct tatc | | 34 |
| 3-106 | Sequences | | |
| 3-106-1 | Sequence Number [ID] | 106 | |
| 3-106-2 | Molecule Type | DNA | |
| 3-106-3 | Length | 51 | |
| 3-106-4-1 | Features Location/ | misc_feature 1 . . . 51 | |
| | Qualifiers | note = JPS00173 | |
| | NonEnglishQualifier Value | | |
| | | source 1 . . . 51 | |
| 3-106-4-2 | Features Location/ | mol_type = other DNA | |
| | Qualifiers | organism = synthetic construct | |
| | NonEnglishQualifier Value | | |
| 3-106-5 | Residues | | |
| | aaggaggaat aaaccgctag cactagtgat cacttgacgg ctagctcagt c | | 51 |
| 3-107 | Sequences | | |
| 3-107-1 | Sequence Number [ID] | 107 | |
| 3-107-2 | Molecule Type | DNA | |
| 3-107-3 | Length | 33 | |
| 3-107-4-1 | Features Location/ | misc_feature 1 . . . 33 | |
| | Qualifiers | note = JPS00174 | |
| | NonEnglishQualifier Value | | |
| | | source 1 . . . 33 | |
| 3-107-4-2 | Features Location/ | mol_type = other DNA | |
| | Qualifiers | organism = synthetic construct | |
| | NonEnglishQualifier Value | | |
| 3-107-5 | Residues | | |
| | tgaacaggtc tgacttcagt gctgcgccga ggc | | 33 |
| 3-108 | Sequences | | |
| 3-108-1 | Sequence Number [ID] | 108 | |
| 3-108-2 | Molecule Type | DNA | |
| 3-108-3 | Length | 51 | |
| 3-108-4-1 | Features Location/ | misc_feature 1 . . . 51 | |
| | Qualifiers | note = JPS00176 | |
| | NonEnglishQualifier Value | | |
| | | source 1 . . . 51 | |
| 3-108-4-2 | Features Location/ | mol_type = other DNA | |
| | Qualifiers | organism = synthetic construct | |
| | NonEnglishQualifier Value | | |
| 3-108-5 | Residues | | |
| | agtcagacct gttcattaaa gaggagaaaa tgcagattta ctacgacaaa g | | 51 |
| 3-109 | Sequences | | |
| 3-109-1 | Sequence Number [ID] | 109 | |
| 3-109-2 | Molecule Type | DNA | |
| 3-109-3 | Length | 34 | |
| 3-109-4-1 | Features Location/ | misc_feature 1 . . . 34 | |
| | Qualifiers | note = JPS00177 | |
| | NonEnglishQualifier Value | | |
| | | source 1 . . . 34 | |
| 3-109-4-2 | Features Location/ | mol_type = other DNA | |
| | Qualifiers | organism = synthetic construct | |
| | NonEnglishQualifier Value | | |
| 3-109-5 | Residues | | |
| | aagtgttggt tgctctcagt tcttgctcgt gtcc | | 34 |
| 3-110 | Sequences | | |
| 3-110-1 | Sequence Number [ID] | 110 | |
| 3-110-2 | Molecule Type | DNA | |
| 3-110-3 | Length | 45 | |
| 3-110-4-1 | Features Location/ | misc_feature 1 . . . 45 | |
| | Qualifiers | note = JPS00157 | |
| | NonEnglishQualifier Value | | |
| | | source 1 . . . 45 | |
| 3-110-4-2 | Features Location/ | mol_type = other DNA | |
| | Qualifiers | organism = synthetic construct | |
| | NonEnglishQualifier Value | | |
| 3-110-5 | Residues | | |
| | gagcaaccaa cacttaaaga ggagaaaatg accgacaagc acccc | | 45 |
| 3-111 | Sequences | | |
| 3-111-1 | Sequence Number [ID] | 111 | |
| 3-111-2 | Molecule Type | DNA | |
| 3-111-3 | Length | 33 | |
| 3-111-4-1 | Features Location/ | misc_feature 1 . . . 33 | |
| | Qualifiers | note = JPS00178 | |
| | NonEnglishQualifier Value | | |
| | | source 1 . . . 33 | |
| 3-111-4-2 | Features Location/ | mol_type = other DNA | |
| | Qualifiers | organism = synthetic construct | |
| | NonEnglishQualifier Value | | |
| 3-111-5 | Residues | | |
| | aaggcttagt ctgattcaga ggccgtcgtc ggt | | 33 |
| 3-112 | Sequences | | |
| 3-112-1 | Sequence Number [ID] | 112 | |
| 3-112-2 | Molecule Type | DNA | |
| 3-112-3 | Length | 430 | |
| 3-112-4-1 | Features Location/ | misc_feature 1 . .. 430 | |
| | Qualifiers | note = IDT gBlock synthesized | |
| | | Me-AM1 PmxaF | |
| | NonEnglishQualifier Value | | |
| | | source 1 . . . 430 | |
| 3-112-4-2 | Features Location/ | mol_type = other DNA | |
| | Qualifiers | organism = synthetic construct | |
| | NonEnglishQualifier Value | | |
| 3-112-5 | Residues | | |
| | atggtgcagg gcgcttcccg cttggtcggg ccgcttcgcg agggcccgtt gacgacaacg | | 60 |
| | gtgcgatggg tcccggcccc ggtcaagacg atgccaatac gttgcgacac tacgccttgg | | 120 |
| | cacttttaga attgccttat cgtcctgata agaaatgtcc gaccagctaa agacatcgcg | | 180 |
| | tccaatcaaa gcctagaaaa tataggcgaa gggacgctaa taagtctttc ataagaccgc | | 240 |
| | gcaaatctaa aaatatcctt agattcacga tgcggcactt cggatgactt ccgagcgagc | | 300 |
| | ctggaacctc agaaaaacgt ctgagagata ccgcgaggcc gaaaggcgag gcggttcagc | | 360 |
| | gaggagacgc aggatgagca ggtttgtgac atcagtctcg gccttggcgg ctagcgagca | | 420 |
| | accaacactt | | 430 |
| 3-113 | Sequences | | |
| 3-113-1 | Sequence Number [ID] | 113 | |
| 3-113-2 | Molecule Type | DNA | |
| 3-113-3 | Length | 34 | |
| 3-113-4-1 | Features Location/ | misc_feature 1 . . . 34 | |
| | Qualifiers | note = JPS00169 | |
| | NonEnglishQualifier Value | | |
| | | source 1 . . . 34 | |
| 3-113-4-2 | Features Location/ | mol_type = other DNA | |
| | Qualifiers | organism = synthetic construct | |
| | NonEnglishQualifier Value | | |
| 3-113-5 | Residues | | |
| | agcgccctgc accattatgt tccggatctg catc | | 34 |
| 3-114 | Sequences | | |
| 3-114-1 | Sequence Number [ID] | 114 | |
| 3-114-2 | Molecule Type | DNA | |
| 3-114-3 | Length | 46 | |
| 3-114-4-1 | Features Location/ | misc_feature 1 . . . 46 | |
| | Qualifiers | note = GMV00251 | |
| | NonEnglishQualifier Value | | |
| | | source 1 . . . 46 | |
| 3-114-4-2 | Features Location/ | mol_type = other DNA | |
| | Qualifiers | organism = synthetic construct | |
| | NonEnglishQualifier Value | | |
| 3-114-5 | Residues | | |
| | gagcaaccaa cacttaaaga ggagaaaatg ggcacggttg agcctg | | 46 |
| 3-115 | Sequences | | |
| 3-115-1 | Sequence Number [ID] | 115 | |
| 3-115-2 | Molecule Type | DNA | |
| 3-115-3 | Length | 32 | |
| 3-115-4-1 | Features Location/ | misc_feature 1 . . . 32 | |
| | Qualifiers | note = (PS00170 | |
| | NonEnglishQualifier Value | | |
| | | source 1 . . . 32 | |
| 3-115-4-2 | Features Location/ | mol_type = other DNA | |
| | Qualifiers | organism = synthetic construct | |
| | NonEnglishQualifier Value | | |
| 3-115-5 | Residues | | |
| | atggtgcagg gcgcttcccg cttggtcggg cc | | 32 |
| 3-116 | Sequences | | |
| 3-116-1 | Sequence Number [ID] | 116 | |
| 3-116-2 | Molecule Type | DNA | |
| 3-116-3 | Length | 19 | |
| 3-116-4-1 | Features Location/ | misc_feature 1 . . . 19 | |
| | Qualifiers | note = JPS00171 | |
| | NonEnglishQualifier Value | | |
| | | source 1 . . . 19 | |
| 3-116-4-2 | Features Location/ | mol_type = other DNA | |
| | Qualifiers | organism = synthetic construct | |
| | NonEnglishQualifier Value | | |
| 3-116-5 | Residues | | |
| | aagtgttggt tgctcgcta | | 19 |
| 3-117 | Sequences | | |
| 3-117-1 | Sequence Number [ID] | 117 | |
| 3-117-2 | Molecule Type | DNA | |
| 3-117-3 | Length | 68 | |
| 3-117-4-1 | Features Location/ | misc_feature 1 . . . 68 | |
| | Qualifiers | note = JPS00153 | |
| | NonEnglishQualifier Value | | |
| | | source 1 . . . 68 | |
| 3-117-4-2 | Features Location/ | mol_type = other DNA | |
| | Qualifiers | organism = synthetic construct | |
| | NonEnglishQualifier Value | | |
| 3-117-5 | Residues | | |
| | accactcaac gatcagctag cactgtacct aggactgagc tagccgtcaa gtcagcgccc | | 60 |
| | tgcaccat | | 68 |
| 3-118 | Sequences | | |
| 3-118-1 | Sequence Number [ID] | 118 | |
| 3-118-2 | Molecule Type | DNA | |
| 3-118-3 | Length | 48 | |
| 3-118-4-1 | Features Location/ | misc_feature 1 . . . 48 | |
| | Qualifiers | note = JPS00151 | |
| | NonEnglishQualifier Value | | |
| | | source 1 . . . 48 | |
| 3-118-4-2 | Features Location/ | mol_type = other DNA | |
| | Qualifiers | organism = synthetic construct | |
| | NonEnglishQualifier Value | | |
| 3-118-5 | Residues | | |
| | tgatcgttga gtggtttaaa gaggagaaaa tgcgtgaaac gatacctc | | 48 |
| 3-119 | Sequences | | |
| 3-119-1 | Sequence Number [ID] | 119 | |
| 3-119-2 | Molecule Type | DNA | |
| 3-119-3 | Length | 33 | |
| 3-119-4-1 | Features Location/ | misc_feature 1 . . . 33 | |
| | Qualifiers | note = JPS00154 | |
| | NonEnglishQualifier Value | | |
| | | source 1 . . . 33 | |
| 3-119-4-2 | Features Location/ | mol_type = other DNA | |
| | Qualifiers | organism = synthetic construct | |
| | NonEnglishQualifier Value | | |
| 3-119-5 | Residues | | |
| | aagtgttggt tgctctcagt gctgcgccga ggc | | 33 |
| 3-120 | Sequences | | |
| 3-120-1 | Sequence Number [ID] | 120 | |
| 3-120-2 | Molecule Type | DNA | |
| 3-120-3 | Length | 33 | |
| 3-120-4-1 | Features Location/ | misc_feature 1 . . . 33 | |
| | Qualifiers | note = JPS00183 | |
| | NonEnglishQualifier Value | | |
| | | source 1 . . . 33 | |
| 3-120-4-2 | Features Location/ | mol_type = other DNA | |
| | Qualifiers | organism = synthetic construct | |
| | NonEnglishQualifier Value | | |
| 3-120-5 | Residues | | |
| | tgagctagct ataaagtgat cactagtgct agc | | 33 |
| 3-121 | Sequences | | |
| 3-121-1 | Sequence Number [ID] | 121 | |
| 3-121-2 | Molecule Type | DNA | |
| 3-121-3 | Length | 55 | |
| 3-121-4-1 | Features Location/ | misc_feature 1 . . . 55 | |
| | Qualifiers | note = JPS00185 | |
| | NonEnglishQualifier Value | | |
| | | source 1 . . . 55 | |
| 3-121-4-2 | Features Location/ | mol_type = other DNA | |
| | Qualifiers | organism = synthetic construct | |
| | NonEnglishQualifier Value | | |
| 3-121-5 | Residues | | |
| | ttatagctag ctcagccctt ggtacaatgc tagctgatcg ttgagtggtt taaag | | 55 |
| 3-122 | Sequences | | |
| 3-122-1 | Sequence Number [ID] | 122 | |
| 3-122-2 | Molecule Type | DNA | |
| 3-122-3 | Length | 35 | |
| 3-122-4-1 | Features Location/ | misc_feature 1 . . . 35 | |
| | Qualifiers | note = J23100 | |
| | NonEnglishQualifier Value | | |
| | | source 1 . . . 35 | |
| 3-122-4-2 | Features Location/ | mol_type = other DNA | |
| | Qualifiers | organism = synthetic construct | |
| | NonEnglishQualifier Value | | |
| 3-122-5 | Residues | | |
| | ttgacggcta gctcagtcct aggtacagtg ctagc | | 35 |
| 3-123 | Sequences | | |
| 3-123-1 | Sequence Number [ID] | 123 | |
| 3-123-2 | Molecule Type | DNA | |
| 3-123-3 | Length | 35 | |
| 3-123-4-1 | Features Location/ | misc_feature 1 . . . 35 | |
| | Qualifiers | note = J23100 hybrid | |
| | NonEnglishQualifier Value | | |
| | | source 1 .. 35 | |
| 3-123-4-2 | Features Location/ | mol_type = other DNA | |
| | Qualifiers | organism = synthetic construct | |
| | NonEnglishQualifier Value | | |
| 3-123-5 | Residues | | |
| | ttgacggcta gctcagccct tggtacaatg ctagc | | 35 |
| 3-124 | Sequences | | |
| 3-124-1 | Sequence Number [ID] | 124 | |
| 3-124-2 | Molecule Type | DNA | |
| 3-124-3 | Length | 35 | |
| 3-124-4-1 | Features Location/ | misc_feature 1 . . . 35 | |
| | Qualifiers | note = J23115 | |
| | NonEnglishQualifier Value | | |
| | | source 1 . . . 35 | |
| 3-124-4-2 | Features Location/ | mol_type = other DNA | |
| | Qualifiers | organism = synthetic construct | |
| | NonEnglishQualifier Value | | |
| 3-124-5 | Residues | | |
| | tttatagcta gctcagccct tggtacaatg ctagc | | 35 |
| 3-125 | Sequences | | |
| 3-125-1 | Sequence Number [ID] | 125 | |
| 3-125-2 | Molecule Type | DNA | |
| 3-125-3 | Length | 22 | |
| 3-125-4-1 | Features Location/ | misc_feature 1 . . . 22 | |
| | Qualifiers | note = GMV00233 | |
| | NonEnglishQualifier Value | | |
| | | source 1 . . . 22 | |
| 3-125-4-2 | Features Location/ | mol_type = other DNA | |
| | Qualifiers | organism = synthetic construct | |
| | NonEnglishQualifier Value | | |
| | Residues | | |
| 3-125-5 | tgccagctgc attaatgaat cg | | 22 |
| 3-126 | Sequences | | |
| 3-126-1 | Sequence Number [ID] | 126 | |
| 3-126-2 | Molecule Type | DNA | |
| 3-126-3 | Length | 45 | |
| 3-126-4-1 | Features Location/ | misc_feature 1 . . . 45 | |
| | Qualifiers | note = GMV00235 | |
| | NonEnglishQualifier Value | | |
| | | source 1 . . . 45 | |
| 3-126-4-2 | Features Location/ | mol_type = other DNA | |
| | Qualifiers | organism = synthetic construct | |
| | NonEnglishQualifier Value | | |
| 3-126-5 | Residues | | |
| | ttaatgcagc tggcagccag cgcttcgtta atacagatgt aggtg | | 45 |
| 3-127 | Sequences | | |
| 3-127-1 | Sequence Number [ID] | 127 | |
| 3-127-2 | Molecule Type | DNA | |
| 3-127-3 | Length | 52 | |
| 3-127-4-1 | Features Location/ | misc_feature 1 . . . 52 | |
| | Qualifiers | note = GMV00433 | |
| | NonEnglishQualifier Value | | |
| | | source 1 . . . 52 | |
| 3-127-4-2 | Features Location/ | mol_type = other DNA | |
| | Qualifiers | organism = synthetic construct | |
| | NonEnglishQualifier Value | | |
| 3-127-5 | Residues | | |
| | gatcgttgag tggtgaactt aaagaggaga aaatgggcac ggttgagcct gg | | 52 |
| 3-128 | Sequences | | |
| 3-128-1 | Sequence Number [ID] | 128 | |
| 3-128-2 | Molecule Type | DNA | |
| 3-128-3 | Length | 19 | |
| 3-128-4-1 | Features Location/ | misc_feature 1 . . . 19 | |
| | Qualifiers | note = GMV00434 | |
| | NonEnglishQualifier Value | | |
| | | source 1 . . . 19 | |
| 3-128-4-2 | Features Location/ | mol_type = other DNA | |
| | Qualifiers | organism = synthetic construct | |
| | NonEnglishQualifier Value | | |
| 3-128-5 | Residues | | |
| | tgcattcgat tcctgtttg | | 19 |
| 3-129 | Sequences | | |
| 3-129-1 | Sequence Number [ID] | 129 | |
| 3-129-2 | Molecule Type | DNA | |
| 3-129-3 | Length | 19 | |
| 3-129-4-1 | Features Location/ | misc_feature 1 . . . 19 | |
| | Qualifiers | note = GMV00435 | |
| | NonEnglishQualifier Value | | |
| | | source 1 . . . 19 | |
| 3-129-4-2 | Features Location/ | mol_type = other DNA | |
| | Qualifiers | organism = synthetic construct | |
| | NonEnglishQualifier Value | | |
| 3-129-5 | Residues | | |
| | caggaatcga atgcaaccg | | 19 |
| 3-130 | Sequences | | |
| 3-130-1 | Sequence Number [ID] | 130 | |
| 3-130-2 | Molecule Type | DNA | |
| 3-130-3 | Length | 49 | |
| 3-130-4-1 | Features Location/ | misc_feature 1 . . . 49 | |
| | Qualifiers | note = GMV00436 | |
| | NonEnglishQualifier Value | | |
| | | source 1 . . . 49 | |
| 3-130-4-2 | Features Location/ | mol_type = other DNA | |
| | Qualifiers | organism = synthetic construct | |
| | NonEnglishQualifier Value | | |
| 3-130-5 | Residues | | |
| | gagcaaccaa cactcacaca ggaaaccatg catattacat acgatctgc | | 49 |
| 3-131 | Sequences | | |
| 3-131-1 | Sequence Number [ID] | 131 | |
| 3-131-2 | Molecule Type | DNA | |
| 3-131-3 | Length | 39 | |
| 3-131-4-1 | Features Location/ | misc_feature 1 . . . 39 | |
| | Qualifiers | note = GMV00437 | |
| | NonEnglishQualifier Value | | |
| | | source 1 . . . 39 | |
| 3-131-4-2 | Features Location/ | mol_type = other DNA | |
| | Qualifiers | organism = synthetic construct | |
| | NonEnglishQualifier Value | | |
| 3-131-5 | Residues | | |
| | gttcaccact caacgatctt aagcgtcaac gaaaccggt | | 39 |
| 3-132 | Sequences | | |
| 3-132-1 | Sequence Number [ID] | 132 | |
| 3-132-2 | Molecule Type | DNA | |
| 3-132-3 | Length | 69 | |
| 3-132-4-1 | Features Location/ | misc_feature 1 . .. 69 | |
| | Qualifiers | note = GMV00438 | |
| | NonEnglishQualifier Value | | |
| | | source 1 . . . 69 | |
| 3-132-4-2 | Features Location/ | mol_type = other DNA | |
| | Qualifiers | organism = synthetic construct | |
| | NonEnglishQualifier Value | | |
| 3-132-5 | Residues | | |
| | tgaacaggtc tgactgctag cattgtacca agggctgagc tagctataaa gatttgtcct | | 60 |
| | actcaggag | | 69 |
| 3-133 | Sequences | | |
| 3-133-1 | Sequence Number [ID] | 133 | |
| 3-133-2 | Molecule Type | DNA | |
| 3-133-3 | Length | 53 | |
| 3-133-4-1 | Features Location/ | misc_feature 1 . . . 53 | |
| | Qualifiers | note = GMV00439 | |
| | NonEnglishQualifier Value | | |
| | | source 1 . . . 53 | |
| 3-133-4-2 | Features Location/ | mol_type = other DNA | |
| | Qualifiers | organism = synthetic construct | |
| | NonEnglishQualifier Value | | |
| 3-133-5 | Residues | | |
| | agtcagacct gttcattaaa gaggagaaaa tgagcggaaa aaccctttac gac | | 53 |
| 3-134 | Sequences | | |
| 3-134-1 | Sequence Number [ID] | 134 | |
| 3-134-2 | Molecule Type | DNA | |
| 3-134-3 | Length | 42 | |
| 3-134-4-1 | Features Location/ | misc_feature 1 . . . 42 | |
| | Qualifiers | note = GMV00440 | |
| | NonEnglishQualifier Value | | |
| | | source 1 . . . 42 | |
| 3-134-4-2 | Features Location/ | mol_type = other DNA | |
| | Qualifiers | organism = synthetic construct | |
| | NonEnglishQualifier Value | | |
| 3-134-5 | Residues | | |
| | aaggcttagt ctgattcaga cggcgcgcaa ggcggcgacg at | | 42 |
| 3-135 | Sequences | | |
| 3-135-1 | Sequence Number [ID] | 135 | |
| 3-135-2 | Molecule Type | DNA | |
| 3-135-3 | Length | 49 | |
| 3-135-4-1 | Features Location/ | misc_feature 1 . . . 49 | |
| | Qualifiers | note = GMV00441 | |
| | NonEnglishQualifier Value | | |
| | | source 1 . . . 49 | |
| 3-135-4-2 | Features Location/ | mol_type = other DNA | |
| | Qualifiers | organism = synthetic construct | |
| | NonEnglishQualifier Value | | |
| 3-135-5 | Residues | | |
| | atcagactaa gcctttcaca caggaaacca tgcacgacag actgatcat | | 49 |
| 3-136 | Sequences | | |
| 3-136-1 | Sequence Number [ID] | 136 | |
| 3-136-2 | Molecule Type | DNA | |
| 3-136-3 | Length | 35 | |
| 3-136-4-1 | Features Location/ | misc_feature 1 . . . 35 | |
| | Qualifiers | note = GMV00442 | |
| | NonEnglishQualifier Value | | |
| | | source 1 . . . 35 | |
| 3-136-4-2 | Features Location/ | mol_type = other DNA | |
| | Qualifiers | organism = synthetic construct | |
| | NonEnglishQualifier Value | | |
| 3-136-5 | Residues | | |
| | ggtttattcc tcctttcaca catccccgac ttgcg | | 35 |
| 3-137 | Sequences | | |
| 3-137-1 | Sequence Number [ID] | 137 | |
| 3-137-2 | Molecule Type | DNA | |
| 3-137-3 | Length | 49 | |
| 3-137-4-1 | Features Location/ | misc_feature 1 . . . 49 | |
| | Qualifiers | note = ESG00084 | |
| | NonEnglishQualifier Value | | |
| | | source 1 . . . 49 | |
| 3-137-4-2 | Features Location/ | mol_type = other DNA | |
| | Qualifiers | organism = synthetic construct | |
| | NonEnglishQualifier Value | | |
| 3-137-5 | Residues | | |
| | gagacagaac gaagtgtgac cagaacgcag aagcggtctg ataaaacag | | 49 |
| 3-138 | Sequences | | |
| 3-138-1 | Sequence Number [ID] | 138 | |
| 3-138-2 | Molecule Type | DNA | |
| 3-138-3 | Length | 70 | |
| 3-138-4-1 | Features Location/ | misc_feature 1 . . . 70 | |
| | Qualifiers | note = ESG00088 | |
| | NonEnglishQualifier Value | | |
| | | source 1 . . . 70 | |
| 3-138-4-2 | Features Location/ | mol_type = other DNA | |
| | Qualifiers | organism = synthetic construct | |
| | NonEnglishQualifier Value | | |
| 3-138-5 | Residues | | |
| | gtgttggttg ctcgctagca ctgtacctag gactgagcta gccgtcaagt cagcgccctg | | 60 |
| | caccattatg | | 70 |
| 3-139 | Sequences | | |
| 3-139-1 | Sequence Number [ID] | 139 | |
| 3-139-2 | Molecule Type | DNA | |
| 3-139-3 | Length | 10705 | |
| 3-139-4-1 | Features Location/ | misc_feature 1 . . . 10705 | |
| | Qualifiers | note = pMZT37 | |
| | NonEnglishQualifier Value | | |
| | | source 1 . . . 10705 | |
| 3-139-4-2 | Features Location/ | mol_type = other DNA | |
| | Qualifiers | organism = synthetic construct | |
| | NonEnglishQualifier Value | | |
| 3-139-5 | Residues | | |
| | tcaccctgtc gggcaatgcc gaggcattct ggcagcagcg ccccctggcc tgtagtggat | | 60 |
| | tacgtgccgg tctgttccat cctaccaccg gctattcact gccgctggcg gttgccgtgg | | 120 |
| | ccgaccgcct gagcgcactt gatgtcttta cgtcggcctc aattcaccag gctattaggc | | 180 |
| | attttgcccg cgagcgctgg cagcagcagc gctttttccg catgctgaat cgcatgctgt | | 240 |
| | ttttagccgg acccgccgat tcacgctggc gggttatgca gcgtttttat ggtttacctg | | 300 |
| | aagatttaat tgcccgtttt tatgcgggaa aactcacgct gaccgatcgg ctacgtattc | | 360 |
| | tgagcggcaa gccgcctgtt ccggtattag cagcattgca agccattatg acgactcatc | | 420 |
| | gttaagagac agaacgaagt gtgaccagaa cgcagaagcg gtctgataaa acagaatttg | | 480 |
| | cctggcggca gtagcgcggt ggtcccacct gaccccatgc cgaactcaga agtgaaacgc | | 540 |
| | cgtagcgccg atggtagtgt ggggtctccc catgcgagag tagggaactg ccaggcatca | | 600 |
| | aataaaacga aaggctcagt cgaaagactg ggcctttcgt tttatctgtt ctttgtcggt | | 660 |
| | gaacgctctc ctgagtagga caaatcttgt aggtggacca gttggtgatt ttgaactttt | | 720 |
| | gctttgccac ggaacggtct gcgttgtcgg gaagatgcgt gatctgatcc ttcaactcag | | 780 |
| | caaaagttcg atttattcaa caaagccgcc gtcccgtcaa gtcagcgtaa tgctctgcca | | 840 |
| | gtgttacaac caattaacca attctgatta gaaaaactca tcgagcatca aatgaaactg | | 900 |
| | caatttattc atatcaggat tatcaatacc atatttttga aaaagccgtt tctgtaatga | | 960 |
| | aggagaaaac tcaccgaggc agttccatag gatggcaaga tcctggtatc ggtctgcgat | | 1020 |
| | tccgactcgt ccaacatcaa tacaacctat taatttcccc tcgtcaaaaa taaggttatc | | 1080 |
| | aagtgagaaa tcaccatgag tgacgactga atccggtgag aatggcaaaa gcttatgcat | | 1140 |
| | ttctttccag acttgttcaa caggccagcc attacgctcg tcatcaaaat cactcgcatc | | 1200 |
| | aaccaaaccg ttattcattc gtgattgcgc ctgagcgaga cgaaatacgc gatcgctgtt | | 1260 |
| | aaaaggacaa ttacaaacag gaatcgaatg caaccggcgc aggaacactg ccagcgcatc | | 1320 |
| | aacaatattt tcacctgaat caggatattc ttctaatacc tggaatgctg ttttcccggg | | 1380 |
| | gatcgcagtg gtgagtaacc atgcatcatc aggagtacgg ataaaatgct tgatggtcgg | | 1440 |
| | aagaggcata aattccgtca gccagtttag tctgaccatc tcatctgtaa catcattggc | | 1500 |
| | aacgctacct ttgccatgtt tcagaaacaa ctctggcgca tcgggcttcc catacaatcg | | 1560 |
| | atagattgtc gcacctgatt gcccgacatt atcgcgagcc catttatacc catataaatc | | 1620 |
| | agcatccatg ttggaattta atcgcggcct cgagcaagac gtttcccgtt gaatatggct | | 1680 |
| | cataacaccc cttgtattac tgtttatgta agcagacagt tttattgttc atgatgatat | | 1740 |
| | atttttatct tgtgcaatgt aacatcagag attttgagac acaacgtggc tttccccccc | | 1800 |
| | ccccctgcag gtccgacacg gggatggatg gcgttcccga tcatggtcct gcttgcttcg | | 1860 |
| | ggtggcatcg gaatgccggc gctgcaagca atgttgtcca ggcaggtgga tgaggaacgt | | 1920 |
| | caggggcagc tgcaaggctc actggcggcg ctcaccagcc tgacctcgat cgtcggaccc | | 1980 |
| | ctcctcttca cggcgatcta tgcggcttct ataacaacgt ggaacgggtg ggcatggatt | | 2040 |
| | gcaggcgctg ccctctactt gctctgcctg ccggcgctgc gtcgcgggct ttggagcggc | | 2100 |
| | gcagggcaac gagccgatcg ctgatcgtgg aaacgatagg cctatgccat gcgggtcaag | | 2160 |
| | gcgacttccg gcaagctata cgcgccctag aattgtcaat tttaatcctc tgtttatcgg | | 2220 |
| | cagttcgtag agcgcgccgt gcgtcccgag cgatactgag cgaagcaagt gcgtcgagca | | 2280 |
| | gtgcccgctt gttcctgaaa tgccagtaaa gcgctggctg ctgaaccccc agccggaact | | 2340 |
| | gaccccacaa ggccctagcg tttgcaatgc accaggtcat cattgaccca ggcgtgttcc | | 2400 |
| | accaggccgc tgcctcgcaa cgcttcgcag gcttcgccga cctgctcgcg ccacttcttc | | 2460 |
| | acgcgggtgg aatccgatcc gcacatgagg cggaaggttt ccagcttgag cgggtacggc | | 2520 |
| | tcccggtgcg agctgaaata gtcgaacatc ctgcgggccg tcggcgacag cttgcggtac | | 2580 |
| | ttctcccata tgaatttcgt gtagtggtcg ccagcaaaca gcacgacgat ttcctcgtcg | | 2640 |
| | atcaggacct ggcaacggga cgttttcttg ccacggtcca ggacgcggaa gcggtgcagc | | 2700 |
| | agcgacaccg attccaggtg cccaacgcgg tcggacgtga agcccatcgc cgtcgcctgt | | 2760 |
| | aggcgcgaca ggcattcctc ggccttcgtg taataccggc cattgatcga ccagcccagg | | 2820 |
| | tcctggcaaa gctcgtagaa cgtgaaggtg atcggctcgc cgataggggt gcgcttcgcg | | 2880 |
| | tactccaaca cctgctgcca caccagttcg tcatcgtcgg cccgcagctc gacgccggtg | | 2940 |
| | taggtgatct tcacgtcctt gttgacgtgg aaaatgacct tgttttgcag cgcctcgcgc | | 3000 |
| | gggattttct tgttgcgcgt ggtgaacagg gcagagcggg ccgtgtcgtt tggcatcgct | | 3060 |
| | cgcatcgtgt ccggccacgg cgcaatatcg aacaaggaaa gctgcatttc cttgatctgc | | 3120 |
| | tgcttcgtgt gtttcagcaa cgcggcctgc ttggcctcgc tgacctgttt tgccaggtcc | | 3180 |
| | tcgccggcgg tttttcgctt cttggtcgtc atagttcctc gcgtgtcgat ggtcatcgac | | 3240 |
| | ttcgccaaac ctgccgcctc ctgttcgaga cgacgcgaac gctccacggc ggccgatggc | | 3300 |
| | gcgggcaggg cagggggagc cagttgcacg ctgtcgcgct cgatcttggc cgtagcttgc | | 3360 |
| | tggaccatcg agccgacgga ctggaaggtt tcgcggggcg cacgcatgac ggtgcggctt | | 3420 |
| | gcgatggttt cggcatcctc ggcggaaaac cccgcgtcga tcagttcttg cctgtatgcc | | 3480 |
| | ttccggtcaa acgtccgatt cattcaccct ccttgcggga ttgccccgac tcacgccggg | | 3540 |
| | gcaatgtgcc cttattcctg atttgacccg cctggtgcct tggtgtccag ataatccacc | | 3600 |
| | ttatcggcaa tgaagtcggt cccgtagacc gtctggccgt ccttctcgta cttggtattc | | 3660 |
| | cgaatcttgc cctgcacgaa taccagctcc gcgaagtcgc tcttcttgat ggagcgcatg | | 3720 |
| | gggacgtgct tggcaatcac gcgcaccccc cggccgtttt agcggctaaa aaagtcatgg | | 3780 |
| | ctctgccctc gggcggacca cgcccatcat gaccttgcca agctcgtcct gcttctcttc | | 3840 |
| | gatcttcgcc agcagggcga ggatcgtggc atcaccgaac cgcgccgtgc gcgggtcgtc | | 3900 |
| | ggtgagccag agtttcagca ggccgcccag gcggcccagg tcgccattga tgcgggccag | | 3960 |
| | ctcgcggacg tgctcatagt ccacgacgcc cgtgattttg tagccctggc cgacggccag | | 4020 |
| | caggtaggcc tacaggctca tgccggccgc cgccgccttt tcctcaatcg ctcttcgttc | | 4080 |
| | gtctggaagg cagtacacct tgataggtgg gctgcccttc ctggttggct gcagagcagg | | 4140 |
| | agccatccgc ttgccctcat ctgttacgcc ggcggtagcc ggccagcctc gcagagcagg | | 4200 |
| | attcccgttg agcaccgcca ggtgcgaata agggacagtg aagaaggaac acccgctcgc | | 4260 |
| | gggtgggcct acttcaccta tcctgcccgg ctgacgccgt tggatacacc aaggaaagtc | | 4320 |
| | tacacgaacc ctttggcaaa atcctgtata tcgtgcgaaa aaggatggat ataccgaaaa | | 4380 |
| | aatcgctata atgaccccga agcagggtta tgcagcggaa aagatccgtc gaccctttcc | | 4440 |
| | gacgctcacc gggctggttg ccctcgccgc tgggctggcg gacgttgaca cttgaggggc | | 4500 |
| | cgcgccagaa acgccgtcga agccgtgtgc gagacaccgc ggccgccggc gttgtggata | | 4560 |
| | cctcgcggaa aacttggccc tcactgacag atgaggggcg gacgttgaca cttgaggggc | | 4620 |
| | cgactcaccc ggcgcggcgt tgacagatga ggggcaggct cgatttcggc cggcgacgtg | | 4680 |
| | gagctggcca gcctcgcaaa tcggcgaaaa cgcctgattt tacgcgagtt tcccacagat | | 4740 |
| | gatgtggaca agcctgggga taagtgccct gcggtattga cacttgaggg gcgcgactac | | 4800 |
| | tgacagatga ggggcgcgat ccttgacact tgaggggcag agtgctgaca gatgaggggc | | 4860 |
| | gcacctattg acatttgagg ggctgtccac aggcagaaaa tccagcattt gcaagggttt | | 4920 |
| | ccgcccgttt ttcggccacc gctaacctgt cttttaacct gcttttaaac caatatttat | | 4980 |
| | aaaccttgtt tttaaccagg gctgcgccct gtgcgcgtga ccgcgcacgc cgaagggggg | | 5040 |
| | tgccccccct tctcgaaccc tcccggcccg ctaacgcggg cctcccatcc ccccaggggc | | 5100 |
| | tgcgcccctc ggccgcgaac ggcctcaccc caaaaatggc agccaagctg accacttctg | | 5160 |
| | cgctcggccc ttccggctgg ctggtttatt gctgataaat ctggagccgg tgagcgtggg | | 5220 |
| | tctcgcggta tcattgcagc actggggcca gatggtaagc cctcccgtat cgtagttatc | | 5280 |
| | tacacgacgg ggagtcaggc aactatggat gaacgaaata gacagatcgc tgagataggt | | 5340 |
| | gcctcactga ttaagcattg gtaactgtca gaccaagttt actcatatat actttagatt | | 5400 |
| | gatttaaaac ttcattttta atttaaaagg atctaggtga agatcctttt tgataatctc | | 5460 |
| | atgaccaaaa tcccttaacg tgagttttcg ttccactgag cgtcagaccc cgtagaaaag | | 5520 |
| | atcaaaggat cttcttgaga tccttttttt ctgcgcgtaa tctgctgctt gcaaacaaaa | | 5580 |
| | aaaccaccgc taccagcggt ggtttgtttg ccggatcaag agctaccaac tctttttccg | | 5640 |
| | aaggtaactg gcttcagcag agcgcagata ccaaatactg tccttctagt gtagccgtag | | 5700 |
| | ttaggccacc acttcaagaa ctctgtagca ccgcctacat acctcgctct gctaatcctg | | 5760 |
| | ttaccagtgg ctgctgccag tggcgataag tcgtgtctta ccgggttgga ctcaagacga | | 5820 |
| | tagttaccgg ataaggcgca gcggtcgggc tgaacggggg gttcgtgcac acagcccagc | | 5880 |
| | ttggagcgaa cgacctacac cgaactgaga tacctacagc gtgagctatg agaaagcgcc | | 5940 |
| | acgcttcccg aagggagaaa ggcggacagg tatccggtaa gcggcagggt cggaacagga | | 6000 |
| | gagcgcacga gggagcttcc agggggaaac gcctggtatc tttatagtcc tgtcgggttt | | 6060 |
| | cgccacctct gacttgagcg tcgatttttg tgatgctcgt caggggggcg gagcctatgg | | 6120 |
| | aaaaacgcca gcaacgcggc ccttttacgg ttcctggcct tttgctggcc tgtcgggttt | | 6180 |
| | atgttctttc ctgcgttatc ccctgattct gtggataacc gtattaccgc ctttgagtga | | 6240 |
| | gctgataccg ctcgccgcag ccgaacgacc gagcgcagcg agtcagtgag cgaggaagcg | | 6300 |
| | gaagagcgcc caatacgcaa accgcctctc cccgcgcgtt ggccgattca ttaatgcagc | | 6360 |
| | tggcagccag cgcttcgtta atacagatgt aggtgttcca cagggtagcc agcagcatcc | | 6420 |
| | tgcgatgcag atccggaaca taatggtgca gggcgctgac ttgacggcta gctcagtcct | | 6480 |
| | aggtacagtg ctagcgagca accaacactt aaagaggaga aaatgtatcc gtttataagg | | 6540 |
| | acagcccgaa tgacggtctg cgcaaaaaaa cacgttcatc tcactcgcga tgctgcggag | | 6600 |
| | cagttactgg ctgatattga tcgacgcctt gatcagttat tgcccgtgga gggagaacgg | | 6660 |
| | gatgttgtgg gtgccgcgat gcgtgaaggt gcgctggcac cgggaaaacg tattcgcccc | | 6720 |
| | atgttgctgt tgctgaccgc ccgcgatctg ggttgcgctg tcagccatga cggattactg | | 6780 |
| | gatttggcct gtgcggtgga aatggtccac gcggcttcgc tgatccttga cgatatgccc | | 6840 |
| | tgcatggacg atgcgaagct gcggcgcgga cgccctacca ttcattctca ttacggagag | | 6900 |
| | catgtggcaa tactggcggc ggttgccttg ctgagtaaag cctttggcgt aattgccgat | | 6960 |
| | gcagatggcc tcacgccgct ggcaaaaaat cgggcggttt ctgaactgtc aaacgccatc | | 7020 |
| | ggcatgcaag gattggttca gggtcagttc aaggatctgt ctgaagggga taagccgcgc | | 7080 |
| | agcgctgaag ctattttgat gacgaatcac tttaaaacca gcacgctgtt ttgtgcctcc | | 7140 |
| | atgcagatgg cctcgattgt tgcgaatgcc tccagcgaag cgcgtgattg cctgcatcgt | | 7200 |
| | ttttcacttg atcttggtca ggcatttcaa ctgctggacg atttgaccga tggcatgacc | | 7260 |
| | gacaccggta aggatagcaa tcaggacgcc ggtaaatcga cgctggtcaa tctgttaggc | | 7320 |
| | cctagggcgg ttgaagaacg tctgagacaa catcttcatc ttgccagtga gcatctctct | | 7380 |
| | gcggcctgcc aacacgggca cgccactcaa cattttattc aggcctggtt tgacaaaaaa | | 7440 |
| | ctcgctgccg tcagttaaag gtctctagac aggatgtgtc acacaggaaa ccatgaaacc | | 7500 |
| | aactacggta attggtgcag gcttcggtgg cctggcactg gcaattcgtc tacaggctgc | | 7560 |
| | ggggatcccc gtcttactgc ttgaacaacg tgataaaccc ggcggtcggg cttatgtcta | | 7620 |
| | cgaggatcag gggtttacct ttgatgcagg cccgacggtt atcaccgatc ccagtgccat | | 7680 |
| | tgaagaactg tttgcactgg caggaaaaca gttaaaagag tatgtcgaac tgctgccggt | | 7740 |
| | tacgccgttt taccgcctgt gttgggagtc agggaaggtc tttaattacg ataacgatca | | 7800 |
| | aacccggctc gaagcgcaga ttcagcagtt taatccccgc gatgtcgaag gttatcgtca | | 7860 |
| | gtttctggac tattcacgcg cggtgtttaa agaaggctat ctgaagctcg gtactgtccc | | 7920 |
| | ttttttatcg ttcagagaca tgcttcgcgc cgcacctcaa ctggcgaaac tgcaggcatg | | 7980 |
| | gagaagcgtt tacagtaagg ttgccagtta catcgaagat gaacatctgc gccaggcgtt | | 8040 |
| | ttctttccac tcgctgttgg tgggcggcaa tcccttcgcc acctcatcca tttatacgtt | | 8100 |
| | gatacacgcg ctggagcgtg agtggggcgt ctggtttccg cgtggcggca ccggcgcatt | | 8160 |
| | agttcagggg atgataaagc tgtttcagga tctgggtggt gaagtcgtgt taaacgccag | | 8220 |
| | agtcagccat atggaaacga caggaaacaa gattgaagcc gtgcatttag aggacggtcg | | 8280 |
| | caggttcctg acgcaagccg tcgcgtcaaa tgcagatgtg gttcatacct atcgcgacct | | 8340 |
| | gttaagccag caccctgccg cggttaagca gtccaacaaa ctgcagacta agcgtatgag | | 8400 |
| | taactctctg tttgtgctct attttggttt gaatcaccat catgatcagc tcgcgcatca | | 8460 |
| | cacggtttgt ttcggcccgc gttaccgcga actgattgac gagattttta atcatgatgg | | 8520 |
| | cctcgcagaa gacttctcac tttatctgca cgcgccctgt gtcacggatt cgtcactggc | | 8580 |
| | gcctgaaggt tgcggcagtt actatgtgtt ggcgccggtg ccgcatttag gcaccgcgaa | | 8640 |
| | cctcgactgg acggttgagg ggccaaaact acgcgaccgt atttttgagt accttgagca | | 8700 |
| | gcattacatg cctggcttac ggagtcagct ggtcacgcac cagatgttta cgccgtttga | | 8760 |
| | ttttcgcgac cagcttaatg cctatcaggg ctcagccttt tctgtggagc ccgttcttac | | 8820 |
| | ccagagcgcc tggtttcggc cgcataaccg cgataaaacc attactaatc tctacctggt | | 8880 |
| | cggcgcaggc acgcatcccg gcgcaggcat tcctggcgtc atcggctcgg caaaagcgac | | 8940 |
| | agcaggtttg atgctggagg atctgattta agtgatcgtt gagtggtgaa cttaaagagg | | 9000 |
| | agaaaatgaa taatccgtcg ttactcaatc atgcggtcga aacgatggca gttggctcga | | 9060 |
| | aaagttttgc gacagcctca aagttatttg atgcaaaaac ccggcgcagc gtactgatgc | | 9120 |
| | tctacgcctg gtgccgccat tgtgacgatg ttattgacga ccagacgctg ggcttccagg | | 9180 |
| | cccggcagcc tgccttacaa acgcccgaac aacgtctgat gcaacttgag atgaaaacgc | | 9240 |
| | gccaggccta tgcaggatcg cagatgcacg aaccggcgtt tgcggctttt caggaagtgg | | 9300 |
| | ctatggctca tgatatcgcc ccggcttacg cgtttgatca tctggaaggc ttcgccatgg | | 9360 |
| | atgtacgcga agcgcaatac agccaactgg acgatacgct gcgctattgc tatcacgttg | | 9420 |
| | caggcgttgt cggcttgatg atggcgcaaa tcatgggcgt acgggataac gccacgctgg | | 9480 |
| | accgcgcctg tgaccttggg ctggcatttc agttgaccaa tattgctcgc gatattgtgg | | 9540 |
| | acgatgcgca tgcgggccgc tgttatctgc cggcaagctg gctggagcat gaaggtctga | | 9600 |
| | acaaagagaa ttatgcggca cctgaaaacc gtcaggcgct gagccgtatc gcccgtcgtt | | 9660 |
| | tggtgcagga agcagaacct tactatttgt ctgccacagc gggcctggct gggttgcccc | | 9720 |
| | tgcgttcggc ctgggcaatc gctacggcga agcaggttta ccggaaaata ggtgtcaaag | | 9780 |
| | ttgaacaggc cggtcagcaa gcctgggatc agcggcagtc aacgaccacg cccgaaaaat | | 9840 |
| | taacgctgct gctggccgcc tctggtcagg cccttacttc ccggatgcgg gctcatcctc | | 9900 |
| | cccgccctgc gcatctctgg cagcgcccgc tctaatcacg tagcaagctg acagtttaaa | | 9960 |
| | gaggagaaaa tgggagcggc tatgcaaccg cattatgatc tgattctcgt gggggctgga | | 10020 |
| | ctcgcgaatg gccttatcgc cctgcgtctt cagcagcagc aacctgatat gcgtattttg | | 10080 |
| | cttatcgacg ccgcacccca ggcgggcggg aatcatacgt ggtcatttca ccacgatgat | | 10140 |
| | ttgactgaga gccaacatcg ttggatagct tcgctggtgg ttcatcactg gcccgactat | | 10200 |
| | caggtacgct ttcccacacg ccgtcgtaag ctgaacagcg gctacttctg tattacttct | | 10260 |
| | cagcgtttcg ctgaggtttt acagcgacag tttggcccgc acttgtggat ggataccgcg | | 10320 |
| | gtcgcagagg ttaatgcgga atctgttcgg ttgaaaaagg gtcaggttat cggtgcccgc | | 10380 |
| | gcggtgattg acgggcgggg ttatgcggca aactcagcac tgagcgtggg cttcccagcg | | 10440 |
| | tttattggcc aggaatggcg attgagccac ccgcatggtt tatcgtctcc cattatcatg | | 10500 |
| | gatgccacgg tcgatcagca aaatggttat cgcttcgtgt acagcctgcc gctctcgccg | | 10560 |
| | accagattgt taattgaaga cacgcactat atcgataatg cgacattaga tcctgaacgc | | 10620 |
| | gcgcggcaaa atatttgcga ctatgccgcg caacagggtt ggcagcttca gacattgctg | | 10680 |
| | cgtgaagaac agggcgcctt accca | | 10705 |
| 3-140 | Sequences | | |
| 3-140-1 | Sequence Number [ID] | 140 | |
| 3-140-2 | Molecule Type | DNA | |
| 3-140-3 | Length | 411 | |
| 3-140-4-1 | Features Location/ | misc_feature 1 . . . 411 | |
| | Qualifiers | note = MaFAR-g1 | |
| | NonEnglishQualifier Value | | |
| | | source 1 . . . 411 | |
| 3-140-4-2 | Features Location/ | mol_type = other DNA | |
| | Qualifiers | organism = synthetic construct | |
| | NonEnglishQualifier Value | | |
| 3-140-5 | Residues | | |
| | gagcaaccaa cacttaaaga ggagaaaatg gcgacccagc agcagcagaa cggcgcctcg | | 60 |
| | gcgagcggcg tcctggaaca gttgcgcggg aagcatgtcc tgataaccgg taccaccggt | | 120 |
| | ttccttggca aggtagtcct ggaaaagctg atccgcacag tcccggacat cggcggcatc | | 180 |
| | cacctcctga tccggggcaa caagaggcat ccggccgccc gtgaacggtt cttgaacgag | | 240 |
| | atcgccagca gttcggtctt cgagcgtctg cgccacgacg acaacgaggc cttcgaaacc | | 300 |
| | ttcctggaag aaagggtgca ctgtataacc ggagaggtca ccgagagtcg tttcggcctt | | 360 |
| | accccggagc gcttccgcgc gctggcgggt caggtggacg ccttcatcaa t | | 411 |
| 3-141 | Sequences | | |
| 3-141-1 | Sequence Number [ID] | 141 | |
| 3-141-2 | Molecule Type | DNA | |
| 3-141-3 | Length | 465 | |
| 3-141-4-1 | Features Location/ | misc_feature 1 . . . 465 | |
| | Qualifiers | note = MaFAR-g2 | |
| | NonEnglishQualifier Value | | |
| 3-141-4-2 | Features Location/ | source 1 . . . 465 | |
| | Qualifiers | mol_type = other DNA | |
| | | organism = synthetic construct | |
| | NonEnglishQualifier Value | | |
| 3-141-5 | Residues | | |
| | gacgccttca tcaattcggc cgcctccgtc aacttccgcg aggaactgga caaggcgctg | | 60 |
| | aagatcaata cgctgtgcct ggagaatgtc gcggcccttg ctgaactcaa cagtgcgatg | | 120 |
| | gcggtcatcc aggtttcgac ctgctacgtt aacggcaaga atagcgggca gatcaccgaa | | 180 |
| | tcggtcatca agcccgcggg ggagtccatc ccgcgtagca ccgatgggta ctatgaaatc | | 240 |
| | gaagaattgg tgcacctgct gcaggacaaa atcagcgatg tgaaggcccg atactccggg | | 300 |
| | aaggttctgg aaaaaaaatt ggtggaccta ggcatccggg aagccaataa ctacgggtgg | | 360 |
| | agcgatacat ataccttcac caagtggctg ggcgaacagc tcctcatgaa ggccctgagc | | 420 |
| | ggcagatcgc tgaccatcgt gcggccgtcg atcatcgagt cggca | | 465 |
| 3-142 | Sequences | | |
| 3-142-1 | Sequence Number [ID] | 142 | |
| 3-142-2 | Molecule Type | DNA | |
| 3-142-3 | Length | 374 | |
| 3-142-4-1 | Features Location/ | misc_feature 1 . . . 374 | |
| | Qualifiers | note = MaFAR-g3 | |
| | NonEnglishQualifier Value | | |
| | | source 1 . . . 374 | |
| 3-142-4-2 | Features Location/ | mol_type = other DNA | |
| | Qualifiers | organism = synthetic construct | |
| | NonEnglishQualifier Value | | |
| 3-142-5 | Residues | | |
| | atcatcgagt cggcattgga agagcccagc ccggggtgga ttgaaggcgt caaggtcgcc | | 60 |
| | gatgccatca tactggccta cgcgagggag aaggtatcgc tctttcctgg caagcggagc | | 120 |
| | ggcatcatcg acgtcatccc agtggatctg gtggccaatt cgatcattct gtccctggcg | | 180 |
| | gaggcgctct ccggttcggg ccagcggcgt atctatcagt gctgcagcgg cggctcgaac | | 240 |
| | cccatctccc tcgggaagtt catcgactat ctgatggcgg aggcgaagac caactacgcg | | 300 |
| | gcctacgatc agctgttcta ccgccgcccc accaagccgt tcgtggccgt caaccgcaaa | | 360 |
| | ctcttcgacg tcgt | | 374 |
| 3-143 | Sequences | | |
| 3-143-1 | Sequence Number [ID] | 143 | |
| 3-143-2 | Molecule Type | DNA | |
| 3-143-3 | Length | 376 | |
| 3-143-4-1 | Features Location/ | misc_feature 1 . . . 376 | |
| | Qualifiers | note = MaFAR-g4 | |
| | NonEnglishQualifier Value | | |
| | | source 1 .. 376 | |
| 3-143-4-2 | Features Location/ | mol_type = other DNA | |
| | Qualifiers | organism = synthetic construct | |
| | NonEnglishQualifier Value | | |
| 3-143-5 | Residues | | |
| | actcttcgac gtcgtcgtgg gcggcatgcg ggtcccgctc tcgatcgcgg gcaaagccat | | 60 |
| | gcgcctggcg ggacaaaacc gcgaactgaa ggtcctgaag aatctggata cgacccggtc | | 120 |
| | cctggccacc attttcgggt tctacaccgc tccggactac atctttcgca atgacagcct | | 180 |
| | gatggccctg gcctcgcgca tgggcgagct ggaccgcgtg ttgttccccg ttgacgcccg | | 240 |
| | tcagatcgac tggcagctgt atctgtgcaa aatccacctc ggcgggctga atcggtacgc | | 300 |
| | gctcaaggaa cgtaagctgt actcgctccg ggccgccgac actcgcaaga aggcagcctg | | 376 |
| | agagacagaa cgaagt | | |
| 3-144 | Sequences | | |
| 3-144-1 | Sequence Number [ID] | 144 | |
| 3-144-2 | Molecule Type | DNA | |
| 3-144-3 | Length | 47 | |
| 3-144-4-1 | Features Location/ | misc_feature 1 . . . 47 | |
| | Qualifiers | note = GMV410 | |
| | NonEnglishQualifier Value | | |
| | | source 1 . . . 47 | |
| 3-144-4-2 | Features Location/ | mol_type = other DNA | |
| | Qualifiers | organism = synthetic construct | |
| | NonEnglishQualifier Value | | |
| 3-144-5 | Residues | | |
| | gagcaaccaa cacttaaaga ggagaaaatg cgccccctgc accccat | | 47 |
| 3-145 | Sequences | | |
| 3-145-1 | Sequence Number [ID] | 145 | |
| 3-145-2 | Molecule Type | DNA | |
| 3-145-3 | Length | 47 | |
| 3-145-4-1 | Features Location/ | misc_feature 1 . . . 47 | |
| | Qualifiers | note = GMV411 | |
| | NonEnglishQualifier Value | | |
| | | source 1 . . . 47 | |
| 3-145-4-2 | Features Location/ | mol_type = other DNA | |
| | Qualifiers | organism = synthetic construct | |
| | NonEnglishQualifier Value | | |
| 3-145-5 | Residues | | |
| | gagcaaccaa cacttaaaga ggagaaaatg cgcctgctga ccgccgt | | 47 |
| 3-146 | Sequences | | |
| 3-146-1 | Sequence Number [ID] | 146 | |
| 3-146-2 | Molecule Type | DNA | |
| 3-146-3 | Length | 47 | |
| 3-146-4-1 | Features Location/ | misc_feature 1 . . . 47 | |
| | Qualifiers | note = GMV412 | |
| | NonEnglishQualifier Value | | |
| | | source 1 . . . 47 | |
| 3-146-4-2 | Features Location/ | mol_type = other DNA | |
| | Qualifiers | organism = synthetic construct | |
| | NonEnglishQualifier Value | | |
| 3-146-5 | Residues | | |
| | gagcaaccaa cacttaaaga ggagaaaatg tccgtgatgt ccccgac | | 47 |
| 3-147 | Sequences | | |
| 3-147-1 | Sequence Number [ID] | 47 | |
| 3-147-2 | Molecule Type | DNA | |
| 3-147-3 | Length | 47 | |
| 3-147-4-1 | Features Location/ | misc_feature 1 . . . 47 | |
| | Qualifiers | note = GMV413 | |
| | NonEnglishQualifier Value | | |
| | | source 1 . . . 47 | |
| 3-147-4-2 | Features Location/ | mol_type = other DNA | |
| | Qualifiers | organism = synthetic construct | |
| | NonEnglishQualifier Value | | |
| 3-147-5 | Residues | | |
| | gagcaaccaa cacttaaaga ggagaaaatg ccggtcaccg actccat | | 47 |
| 3-148 | Sequences | | |
| 3-148-1 | Sequence Number [ID] | 148 | |
| 3-148-2 | Molecule Type | DNA | |
| 3-148-3 | Length | 47 | |
| 3-148-4-1 | Features Location/ | misc_feature 1 . .. 47 | |
| | Qualifiers | note = GMV414 | |
| | NonEnglishQualifier Value | | |
| | | source 1 . . . 47 | |
| 3-148-4-2 | Features Location/ | mol_type = other DNA | |
| | Qualifiers | organism = synthetic construct | |
| | NonEnglishQualifier Value | | |
| 3-148-5 | Residues | | |
| | gagcaaccaa cacttaaaga ggagaaaatg gccccgaccg actccct | | 47 |
| 3-149 | Sequences | | |
| 3-149-1 | Sequence Number [ID] | 149 | |
| 3-149-2 | Molecule Type | DNA | |
| 3-149-3 | Length | 47 | |
| 3-149-4-1 | Features Location/ | misc_feature 1 . . . 47 | |
| | Qualifiers | note = GMV415 | |
| | NonEnglishQualifier Value | | |
| | | source 1 . . . 47 | |
| 3-149-4-2 | Features Location/ | mol_type = other DNA | |
| | Qualifiers | organism = synthetic construct | |
| | NonEnglishQualifier Value | | |
| 3-149-5 | Residues | | |
| | gagcaaccaa cacttaaaga ggagaaaatg cccctgccga tgtcccc | | 47 |
| 3-150 | Sequences | | |
| 3-150-1 | Sequence Number [ID] | 150 | |
| 3-150-2 | Molecule Type | DNA | |
| 3-150-3 | Length | 35 | |
| 3-150-4-1 | Features Location/ | misc_feature 1 . . . 35 | |
| | Qualifiers | note = GMV416 | |
| | NonEnglishQualifier Value | | |
| | | source 1 . . . 35 | |
| 3-150-4-2 | Features Location/ | mol_type = other DNA | |
| | Qualifiers | organism = synthetic construct | |
| | NonEnglishQualifier Value | | |
| 3-150-5 | Residues | | |
| | acttcgttct gtctctcagt tggcggtctt gatgt | | 35 |
| 3-151 | Sequences | | |
| 3-151-1 | Sequence Number [ID] | 151 | |
| 3-151-2 | Molecule Type | DNA | |
| 3-151-3 | Length | 35 | |
| 3-151-4-1 | Features Location/ | misc_feature 1 . . . 35 | |
| | Qualifiers | note = | |
| | | ACTTCGTTCTGTCTCTCACGGGGCCAGCTTCTTCA | |
| | NonEnglishQualifier Value | | |
| | | source 1 . . . 35 | |
| 3-151-4-2 | Features Location/Q | mol_type = other DNA | |
| | ualifiers | organism = synthetic construct | |
| | NonEnglishQualifier Value | | |
| 3-151-5 | Residues | | |
| | acttcgttct gtctctcacg gggccagctt cttca | | 35 |
| 3-152 | Sequences | | |
| 3-152-1 | Sequence Number [ID] | 152 | |
| 3-152-2 | Molecule Type | DNA | |
| 3-152-3 | Length | 35 | |
| 3-152-4-1 | Features Location/ | misc_feature 1 . . . 35 | |
| | Qualifiers | note = GMV418 | |
| | NonEnglishQualifier Value | source 1 . . . 35 | |
| 3-152-4-2 | Features Location/ | mol_type = other DNA | |
| | Qualifiers | organism = synthetic construct | |
| | NonEnglishQualifier Value | | |
| 3-152-5 | Residues | | |
| | acttcgttct gtctctcagg tgccgctcgc ggcca | | 35 |
| 3-153 | Sequences | | |
| 3-153-1 | Sequence Number [ID] | 153 | |
| 3-153-2 | Molecule Type | DNA | |
| 3-153-3 | Length | 35 | |
| 3-153-4-1 | Features Location/ | misc_feature 1 . . . 35 | |
| | Qualifiers | note = GMV419 | |
| | NonEnglishQualifier Value | | |
| | | source 1 . . . 35 | |
| 3-153-4-2 | Features Location/ | mol_type = other DNA | |
| | Qualifiers | organism = synthetic construct | |
| | NonEnglishQualifier Value | | |
| 3-153-5 | Residues | | |
| | acttcgttct gtctctcaca gcagggccgc ttcca | | 35 |
| 3-154 | Sequences | | |
| 3-154-1 | Sequence Number [ID] | 154 | |
| 3-154-2 | Molecule Type | DNA | |
| 3-154-3 | Length | 35 | |
| 3-154-4-1 | Features Location/ | misc feature 1 . . . 35 | |
| | Qualifiers | note = GMV420 | |
| | NonEnglishQualifier Value | | |
| | | source 1 . . . 35 | |
| 3-154-4-2 | Features Location/ | mol_type = other DNA | |
| | Qualifiers | organism = synthetic construct | |
| | NonEnglishQualifier Value | | |
| 3-154-5 | Residues | | |
| | acttcgttct gtctctcaca ggccgaccgc ggttt | | 35 |
| 3-155 | Sequences | | |
| 3-155-1 | Sequence Number [ID] | 155 | |
| 3-155-2 | Molecule Type | DNA | |
| 3-155-3 | Length | 35 | |
| 3-155-4-1 | Features Location/ | misc_feature 1 . . . 35 | |
| | Qualifiers | note = GMV421 | |
| | NonEnglishQualifier Value | | |
| | | source 1 . . . 35 | |
| 3-155-4-2 | Features Location/ | mol_type = other DNA | |
| | Qualifiers | organism = synthetic construct | |
| | NonEnglishQualifier Value | | |
| 3-155-5 | Residues | | |
| | acttcgttct gtctctcaga tgcccaccgc gcgtt | | 35 |
| 3-156 | Sequences | | |
| 3-156-1 | Sequence Number [ID] | 156 | |
| 3-156-2 | Molecule Type | DNA | |
| 3-156-3 | Length | 1149 | |
| | | source 1 . . . 1149 | |
| 3-156-4-1 | Features Location/ | mol_type = other DNA | |
| | Qualifiers | organism = Saccharomyces cerevisiae | |
| | NonEnglishQualifier Value | | |
| 3-156-5 | Residues | | |
| | atgcgggcgc tggcgtattt caagaagggc gacatccatt tcaccaacga catcccccgg | | 60 |
| | ccggagatcc agaccgacga cgaagtgatc atcgacgtct cctggtgcgg catctgcggc | | 120 |
| | agcgacctgc acgagtacct ggacggcccc atcttcatgc cgaaggacgg cgaatgccac | | 180 |
| | aagctgagca acgccgcgct gcccctggcg atgggccatg agatgtcggg catcgtctcc | | 240 |
| | aaagtgggcc cgaaggtgac caaggtcaaa gtgggcgacc acgtcgtggt cgacgccgcg | | 300 |
| | agctcgtgcg ccgacctgca ctgctggccc cattccaagt tctataacag caagccgtgc | | 360 |
| | gacgcctgcc agcgcggctc ggagaacctg tgcacccatg cgggcttcgt cggcctgggc | | 420 |
| | gtgatcagcg gcggcttcgc cgaacaggtg gtcgtgtcgc agcaccatat catcccggtc | | 480 |
| | cccaaggaga tccccctgga cgtcgccgcc ctggtcgagc cgctgtcggt cacctggcac | | 540 |
| | gccgtgaaga tctccggctt caagaagggc tccagcgccc tggtcctggg cgcgggcccc | | 600 |
| | atcggcctgt gcaccatcct ggtgctgaag ggcatgggcg cgtcgaagat cgtcgtgtcc | | 660 |
| | gagatcgccg aacgtcgcat cgagatggcg aagaagctgg gcgtcgaagt gttcaacccg | | 720 |
| | agcaagcacg gccataagtc gatcgagatc ctgcggggcc tgaccaagtc ccacgacggc | | 780 |
| | ttcgactaca gctatgactg ctcgggcatc caggtcacct tcgaaaccag cctgaaggcc | | 840 |
| | ctgaccttca agggcaccgc caccaacatc gcggtctggg gcccgaagcc cgtgccgttc | | 900 |
| | cagccgatgg acgtcaccct gcaggagaag gtgatgaccg gctcgatcgg ctacgtcgtg | | 960 |
| | gaagacttcg aggaagtcgt gcgcgccatc cataacggcg acatcgcgat ggaggactgc | | 1020 |
| | aagcagctga tcaccggcaa gcagcggatc gaggacggct gggaaaaggg cttccaggag | | 1080 |
| | ctgatggacc acaaggaatc caacgtgaag atcctgctga ccccgaacaa ccacggcgaa | | 1140 |
| | atgaagtga | | 1149 |
| 3-157 | Sequences | | |
| 3-157-1 | Sequence Number [ID] | 157 | |
| 3-157-2 | Molecule Type | AA | |
| 3-157-3 | Length | 382 | |
| | | source 1 . . . 382 | |
| 3-157-4-1 | Features Location/ | mol_type = protein | |
| | Qualifiers | organism = Saccharomyces cerevisiae | |
| | NonEnglishQualifier Value | | |
| 3-157-5 | Residues | | |
| | MRALAYFKKG DIHFTNDIPR PEIQTDDEVI IDVSWCGICG SDLHEYLDGP IFMPKDGECH | | 60 |
| | KLSNAALPLA MGHEMSGIVS KVGPKVTKVK VGDHVVVDAA SSCADLHCWP HSKFYNSKPC | | 120 |
| | DACQRGSENL CTHAGFVGLG VISGGFAEQV VVSQHHIIPV PKEIPLDVAA LVEPLSVTWH | | 180 |
| | AVKISGFKKG SSALVLGAGP IGLCTILVLK GMGASKIVVS EIAERRIEMA KKLGVEVFNP | | 240 |
| | SKHGHKSIEI LRGLTKSHDG FDYSYDCSGI QVTEFTSKLA LTFKGTATNI AVWGPKPVPF | | 300 |
| | QPMDVTLQEK VMTGSIGYVV EDFEEVVRAI HNGDIAMEDC KQLITGKQRI EDGWEKGFQE | | 360 |
| | LMDHKESNVK ILLTPNNHGE MK | | 382 |
| 3-158 | Sequences | | |
| 3-158-1 | Sequence Number [ID] | 158 | |
| 3-158-2 | Molecule Type | DNA | |
| 3-158-3 | Length | 47 | |
| 3-158-4-1 | Features Location/ | misc_feature 1 . . . 47 | |
| | Qualifiers | note = GMV00268 | |
| | NonEnglishQualifier Value | | |
| | | source 1 . . . 47 | |
| 3-158-4-2 | Features Location/ | mol_type = other DNA | |
| | Qualifiers | organism = synthetic construct | |
| | NonEnglishQualifier Value | | |
| 3-158-5 | Residues | | |
| | gagcaaccaa cacttaaaga ggagaaaatg cgggcgctgg cgtattt | | 47 |
| 3-159 | Sequences | | |
| 3-159-1 | Sequence Number [ID] | 159 | |
| 3-159-2 | Molecule Type | DNA | |
| 3-159-3 | Length | 35 | |
| 3-159-4-1 | Features Location/ | misc_feature 1 . . . 35 | |
| | Qualifiers | note = GMV00271 | |
| | NonEnglishQualifier Value | | |
| | | source 1 . . . 35 | |
| 3-159-4-2 | Features Location/ | mol_type = other DNA | |
| | Qualifiers | organism = synthetic construct | |
| | NonEnglishQualifier Value | | |
| 3-159-5 | Residues | | |
| | acttcgttct gtctctcact tcatttcgcc gtggt | | 35 |
| 3-160 | Sequences | | |
| 3-160-1 | Sequence Number [ID] | 160 | |
| 3-160-2 | Molecule Type | DNA | |
| 3-160-3 | Length | 990 | |
| | | source 1 . . . 990 | |
| 3-160-4-1 | Features Location/ | mol_type = other DNA | |
| | Qualifiers | organism = Escherichia coli | |
| | NonEnglishQualifier Value | | |
| 3-160-5 | Residues | | |
| | atgcatatta catacgatct gccggttgct attgatgaca ttattgaagc gaaacaacga | | 60 |
| | ctggctgggc gaatttataa aacaggcatg cctcgctcca actattttag tgaacgttgc | | 120 |
| | aaaggtgaaa tattcctgaa gtttgaaaat atgcagcgta cgggttcatt taaaattcgt | | 180 |
| | ggcgcattta ataaattaag ttcactgacc gatgcggaaa aacgcaaagg cgtggtggcc | | 240 |
| | tgttctgcgg gcaaccatgc gcaaggggtt tccctctcct gcgcgatgct gggtatcgac | | 300 |
| | ggtaaagtgg tgatgccaaa aggtgcgcca aaatccaaag tagcggcaac gtgcgactac | | 360 |
| | tccgcagaag tcgttctgca tggtgataac ttcaacgaca ctatcgctaa agtgagcgaa | | 420 |
| | attgtcgaaa tggaaggccg tatttttatc ccaccttacg atgatccgaa agtgattgct | | 480 |
| | ggccagggaa cgattggtct ggaaattatg gaagatctct atgatgtcga taacgtgatt | | 540 |
| | gtgccaattg gtggtggcgg tttaattgct ggtattgcgg tggcaattaa atctattaac | | 600 |
| | ccgaccattc gtgttattgg cgtacagtct gaaaacgttc acggcatggc ggcttctttc | | 660 |
| | cactccggag aaataaccac gcaccgaact accggcaccc tggcggatgg ttgtgatgtc | | 720 |
| | tcccgcccgg gtaatttaac ttacgaaatc gttcgtgaat tagtcgatga catcgtgctg | | 780 |
| | gtcagcgaag acgaaatcag aaacagtatg attgccttaa ttcagcgcaa taaagtcgtc | | 840 |
| | accgaaggcg caggcgctct ggcatgtgct gcattattaa gcggtaaatt agaccaatat | | 900 |
| | attcaaaaca gaaaaaccgt cagtattatt tccggcggca atatcgatct ttctcgcgtc | | 960 |
| | tctcaaatca ccggtttcgt tgacgcttaa | | 990 |
| 3-161 | Sequences | | |
| 3-161-1 | Sequence Number [ID] | 161 | |
| 3-161-2 | Molecule Type | AA | |
| 3-161-3 | Length | 329 | |
| | | source 1 . .. 329 | |
| 3-161-4-1 | Features Location/ | mol_type = protein | |
| | Qualifiers | organism = Escherichia coli | |
| | NonEnglishQualifier Value | | |
| 3-161-5 | Residues | | |
| | MHITYDLPVA IDDIIEAKQR LAGRIYKTGM PRSNYFSERC KGEIFLKFEN MQRTGSFKIR | | 60 |
| | GAFNKLSSLT DAEKRKGVVA CSAGNHAQGV SLSCMALGID GKVVMPKGAP KSKVAATCDY | | 120 |
| | SAEVVLHGDN FNDTIAKVSE IVEMEGRIFI PPYDDPKVIA GQGTIGLEIM EDLYDVDNVI | | 180 |
| | VPIGGGGLIA GIAVAIKSIN PTIRVIGVQS ENVHGMAASF HSGEITTHRT TGTLADGCDV | | 240 |
| | SRPGNLTYEI VRELVDDIVL VSEDEIRNSM IALIQRNKVV TEGAGALACA ALLSGKLDQY | | 300 |
| | IQNRKTVSII SGGNIDLSRV SQITGFVDA | | 329 |
| 3-162 | Sequences | | |
| 3-162-1 | Sequence Number [ID] | 162 | |
| 3-162-2 | Molecule Type | AA | |
| 3-162-3 | Length | 590 | |
| | | source 1 . . . 590 | |
| 3-162-4-1 | Features Location/ | mol_type = protein | |
| | Qualifiers | organism = | |
| | | Methylosinus trichosporium | |
| | NonEnglishQualifier Value | | |
| 3-162-5 | Residues | | |
| | MARKMTGAEM VVEALKDQGV EIIFGYPGGA VLPIYDALFH QEKVQHILVR HEQGAAHAAE | | 60 |
| | GYARSSGKVG VLLVTSGPGA ENTITGLTDA LMDSIPVVCI TGQVPTHLIG SDAFGECDTV | | 120 |
| | GITRHCTKHN YLVKSVDDLP RILHEAFYVA SSGRPGPVVI DIPKDVQFAS GTYTGPRNVH | | 180 |
| | HKTYQPKLEG DTESIRRAVK MMAAAKRPIF YTGGGVINSG PAASTLLREL VSLTGFPITS | | 240 |
| | TLMGLGAYPG SGPNWLGMLG MHGTFEANNA MHDCDLMIAV GARFDDRITG RLDAFSPGSK | | 300 |
| | KIHIDIDRSS INKNVKIDLP IVGDCGHVLE SLVRVWRSEA MHAEKQPLDG WMKTIDHWRE | | 360 |
| | RKSLAFRNSD KVIKPQYAVQ RLYALTKDRD PYITTEVGQH QMWAAQHYHF DEPNRWMTSG | | 420 |
| | GLGTMGYGLP AAIGAQLAHP KSLVVDIAGE ASILMNIQEM STAIQYRLPV KVFILNNEYM | | 480 |
| | GMVRQWQELL HGGRYSHSYS EALPDFVKLA EAFGGKGIRC SDPAELDSAI LEMIDYDGPV | | 540 |
| | IFDCLVEKNE NCFPMIPSGK AHNDMLLADL GDDAGVELGS IIDEKGKMLV | | 590 |
| 3-163 | Sequences | | |
| 3-163-1 | Sequence Number [ID] | 163 | |
| 3-163-2 | Molecule Type | AA | |
| 3-163-3 | Length | 351 | |
| | | source 1 . . . 351 | |
| 3-163-4-1 | Features Location/ | mol_type = protein | |
| | Qualifiers | organism = | |
| | NonEnglishQualifier Value | Methylosinus trichosporium | |
| 3-163-5 | Residues | | |
| | MSTKAYAVAS AEALFGPLAI ERRALGPEDV EIDILYCGVC HSDLHTARSE WPGTRYPCVP | | 60 |
| | GHEIVGRVTA VGAKVTKFSV GDLAAVGDMV DSCRRCLSCD DGLEQYCEHG FTATYNGPIY | | 120 |
| | GSGENTFGGY SEKIVVDAHF VLAIHHSETQ LAGVAPLLCA GITTWSPLKH WGVGPGKSVG | | 180 |
| | IVGIGGLGHM GVKLAHALGA HVVAFTTSPS KRDAALALGA DEVVVSTDPA AMAARAGSLD | | 240 |
| | FILDTVAVAH DLDAYVNLLK RDGALVLVGV PATPHPSPSA GGLIFKRRQV AGSLIGGVKE | | 300 |
| | TQEMLDECAE RGIVADIETI AMQQIETAYA RMLKNDVKYR FVIDMATKLA A | | 351 |
| 3-164 | Sequences | | |
| 3-164-1 | Sequence Number [ID] | 164 | |
| 3-164-2 | Molecule Type | AA | |
| 3-164-3 | Length | 342 | |
| | | source 1 . . . 342 | |
| 3-164-4-1 | Features Location/ | mol_type = protein | |
| | Qualifiers | organism = Methylococcus capsulatus | |
| | NonEnglishQualifier Value | | |
| 3-164-5 | Residues | | |
| | MKAWVIDRIG PLDSSRTLLR ATDLPVPEPG PGEILLQVAV CGVCHTEIDE IEGRTAPPRL | | 60 |
| | PVVPGHQAVG RIAALGSGVA EFALGDRVGV AWIFSACGEC EFCRSGRENL CFAFCATGRD | | 120 |
| | VDGGYAQYMT VPAAFAFRIP EGFTDAEAAP LLCAGAIGYR SLNLSGLKNG QPLGLTGFGA | | 180 |
| | SAHLVLMMAR YRFPDSEVYV FARHPEERAF ALQLGAVWAG DTADIAPAPL AAIIDTTPAW | | 240 |
| | KPVVAALANL APGGRLVVNA IRKAPDDRAC LAELDYARHL WMEREIKSVA NVARSDVAGF | | 300 |
| | LALAAEMGIR PETEEYPFED ADRALLDLKQ RRIRGAKVLR VT | | 342 |
| 3-165 | Sequences | | |
| 3-165-1 | Sequence Number [ID] | 165 | |
| 3-165-2 | Molecule Type | AA | |
| 3-165-3 | Length | 355 | |
| | | source 1 . . . 355 | |
| | Features Location/ | mol_type = protein | |
| | Qualifiers | organism = Methylococcus capsulatus | |
| | NonEnglishQualifier Value | | |
| 3-165-5 | Residues | | |
| | MPTAKAYAAF SADSALAPFV LQRRDPLPQD IRIGILYCGV CHSDLHQARN EWNATTYPCV | | 60 |
| | PGHEIVGKVL EVGRSVTKFK PGDTVAVGCM VDSCRTCPNC VDALEQHCEH GPVFTYNSPC | | 120 |
| | PHGGGMTFGG YAESIVVDEA FVLRIPDGLD LAAAAPLLCA GITTYSPLRH WKVGAGQRVG | | 180 |
| | VVGLGGLGHM ALKFAHTFGA ETVLFTTTPD KAEDARRLGA DEVVVSRDPE AMARQAGRFD | | 240 |
| | FILDTVSAPH DIDAYLNLLR RDGTLTLVGV PPQGVQVMPF SLIGGRRRLA GSLIGGIRET | | 300 |
| | QEMLDFCGEH GIVCDIELIP IQGINDAFER MLKSDVKYRF VIDMATLNGE SSGGR | | 355 |
| 3-166 | Sequences | | |
| 3-166-1 | Sequence Number [ID] | 166 | |
| 3-166-2 | Molecule Type | AA | |
| 3-166-3 | Length | 548 | |
| | | source 1 . . . 548 | |
| 3-166-4-1 | Features Location/ | mol_type = protein | |
| | Qualifiers | organism = Lactococcus lactis | |
| | NonEnglishQualifier Value | | |
| 3-166-5 | Residues | | |
| | MYTVGDYLLD RLHELGIEEI FGVPGDYNLQ FLDQIISRKD MKWVGNANEL NASYMADGYA | | 60 |
| | RTKKAAAFLT TFGVGELSAV NGLAGSYAEN LPVVEIVGSP TSKVQNEGKF VHHTLADGDF | | 120 |
| | KHFMKMHEPV TAARTLLTAE NATVEIDRVL SALLKERKPV YINLPVDVAA AKAEKPSLPL | | 180 |
| | KKENPTSNTS DQEILNKIQE SLKNAKKPIV ITGHEIISFG LENTVTQFIS KTKLPITTLN | | 240 |
| | FGKSSVDETL PSFLGIYNGK LSEPNLKEFV ESADFILMLG VKLTDSSTGA FTHHLNENKM | | 300 |
| | ISLNIDEGKI FNESIQNFDF ESLISSLLDL SGIEYKGKYI DKKQEDFVPS NALLSQDRLW | | 360 |
| | QAVENLTQSN ETIVAEQGTS FFGASSIFLK PKSHFIGQPL WGSIGYTFPA SYNDIPMWNY | | 420 |
| | SRHLLFIGDG SLQLTVQELG LAIREKINPI CFIINNDGYT VEREIHGPNQ SYNDIPMWNY | | 480 |
| | SKLPESFGAT EERVVSKIVR TENEFVSVMK EAGADPNRMY WIELVLAKED APKVLKKMGK | | 540 |
| | LFAEQNKS | | 548 |
| 3-167 | Sequences | | |
| 3-167-1 | Sequence Number [ID] | 167 | |
| 3-167-2 | Molecule Type | AA | |
| 3-167-3 | Length | 563 | |
| | | source 1 . . . 563 | |
| 3-167-4-1 | Features Location/ | mol_type = protein | |
| | Qualifiers | organism = Saccharomyces cerevisiae | |
| | NonEnglishQualifier Value | | |
| 3-167-5 | Residues | | |
| | MSEITLGKYL FERLKQVNVN TIFGLPGDFN LSLLDKIYEV DGLRWAGNAN ELNAAYAADG | | 60 |
| | YARIKGLSVL VTTFGVGELS ALNGIAGSYA EHVGVLHVVG VPSISAGAKQ LLLHHTLGNG | | 120 |
| | DFTVFHRMSA NISETTSMIT DIATAPSEID RLIRTTFITQ RPSYLGLPAN LVDLKVPGSL | | 180 |
| | LEKPIDLSLK PNDPEAEKEV IDTVLELIQN SKNPVILSDA CASRHNVKKE TQKLIKLTQF | | 240 |
| | PAFVTPLGKG SIDEQHPRYG GVYVGTLSKQ DVLQAVESAD LILSVGALLS DFNTGSFSYS | | 300 |
| | YKTKNVVEFH SDYVKVKNAT FLGVQMKFAL QNLLKVIPDV VKGYKSVPVP TKTPANKGVP | | 360 |
| | ASTPLKQEWL WNELSKFLQE GDVIISETGT SAFGINQTIF PKDAYGISQV LWGSIGFTTG | | 420 |
| | ATLGAAFAAE EIDPNKRVIL FIGDGSLQLT VQEISTMIRW GLKPYLFVLN NDGYTIEKLI | | 480 |
| | HGPHAEYNEI QTWDHLALLP AFGAKKYENH KIATTGEWDA LTTDSEFQKN SVIRLIELKL | | 540 |
| | PVFDAPESLI KQAQLTAATN AKQ | | 563 |
| 3-168 | Sequences | | |
| 3-168-1 | Sequence Number [ID] | 168 | |
| 3-168-2 | Molecule Type | AA | |
| 3-168-3 | Length | 635 | |
| | | source 1 . . . 635 | |
| 3-168-4-1 | Features Location/ | mol_type = protein | |
| | Qualifiers | organism = Saccharomyces cerevisiae | |
| | NonEnglishQualifier Value | | |
| 3-168-5 | Residues | | |
| | MAPVTIEKFV NQEERHLVSN RSATIPFGEY IFKRLLSIDT KSVFGVPGDF NLSLLEYLYS | | 60 |
| | PSVESAGLRW VGTCNELNAA YAADGYSRYS NKIGCLITTY GVGELSALNG IAGSFAENVK | | 120 |
| | VLHIVGVAKS IDSRSSNFSD RNLHHLVPQL HDSNFKGPNH KVYHDMVKDR VACSVAYLED | | 180 |
| | IETACDQVDN VIRDIYKYSK PGYIFVPADF ADMSVTCDNL VNVPRISQQD CIVYPSENQL | | 240 |
| | SDIINKITSW IYSSKTPAIL GDVLTDRYGV SNFLNKLICK TGIWNFSTVM GKSVIDESNP | | 300 |
| | TYMGQYNGKE GLKQVYEHFE LCDLVLHFGV DINEINNGHY TFTYKPNAKI IQFHPNYIRL | | 360 |
| | VDTRQGNEQM FKGINFAPIL KELYKRIDVS KLSLQYDSNV TQYTNETMRL EDPTNGQSSI | | 420 |
| | ITQVHLQKTM PKFLNPGDVV VCETGSFQFS VRDFAFPSQL KYISQGFFLS IGMALPAALG | | 480 |
| | VGIAMQDHSN AHINGGNVKE DYKPRLILFE GDGAAQMTIQ ELSTILKCNI PLEVIIWNNN | | 540 |
| | GYTIERAIMG PTRSYNDVMS WKWTKLFEAF GDFDGKYTNS TLIQCPSKLA LKLEELKNSN | | 600 |
| | KRSGIELLEV KLGELDFPEQ LKCMVEAAAL KRNKK | | 635 |
| 3-169 | Sequences | | |
| 3-169-1 | Sequence Number [ID] | 169 | |
| 3-169-2 | Molecule Type | AA | |
| 3-169-3 | Length | 348 | |
| | | source 1 . . . 348 | |
| 3-169-4-1 | Features Location/ | mol_type = protein | |
| | Qualifiers | organism = Saccharomyces cerevisiae | |
| | NonEnglishQualifier Value | | |
| 3-169-5 | Residues | | |
| | MSIPETQKAI IFYESNGKLE HKDIPVPKPK PHELLINVKY SGVCHTDLHA WHGDWPLPTK | | 60 |
| | LPLVGGHEGA GVVVGMGENV KGWKIGDYAG IKWLNGSCMA CEYCELGNES NCPHADLSGY | | 120 |
| | THDGSEQEYA TADAVQAAHI PQGTDLAEVA PILCAGITVY KALKSANLRA GHWAAISGAA | | 180 |
| | GGLGSLAVQY AKAMGYRVLG IDGGPGKEEL FTSLGGEVFI DFTKEKDIVS AVVKATNGGA | | 240 |
| | HGIINVSVSE AAIEASTRYC RANGTVVLVG LPAGAKCSSD VFNHVVKSIS IVGSYVGNRA | | 300 |
| | DTREALDEFA RGLVKSPIKV VGLSSLPEIY EKMEKGQIAG RYVVTDSK | | 348 |
| 3-170 | Sequences | | |
| 3-170-1 | Sequence Number [ID] | 170 | |
| 3-170-2 | Molecule Type | AA | |
| 3-170-3 | Length | 563 | |
| | | source 1 . . . 563 | |
| 3-170-4-1 | Features Location/ | mol_type = protein | |
| | Qualifiers | organism = Saccharomyces cerevisiae | |
| | NonEnglishQualifier Value | | |
| 3-170-5 | Residues | | |
| | MSEITLGKYL FERLKQVNVN TVFGLPGDFN LSLLDKIYEV EGMRWAGNAN ELNAAYAADG | | 60 |
| | YARIKGMSCI ITTFGVGELS ALNGIAGSYA EHVGVLHVVG VPSISAQAKQ LLLHHTLGNG | | 120 |
| | DFTVFHRMSA NISETTAMIT DIATAPAEID RCIRTTYVTQ RPVYLGLPAN LVDLNVPAKL | | 180 |
| | LQTPIDMSLK PNDAESEKEV IDTILALVKD AKNPVILADA CCSRHDVKAE TKKLIDLTQF | | 240 |
| | PAFVTPMGKG SIDEQHPRYG GVYVGTLSKP EVKEAVESAD LILSVGALLS DFNTGSFSYS | | 300 |
| | YKTKNIVEFH SDHMKIRNAT FPGVQMKFVL QKLLTTIADA AKGYKPVAVP ARTPANAAVP | | 360 |
| | ASTPLKQEWM WNQLGNFLQE GDVVIAETGT SAFGINQTTF PNNTYGISQV LWGSIGFTTG | | 420 |
| | ATLGAAFAAE EIDPKKRVIL FIGDGSLQLT VQEISTMIRW GLKPYLFVLN NDGYTIEKLI | | 480 |
| | HGPKAQYNEI QGWDHLSLLP TFGAKDYETH RVATTGEWDK LTQDKSFNDN SKIRMIEIML | | 540 |
| | PVFDAPQNLV EQAKLTAATN AKQ | | 563 |
| 3-171 | Sequences | | |
| 3-171-1 | Sequence Number [ID] | 171 | |
| 3-171-2 | Molecule Type | AA | |
| 3-171-3 | Length | 1048 | |
| | | source 1 . . . 1048 | |
| 3-171-4-1 | Features Location/ | mol_type = protein | |
| | Qualifiers | organism = | |
| | | Clostridium acetobutylicum | |
| | NonEnglishQualifier Value | | |
| 3-171-5 | Residues | | |
| | MKSEYTIGRY LLDRLSELGI RHIFGVPGDY NLSFLDYIME YKGIDWVGNC NELNAGYAAD | | 60 |
| | GYARINGIGA ILTTFGVGEL SAINAIAGAY AEQVPVVKIT GIPTAKVRDN GLYVHHTLGD | | 120 |
| | GRFDHFFEMF REVTVAEALL SEENAAQEID RVLISCWRQK RRVLINLPID VYDKPINKPL | | 180 |
| | KPLLDYTISS NKEAACEFVT EIVPIINRAK KPVILADYGV YRYQVQHVLK NLAEKTGFPV | | 240 |
| | ATLSMGKGVF NEAHPQFIGV YNGDVSSPYL RQRVDEADCI ISVGVKLTDS TTGGFSHGFS | | 300 |
| | KRNVIHIDPF SIKAKGKKYA PITMKDALTE LTSKIEHRNF EDLDIKPYKS DNQKYFAKEK | | 360 |
| | PITQKRFFER IAHFIKEKDV LLAEQGTCFF GASTIQLPKD ATFIGQPLWG SIGYTLPALL | | 420 |
| | GSQLADQKRR NILLIGDGAF QMTAQEISTM LRLQIKPIIF LINNDGYTIE RAIHGREQVY | | 480 |
| | NNIQMWRYHN VPKVLGPKEC SLTFKVQSET ELEKALLVAD KDCEHLIFIE VVMDRYDKPE | | 540 |
| | PLERLSKRFA NQNNGYARIN GIGAILTTFG VGELSAINAI AGAYAEQVPV VKITGIPTAK | | 600 |
| | VRDNGLYVHH TLGDGRFDHF FEMFREVTVA EALLSEENAA QEIDRVLISC WRQKRPVLIN | | 660 |
| | LPIDVYDKPI NKPLKPLLDY TISSNKEAAC EFVTEIVPII NRAKKPVILA DYGVYRVQVQ | | 720 |
| | HVLKNLAEKT GFPVATLSMG KGVFNEAHPQ FIGVYNGDVS SPYLRQRVDE ADCIISVGVK | | 780 |
| | LTDSTTGGFS HGFSKRNVIH IDPFSIKAKG KKYAPITMKD ALTELTSKIE HRNFEDLDIK | | 840 |
| | PYKSDNQKYF AKEKPITQKR FFERIAHFIK EDKVLLAEQG TCFFGASTIQ LPKDATFIGQ | | 900 |
| | PLWGSIGYTL PALLGSQLAD QKRRNILLIG DGAFQMTAQE ISTMLRLQIK PIIFLINNDG | | 960 |
| | YTIERAIHGR EQVYNNIQMW RYHNVPKVLG PKECSLTFKV QSETELEKAL IVADKDCEHL | | 1020 |
| | IFIEVVMDRY DKPEPLERLS KRFANQNN | | 1048 |
| 3-172 | Sequences | | |
| 3-172-1 | Sequence Number [ID] | 172 | |
| 3-172-2 | Molecule Type | DNA | |
| 3-172-3 | Length | 645 | |
| | | source 1 . . . 645 | |
| 3-172-4-1 | Features Location/ | mol_type = other DNA | |
| | Qualifiers | organism = Methylococcus capsulatus | |
| | NonEnglishQualifier Value | | |
| 3-172-5 | Residues | | |
| | atggcaagac cattgattca gctcgccctg gacacgctgg acatcccgca gaccctgaag | | 60 |
| | ctcgcaagcc tcaccgcgcc ctatgtcgat atcttcgaaa tcggcacccc cagcatcaag | | 120 |
| | cacaacggca tcgccctggt gaaggagttc aaaaaacgct tccccaacaa gctgctcctg | | 180 |
| | gtcgacctca aaaccatgga cgccggtgaa tacgaagcca cccccttctt cgccgccggc | | 240 |
| | gccgacatca ccaccgtcct cggcgtcgca ggactggcca ccatcaaggg cgtcatcaac | | 300 |
| | gccgccaaca agcacaacgc cgaggtccag gtcgacctga tcaacgtccc cgacaaggcc | | 360 |
| | gcctgcgccc gtgagtccgc caaggccggc gcccagatcg tcggcatcca caccggcctc | | 420 |
| | gacgcccagg ccgccggcca gacccccttc gccgacctcc aggccatcgc caagctcggc | | 480 |
| | ctccccgtcc gcatctccgt cgccggcggc atcaaggcct ccaccgccca acaggtcgtc | | 540 |
| | aaaaccggtg ccaacatcat cgtcgtcgga gccgccatct acggcgccgc ctcccccgcc | | 600 |
| | gatgccgcgc gcgaaatcta cgaacaggtc gtcgccgctt ccgcc | | 645 |
| 3-173 | Sequences | | |
| 3-173-1 | Sequence Number [ID] | 17 | |
| 3-173-2 | Molecule Type | AA | |
| 3-173-3 | Length | 215 | |
| | | source 1 . . . 215 | |
| 3-173-4 | Features Location/ | mol_type = protein | |
| | Qualifiers | organism = Methylococcus capsulatus | |
| | NonEnglishQualifier Value | | |
| 3-173-5 | Residues | | |
| | MARPLIQLAL DTLDIPQTLK LASLTAPYVD IFEIGTPSIK HNGIALVKEF KKRFPNKLLL | | 60 |
| | VDLKTMDAGE YEATPFFAAG ADITTVLGVA GLATIKGVIN AANKNHAEVQ VDLINVPDKA | | 120 |
| | ACARESAKAG AQIVGIHTGL DAQAAGQTPF ADLQAIAKLG LPVRISVAGG IKASTAQQVV | | 180 |
| | KTGANIIVVG AAIYGAASPA DAAREIYEQV VAASA | | 215 |
| 3-174 | Sequences | | |
| 3-174-1 | Sequence Number [ID] | 174 | |
| 3-174-2 | Molecule Type | DNA | |
| 3-174-3 | Length | 531 | |
| | | source 1 . . 531 | |
| 3-174-4-1 | Features Location/ | mol_type = other DNA | |
| | Qualifiers | organism = Methylococcus capsulatus | |
| | NonEnglishQualifier Value | | |
| 3-174-5 | Residues | | |
| | atgcatcaga aactgatcat agacaaaatc tccggcatcc tcgccgccac cgatgccggc | | 60 |
| | tatgatgcaa aactgactgc catgctcgac caggcctccc gcatcttcgt cgcgggggcc | | 120 |
| | ggccggtcgg ggctggtcgc caagttcttc gccatgcgcc tcatgcacgg cggctatgac | | 180 |
| | gtcttcgtcg tcggcgaaat cgtcaccccc agcatccgca agggcgactt gctgatcgtg | | 240 |
| | atctccggct ccggtgaaac cgaaaccatg ctcgccttca ccaaaaaagc caaggagcag | | 300 |
| | ggcgcctcca tcgccctcat ctccacccgc gacagctcct ccctcggcga cctcgccgac | | 360 |
| | tccgtcttcc gcatcggctc cccagagctc ttcggaaaag tcgtcggcat gcccatgggc | | 420 |
| | accgtcttcg agctctccac cctcctcttc ctcgaggcca ccatctctca catcatccac | | 480 |
| | gagaaaggca tccccgaaga agaaatgaga actcgtcacg ccaacctgga a | | 531 |
| 3-175 | Sequences | | |
| 3-175-1 | Sequence Number [ID] | 175 | |
| 3-175-2 | Molecule Type | AA | |
| 3-175-3 | Length | 177 | |
| | | source 1 . . 77 | |
| 3-175-4-1 | Features Location/ | mol_type = protein | |
| | Qualifiers | organism = Methylococcus capsulatus | |
| | NonEnglishQualifier Value | | |

| | | |
|---|---|---|
| 3-175-5 | Residues | |
| | MHQKLIIDKI SGILAATDAG YDAKLTAMLD QASRIFVAGA GRSGLVAKFF AMRLMHGGYD | 60 |
| | VFVVGEIVTP SIRKGDLLIV ISGSGETETM LAFTKKAKEQ GASIALISTR DSSSLGDLAD | 120 |
| | SVFRIGSPEL FGKVVGMPMG TVFELSTLLF LEATISHIIH EKGIPEEEMR TRHANLE | 177 |

1-90. (canceled)

91. A genetically modified methanotroph comprising a heterologous polynucleotide encoding for an acetolactate synthase (ALS), wherein the acetolactate synthase can catalyze the conversion of pyruvate to acetolactate and comprises an amino acid sequence having at least 90% sequence homology to SEQ ID NO: 2, and wherein said methanotroph is capable of converting formaldehyde to pyruvate through a type I RuMP pathway or a type II serine pathway.

92. The methanotroph of claim 91, wherein the methanotroph further comprises a heterologous polynucleotide encoding a ketoacid decarboxylase (KDC), wherein the ketoacid decarboxylase can catalyze the conversion of ketoisovalerate to isobutryaldehyde.

93. The methanotroph of claim 92, wherein the methanotroph further comprises a a heterologous polynucleotide encoding a ketol-acid reductoisomerase (KARI), a heterologous polynucleotide encoding a dihydroxy-acid dehydratase (DHAD), and a heterologous polynucleotide encoding an alcohol dehydrogenase (ADH); wherein the ketol-acid reductoisomerase can catalyze the conversion of acetolactate to 2,3-dihydroxyisovalerate, wherein the dihydroxy-acid dehydratase can catalyze the conversion of 2,3-dihydroxyisovalerate to ketoisovalerate, and wherein the alcohol dehydrogenase can catalyze the conversion of isobutyraldehyde to isobutanol.

94. The methanotroph of claim 93, wherein the methanotroph further comprises a heterologous polynucleotide encoding an alcohol dehydrogenase (ADH), wherein the ketoacid decarboxylase can catalyze the conversion of isobutyraldehyde to isobutanol.

95. The methanotroph of claim 94, wherein the ketoacid decarboxylase (KDC) comprises an amino acid sequence having at least 90% sequence homology to SEQ ID NO: 8.

96. The methanotroph of claim 91, wherein the methanotroph further comprises a a heterologous polynucleotide encoding a ketol-acid reductoisomerase (KARI), heterologous polynucleotide encoding a dihydroxy-acid dehydratase (DHAD), and a heterologous polynucleotide encoding a ketoacid decarboxylase (KDC); and a heterologous polynucleotide encoding an alcohol dehydrogenase (ADH); wherein the acetolactate synthase can catalyze the conversion of pyruvate to acetolactate and comprises an amino acid sequence having at least 90% sequence homology to SEQ ID NO: 2, wherein the ketol-acid reductoisomerase can catalyze the conversion of acetolactate to 2,3-dihydroxyisovalerate and comprises an amino acid sequence having at least 90% sequence homology to SEQ ID NO: 4, wherein the dihydroxy-acid dehydratase can catalyze the conversion of 2,3-dihydroxyisovalerate to ketoisovalerate and comprises an amino acid sequence having at least 90% sequence homology to SEQ ID NO: 6, wherein the ketoacid decarboxylase can catalyze the conversion of ketoisovalerate to isobutryaldehyde and comprises an amino acid sequence having at least 90% sequence homology to SEQ ID NO: 8, and wherein the alcohol dehydrogenase can catalyze the conversion of isobutyraldehyde to isobutanol and comprises an amino acid sequence having at least 90% sequence homology to SEQ ID NO: 10.

97. The methanotroph of claim 96, wherein the methanotroph further comprises a polynucleotide sequence encoding for the alcohol dehydrogenase (ADH) and a promoter, wherein said promoter can direct the expression of the alcohol dehydrogenase in the methanotroph.

98. The methanotroph of claim 92, wherein the methanotroph further comprises a polynucleotide sequence encoding for the ketoacid decarboxylase (KDC), the alcohol dehydrogenase (ADH), and a promoter, wherein said promoter can direct the expression of the ketoacid decarboxylase and the alcohol dehydrogenase (ADH) in the methanotroph.

99. The methanotroph of claim 93, wherein the methanotroph further comprises a polynucleotide sequence encoding for the acetolactate synthase (ALS), the ketol-acid reductoisomerase (KARI), the dihydroxy-acid dehydratase (DHAD), the ketoacid decarboxylase (KDC), the alcohol dehydrogenase (ADH) and a promoter, wherein said promoter can direct the expression of the acetolactate synthase (ALS), the ketol-acid reductoisomerase (KARI), the dihydroxy-acid dehydratase (DHAD), the ketoacid decarboxylase (KDC), and the alcohol dehydrogenase (ADH) in a methanotroph.

100. The methanotroph of claim 99, wherein said promoter is constitutive.

101. The methanotroph of claim 99, wherein said promoter is inducible.

102. The methanotroph of claim 91, wherein said methanotroph is from the genus Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, or Methyloacidophilum.

103. The methanotroph of claim 91, wherein said methanotroph is from the genus Methylococcus.

104. The methanotroph of claim 91, wherein said methanotroph is from the species Methylococcus capsulatus.

105. The methanotroph of claim 91, wherein said methanotroph is from the strain Methylococcus capsulatus strain Bath.

106. A method of making a multi-carbon compound comprising:
(a) contacting a genetically modified methanotroph with a multi-carbon product precursor comprising a heterologous polynucleotide encoding for an acetolactate synthase (ALS), wherein the ALS can catalyze the conversion of pyruvate to acetolactate and comprises an amino acid sequence having at least 90% sequence homology to SEQ ID NO: 2, and wherein said methanotroph is capable of converting formaldehyde to pyruvate through a type I RuMP pathway or a type II serine pathway; and
(b) growing said methanotroph in conditions to produce a multi-carbon compound.

107. The method of claim 105 wherein said precursor is methane.

108. The method of claim 106 wherein said multi-carbon compound is isobutanol.

109. The method of claim 106 wherein said multi-carbon product is 1-butanol.

110. A genetically modified methanotroph capable of converting methane to a multi-carbon product comprising a heterologous polynucleotide encoding for an acetolactate synthase (ALS), wherein the ALS has acetolactate synthase can catalyze the conversion of pyruvate to acetolactate and comprises an amino acid sequence having at least 90% sequence homology to SEQ ID NO: 2, wherein said methanotroph is capable of converting formaldehyde to pyruvate through a type I RuMP pathway or a type II serine pathway.
